(19) United States
(12) Patent Application Publication
(10) Pub. No.: US 2007/0196853 A1
(43) Pub. Date: Aug. 23, 2007

(54) Methods For Rapid Genotype Screening

(75) Inventor: Timothy A. Hodge, Eads, TN (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: TRANSNETYX, INC., Memphis, TN (US)

(21) Appl. No.: 11/739,872

(22) Filed: Apr. 25, 2007

Related U.S. Application Data
(62) Division of application No. 11/166,990, filed on Jun. 24, 2005.
Application No. 11/166,990 is a continuation-in-part of application No. 11/074,995, filed on Mar. 8, 2005.
Application No. 11/074,995 is a continuation of application No. 09/945,952, filed on Sep. 4, 2001, now Pat. No. 7,011,943.
(60) Provisional application No. 60/230,371, filed on Sep. 6, 2000.

(51) Int. Cl. C12Q 1/68

(52) U.S. Cl. 435/6

(57) ABSTRACT

The present invention provides a method to rapidly provide genotype screening of a plurality of biological samples in a designated well of a microwell container for remote user by a screening laboratory. The screening method can be used to determine if a biological sample is heterozygous, homozygous or wild for a designated genetic sequence.

### CROSS-REFERENCE TO RELATED APPLICATIONS

This application is a divisional application of U.S. patent application Ser. No. 11/166,990 entitled Method for Genotype Screening filed Jun. 24, 2005.

### FIELD OF THE INVENTION

This invention relates to methods for genotype screening. More specifically, this invention relates to various methods to detect or screen for at least one designated genetic sequences in a plurality of biological samples, such as tissues and cells.

### BACKGROUND OF THE INVENTION

Genomic modification resulting from mutations in the DNA of an organism can be transferred to the progeny if such mutations are present in the gametes of the organism, referred to as germ-line mutations. These mutations may arise from genetic manipulation of the DNA using recombinant DNA technology or may be introduced by challenging the DNA by chemical or physical means. DNA introduced via recombinant DNA technology can be derived from many sources, including but not limited to DNA from viruses, mycoplasm, bacteria, fungi, yeast, and chordates including mammals such as humans.

Recombinant DNA technology allows for the introduction, deletion or replacement of DNA of an organism. Random introduction of DNA into a cell can be achieved by technologies such as transfection (including electroporation, lipofection), injection (pronuclear injection, nuclear transplantation) or transduction (viral infection). Random mutations (point mutations, deletions, amplifications) can be generated by treatment of cells with chemical mutagens or submitting them to physical insult such as X-irradiation or linear energy transfer irradiation (LET). Targeted addition, deletion or replacement of DNA in an organism (either inducible or non-inducible) is achieved via homologous recombination. Inducible systems employ sequence-specific recombinases such as Cre-LoxP (U.S. Pat. Nos. 5,654,182 and 5,677,177) and FLP/FRT (U.S. Pat. No. 5,527,695).

Transgenic organisms are organisms that carry DNA sequences (be it genes or gene segments) derived from another or the same species, stably integrated randomly into their genome. Transgenic mammals are generally created by microinjection of DNA into the pronucleus of fertilized eggs, a technique in which the number of DNA copies or the integration site of the DNA into the host genome is uncontrollable. A transgenic line or strain refers to an organism that transmits the foreign DNA sequences to its offspring.

Genotype screening is used to determine if a genome possesses specific genetic sequences that exist endogenously or have been modified, mutated or genetically engineered. Genomic nucleic acid is screened for these modifications, mutations or endogenous conditions. Genomic nucleic acid is challenging to work with because of its size. The genomic nucleic acid includes both coding and noncoding regions. Therefore, the genomic nucleic acid contains exons and introns, promoter and gene regulation regions, telomeres, origins or replication and nonfunctional intergenic nucleic acid. The genomic nucleic acid is a double stranded molecule which is methylated. cDNA and PCR-amplicons differs in that the molecules are much smaller. Additionally, biochemical modification events, such as methylation, do not occur with the smaller molecules. Shena, M (2000) DNA Microarrays: A Practical Approach. Oxford University Press, New York, N.Y.

Genotype screening is currently done manually. The present manual system is time-consuming and can provide variable results depending on the laboratory and even depending on skill of laboratory workers. Presently, a researcher using Southern blot technology may require greater than a week to screen a tissue sample for a transgene or a targeted mutation.

In an alternative technology, up to thirty PCR (polymerase chain reaction) can be conducted in an Eppendorf microtube® (Brinkmann Instruments, Westbury, N.Y.) and separated on a gel. This process in most laboratories requires 3 to 7 days. A need exists in the industry to provide a system and method for more accurate, faster and high volume genotype screening.

Additionally, as researchers continue to use transgenic species in research specific information about the progeny of the transgenic species is of vital importance. An emerging technique in mouse mutant breeding is producing ‘homozygous’ transgenic conditions. During the initial creation of transgenic animals the transgene sequence integrates randomly into the host genome. Moreover, the number of transgene insertions also varies. Once the transgene is established in the genome, some investigators are interested in having this/these transgene(s) on the corresponding chromosome. The preferred mechanism for getting both chromosomes to have the transgene(s), is by breeding two transgenic animal from the same strain together. The goal is to identify homozygous animals that can then be bred to each other to ensure continual homozygous progeny. Typically, such transgenic animals are difficult to genotype by traditional PCR methods as accurate quantification is not possible with fragment-based analysis.

### SUMMARY OF THE INVENTION

The present invention provides a unique solution to the above-described problems by providing a method for rapid genotype screening. In particular, this invention provides a method to rapidly report screening results to a remote user from a screening laboratory for a plurality of biological samples. Efficient screening of a plurality of biological samples can be achieved by placing the sample to be screened in a well of a microwell container. The biological samples in the microwell containers are lysed to release at least a portion of intact genomic nucleic acid and cellular debris. A standard concentration of purified genomic nucleic acid is obtained by saturating the binding ability of the magnetic particles and by regulating the amount of genomic nucleic acid released. The purified genomic nucleic acid is screened to obtain screening results. The screening results are reported to a remote user. These screening results can include information on whether a designated genetic sequence is present in an organism and the zygosity of designated genetic sequences. Additionally, the zygosity of a transgene can be quantitatively determined and reported to a remote user.

Additionally, rapid screening can be obtained by using methods to evaluate the validity of the data obtained from screening. This method to evaluate the screening results includes comparing the screening results for a sample with a designated genetic sequence with a sample including a housekeeping sequence.

### BRIEF DESCRIPTION OF THE DRAWINGS

A more complete understanding of the invention and its advantages will be apparent from the following Description of the Preferred Embodiment(s) taken in conjunction with the accompanying drawings, wherein:

FIG. 1 is an illustrative overview of the remote automated testing procedures of the present invention.

FIG. 2 is a block diagram of one embodiment of the system.

FIG. 3 is a block diagram of the ordering procedure.

FIG. 4 is a block diagram of account registration.

FIGS. 5-6 illustrate the survey of work and sample identification sections.

FIG. 7A is a block diagram of the laboratory process system.

FIG. 7B is a block diagram of the laboratory process system.

FIG. 7C is a block diagram of the laboratory process system.

FIG. 7D is a block diagram of the laboratory process system.

FIG. 8 is a block diagram of standard laboratory stations.

FIG. 9 is a screen display illustrating a document on the transgenic screening laboratory 20's web site relating to an outcome file.

FIG. 10 is a graphical representation of the results.

FIG. 11 is a graphical representation of signal magnitude.

FIG. 12 is a graphical representation of signal magnitude.

FIG. 13 is a graphical representation of signal magnitude.

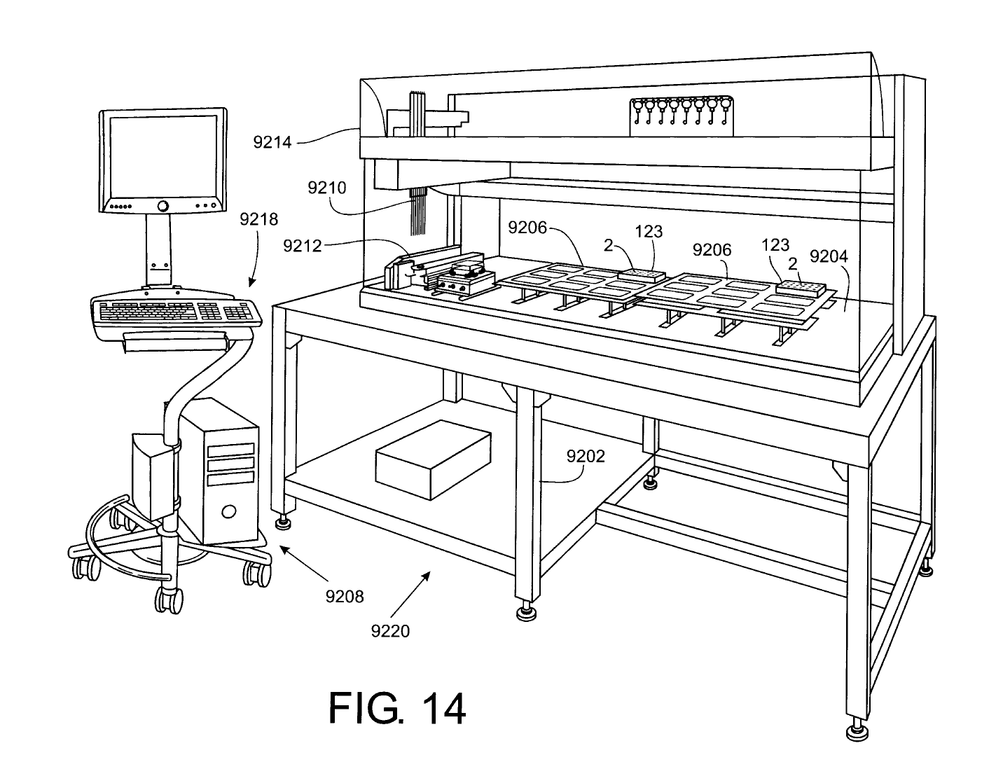

FIGS. 14 and 15 illustrate a preferred device for performing the functions of a Lysing Station and an Automated Accessioning Station as described herein, including an oven (FIG. 15) for incubating the samples.

FIG. 16 illustrates a preferred device for performing the functions of an Isolation/Purification Station as described herein.

FIG. 17 illustrates a preferred device for drying samples.

FIG. 18 illustrates a preferred device for performing the functions of a Screening Station as described herein.

FIG. 19 illustrates a preferred device for performing the functions of a Detection Station as described herein.

### DETAILED DESCRIPTION OF THE PREFERRED EMBODIMENTS

The present invention provides a method for high volume genotype screening. This invention provides a method for rapid identification of an organism, whose genome possesses specific genetic sequences that exist endogenously or has been modified, mutated or genetically engineered. All patents, patent applications and articles discussed or referred to in this specification are hereby incorporated by reference.

### 1. DEFINITIONS

The following terms and acronyms are used throughout the detailed description.

Alox5-KO

(SEQ ID NO. 1)

TGCCCAGCGGTCCTATCTAGAGGTCATTCTCTCCACAGAGCGAGTCAAGA
ACCACTGGCAGGAAGACCTCATGTTTGGCTACCAGTTCCTGAATGGCTGC
AACCCAGTAATTCTACCGGGTAGGGGAGGCGCTTTTCCCAAGGCAGTCTG
GAGCATGCGCTTTAGCAGCCCCGCTGGCACTTGGCGCTACACAAGTGGCC
TCTGGCCTCGCACACATTCCACATCCACCGGTAGCGCCAACCGGCTCCGT
TCTTTGGTGGCCCCTTCGCGCCACCTTCTACTCCTCCCCTAGTCAGGAAG
TTCCCCCCCGCCCCGCAGCTCGCGTCGTGCAGGACGTGACAAATGGAAGT
AGCACGTCTCACTAGTCTCGTGCAGATGGACAGCACCGCTGAGCAATGGA
AGCGGGTAGGCCTTTGGGGCAGCGGCCAATAGCAGCTTTGCTCCTTCGCT
TTCTGGGCTCAGAGGCTGGGAAGGGGTGGGTCCGGGGGCGGGCTCAGGG

(SEQ ID NO. 2)

Forward Primer Seq.: TTGGCTACCAGTTCCTGAATGG

(SEQ ID NO. 3)

Reverse Primer Seq.: CAGACTGCCTTGGGAAAAGC

(SEQ ID NO. 4)

Probe: CTGCAACCCAGTAATTC
ALOX5-WT

(SEQ ID NO. 5)

AAGAACCACTGGCAGGAAGACCTCATGTTTGGCTACCAGTTCCTGAATGG
CTGCAACCCAGTACTCATCAAGCGCTGCACAGCGTTGCCCCCGAAGCTCC
CAGTGACCACAGAGATGGTGGAGTGCAGCCTAGAGCGGCAGCTCAGTTTA
GAACA

(SEQ ID NO. 6)

Forward Primer Seq.: TTGGCTACCAGTTCCTGAATGG

(SEQ ID NO. 7)

Reverse Primer Seq.: CTGTGGTCACTGGGAGCTT

(SEQ ID NO. 8)

Probe: CTGCAACCCAGTACTCAT
APC Min

(SEQ ID NO. 9)

TATCATGTCTCCCGGCTCAAGTCTGCCATCCCTTCACGTTAGGAAACAGA
AAGCTCTAGAAGCTGAGCTAGATGCTCAGCATTTATCAGAAACCTTCGAC
AACATTGACAACCTAAGTCCCAAGGCCTCTCACCGGAGTAAGCAGAGACA
CAAGCAGAATCTTTATGGTGACTATGCTTTTGACGCCAATCGACATGATG
ATAGTAGGTCAGACAATTTCAATACTGGAAACATGACTGTTCTTTCACCA
TATTTAAATACTACGGTATTGCCCAGCTCTTCTTCCTCAAGGGGAAGTTT
AGACAGTTCTCGTTCTGAGAAAGACAGAAGTTAGGAGAGAGAGCGAGGTA
TTGGCCTCAGTGCTTACCATCCAACAACAGAAAATGCAGGAACCTCATCA
AAACGAGGTCTGCAGATCACTACCACTGCAGCCCAGATAGCCAAAGTTAT
GGAAGAAGTATCAGCCATTCATACCTCCCAGGACGACAGAAGTTCTGCTT
CTACCACCGAGTTCCATTGTGTGGCAGACGACAGGAGTGCGGCACGAAGA
AGCTCTGCCTNNNNNNNNNNNNNNNNNNNNNNNNNCTTCACTAAGTCGGA
AAATTCAAATAGGACATGCTCTATGCCTTATGCCAAAGTGGAATATAAAC
GATCTTCAAATGACAGTTTAAATAGTGTCACTAGTA

(SEQ ID NO. 10)

Forward Primer: GGGAAGTTTAGACAGTTCTCGTTCT

(SEQ ID NO. 11)

Reverse Primer: GTAAGCACTGAGGCCAATACCT

(SEQ ID NO. 12)

Probe 1: CTCTCTCCAAACTTC

(SEQ ID NO. 13)

Probe 2: TCTCTCTCCTAACTTC
Bgal

(SEQ ID NO. 14)

GTTGAGAATGAGTACGGGTCCTACTTTGCCTGCGATTACGACTACCTACG
CTTCCTGGTGCACCGCTTCCGCTACCATCTGGGTAATGACGTCATTCTCT
TCACCACCGACGGAGCAAGTGAAAAAATGCTGAAGTGTGGGACCCTGCAG
GACCTGTACGCCACAGTGGATTTTGGAACAG

(SEQ ID NO. 15)

Forward Primer Seq.: CACCGCTTCCGCTACCAT

(SEQ ID NO. 16)

Reverse Primer Seq.: GCTCCGTCGGTGGTGAAG

(SEQ ID NO. 17)

Probe: CTGGGTAATGACGTCATTCT

complementary—chemical affinity between nitrogenous bases as a result of hydrogen bonding. Responsible for the base pairing between nucleic acid strands. Klug, W. S. and Cummings, M. R. (1997) Concepts of Genetics, fifth ed., Prentice-Hall, Upper Saddle River, N.J.

copy number—the number of transgenes that have randomly integrated into the genome.

Cjun - (housekeeping or reference sequence)

(SEQ ID NO. 18)

GACCGGTAACAAGTGGCCGGGAGCGAACTTTTGCAAATCTCTTCTGCGCC
TTAAGGCTGCCACCGAGACTGTAAAGAAAAGGGAGAAGAGGAACCTATAC
TCATACCAGTTCGCACAGGCGGCTGAAGTTGGGCGAGCGCTAGCCGCGGC
TGCCTAGCGTCCCCCTCCCCCTCACAGCGGAGGAGGGGACAGTTGTCGGA
GGCCGGGCGGCAGAGCCCGATCGCGGGCTTCCACCGAGAATTCCGTGACG
ACTGGTCAGCACCGCCGGAGAGCCGCTGTTGCTGGGACTGGTCTGCGGGC
TCCAAGGAACCGCTGCTCCCCGAGAGCGCTCCGTGAGTGACCGCGACTTT
TCAAAGCTCGGCATCGCGCGGGAGCCTACCAACGTGAGTGCTAGCGGAGT
CTTAACCCTGCGCTCCCTGGAGCGAACTGGGGAGGAGGGCTCAGGGGGAA
GCACTGCCGTCTGGAGCGCACGCTCCTAAACAAACTTTGTTACAGAAGCG
GGGACGCGCGGGTATCCCCCCGCTTCCCGGCGCGCTGTTGCGGCCCCGAA
ACTTCTGCGCACAGCCCAGGCTAACCCCGCGTGAAGTGACGGACCGTTCT
ATGACTGCAAAGATGGAAACGACCTTCTACGACGATGCCCTCAACGCCTC
GTTCCTCCAGTCCGAGAGCGGTGCCTACGGCTACAGTAACCCTAAGATCC
TAAAACAGAGCATGACCTTGAACCTGGCCGACCCGGTGGGCAGTCTGAAG
CCGCACCTCCGCGCCAAGAACTCGGACCTTCTCACGTCGCCCGACGTCGG
GCTGCTCAAGCTGGCGTCGCCGGAGCTGGAGCGCCTGATCATCCAGTCCA
GCAATGGGCACATCACCACTACACCGACCCCCACCCAGTTCTTGTGCCCC
AAGAACGTGACCGACGAGCAGGAGGGCTTCGCCGAGGGCTTCGTGCGCGC
CCTGGCTGAACTGCATAGCCAGAACACGCTTCCCAGTGTCACCTCCGCGG
CACAGCCGGTCAGCGGGGCGGGCATGGTGGCTCCCGCGGTGGCCTCAGTA
GCAGGCGCTGGCGGCGGTGGTGGCTACAGCGCCAGCCTGCACAGTGAGCC
TCCGGTCTACGCCAACCTCAGCAACTTCAACCCGGGTGCGCTGAGCAGCG
GCGGTGGGGCGCCCTCCTATGGCGCGGCCGGGCTGGCCTTTCCCTCGCAG
CCGCAGCAGCAGCAGCAGCCGCCTCAGCCGCCGCACCACTTGCCCCAACA
GATCCCGGTGCAGCACCCGCGGCTGCAAGCCCTGAAGGAAGAGCCGCAGA
CCGTGCCGGAGATGCCGGGAGAGACGCCGCCCCTGTCCCCTATCGACATG
GAGTCTCAGGAGCGGATCAAGGCAGAGAGGAAGCGCATGAGGAACCGCAT
TGCCGCCTCCAAGTGCCGGAAAAGGAAGCTGGAGCGGATCGCTCGGCTAG
AGGAAAAAGTGAAAACCTTGAAAGCGCAAAACTCCGAGCTGGCATCCACG
GCCAACATGCTCAGGGAACAGGTGGCACAGCTTAAGCAGAAAGTCATGAA
CCACGTTAACAGTGGGTGCCAACTCATGCTAACGCAGCAGTTGCAAACGT
TTTGAGAACAGACTGTCAGGGCTGAGGGGCAATGGAAGAAAAAAAATAAC
AGAGACAAACTTGAGAACTTGACTGGTTGCGACAGAGAAAAAAAAAGTGT
CCGAGTACTGAAGCCAAGGGTACACAAGATGGACTGGGTTGCGACCTGAC
GGCGCCCCCAGTGTGCTGGAGTGGGAAGGACGTGGCGCGCCTGGCTTTGG
CGTGGAGCCAGAGAGCAGCGGCCTATTGGCCGGCAGACTTTGCGGACGGG
CTGTGCCCGCGCGCGACCAGAACGATGGACTTTTCGTTAACATTGACCAA
GAACTGCATGGACCTAACATTCGATCTCATTCAGTATTAAAGGGGGGTGG
GAGGGGTTACAAACTGCAATAGAGACTGTAGATTGCTTCTGTAGTGCTCC
TTAACACAAAGCAGGGAGGGCTGGGAAGGGGGGGGAGGCTTGTAAGTGCC
AGGCTAGACTGCAGATGAACTCCCCTGGCCTGCCTCTCTCAACTGTGTAT
GTACATATATATTTTTTTTTAATTTGATGAAAGCTGATTACTGTCAATAA
ACAGCTTCCTGCCTTTGTAAGTTATTCCATGTTTGTTTGTTTGGGTGTCC
TGCCC

(SEQ ID NO. 19)

Forward Primer: GAGTGCTAGCGGAGTCTTAACC

(SEQ ID NO. 20)

Reverse Primer: CTCCAGACGGCAGTGCTT

(SEQ ID NO. 21)

Probe: AAGCACTGCCGTCTGGAG
Cre

(SEQ ID: NO. 22)

ATGCCCAAGAAGAAGAGGAAGGTGTCCAATTTACTGACCGTACACCAAAA
TTTGCCTGCATTACCGGTCGATGCAACGAGTGATGAGGTTCGCAAGAACC
TGATGGACATGTTCAGGGATCGCCAGGCGTTTTCTGAGCATACCTGGAAA
ATGCTTCTGTCCGTTTGCCGGTCGTGGGCGGCATGGTGCAAGTTGAATAA
CCGGAAATGGTTTCCCGCAGAACCTGAAGATGTTCGCGATTATCTTCTAT
ATCTTCAGGCGCGCGGTCTGGCAGTAAAAACTATCCAGCAACATTTGGGC
CAGCTAAACATGCTTCATCGTCGGTCCGGGCTGCCACGACCAAGTGACAG
CAATGCTGTTTCACTGGTTATGCGGCGGATCCGAAAAGAAAACGTTGATG
CCGGTGAACGTGCAAAACAGGCTCTAGCGTTCGAACGCACTGATTTCGAC
CAGGTTCGTTCACTCATGGAAAATAGCGATCGCTGCCAGGATATACGTAA
TCTGGCATTTCTGGGGATTGCTTATAACACCCTGTTACGTATAGCCGAAA
TTGCCAGGATCAGGGTTAAAGATATCTCACGTACTGACGGTGGGAGAATG
TTAATCCATATTGGCAGAACGAAAACGCTGGTTAGCACCGCAGGTGTAGA
GAAGGCACTTAGCCTGGGGGTAACTAAACTGGTCGAGCGATGGATTTCCG
TCTCTGGTGTAGCTGATGATCCGAATAACTACCTGTTTTGCCGGGTCAGA
AAAAATGGTGTTGCCGCGCCATCTGCCACCAGCCAGCTATCAACTCGCGC
CCTGGAAGGGATTTTTGAAGCAACTCATCGATTGATTTACGGCGCTAAGG
ATGACTCTGGTCAGAGATACCTGGCCTGGTCTGGACACAGTGCCCGTGTC
GGAGCCGCGCGAGATATGGCCCGCGCTGGAGTTTCAATACCGGAGATCAT
GCAAGCTGGTGGCTGGACCAATGTAAATATTGTCATGAACTATATCCGTA
ACCTGGATAGTGAAACAGGGGCAATGGTGCGCCTGCTGGAAGATGGCGAT
TAGCCATTAACGCGTAAATGATTGCTATAATTATTTGATAT

(SEQ ID: NO. 23)

Forward Primer: TTAATCCATATTGGCAGAACGAAAACG

(SEQ ID: NO. 24)

Reverse Primer: CAGGCTAAGTGCCTTCTCTACA

(SEQ ID: NO. 25)

Probe: CCTGCGGTGCTAACC

designated genetic sequence—includes a transgenic insert, a selectable marker, microsatellite loci, recombinant site or any gene or gene segment.

DNA (deoxyribonucleic acid)—One of the two main types of nucleic acid, consisting of a long, unbranched macromolecule formed from one, or more commonly, two, strands of linked deoxyribonucleotides, the 3″-phosphate group of each constituent deoxyribonucleotide being joined in 3′, 5′-phosphodiester linkage to the 5′-hydroxyl group of the deoxyribose moiety of the next one. Oxford Dictionary of Biochemistry and Molecular Biology; p. 182.

embryonic stem cells (ES cells)—a cell of the early embryo that can replicate indefinitely and which can differentiate into other cells; stem cells serve as a continuous source of new cells.

genome—all the genetic material in the chromosomes of a particular organism; its size is generally given as its total number of base pairs.

genomic nucleic acid—The genomic nucleic acid includes both coding and noncoding regions. Therefore, the genomic nucleic acid contains exons and introns, promoter and gene regulation regions, telomeres, origins or replication and nonfunctional intergenic nucleic acid. The genomic nucleic acid is a double stranded molecule which is methylated. cDNA and PCR-amplicons differs in that the molecules are much smaller. Additionally, biochemical modification events, such as methylation, do not occur with the smaller molecules. Shena, M (2000) DNA Microarrays: A Practical Approach. Oxford University Press, New York, N.Y.

genotype—genetic constitution of an individual cell or organism that can include at least one designated gene sequence.

hemizygous—a situation within a cell or organism where only one copy of a gene, group of genes or genetic sequence is present instead of two copies in a diploid genome.

heterozygosity—the state of having two different genes (alleles) at one or more corresponding loci on homologous chromosomes.

homozygosity—The state of having the same genes (alleles) at one or more corresponding homologous chromosomes.

HumanTTTy8

(SEQ ID NO. 26)

AAAGAAGAGCAGCACGTCATACCCAAGACCAACATCTCTCAGTGTTTCAC
GCTAACCCAAGGAGAGACACTAGCAGTCTTCTCTGCAGGACCCCTTGAAT
TTACATTGAATTCCATCCCCAGCCGAGCAGGTGCTTAAAGTCAACAGGGG
ACACTCCATTTTCTTGGAATTTCATTCTGGCAAAGAGGGTGTGAGCAGCA
ATAAG

(SEQ ID NO. 27)

Forward Primer Seq.: GCAGGACCCCTTGAATTTACATTGA

(SEQ ID NO. 28)

Reverse Primer Seq.: TGGAGTGTCCCCTGTTGACT

(SEQ ID NO. 29)

Probe: CCGAGCAGGTGCTTAA
Hygromycin

(SEQ ID: No. 30)

ATGAAAAAGCCTGAACTCACCGCGACGTCTGTCGAGAAGTTTCTGATCGA
AAAGTTCGACAGCGTCTCCGACCTGATGCAGCTCTCGGAGGGCGAAGAAT
CTCGTGCTTTCAGCTTCGATGTAGGAGGGCGTGGATATGTCCTGCGGGTA
AATAGCTGCGCCGATGGTTTCTACAAAGATCGTTATGTTTATCGGCACTT
TGCATCGGCCGCGCTCCCGATTCCGGAAGTGCTTGACATTGGGGAATTCA
GCGAGAGCCTGACCTATTGCATCTCCCGCCGTGCACAGGGTGTCACGTTG
CAAGACCTGCCTGAAACCGAACTGCCCGCTGTTCTGCAGCCGGTCGCGGA
GGCCATGGATGCGATCGCTGCGGCCGATCTTAGCCAGACGAGCGGGTTCG
GCCCATTCGGACCGCAAGGAATCGGTCAATACACTACATGGCGTGATTTC
ATATGCGCGATTGCTGATCCCCATGTGTATCACTGGCAAACTGTGATGGA
CGACACCGTCAGTGCGTCCGTCGCGCAGGCTCTCGATGAGCTGATGCTTT
GGGCCGAGGACTGCCCCGAAGTCCGGCACCTCGTGCACGCGGATTTCGGC
TCCAACAATGTCCTGACGGACAATGGCCGCATAACAGCGGTCATTGACTG
GAGCGAGGCGATGTTCGGGGATTCCCAATACGAGGTCGCCAACATCTTCT
TCTGGAGGCCGTGGTTGGCTTGTATGGAGCAGCAGACGCGCTACTTCGAG
CGGAGGCATCCGGAGCTTGCAGGATCGCCGCGGCTCCGGGCGTATATGCT
CCGCATTGGTCTTGACCAACTCTATCAGAGCTTGGTTGACGGCAATTTCG
ATGATGCAGCTTGGGCGCAGGGTCGATGCGACGCAATCGTCCGATCCGGA
GCCGGGACTGTCGGGCGTACACAAATCGCCCGCAGAAGCGCGGCCGTCTG
GACCGATGGCTGTGTAGAAGTACTCGCCGATAGTGGAAACCGACGCCCCA
GCACTCGTCCGAGGGCAAAGGAATAG

(SEQ ID NO.: 31)

Forward Primer: CGCAAGGAATCGGTCAATACACTA

(SEQ ID NO.: 32)

Reverse Primer: CACAGTTTGCCAGTGATACACATG

(SEQ ID NO.: 33)

Probe: CATGGCGTGATTTCAT

internet—a collection of interconnected (public and/or private) networks that are linked together by a set of standard protocols to form a global, distributed network. The World Wide Web (hereinafter web) refers to both a distributed collection of interlinked, user viewable hypertext documents (commonly referred to as web pages) that are accessible via the Internet and the user and server software components which provide user access to such documents using standard Internet protocols.

line—A line is a group of organisms bred for a genotype (i.e. at least one designated genetic sequence).

MHV

(SEQ ID NO.: 34)

TATAAGAGTGATTGGCGTCCGTACGTACCCTCTCAACTCTAAAACTCTTG
TAGTTTAAATCTAATCTAAACTTTATAAACGGCACTTCCTGCGTGTCCAT
GCCCGCGGGCCTGGTCTTGTCATAGTGCTGACATTTGTAGTTCCTTGACT
TTCGTTCTCTGCCAGTGACGTGTCCATTCGGCGCCAGCAGCCCACCCATA
GGTTGCATAATGGCAAAGATGGGCAAATACGGTCTCGGCTTCAAATGGGC
CCCAGAATTTCCATGGATGCTTCCGAACGCATCGGAGAAGTTGGGTAACC
CTGAGAGGTCAGAGGAGGATGGGTTTTGCCCCTCTGCTGCGCAAGAACCG
AAAGTTAAAGGAAAAACTTTGGTTAATCACGTGAGGGTGAATTGTAGCCG
GCTTCCAGCTTTGGAATGCTGTGTTCAGTCTGCCATAATCCGTGATATTT
TTGTAGATGAGGATCCCCAGAAGGTGGAGGCCTCAACTATGATGGCATTG
CAGTTCGGTAGTGCCGTCTTGGTTAAGCCATCCAAGCGCTTGTCTATTCA
GGCATGGACTAATTTGGGTGTGCTTCCCAAAACAGCTGCCATGGGGTTGT
TCAAGCGCGTCTGCCTGTGTAACACCAGGGAGTGCTCTTGTGACGCCCAC
GTGGCCTTTCACCTTTTTACGGTCCAACCCGATGGTGTATGCCTGGGTAA
TGGCCGTTTTATAGGCTGGTTCGTTCCAGTCACAGCCATACCGGAGTATG
CGAAGCAGTGGTTGCAACCCTGGTCCATCCTTCTTCGTAAGGGTGGTAAC
AAAGGGTCTGTGACATCCGGCCACTTCCGCCGCGCTGTTACCATGCCTGT
GTATGACTTTAATGTAGAGGATGCTTGTGAGGAGGTTCATCTTAACCCGA
AGGGTAAGTACTCCTGCAAGGCGTATGCTCTTCTTAAGGGCTATCGCGGT
GTTAAGCCCATCCTGTTTGTGGACCAGTATGGTTGCGACTATACTGGATG
TCTCGCCAAGGGTCTTGAGGACTATGGCGATCTCACCTTGAGTGAGATGA
AGGAGTTGTTCCCTGTGTGGCGTGACTCCTTGGATAGTGAAGTCCTTGTG
GCTTGGCACGTTGATCGAGATCCTCGGGCTGCTATGCGTCTGCAGACTCT
TGCTACTGTACGTTGCATTGATTATGTGGGCCAACCGACCGAGGATGTGG
TGGATGGAGATGTGGTAGTGCGTGAGCCTGCTCATCTTCTCGCAGCCAAT
GCCATTGTTAAAAGACTCCCCCGTTTGGTGGAGACTATGCTGTATACGGA
TTCGTCCGTTACAGAATTCTGTTATAAAACCAAGCTGTGTGAATGCGGTT
TTATCACGCAGTTTGGCTATGTGGATTGTTGTGGTGACACCTGCGATTTT
CGTGGGTGGGTTGCCGGCAATATGATGGATGGCTTTCCATGTCCAGGGTG
TACCAAAAATTATATGCCCTGGGAATTGGAGGCCCAGTCATCAGGTGTTA
TACCAGAAGGAGGTGTTCTATTCACTCAGAGCACTGATACAGTGAATCGT
GAGTCCTTTAAGCTCTACGGTCATGCTGTTGTGCCTTTTGGTTCTGCTGT
GTATTGGAGCCCTTGCCCAGGTATGTGGCTTCCAGTAATTTGGTCTTCTG
TTAAGTCATACTCTGGTTTGACTTATACAGGAGTAGTTGGTTGTAAGGCA
ATTGTTCAAGAGACAGACGCTATATGTCGTTCTCTGTATATGGATTATGT
CCAGCACAAGTGTGGCAATCTCGAGCAGAGAGCTATCCTTGGATTGGACG
ATGTCTATCATAGACAGTTGCTTGTGAATAGGGGTGACTATAGTCTCCTC
CTTGAGAATGTGGATTTGTTTGTTAAGCGGCGCGCTGAATTTGCTTGCAA
ATTCGCCACCTGTGGAGATGGTCTTGTACCCCTCCTACTAGATGGTTTAG
TGCCCCGCAGTTATTATTTGATTAAGAGTGGTCAAGCTTTCACCTCTATG
ATGGTTAATTTTAGCCATGAGGTGACTGACATGTGTATGGACATGGCTTT
ATTGTTCATGCATGATGTTAAAGTGGCCACTAAGTATGTTAAGAAGGTTA
CTGGCAAACTGGCCGTGCGCTTTAAAGCGTTGGGTGTAGCCGTTGTCAGA
AAAATTACTGAATGGTTTGATTTAGCCGTGGACATTGCTGCTAGTGCCGC
TGGATGGCTTTGCTACCAGCTGGTAAATGGCTTATTTGCAGTGGCCAATG
GTGTTATAACCTTTGTACAGGAGGTGCCTGAGCTTGTCAAGAATTTTGTT
GACAAGTTCAAGGCATTTTTCAAGGTTTTGATCGACTCTATGTCGGTTTC
TATCTTGTCTGGACTTACTGTTGTCAAGACTGCCTCAAATAGGGTGTGTC
TTGCTGGCAGTAAGGTTTATGAAGTTGTGCAGAAATCTTTGTCTGCATAT
GTTATGCCTGTGGGTTGCAGTGAAGCCACTTGTTTGGTGGGTGAGATTGA
ACCTGCAGTTTTTGAAGATGATGTTGTTGATGTGGTTAAAGCCCCATTAA
CATATCAAGGCTGTTGTAAGCCACCCACTTCTTTCGAGAAGATTTGTATT
GTGGATAAATTGTATATGGCCAAGTGTGGTGATCAATTTTACCCTGTGGT
TGTTGATAACGACACTGTTGGCGTGTTAGATCAGTGCTGGAGGTTTCCCT
GTGCGGGCAAGAAAGTCGAGTTTAACGACAAGCCCAAAGTCAGGAAGATA
CCCTCCACCCGTAAGATTAAGATCACCTTCGCACTGGATGCGACCTTTGA
TAGTGTTCTTTCGAAGGCGTGTTCAGAGTTTGAAGTTGATAAAGATGTTA
CATTGGATGAGCTGCTTGATGTTGTGCTTGACGCAGTTGAGAGTACGCTC
AGCCCTTGTAAGGAGCATGATGTGATAGGCACAAAAGTTTGTGCTTTACT
TGATAGGTTGGCAGGAGATTATGTCTATCTTTTTGATGAGGGAGGCGATG
AAGTGATCGCCCCGAGGATGTATTGTTCCTTTTCTGCTCCTGATGATGAA
GACTGCGTTGCAGCGGATGTTGTAGATGCAGATGAAAACCAAGATGATGA
TGCTGAAGACTCAGCAGTCCTTGTCGCTGATACCCAAGAAGAGGACGGCG
TTGCCAAGGGGCAGGTTGAGGCGGATTCGGAAATTTGCGTTGCGCATACT
GGTAGTCAAGAAGAATTGGCTGAGCCTGATGCTGTCGGATCTCAAACTCC
CATCGCCTCTGCTGAGGAAACCGAAGTCGGAGAGGCAAGCGACAGGGAAG
GGATTGCTGAGGCGAAGGCAACTGTGTGTGCTGATGCTGTAGATGCCTGC
CCCGATCAAGTGGAGGCATTTGAAATTGAAAAGGTTGAAGACTCTATCTT
GGATGAGCTTCAAACTGAACTTAATGCGCCAGCGGACAAGACCTATGAGG
ATGTCTTGGCATTCGATGCCGTATGCTCAGAGGCGTTGTCTGCATTCTAT
GCTGTGCCGAGTGATGAGACGCACTTTAAAGTGTGTGGATTCTATTCGCC
TGCTATAGAGCGCACTAATTGTTGGCTGCGTTCTACTTTGATAGTAATGC
AGAGTCTACCTTTGGAATTTAAAGACTTGGAGATGCAAAAGCTCTGGTTG
TCTTACAAGGCCGGCTATGACCAATGCTTTGTGGACAAACTAGTTAAGAG
CGTGCCCAAGTCTATTATCCTTCCACAAGGTGGTTATGTGGCAGATTTTG
CCTATTTCTTTCTAAGCCAGTGTAGCTTTAAAGCTTATGCTAACTGGCGT
TGTTTAGAGTGTGACATGGAGTTAAAGCTTCAAGGCTTGGACGCCATGTT
TTTCTATGGGGACGTTGTGTCTCATATGTGCAAGTGTGGTAATAGCATGA
CCTTGTTGTCTGCAGATATACCCTACACTTTGCATTTTGGAGTGCGAGAT
GATAAGTTTTGCGCTTTTTACACGCCAAGAAAGGTCTTTAGGGCTGCTTG
TGCGGTAGATGTTAATGATTGTCACTCTATGGCTGTAGTAGAGGGCAAGC
AAATTGATGGTAAAGTGGTTACCAAATTTATTGGTGACAAATTTGATTTT
ATGGTGGGTTACGGGATGACATTTAGTATGTCTCCTTTTGAACTCGCCCA
GTTATATGGTTCATGTATAACACCAAATGTTTGTTTTGTTAAAGGAGATG
TTATAAAGGTTGTTCGCTTAGTTAATGCTGAAGTCATTGTTAACCCTGCT
AATGGGCGTATGGCTCATGGTGCAGGTGTTGCAGGTGCTATAGCTGAAAA
GGCGGGCAGTGCTTTTATTAAAGAAACCTCCGATATGGTGAAGGCTCAGG
GCGTTTGCCAGGTTGGTGAATGCTATGAATCTGCCGGTGGTAAGTTATGT
AAAAAGGTGCTTAACATTGTAGGGCCAGATGCGCGAGGGCATGGCAAGCA
ATGCTATTCACTTTTAGAGCGTGCTTATCAGCATATTAATAAGTGTGACA
ATGTTGTCACTACTTTAATTTCGGCTGGTATATTTAGTGTGCCTACTGAT
GTCTCCCTAACTTACTTACTTGGTGTAGTGACAAAGAATGTCATTCTTGT
CAGTAACAACCAGGATGATTTTGATGTGATAGAGAAGTGTCAGGTGACCT
CCGTTGCTGGTACCAAAGCGCTATCACTTCAATTGGCCAAAAATTTGTGC
CGTGATGTAAAGTTTGTGACGAATGCATGTAGTTCGCTTTTTAGTGAATC
TTGCTTTGTCTCAAGCTATGATGTGTTGCAGGAAGTTGAAGCGCTGCGAC
ATGATATACAATTGGATGATGATGCTCGTGTCTTTGTGCAGGCTAATATG
GACTGTCTGCCCACAGACTGGCGTCTCGTTAACAAATTTGATAGTGTTGA
TGGTGTTAGAACCATTAAGTATTTTGAATGCCCGGGCGGGATTTTTGTAT
CCAGCCAGGGCAAAAAGTTTGGTTATGTTCAGAATGGTTCATTTAAGGAG
GCGAGTGTTAGCCAAATAAGGGCTTTACTCGCTAATAAGGTTGATGTCTT
GTGTACTGTTGATGGTGTTAACTTCCGCTCCTGCTGCGTAGCAGAGGGTG
AAGTTTTTGGCAAGACATTAGGTTCAGTCTTTTGTGATGGCATAAATGTC
ACCAAAGTTAGGTGTAGTGCCATTTACAAGGGTAAGGTTTTCTTTCAGTA
CAGTGATTTGTCCGAGGCAGATCTTGTGGCTGTTAAAGATGCCTTTGGTT
TTGATGAACCACAACTGCTGAAGTACTACACTATGCTTGGCATGTGTAAG
TGGTCAGTAGTTGTTTGTGGCAATTATTTTGCTTTCAAGCAGTCAAATAA
TAATTGCTATATAAATGTGGCATGTTTAATGCTGCAACACTTGAGTTTAA
AGTTTCCTAAGTGGCAATGGCAAGAGGCTTGGAACGAGTTCCGCTCTGGT
AAACCACTAAGGTTTGTGTCCTTGGTATTAGCAAAGGGCAGCTTTAAATT
TAATGAACCTTCTGATTCTATCGATTTTATGCGTGTGGTGCTACGTGAAG
CAGATTTGAGTGGTGCCACGTGCAATTTGGAATTTGTTTGTAAATGTGGT
GTGAAGCAAGAGCAGCGCAAAGGTGTTGACGCTGTTATGCATTTTGGTAC
GTTGGATAAAGGTGATCTTGTCAGGGGTTATAATATCGCATGTACGTGCG
GTAGTAAACTTGTGCATTGCACCCAATTTAACGTACCATTTTTAATTTGC
TCCAACACACCAGAGGGTAGGAAACTGCCCGACGATGTTGTTGCAGCTAA
TATTTTTACTGGTGGTAGTGTGGGCCATTACACGCATGTGAAATGTAAAC
CCAAGTACCAGCTTTATGATGCTTGTAATGTTAATAAGGTTTCGGAGGCT
AAGGGTAATTTTACCGATTGCCTCTACCTTAAAAATTTAAAGCAAACTTT
TTCGTCTGTGCTGACGACTTTTTATTTAGATGATGTAAAGTGTGTGGAGT
ATAAGCCAGATTTATCGCAGTATTACTGTGAGTCTGGTAAATATTATACA
AAACCCATTATTAAGGCCCAATTTAGAACATTTGAGAAGGTTGATGGTGT
CTATACCAACTTTAAATTGGTGGGACATAGTATTGCTGAAAAACTCAATG
CTAAGCTGGGATTTGATTGTAATTCTCCCTTTGTGGAGTATAAAATTACA
GAGTGGCCAACAGCTACTGGAGATGTGGTGTTGGCTAGTGATGATTTGTA
TGTAAGTCGGTACTCAAGCGGGTGCATTACTTTTGGTAAACCGGTTGTCT
GGCTTGGCCATGAGGAAGCATCGCTGAAATCTCTCACATATTTTAATAGA
CCTAGTGTCGTTTGTGAAAATAAATTTAATGTGTTGCCCGTTGATGTCAG
TGAACCCACGGACAAGGGGCCTGTGCCTGCTGCAGTCCTTGTTACCGGCG
TCCCTGGAGCTGATGCGTCAGCTGGTGCCGGTATTGCCAAGGAGCAAAAA
GCCTGTGCTTCTGCTAGTGTGGAGGATCAGGTTGTTACGGAGGTTCGTCA
AGAGCCATCTGTTTCAGCTGCTGATGTCAAAGAGGTTAAATTGAATGGTG
TTAAAAAGCCTGTTAAGGTGGAAGGTAGTGTGGTTGTTAATGATCCCACT
AGCGAAACCAAAGTTGTTAAAAGTTTGTCTATTGTTGATGTCTATGATAT
GTTCCTGACAGGGTGTAAGTATGTGGTTTGGACTGCTAATGAGTTGTCTC
GACTAGTAAATTCACCGACTGTTAGGGAGTATGTGAAGTGGGGTAAGGGA
AAGATTGTAACACCCGCTAAGTTGTTGTTGTTAAGAGATGAGAAGCAAGA
GTTCGTAGCGCCAAAAGTAGTCAAGGCGAAAGCTATTGCCTGCTATTGTG
CTGTGAAGTGGTTTCTCCTCTATTGTTTTAGTTGGATAAAGTTTAATACT
GATAATAAGGTTATATACACCACAGAAGTAGCTTCAAAGCTTACTTTCAA
GTTGTGCTGTTTGGCCTTTAAGAATGCCTTACAGACGTTTAATTGGAGCG
TTGTGTCTAGGGGCTTTTTCCTAGTTGCAACGGTCTTTTTATTATGGTTT
AACTTTTTGTATGCTAATGTTATTTTGAGTGACTTCTATTTGCCTAATAT
TGGGCCTCTCCCTACGTTTGTGGGACAGATAGTTGCGTGGTTTAAGACTA
CATTTGGTGTGTCAACCATCTGTGATTTCTACCAGGTGACGGATTTGGGC
TATAGAAGTTCGTTTTGTAATGGAAGTATGGTATGTGAACTATGCTTCTC
AGGTTTTGATATGCTGGACAACTATGATGCTATAAATGTTGTTCAACACG
TTGTAGATAGGCGTTTGTCCTTTGACTATATTAGCCTATTTAAATTAGTA
GTTGAGCTTGTAATCGGCTACTCTCTTTATACTGTGTGCTTCTACCCACT
GTTTGTCCTTATTGGAATGCAGTTGTTGACCACATGGTTGCCTGAATTCT
TTATGCTGGAGACTATGCATTGGAGTGCTCGTTTGTTTGTGTTTGTTGCC
AATATGCTTCCAGCTTTTACGTTACTGCGATTTTACATCGTGGTGACAGC
TATGTATAAGGTCTATTGTCTTTGTAGACATGTTATGTATGGATGTAGTA
AGCCTGGTTGCTTGTTTTGTTATAAGAGAAACCGTAGTGTCCGTGTTAAG
TGTAGCACCGTTGTTGGTGGTTCACTACGCTATTACGATGTAATGGCTAA
CGGCGGCACAGGTTTCTGTACAAAGCACCAGTGGAACTGTCTTAATTGCA
ATTCCTGGAAACCAGGCAATACATTCATAACTCATGAAGCAGCGGCGGAC
CTCTCTAAGGAGTTGAAACGCCCTGTGAATCCAACAGATTCTGCTTATTA
CTCGGTCACAGAGGTTAAGCAGGTTGGTTGTTCCATGCGTTTGTTCTACG
AGAGAGATGGACAGCGTGTTTATGATGATGTTAATGCTAGTTTGTTTGTG
GACATGAATGGTCTGCTGCATTCTAAAGTTAAAGGTGTGCCTGAAACGCA
TGTTGTGGTTGTTGAGAATGAAGCTGATAAAGCTGGTTTTCTCGGCGCCG
CAGTGTTTTATGCACAATCGCTCTACAGACCTATGTTGATGGTGGAAAAG
AAATTAATAACTACCGCCAACACTGGTTTGTCTGTTAGTCGAACTATGTT
TGACCTTTATGTAGATTCATTGCTGAACGTCCTCGACGTGGATCGCAAGA
GTCTAACAAGTTTTGTAAATGCTGCGCACAACTCTCTAAAGGAGGGTGTT
CAGCTTGAACAAGTTATGGATACCTTTATTGGCTGTGCCCGACGTAAGTG
TGCTATAGATTCTGATGTTGAAACCAAGTCTATTACCAAGTCCGTCATGT
CGGCAGTAAATGCTGGCGTTGATTTTACGGATGAGAGTTGTAATAACTTG
GTGCCTACCTATGTTAAAAGTGACACTATCGTTGCAGCCGATTTGGGTGT
TCTTATTCAGAATAATGCTAAGCATGTACAGGCTAATGTTGCTAAAGCCG
CTAATGTGGCTTGCATTTGGTCTGTGGATGCTTTTAACCAGCTATCTGCT
GACTTACAGCATAGGCTGCGAAAAGCATGTTCAAAAACTGGCTTGAAGAT
TAAGCTTACTTATAATAAGCAGGAGGCAAATGTTCCTATTTTAACTACAC
CGTTCTCTCTTAAAGGGGGCGCTGTTTTTAGTAGAATGTTACAATGGTTG
TTTGTTGCTAATTTGATTTGTTTCATTGTGTTGTGGGCCCTTATGCCAAC
ATATGCAGTGCACAAATCGGATATGCAGTTGCCTTTATATGCCAGTTTTA
AAGTTATAGATAATGGTGTGCTAAGGGATGTGTCTGTTACTGACGCATGC
TTCGCAAACAAATTTAATCAATTTGATCAATGGTATGAGTCTACTTTTGG
TCTTGCTTATTACCGCAACTCTAAGGCTTGTCCTGTTGTGGTTGCTGTAA
TAGATCAAGACATTGGCCATACCTTATTTAATGTTCCTACCACAGTTTTA
AGATATGGATTTCATGTGTTGCATTTTATAACCCATGCATTTGCTACTGA
TAGCGTGCAGTGTTACACGCCACATATGCAAATCCCCTATGATAATTTCT
ATGCTAGTGGTTGCGTGTTGTCATCCCTCTGTACTATGCTTGCGCATGCA
GATGGAACCCCGCATCCTTATTGTTATACAGGGGGTGTTATGCACAATGC
CTCTCTGTATAGTTCTTTGGCTCCTCATGTCCGTTATAACCTGGCTAGTT
CAAATGGTTATATACGTTTTCCCGAAGTGGTTAGTGAAGGCATTGTGCGT
GTTGTGCGCACTCGCTCTATGACCTACTGCAGGGTTGGTTTATGTGAGGA
GGCCGAGGAGGGTATCTGCTTTAATTTTAATCGTTCATGGGTATTGAACA
ACCCGTATTATAGGGCCATGCCTGGAACTTTTTGTGGTAGGAATGCTTTT
GATTTAATACATCAAGTTTTAGGAGGATTAGTGCGGCCTATTGATTTCTT
TGCCTTAACGGCGAGTTCAGTGGCTGGTGCTATCCTTGCAATTATTGTCG
TTTTGGCTTTCTATTATTTAATAAAGCTTAAACGTGCCTTTGGTGACTAC
ACTAGTGTTGTGGTTATCAATGTAATTGTGTGGTGTATAAATTTTCTGAT
GCTTTTTGTGTTTCAGGTTTATCCCACATTGTCTTGTTTATATGCTTGTT
TTTATTTCTACACAACGCTTTATTTCCCTTCGGAGATAAGTGTTGTTATG
CATTTGCAATGGCTTGTCATGTATGGTGCTATTATGCCCTTGTGGTTTTG
CATTATTTACGTGGCAGTCGTTGTTTCAAACCATGCATTGTGGTTGTTCT
CTTACTGCCGCAAAATTGGTACCGAGGTTCGTAGTGACGGCACATTTGAG
GAAATGGCCCTTACTACCTTTATGATTACTAAAGAATCTTATTGTAAGTT
GAAAAATTCTGTTTCTGATGTTGCTTTTAACAGGTACTTGAGTCTTTATA
ACAAGTATCGTTATTTTAGTGGCAAAATGGATACTGCCGCTTATAGAGAG
GCTGCCTGTTCACAACTGGCAAAGGCAATGGAAACATTTAACCATAATAA
TGGTAATGATGTTCTCTATCAGCCTCCAACCGCCTCTGTTACTACATCAT
TTTTACAGTCTGGTATAGTGAAGATGGTGTCGCCCACCTCTAAAGTGGAG
CCTTGTATTGTTAGTGTTACTTATGGTAACATGACACTTAATGGGTTGTG
GTTGGATGATAAAGTTTATTGCCCAAGACATGTTATCTGTTCTTCAGCTG
ACATGACAGACCCTGATTATCCTAATTTGCTTTGTAGAGTGACATCAAGT
GATTTTTGTGTTATGTCTGGTCGTATGAGCCTTACTGTAATGTCTTATCA
AATGCAGGGCTGCCAACTTGTTTTGACTGTTACACTGCAAAATCCTAACA
CGCCTAAGTATTCCTTCGGTGTTGTTAAGCCTGGTGAGACATTTACTGTA
CTGGCTGCATACAATGGCAGACCTCAAGGAGCCTTCCATGTTACGCTTCG
TAGTAGCCATACCATAAAGGGCTCCTTTCTATGTGGATCCTGCGGTTCTG
TAGGATATGTTTTAACTGGCGATAGTGTACGATTTGTTTATATGCATCAG
CTAGAGTTGAGTACTGGTTGTCATACCGGTACTGACTTTAGTGGGAACTT
TTATGGTCCCTATAGAGATGCGCAAGTTGTACAATTGCCTGTTCAGGATT
ATACGCAGACTGTTAATGTTGTAGCTTGGCTTTATGCTGCTATTTTTAAC
AGATGCAACTGGTTTGTGCAAAGTGATAGTTGTTCCCTGGAGGAGTTTAA
TGTTTGGGCTATGACCAATGGTTTTAGCTCAATCAAAGCCGATCTTGTCT
TGGATGCGCTTGCTTCTATGACAGGCGTTACAGTTGAACAGGTGTTGGCC
GCTATTAAGAGGCTGCATTCTGGATTCCAGGGCAAACAAATTTTAGGTAG
TTGTGTGCTTGAAGATGAGCTGACACCAAGTGATGTTTATCAACAACTAG
CTGGTGTCAAGCTACAGTCAAAGCGCACAAGAGTTATAAAAGGTACATGT
TGCTGGATATTGGCTTCAACGTTTTTGTTCTGTAGCATTATCTCAGCATT
TGTAAAATGGACTATGTTTATGTATGTTACTACCCATATGTTGGGAGTGA
CATTGTGTGCACTTTGTTTTGTAAGCTTTGCTATGTTGTTGATCAAGCAT
AAGCATTTGTATTTAACTATGTATATTATGCCTGTGTTATGCACACTGTT
TTACACCAACTATTTGGTTGTGTACAAACAGAGTTTTAGAGGTCTAGCTT
ATGCTTGGCTTTCACACTTTGTCCCTGCTGTAGATTATACATATATGGAT
GAAGTTTTATATGGTGTTGTGTTGCTAGTAGCTATGGTGTTTGTTACCAT
GCGTAGCATAAACCACGACGTCTTTTCTATTATGTTCTTGGTTGGTAGAC
TTGTCAGCCTGGTATCCATGTGGTATTTTGGAGCCAATTTAGAGGAAGAG
GTACTATTGTTCCTCACATCCCTATTTGGCACGTACACATGGACTACTAT
GTTGTCATTGGCTACCGCTAAGGTTATTGCTAAATGGTTGGCTGTGAATG
TCTTGTACTTCACAGACGTACCGCAAATTAAATTAGTTCTTTTGAGCTAC
TTGTGTATTGGTTATGTGTGTTGTTGTTATTGGGGAATCTTGTCACTCCT
TAATAGCATTTTTAGGATGCCATTGGGCGTCTACAATTATAAAATCTCCG
TTCAGGAGTTACGTTATATGAATGCTAATGGCTTGCGCCCACCTAGAAAT
AGTTTTGAGGCCCTGATGCTTAATTTTAAGCTGTTGGGAATTGGTGGTGT
GCCAGTCATTGAAGTATCTCAAATTCAATCAAGATTGACGGATGTTAAAT
GTGCTAATGTTGTGTTGCTTAATTGCCTCCAGCACTTGCATATTGCATCT
AATTCTAAGTTGTGGCAGTATTGTAGTACTTTGCACAATGAAATACTGGC
TACATCTGATTTGAGCGTGGCCTTCGATAAGTTGGCTCAGCTCTTAGTTG
TTTTATTTGCTAATCCAGCAGCAGTGGATAGCAAGTGCCTTGCAAGTATT
GAAGAAGTGAGCGATGATTACGTTCGCGACAATACTGTCTTGCAAGCCTT
ACAGAGTGAATTTGTTAATATGGCTAGCTTCGTTGAGTATGAACTTGCTA
AGAAGAATCTAGATGAGGCTAAGGCTAGCGGCTCTGCCAATCAACAGCAG
ATTAAGCAGCTAGAGAAGGCGTGTAATATTGCTAAGTCAGCATATGAGCG
CGACAGAGCTGTTGCTCGTAAGCTGGAACGTATGGCTGATTTAGCTCTTA
CAAACATGTATAAAGAAGCTAGAATTAATGATAAGAAGAGTAAGGTAGTG
TCTGCATTGCAAACCATGCTCTTTAGTATGGTGCGTAAGCTAGATAACCA
AGCTCTTAATTCTATTTTAGATAATGCAGTTAAGGGTTGTGTACCTTTGA
ATGCAATACCATCATTGACTTCGAACACTCTGACTATAATAGTGCCAGAT
AAGCAGGTTTTTGATCAGGTTGTGGATAATGTGTATGTCACCTATGCTGG
GAATGTATGGCATATACAGTTTATTCAAGATGCTGATGGTGCTGTTAAAC
AATTGAATGAGATAGATGTTAATTCAACCTGGCCTCTAGTCATTGCTGCA
AATAGGCATAATGAAGTGTCTACTGTTGTTTTGCAGAACAATGAGTTGAT
GCCTCAGAAGTTGAGAACTCAGGTTGTCAATAGTGGCTCAGATATGAATT
GTAATACTCCTACCCAGTGTTACTATAATACTACTGGCACGGGTAAGATT
GTGTATGCTATACTTAGTGACTGTGATGGTCTCAAGTACACTAAGATAGT
AAAAGAAGATGGAAATTGTGTTGTTTTGGAATTGGATCCTCCCTGTAAGT
TTTCTGTTCAGGATGTGAAGGGCCTTAAAATTAAGTACCTTTACTTTGTG
AAGGGGTGTAATACACTGGCTAGAGGCTGGGTTGTAGGCACCTTATCCTC
GACAGTGAGATTGCAGGCGGGTACGGCAACTGAGTATGCCTCCAACTCTG
CAATACTGTCGCTGTGTGCGTTTTCTGTAGATCCTAAGAAAACGTACTTG
GATTATATAAAACAGGGTGGAGTTCCCGTTACTAATTGTGTTAAGATGTT
ATGTGACCATGCTGGCACTGGTATGGCCATTACTATTAAGCCGGAGGCAA
CCACTAATCAGGATTCTTATGGTGGTGCTTCCGTTTGTATATATTGCCGC
TCGCGTGTTGAACATCCAGATGTTGATGGATTGTGCAAATTACGCGGCAA
GTTTGTCCAAGTGCCCTTAGGCATAAAAGATCCTGTGTCATATGTGTTGA
CGCATGATGTTTGTCAGGTTTGTGGCTTTTGGCGAGATGGTAGCTGTTCC
TGTGTAGGCACAGGCTCCCAGTTTCAGTCAAAAGACACGAACTTTTTAAA
CGGGTTCGGGGTACAAGTGTAAATGCCCGTCTTGTACCCTGTGCCAGTGG
CTTGGACACTGATGTTCAATTAAGGGCATTTGACATTTGTAATGCTAATC
GAGCTGGCATTGGTTTGTATTATAAAGTGAATTGCTGCCGCTTCCAGCGT
GTAGATGAGGACGGCAACAAGTTGGATAAGTTCTTTGTTGTTAAAAGAAC
TAATTTAGAAGTGTATAATAAGGAGAAAGAATGCTATGAGTTGACAAAAG
AATGCGGTGTTGTGGCTGAACACGAGTTCTTCACATTTGATGTGGAGGGA
AGTCGGGTACCACACATAGTCCGTAAAGATCTTTCAAAGTTTACTATGTT
AGATCTTTGCTATGCATTGCGTCATTTTGACCGCAATGATTGTTCAACTC
TTAAGGAAATTCTCCTTACATATGCTGAGTGTGAAGAGTCCTACTTCCAA
AAGAAGGACTGGTATGATTTTGTTGAGAATCCTGATATAATTAATGTGTA
TAAAAAGCTTGGTCCTATATTTAATAGAGCCCTGCTTAACACTGCCAAGT
TTGCAGACGCATTAGTGGAGGCAGGCTTAGTAGGTGTTTTAACACTTGAT
AATCAAGATTTATATGGTCAATGGTATGACTTTGGAGATTTTGTCAAGAC
AGTACCTGGTTGTGGTGTTGCCGTGGCAGACTCTTATTATTCATATATGA
TGCCAATGCTGACTATGTGTCATGCGTTGGATAGTGAGTTGTTTGTTAAT
GGTACTTATAGGGAGTTTGACCTTGTTCAGTATGATTTTACTGATTTCAA
GCTAGAGCTCTTCACTAAGTATTTTAAGCATTGGAGTATGACCTACCACC
CGAACACCTGTGAGTGCGAGGATGACAGGTGCATTATTCATTGCGCCAAT
TTTAATATACTTTTTAGTATGGTCTTACCTAAGACCTGTTTTGGGCCTCT
TGTTAGGCAGATATTTGTGGATGGTGTTCCTTTCGTTGTGTCGATCGGTT
ACCATTATAAAGAATTAGGTGTTGTTATGAATATGGATGTGGATACACAT
CGTTATCGCTTGTCTCTTAAGGACTTGCTTTTGTATGCTGCAGACCCTGC
CCTTCATGTGGCGTCTGCTAGTGCACTGCTTGATTTGCGCACATGTTGTT
TTAGCGTTGCAGCTATTACAAGTGGCGTAAAATTTCAAACAGTTAAACCT
GGAAATTTTAATCAGGATTTTTATGAGTTTATTTTGAGTAAAGGCCTGCT
TAAAGAGGGGAGCTCCGTTGATTTGAAGCACTTCTTCTTTACGCAGGATG
GTAATGCTGCTATTACTGATTATAATTATTACAAGTATAATCTACCCACC
ATGGTGGATATTAAGCAGTTGTTGTTTGTTTTAGAAGTTGTTAATAAGTA
TTTTGAGATCTATGAGGGTGGGTGTATACCCGCAACACAGGTCATTGTTA
ATAATTATGATAAGAGTGCTGGCTATCCATTTAATAAATTTGGAAAGGCC
AGGCTCTATTATGAGGCATTATCATTTGAGGAGCAGGATGAAATTTATGC
GTATACCAAACGCAATGTCCTGCCGACCCTAACTCAAATGAATCTTAAAT
ATGCTATTAGTGCTAAGAATAGGGCCCGCACCGTTGCTGGTGTCTCTATT
CTCAGTACTATGACTGGCAGAATGTTTCATCAAAAGTGTCTAAAGAGTAT
AGCAGCTACTCGCGGTGTTCCTGTAGTTATAGGCACCACGAAGTTCTATG
GCGGTTGGGATGATATGTTACGCCGCCTTATTAAAGATGTTGATAGTCCT
GTACTCATGGGTTGGGACTATCCTAAATGTGATCGTGCTATGCCAAACAT
ACTGCGTATTGTTAGTAGTTTGGTGCTAGCCCGTAAACATGATTCGTGCT
GTTCGCATACGGATAGATTCTATCGTCTTGCGAACGAGTGCGCCCAAGTT
TTGAGTGAAATTGTTATGTGTGGTGGTTGTTATTATGTTAAACCAGGTGG
CACTAGTAGTGGGGATGCAACCACTGCTTTTGCTAATTCTGTGTTTAACA
TTTGTCAAGCTGTTTCCGCCAATGTATGCTCGCTTATGGCATGCAATGGA
CACAAAATTGAAGATTTGAGTATACGCGAGTTACAAAAGCGCCTATACTC
TAATGTCTATCGTGCGGACCATGTTGACCCCGCATTTGTTAGTGAGTATT
ATGAGTTTTTAAATAAGCATTTTAGTATGATGATTTTGAGTGATGATGGT
GTTGTGTGTTATAATTCAGAGTTTGCGTCCAAGGGTTATATTGCTAATAT
AAGTGCCTTTCAACAGGTATTATATTATCAAAATAATGTGTTTATGTCTG
AGGCCAAATGTTGGGTAGAAACAGACATCGAAAAGGGACCGCATGAATTT
TGTTCTCAACATACAATGCTAGTCAAGATGGATGGTGATGAAGTCTACCT
TCCATACCCTGATCCTTCGAGAATCTTAGGAGCAGGCTGTTTTGTTGATG
ATTTATTAAAGACTGATAGCGTTCTCTTGATAGAGCGTTTCGTAAGTCTT
GCAATTGATGCTTATCCTTTAGTATACCATGAGAACCCAGAGTATCAAAA
TGTGTTCCGGGTATATTTAGAATATATAAAGAAGCTGTACAATGATCTCG
GTAATCAGATCCTGGACAGCTACAGTGTTATTTTAAGTACTTGTGATGGT
CAAAAGTTTACTGATGAGACCTTTTACAAGAACATGTATTTAAGAAGTGC
AGTGCTGCAAAGCGTTGGTGCCTGCGTTGTCTGTAGTTCTCAAACATCAT
TACGTTGTGGCAGTTGCATACGCAAGCCTTTGCTGTGTTGCAAATGCGCC
TATGATCATGTTATGTCCACTGATCATAAATATGTCCTGAGTGTGTCACC
ATATGTGTGTAATTCACCGGGATGTGATGTAAATGATGTTACCAAATTGT
ATTTAGGTGGTATGTCATATTATTGTGAGGACCATAAACCACAGTATTCA
TTCAAATTGGTGATGAATGGTATGGTTTTTGGTTTATATAAACAATCTTG
TACTGGTTCGCCCTACATAGAGGATTTTAATAAAATAGCTAGTTGCAAAT
GGACAGAAGTCGATGATTATGTGCTAGCTAATGAATGCACCGAACGCCTT
AAATTGTTTGCCGCAGAAACGCAGAAGGCCACAGAAGAGGCCTTTAAGCA
ATGTTATGCGTCAGCAACGATCCGTGAGATCGTGAGCGATCGGGAGTTAA
TTTTATCTTGGGAAATTGGTAAAGTGAGACCACCACTTAATAAAAATTAT
GTTTTTACTGGCTACCATTTTACTAATAATGGTAAGACAGTTTTAGGTGA
GTATGTTTTTGATAAGAGTGAGTTGACTAATGGTGTGTACTATCGCGCCA
CAACCACTTATAAGTTATCTGTAGGTGATGTGTTCATTTTAACATCACAC
GCAGTGTCTAGTTTAAGTGCTCCTACATTAGTACCGCAGGAGAATTATAC
TAGCATTCGTTTTGCTAGTGTTTATAGTGTGCCTGAGACGTTTCAGAATA
ATGTGCCTAATTATCAGCACATTGGAATGAAGCGCTATTGTACTGTACAG
GGACCGCCTGGTACTGGTAAGTCCCATCTAGCCATTGGGCTAGCTGTTTA
TTATTGTACAGCGCGCGTGGTGTATACCGCTGCTAGCCATGCTGCAGTTG
ACGCGCTGTGTGAAAAGGCACATAAATTTTTAAATATTAATGACTGCACG
CGTATTGTTCCTGCAAAGGTGCGTGTAGATTGTTATGATAAATTTAAGGT
CAATGACACCACTCGCAAGTATGTGTTTACTACAATAAATGCATTACCTG
AGTTGGTGACTGACATTATTGTCGTTGATGAAGTTAGTATGCTTACCAAC
TATGAGCTGTCTGTTATTAACAGTCGTGTTAGTGCTAAGCATTATGTGTA
TATTGGAGACCCTGCGCAGTTACCTGCACCACGTGTGCTACTGAATAAGG
GAACTCTAGAACCTAGATATTTTAATTCCGTTACCAAGCTAATGTGTTGT
TTGGGTCCAGATATTTTCTTGGGCACCTGTTATAGATGCCCTAAGGAGAT
TGTGGATACGGTGTCAGCCTTGGTTTATAATAATAAGCTGAAGGCTAAAA
ATGATAATAGCTCCATGTGCTTTAAGGTTTATTATAAGGGCCAGACTACA
CATGAGAGTTCTAGTGCTGTTAATATGCAGCAAATACATTTAATTAGTAA
GTTTTTAAAGGCAAACCCCAGTTGGAGTAACGCCGTATTTATTAGTCCTT
ATAATAGTCAGAACTATGTTGCTAAGAGAGTCTTGGGATTACAAACCCAG
ACAGTAGACTCAGCGCAGGGTTCTGAATATGATTTTGTTATTTATTCACA
GACTGCGGAAACAGCGCATTCTGTCAATGTAAATAGATTCAATGTTGCTA
TTACACGTGCTAAGAAGGGTATTCTCTGTGTCATGAGTAGTATGCAATTA
TTTGAGTCTCTTAATTTTACTACACTGACGTTGGATAAGATTAACAATCC
ACGATTACAGTGTACTACAAATTTGTTTAAGGATTGTAGCAGGAGCTATG
TAGGATATCACCCAGCCCATGCACCATCCTTTTTGGCAGTTGATGACAAA
TATAAGGTAGGCGGTGATTTAGCCGTTTGCCTTAATGTTGCTGATTCTGC
TGTCACTTATTCGCGGCTTATATCACTCATGGGATTCAAGCTTGACTTGA
CCCTTGATGGTTATTGTAAGCTGTTTATAACTAGAGATGAAGCTATCAAA
CGTGTTAGAGCCTGGGTTGGCTTCGATGCAGAAGGTGCCCATGCGATACG
TGATAGCATTGGGACAAATTTCCCATTACAATTAGGCTTTTCGACTGGAA
TTGATTTTGTTGTCGAAGCCACTGGAATGTTTGCTGAGAGAGATGGTTAT
GTCTTTAAAAAGGCAGCCGCACGAGCTCCTCCTGGCGAACAATTTAAACA
CCTTATCCCACTTATGTCAAGAGGGCAGAAATGGGATGTGGTTCGAATTA
GAATAGTACAAATGTTGTCAGACCACCTAGCGGATTTGGCAGACAGTGTT
GTACTTGTGACGTGGGCTGCCAGCTTTGAGCTCACATGTTTGCGATATTT
CGCTAAAGTTGGAAGAGAAGTTGTGTGTAGTGTCTGCACCAAGCGTGCGA
CATGTTTTAATTCTAGAACTGGATACTATGGATGCTGGCGACATAGTTAT
TCCTGTGATTACCTGTACAACCCACTAATAGTTGACATTCAACAGTGGGG
ATATACAGGATCTTTAACTAGCAATCATGATCCTATTTGCAGCGTGCATA
AGGGTGCTCATGTTGCATCATCTGATGCTATCATGACCCGGTGTCTAGCT
GTTCATGATTGCTTTTGTAAGTCTGTTAATTGGAATTTAGAATACCCCAT
TATTTCAAATGAGGTCAGTGTTAATACCTCCTGCAGGTTATTGCAGCGCG
TAATGTTTAGGGCTGCGATGCTATGCAATAGGTATGATGTGTGTTATGAC
ATTGGCAACCCTAAAGGTCTTGCCTGTGTCAAAGGATATGATTTTAAGTT
TTATGATGCCTCCCCTGTTGTTAAGTCTGTTAAACAGTTTGTTTATAAAT
ACGAGGCACATAAAGATCAATTTTTAGATGGTTTGTGTATGTTTTGGAAC
TGCAATGTGGATAAGTATCCAGCGAATGCAGTTGTGTGTAGGTTTGACAC
GCGTGTGTTGAACAAATTAAATCTCCCTGGCTGTAATGGTGGCAGTTTGT
ATGTTAACAAACATGCATTCCACACCAGTCCCTTTACCCGGGCTGCCTTC
GAGAATTTGAAGCCTATGCCTTTCTTTTATTATTCAGATACGCCCTGTGT
GTATATGGAAGGCATGGAATCTAAGCAGGTCGATTATGTCCCATTGAGAA
GCGCTACATGCATCACAAGATGCAATTTAGGTGGCGCTGTTTGTTTAAAA
CATGCTGAGGAGTATCGTGAGTACCTTGAGTCTTACAATACGGCAACCAC
AGCGGGTTTTACTTTTTGGGTCTATAAGACTTTTGATTTTTATAACCTTT
GGAATACTTTTACTAGGCTCCAAAGTTTAGAAAATGTAGTGTATAATTTG
GTCAATGCTGGACACTTTGATGGCCGGGCGGGTGAACTGCCTTGTGCTGT
TATAGGTGAGAAAGTCATTGCCAAGATTCAAAATGAGGATGTCGTGGTCT
TTAAAAATAACACGCCATTCCCCACTAATGTGGCTGTCGAATTATTTGCT
AAGCGCAGTATTCGGCCCCACCCCGAGCTTAAGCTCTTTAGAAATTTGAA
TATTGACGTGTGCTGGAGTCACGTCCTTTGGGATTATGCTAAGGATAGTG
TGTTTTGCAGTTCGACGTATAAGGTCTGCAAATACACAGATTTACAGTGC
ATTGAAAGCTTGAATGTACTTTTTGATGGTCGTGATAATGGTGCTCTTGA
AGCTTTTAAGAAGTGCCGGAATGGCGTCTACATTAACACGACAAAAATTA
AAAGTCTGTCGATGATTAAAGGCCCACAACGTGCCGATTTGAATGGCGTA
GTTGTGGAGAAAGTTGGAGATTCTGATGTGGAATTTTGGTTTGCTGTGCG
TAAAGACGGTGACGATGTTATCTTCAGCCGTACAGGGAGCCTTGAACCGA
GCCATTACCGGAGCCCACAAGGTAATCCGGGTGGTAATCGCGTGGGTGAT
CTCAGCGGTAATGAAGCTCTAGCGCGTGGCACTATCTTTACTCAAAGCAG
ATTATTATCTTCTTTCACACCTCGATCAGAGATGGAGAAAGATTTTATGG
ATTTAGATGATGATGTGTTCATTGCAAAATATAGTTTACAGGACTACGCG
TTTGAACACGTTGTTTATGGTAGTTTTAACCAGAAGATTATTGGAGGTTT
GCATTTGCTTATTGGCTTAGCCCGTAGGCAGCAAAAATCCAATCTGGTAA
TTCAAGAGTTCGTGACATACGACTCTAGCATTCATTCGTACTTTATCACT
GACGAGAACAGTGGTAGTAGTAAGAGTGTGTGCACTGTTATTGATTTATT
GTTAGATGATTTTGTGGACATTGTAAAGTCCCTGAATCTAAAGTGTGTGA
GTAAGGTTGTTAATGTTAATGTTGATTTTAAAGATTTCCAGTTTATGTTG
TGGTGCAATGAGGAGAAGGTCATGACTTTCTATCCTCGTTTGCAGGCTGC
TGCTGACTGGAAACCTGGTTATGTTATGCCTGTCTTATATAAGTATTTGG
AATCGCCTCTGGAAAGAGTAAACCTCTGGAATTATGGCAAGCCGATTACT
TTACCTACAGGATGTATGATGAATGTTGCTAAGTATACTCAATTATGTCA
ATATTTGAGCACTACAACATTAGCAGTTCCGGCTAATATGCGTGTCTTAC
ACCTTGGTGCCGGTTCGGATAAGGGTGTTGCCCCTGGGTCTGCAGTTCTT
AGGCAGTGGCTACCAGCGGGAAGTATTCTTGTAGATAATGATGTGAATCC
ATTTGTGAGTGACAGTGTCGCCTCATATTATGGAAATTGTATAACCTTAC
CCTTTGATTGTCAGTGGGATCTGATAATTTCTGATATGTACGACCCTCTT
ACTAAGAACATTGGGGAGTACAACGTGAGTAAAGATGGATTCTTTACTTA
CCTCTGTCATTTAATTCGTGACAAGTTGGCTCTGGGTGGCAGTGTTGCCA
TAAAAATAACAGAGTTTTCTTGGAACGCTGAGTTATATAGTTTAATGGGG
AAGTTTGCGTTCTGGACAATCTTTTGCACCAACGTAAACGCCTCTTCAAG
TGAAGGATTTTTGATTGGCATAAATTGGTTGAATAAGACCCGTACCGAAA
TTGACGGTAAAACCATGCATGCCAATTATCTGTTTTGGAGAAATAGTACA
ATGTGGAATGGAGGGGCTTACAGTCTCTTTGACATGAGTAAGTTCCCTTT
GAAAGCGGCTGGTACGGCTGTTGTTAGCCTTAAACCAGACCAAATAAATG
ACTTAGTCCTCTCCTTGATTGAGAAGGGCAAGTTATTAGTGCGTGATACA
CGCAAAGAAGTTTTTGTTGGCGATAGCCTAGTAAATGTCAAATAAATCTA
TACTTGTCGTGGCTGTGAAAATGGCCTTTGCTGACAAGCCTAATCATTTC
ATAAACTTTCCCCTGGCCCAATTTAGTGGCTTTATGGGTAAGTATTTAAA
GCTACAGTCTCAACTTGTGGAAATGGGTTTAGACTGTAAATTACAGAAGG
CACCACATGTTAGTATTACCCTGCTTGATATTAAAGCAGACCAATACAAA
CAGGTGGAATTTGCAATACAAGAAATAATAGATGATCTGGCGGCATATGA
GGGAGATATTGTCTTTGACAACCCTCACATGCTTGGCAGATGCCTTGTTC
TTGATGTTAGAGGATTTGAAGAGTTGCATGAAGATATTGTTGAAATTCTC
CGCAGAAGGGGTTGCACGGCAGATCAATCCAGACACTGGATTCCGCACTG
CACTGTGGCCCAATTTGACGAAGAAAGAGAAACAAAAGGAATGCAATTCT
ATCATAAAGAACCCTTCTACCTCAAGCATAACAACCTATTAACGGATGCT
GGGCTTGAGCTCGTGAAGATAGGTTCTTCCAAAATAGATGGGTTTTATTG
TAGTGAACTGAGTGTTTGGTGTGGTGAGAGGCTTTGTTATAAGCCTCCAA
CACCCAAATTCAGTGATATATTTGGCTATTGCTGCATAGATAAAATACGT
GGTGATTTAGAAATAGGAGACCTACCGCAGGATGATGAGGAAGCGTGGGC
CGAGCTAAGTTACCACTATCAAAGAAACACCTACTTCTTCAGACATGTGC
ACGATAATAGCATCTATTTTCGTACCGTGTGTAGAATGAAGGGTTGTATG
TGTTGATTTGTTTTTACACTATTAGTGTAATAAGCTTATTATTTTGTTGA
AAAGGGCAGGATGTGCATAGCTATGGCTCCTCGCACACTGCTTTTGCTGA
TTTGATGTCAGCTGGTGTTTGGGTTCAATGAACCTCTTAACATCGTTTCA
CATTTAAATGATGACTGGTTTCTATTTGGTGACAGTCGTTCTGACTGTAC
CTATGTAGAAAATAACGGTCATCCTAAATTAGATTGGCTTGACCTCGACC
CAAAGTTGTGTAATTCAGGAAAGATTTCCGCAAAGAGTGGTAACTCTCTC
TTTAGGAGTTTTCACTTCACTGATTTTTACAATTATACGGGTGAGGGAGA
CCAAATTGTATTTTATGAAGGAGTTAATTTTAGTCCCAGCCATGGCTTTA
AATGCCTGGCTCATGGAGATAATAAAAGATGGATGGGCAATAAAGCTCGA
TTTTATGCCCGAGTGTATGAGAAGATGGCCCAATATAGGAGCCTATCGTT
TGTTAATGTGTCTTATGCCTATGGAGGTAATGCAAAGCCCGCCTCCATTT
GCAAAGACAATACTTTAACACTCAATAACCCCACCTTCATATCGAAGGAG
TCTAATTATGTTGATTATTACTATGAGAGTGAGGCTAATTTCACACTAGA
AGGTTGTGATGAATTTATAGTACCGCTCTGTGGTTTTAATGGCCATTCCA
AGGGCAGCTCTTCGGATGCTGCCAATAAATATTATACTGACTCTCAGAGT
TACTATAATATGGATATTGGTGTCTTATATGGGTTCAATTCGACCTTGGA
TGTTGGCAACACTGCTAAGGATCCGGGTCTTGATCTCACTTGCAGGTATC
TTGCATTGACTCCTGGTAATTATAAGGCTGTGTCCTTAGAATATTTGTTA
AGCTTACCCTCAAAGGCTATTTGCCTCCATAAGACAAAGCGCTTTATGCC
TGTGCAGGTAGTTGACTCAAGGTGGAGTAGCATCCGCCAGTCAGACAATA
TGACCGCTGCAGCCTGTCAGCTGCCATATTGTTTCTTTCGCAACACATCT
GCGAATTATAGTGGTGGCACACATGATGCGCACCATGGTGATTTTCATTT
CAGGCAGTTATTGTCTGGTTTGTTATATAATGTTTCCTGTATTGCCCAGC
AGGGTGCATTTCTTTATAATAATGTTAGTTCCTCTTGGCCAGCCTATGGG
TACGGTCATTGTCCAACGGCAGCTAACATTGGTTATATGGCACCTGTTTG
TATCTATGACCCTCTCCCGGTCATACTGCTAGGTGTGTTATTGGGTATAG
CTGTGTTGATTATTGTGTTTTTGATGTTTTATTTTATGACGGATAGCGGT
GTTAGATTGCATGAGGCATAATCTAAACATGCTGTTCGTGTTTATTCTAT
TTTTGCCCTCTTGTTTAGGGTATATTGGTGATTTTAGATGTATCCAGCTT
GTGAATTCAAACGGTGCTAATGTTAGTGCTCCAAGCATTAGCACTGAGAC
CGTTGAAGTTTCACAAGGCCTGGGGACATATTATGTGTTAGATCGAGTTT
ATTTAAATGCCACATTATTGCTTACTGGTTACTACCCGGTCGATGGTTCT
AAGTTTAGAAACCTCGCTCTTACGGGAACTAACTCAGTTAGCTTGTCGTG
GTTTCAACCACCCTATTTAAGTCAGTTTAATGATGGCATATTTGCGAAGG
TGCAGAACCTTAAGACAAGTACGCCATCAGGTGCAACTGCATATTTTCCT
ACTATAGTTATAGGTAGTTTGTTTGGCTATACTTCCTATACCGTTGTAAT
AGAGCCATATAATGGTGTTATAATGGCCTCAGTGTGCCAGTATACCATTT
GTCTGTTACCTTACACTGATTGTAAGCCTAACACTAATGGTAATAAGCTT
ATAGGGTTTTGGCACACGGATGTAAAACCCCCAATTTGTGTGTTAAAGCG
AAATTTCACGCTTAATGTTAATGCTGATGCATTTTATTTTCATTTTTACC
AACATGGTGGTACTTTTTATGCGTACTATGCGGATAAACCCTCCGCTACT
ACGTTTTTGTTTAGTGTATATATTGGCGATATTTTAACACAGTATTATGT
GTTACCTTTCATCTGCAACCCAACAGCTGGTAGCACTTTTGCTCCGCGCT
ATTGGGTTACACCTTTGGTTAAGCGCCAATATTTGTTTAATTTCAACCAG
AAGGGTGTCATTACTAGTGCTGTTGATTGTGCTAGTAGTTATACCAGTGA
AATAAAATGTAAGACCCAGAGCATGTTACCTAGCACTGGTGTCTATGAGT
TATCCGGTTATACGGTCCAACCAGTTGGAGTTGTATACCGGCGTGTTGCT
AACCTCCCAGCTTGTAATATAGAGGAGTGGCTTACTGCTAGGTCAGTCCC
CTCCCCTCTCAACTGGGAGCGTAAGACTTTTCAGAATTGTAATTTTAATT
TAAGCAGCCTGTTACGTTATGTTCAGGCTGAGAGTTTGTTTTGTAATAAT
ATCGATGCTTCCAAAGTGTATGGCAGGTGCTTTGGTAGTATTTCAGTTGA
TAAGTTTGCTGTACCCCGAAGTAGGCAAGTTGATTTACAGCTTGGTAACT
CTGGATTTCTGCAGACTGCTAATTATAAGATTGATACAGCTGCCACTTCG
TGTCAGCTGCATTACACCTTGCCTAAGAATAATGTCACCATAAACAACCA
TAACCCCTCGTCTTGGAATAGGAGGTATGGCTTTAATGATGCTGGCGTCT
TTGGCAAAAACCAACATGACGTTGTTTACGCTCAGCAATGTTTTACTGTA
AGATCTAGTTATTGCCCGTGTGCTCAACCGGACATAGTTAGCCCTTGCAC
TACTCAGACTAAGCCTAAGTCTGCTTTTGTTAATGTGGGTGACCATTGTG
AAGGCTTAGGTGTTTTAGAAGATAATTGTGGCAATGCTGATCCACATAAG
GGTTGTATCTGTGCCAACAATTCATTTATTGGATGGTCACATGATACCTG
CCTTGTTAATGATCGCTGCCAAATTTTTGCTAATATATTGTTAAATGGCA
TTAATAGTGGTACCACATGTTCCACAGATTTGCAGTTGCCTAATACTGAA
GTGGTTACTGGCATTTGTGTCAAATATGACCTCTACGGTATTACTGGACA
AGGTGTTTTTAAAGAGGTTAAGGCTGACTATTATAATAGCTGGCAAACCC
TTCTGTATGATGTTAATGGTAATTTGAATGGTTTTCGTGATCTTACCACT
AACAAGACTTATACGATAAGGAGCTGTTATAGTGGCCGTGTTTCTGCTGC
ATTTCATAAAGATGCACCCGAACCGGCTCTGCTCTATCGTAATATAAATT
GTAGCTATGTTTTTAGCAATAATATTTCCCGTGAGGAGAACCCACTTAAT
TACTTTGATAGTTATTTGGGTTGTGTTGTTAATGCTGATAACCGCACGGA
TGAGGCGCTTCCTAATTGTGATCTCCGTATGGGTGCTGGCTTATGCGTTG
ATTATTCAAAATCACGCAGGGCTGACCGATCAGTTTCTACTGGCTATCGG
TTAACTACATTTGAGCCATACACTCCGATGTTAGTTAATGATAGTGTCCA
ATCCGTTGATGGATTATATGAGATGCAAATACCAACCAATTTTACTATTG
GGCACCATGAGGAGTTCATTCAAACTAGATCTCCAAAGGTGACTATAGAT
TGTGCTGCATTTGTCTGTGGTGATAACACTGCATGCAGGCAGCAGTTGGT
TGAGTATGGCTCTTTCTGTGTTAATGTTAATGCCATTCTTAATGAGGTTA
ATAACCTCTTGGATAATATGCAACTACAAGTTGCTAGTGCATTAATGCAG
GGTGTTACTATAAGCTCGAGACTGCCAGACGGCATCTCAGGCCCTATAGA
TGACATTAATTTTAGTCCTCTACTTGGATGCATAGGTTCAACATGTGCTG
AAGACGGCAATGGACCTAGTGCAATCCGAGGGCGTTCTGCTATAGAGGAT
TTGTTATTTGACAAGGTCAAATTATCTGATGTTGGCTTTGTCGAGGCTTA
TAATAATTGCACCGGTGGTCAAGAAGTTCGTGACCTCCTTTGTGTACAAT
CTTTTAATGGCATCAAAGTATTACCTCCTGTGTTGTCAGAGAGTCAGATC
TCTGGCTACACAACCGGTGCTACTGCGGCAGCTATGTTCCCACCGTGGTC
AGCAGCTGCCGGTGTGCCATTTAGTTTAAGTGTTCAATATAGAATTAATG
GTTTAGGTGTCACTATGAATGTGCTTAGTGAGAACCAAAAGATGATTGCT
AGTGCTTTTAACAATGCGCTGGGTGCTATCCAGGATGGGTTTGATGCAAC
CAATTCTGCTTTAGGTAAGATCCAGTCCGTTGTTAATGCAAATGCTGAAG
CACTCAATAACTTACTAAATCAACTTTCTAACAGGTTTGGTGCTATTAGT
GCTTCTTTACAAGAAATTCTAACTCGGCTTGAGGCTGTAGAAGCAAAAGC
CCAGATAGATCGTCTTATTAATGGCAGGTTAACTGCACTTAATGCGTATA
TATCCAAGCAACTTAGTGATAGTACGCTTATTAAAGTTAGTGCTGCTCAG
GCCATAGAAAAGGTCAATGAGTGCGTTAAGAGCCAAACCACGCGTATTAA
TTTCTGTGGCAATGGTAATCATATATTATCTCTTGTCCAGAATGCGCCTT
ATGGCTTATATTTTATACACTTCAGCTATGTGCCAATATCCTTTACAACC
GCAAATGTGAGTCCTGGACTTTGCATTTCTGGTGATAGAGGATTAGCACC
TAAAGCTGGATATTTTGTTCAAGATGATGGAGAATGGAAGTTCACAGGCA
GTTCATATTACTACCCTGAACCCATTACAGATAAAAACAGTGTCATTATG
AGTAGTTGCGCAGTAAACTACACAAAGGCACCTGAAGTTTTCTTGAACAC
TTCAATACCTAATCCACCCGACTTTAAGGAGGAGTTAGATAAATGGTTTA
AGAATCAGACGTCTATTGCGCCTGATTTATCTCTCGATTTCGAGAAGTTA
AATGTTACTTTGCTGGACCTGACGTATGAGATGAACAGGATTCAGGATGC
AATTAAGAAGTTAAATGAGAGCTACATCAACCTCAAGGAAGTTGGCACAT
ATGAAATGTATGTGAAATGGCCTTGGTATGTTTGGTTGCTAATTGGATTA
GCTGGTGTAGCTGTTTGTGTGTTGTTATTCTTTATATGTTGCTGCACAGG
TTGTGGCTCATGTTGTTTTAAGAAGTGTGGAAATTGTTGTGATGAGTATG
GAGGACACCAGGACAGTATTGTGATACATAATATTTCCTCTCATGAGGAT
TGACTATCACAGCCTCTCCTGGAAAGACAGAAAATCTAAACAATTTATAG
CATTCTCATTGCTACCTGGCCCCGTAAGAGGCAGTCATAGCTATGGCCGT
GTTGGTCCTAAGGCTACATTGGCTGCTGTCTTTATTGGTCCATTTATTGT
AGCATGTATGCTAGGCATTGGCCTAGTTTATTTATTGCAATTGCAAGTTC
AAATTTTTCATGTTAAGGATACCATACGTGTGACTGGCAAGCCAGCCACT
GTGTCTTATACTACAAGTACACCAGTAACACCGAGCGCGACGACGCTCGA
TGGTACTACGTATACTTTAATTAGACCCACTAGCTCTTATACAAGAGTTT
ATCTTGGTACTCCAAGAGGTTTTGATTATAGTACATTTGGGCCTAAGACC
CTAGATTATGTTACTAATCTAAACCTCATCTTAATTCTGGTCGTCCATAT
ACTTTTAAGGCATTGTCCAGGCATATGAGACCAACAGCCACATGGATTTG
GCATGTGAGTGATGCATGGTTACGCCGCACGCGGGACTTTGGTGTCATTC
GCCTAGAAGATTTTTGTTTTCAATTTAATTATAGCCAACCCCGAGTTGGT
TATTGTAGAGTTCCTTTAAAGGCTTGGTGTAGCAACCAGGGTAAATTTGC
AGCGCAGTTTACCCTAAAAAGTTGCGAAAAACCAGGTCACGAAAAATTTA
TTACTAGCTTCACGGCCTACGGCAGAACTGTCCAACAGGCCGTTAGCAAG
TTAGTAGAAGAAGCTGTTGATTTTATTCTTTTTAGGGCCACGCAGCTCGA
AAGAAATGTTTAATTTATTCCTTACAGACACAGTATGGTATGTGGGGCAG
ATTATTTTTATATTCGCAGTGTGTTTGATGGTCACCATAATTGTGGTTGC
CTTCCTTGCGTCTATCAAACTTTGTATTCAACTTTGCGGTTTATGTAATA
CTTTGGTGCTGTCCCCTTCTATTTATTTGTATGATAGGAGTAAGCAGCTT
TATAAGTATTATAATGAAGAAATGAGACTGCCCCTATTAGAGGTGGATGA
TATCTAATCTAAACATTATGAGTAGTACTACTCAGGCCCCAGAGCCCGTC
TATCAATGGACGGCCGACGAGGCAGTTCAATTCCTTAAGGAATGGAACTT
CTCGTTGGGCATTATACTACTCTTTATTACTATCATACTACAGTTCGGTT
ACACGAGCCGTAGCATGTTTATTTATGTTGTGAAAATGATAATCTTGTGG
TTAATGTGGCCACTGACTATTGTTTTGTGTATTTTCAATTGCGTGTATGC
GCTAAATAATGTGTATCTTGGATTTTCTATAGTGTTTACTATAGTGTCCA
TTGTAATCTGGATTATGTATTTTGTTAATAGCATAAGGTTGTTTATCAGG
ACTGGTAGCTGGTGGAGCTTCAACCCCGAAACAAACAACCTTATGTGTAT
AGATATGAAAGGTACCGTGTATGTTAGACCCATTATTGAGGATTACCATA
CACTAACAGCCACTATTATTCGTGGCCACCTCTACATGCAAGGTGTTAAG
CTAGGCACCGGTTTCTCTTTGTCTGACTTGCCCGCTTATGTTACAGTTGC
TAAGGTGTCACACCTTTGCACTTATAAGCGCGCATTCTTAGACAAGGTAG
ACGGTGTTAGCGGTTTTGCTGTTTATGTGAAGTCCAAGGTCGGAAATTAC
CGACTGCCCTCAAACAAACCGAGTGGCGCGGACACCGCATTGTTGAGAAT
CTAATCTAAACTTTAAGGATGTCTTTTGTTCCTGGGCAAGAAAATGCCGG
TGGCAGAAGCTCCTCTGTAAACCGCGCTGGTAATGGAATCCTCAAGAAGA
CCACTTGGGCTGACCAAACCGAGCGTGGACCAAATAATCAAAATAGAGGC
AGAAGGAATCAGCCAAAGCAGACTGCAACTACTCAACCCAACTCCGGGAG
TGTGGTTCCCCATTACTCCTGGTTTTCTGGCATTACCCAGTTCCAAAAGG
GAAAGGAGTTTCAGTTTGCAGAAGGACAAGGAGTGCCTATTGCCAATGGA
ATCCCCGCTTCAGAGCAAAAGGGATATTGGTATAGACACAACCGCCGTTC
TTTTAAAACACCTGATGGGCAGCAGAAGCAATTACTGCCCAGATGGTATT
TTTACTATCTTGGCACAGGGCCCCATGCTGGAGCCAGTTATGGAGACAGC
ATTGAAGGTGTCTTCTGGGTTGCAAACAGCCAAGCGGACACCAATACCCG
CTCTGATATTGTCGAAAGGGACCCAAGCAGTCATGAGGCTATTCCTACTA
GGTTTGCGCCCGGCACGGTATTGCCTCAGGGCTTTTATGTTGAAGGCTCT
GGAAGGTCTGCACCTGCTAGCCGATCTGGTTCGCGGTCACAATCCCGTGG
GCCAAATAATCGCGCTAGAAGCAGTTCCAACCAGCGCCAGCCTGCCTCTA
CTGTAAAACCTGATATGGCCGAAGAAATTGCTGCTCTTGTTTTGGCTAAG
CTCGGTAAAGATGCCGGCCAGCCCAAGCAAGTAACGAAGCAAAGTGCCAA
AGAAGTCAGGCAGAAAATTTTAAACAAGCCTCGCCAAAAGAGGACTCCAA
ACAAGCAGTGCCCAGTGCAGCAGTGTTTTGGAAAGAGAGGCCCCAATCAG
AATTTTGGAGGCTCTGAAATGTTAAAACTTGGAACTAGTGATCCACAGTT
CCCCATTCTTGCAGAGTTGGCTCCAACAGTTGGTGCCTTCTTCTTTGGAT
CTAAATTAGAATTGGTCAAAAAGAATTCTGGTGGTGCTGATGAACCCACC
AAAGATGTGTATGAGCTGCAATATTCAGGTGCAGTTAGATTTGATAGTAC
TCTACCTGGTTTTGAGACTATCATGAAAGTGTTGAATGAGAATTTGAATG
CCTACCAGAAGGATGGTGGTGCAGATGTGGTGAGCCCAAAGCCCCAAAGA
AAAGGGCGTAGACAGGCTCAGGAAAAGAAAGATGAAGTAGATAATGTAAG
CGTTGCAAAGCCCAAAAGCTCTGTGCAGCGAAATGTAAGTAGAGAATTAA
CCCCAGAGGATAGAAGTCTGTTGGCTCAGATCCTTGATGATGGCGTAGTG
CCAGATGGGTTAGAAGATGACTCTAATGTGTAAAGAGAATGAATCCTATG
TCGGCGCTCGGTGGTAACCCCTCGCGAGAAAGTCGGGATAGGACACTCTC
TATCAGAATGGATGTCTTGCTGTCATAACAGATAGAGAAGGTTGTGGCAG
ACCCTGTATCAATTAGTTGAAAGAGATTGCAAAATAGAGAATGTGTGAGA
GAAGTTAGCAAGGTCCTACGTCTAACCATAAGAACGGCGATAGGCGCCCC
CTGGGAAGAGCTCACATCAGGGTACTATTCCTGCAATGCCCTAGTAAATG
AATGAAGTTGATCATGGCCAATTGGAAGAATCACAAAAAAAAAAAAAAAA
AAAAAAA

(SEQ ID: No. 35)

Forward Primer: TGAACCCACCAAAGATGTGTATGAG

(SEQ ID: No. 36)

Reverse Primer: CCATCCTTCTGGTAGGCATTCAAAT

(SEQ ID: No. 37)

Probe: CTGCACCTGAATATTG
Mn1Tel

(SEQ ID: No. 38)

GGCAGCTGCTGCTCCGAGGCGGTCAAGAGCGCCATGAGCACCATTGACCT
GGACTCGCTGATGGCAGAGCACAGCGCTGCCTGGTACATGCCCGCTGACA
AGGCCCTGGTGGACAGCGCGGACGACGACAAGACGTTGGCGCCCTGGGAG
AAGGCCAAACCCCAGAACCCCAACAGCAAAGAAGGCTTGCAGCCAATTTA
CTGGAGCAGGGATGACGTAGCCCAGTGGCTCAAGTGGGCTGAAAATGAGT
TTTCTTTAAGGCCAATTGACAGCAACACGTTTGAAATGAATGGCAAAGCT
CTCCTGCTGCTGACCAAAGAGGACTTTCGCTATCGATCTCCTCATTCAGG
TGATGTGCTCTATGAACTCCTTCAGCATATTCTGAAGCAGAGGAAACCTC
GGATTCTTTTTTCACC

(SEQ ID: No. 39)

Forward Primer: AAACCCCAGAACCCCAACAG

(SEQ ID: No. 40)

Reverse Primer: TCATCCCTGCTCCAGTAAATTGG

(SEQ ID: No. 41)

Probe: CTGCAAGCCTTCTTTG

mutation—a heritable change in DNA sequence resulting from mutagens. Various types of mutations including frame-shift mutations, missense mutations, and nonsense mutations.

Neomycin

(SEQ ID: No. 42)

CATTGAACAAGATGGATTGCACGCAGGTTCTCCGGCCGCTTGGGTGGAGA
GGCTATTCGGCTATGACTGGGCACAACAGACAATCGGCTGCTCTGATGCC
GCCGTGTTCCGGCTGTCAGCGCAGGGGCGCCCGGTTCTTTTTGTCAAGAC
CGACCTGTCCGGTGCCCTGAATGAACTGCAGGACGAGGCAGCGCGGCTAT
CGTGGCTGGCCACGACGGGCGTTCCTTGCGCAGCTGTGCTCGACGTTGTC
ACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTG
ATGCAATGCGGCGGCTGCATACGCTTGATCCGGCTACCTGCCCATTCGAC
CACCAAGCGAAACATCGCATCGAGCGAGCACGTACTCGGATGGAAGCCGG
TCTTGTCGATCAGGATGATCTGGACGAAGAGCATCAGGGGCTCGCGCCAG
CCGAACTGTTCGCCAGGCTCAAGGCGCGCATGCCCGACGGCGAGGATCTC
GTCGTGACCCATGGCGATGCCTGCTTGCCGAATATCATGGTGGAAAATGG
CCGCTTTTCTGGATTCATCGACTGTGGCCGGCTGGGTGTGGCGGACCGCT
ATCAGGACATAGCGTTGGCTACCCGTGATATTGCTGAAGAGCTTGGCGGC
GAATGGGCTGACCGCTTCCTCGTGCTTTACGGTATCGCCGCTCCCGATTC
GCAGCGCATCGCCTTCTATCGCCTTCTTGACGAGTTCTTCTG

(SEQ ID: No. 43)

Forward Primer: GGGCGCCCGGTTCTT

(SEQ ID: No. 44)

Reverse Primer: CCTCGTCCTGCAGTTCATTCA

(SEQ ID: No. 45)

Probe: ACCTGTCCGGTGCCC
OPN4ES

(SEQ ID NO.: 46)

TTAAAGCTCATGCCTAGACCTGATGCTATAGAAGGTGTGCTCCTCGCTTC
TCTGCCAATCTTAAGGTGCCCTGGATGGAGCTGGGTGACGTGTTTACCCT
TGTAGTCTGTCCTGTCTATATGCATGGATATGCACAGTGCCCTTGACCCA
ACCCTGCCAACCAGGCACCTGCAGAAGGTGTAGATGACCGTCAGATTGCC
CAGCATCCCTGTGAGTCCCACCAGCAGGATCACCGTGCCTAGGGTATAGT
GAGCATGGTCTGGGACATCGACTGTGGGGAAGGGGACCCAGGCAGCAGCC
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNAGCCCATAGAAGAAAGTGCAAGTCTTCCA
AAATTTAACCCCACGCCCATATATGTGTGGATACTGAGCTTCTAAGAGGG
AGTGAAAGGCTCAGATGGCCTGCTGGAGGTTAACAGGACAAATGCGTGCC
TGCAGGACAGAGCACAGCTTGGGTGACCTTAAGGAATGAGTAGAGCCAGG
TCCTGGGTACTGCCCTCCCAACGAATGGATACCCCACAGCAAGCCTCCAA
GGAGAACTTGCAACCCCTGTNNNNNNNNNNNNNNNNNNNNNNNNNNNNNN
NNAAACGAGGGAGGAGAACTTTCCACTAGAAAGAGAGTTTAGGTTCCCCC
AGGCTGCTGGGAGGCCATTTCCCCCATGAGGTTAGTACACAGGGACTAAG
GATAGCTCCCAGGGAGAGGCAGGAGTCTGCCCAATGTCCTGCCCAGCATC
CCACTCTGGCCTGTACAAGTCCAGAAGCCTAGGGCATGCCTTTCCCCCTA
GGATACTCCCCCAGGGGATNNNNNNNNNNNNNNNNNNNNNNNNNNNNNNN
NNNNGAAGAGCAGGTCAGCCCCTGCCTTTCTGGTTCTCCAGTGGTCTCTG
CCAACAAAGACATTGCCTGTGCCCTCTTGTCTCAGCCACTGTGTAGAGAA
AGCTTAGAGAACTTCAGTGACGCTCAAGGTCCTTCGTCTAAGCTCAGACC
TTTTCTATCTCCCTGTTAAAACAAGGGTGGGGACAGGAGTCTCTGTGTAC
ACACATGCTCCCCAAACTTACCGTGGGGCTAACAGAGAGAAGCTGGGCTC
TTACGGAGACGTTCTGAGTGCCGTTCCAAATGCCTTGCAGGGCAGGACTG
GTTGTGAAGCTGGGATCCTGAGTTAAGCTTGACAAGAC

(SEQ ID: No. 47)

Forward Primer: TGGGTGACCTTAAGGAATGAGTAGA

(SEQ ID: No. 48)

Reverse Primer: GTTCTCCTTGGAGGCTTGCT

(SEQ ID: No. 49)

Probe: CTGCCCTCCCAACGAA
p16

(SEQ ID: No. 50)

GTGATGATGATGGGCAACGTTCACGTAGCAGCTCTTCTGCTCAACTACGG
TGCAGATTCGAACTGCGAGGACCCCACTACCTTCTCCCGCCCGGTGCACG
ACGCAGCGCGGGAAGGCTTCCTGGACACGCTGGTGGTGCTGCACGGGTCA
GGGGCTCGGCTGGATGTGCGCGATGCCTGGGGTCGCCTGCCGCTCGACTT
GGCCCAAGAGCGGGGACATCAAGACATCGTGCGATATTTGCGTTCCGCTG
GGTGCTCTTTGTGTTCCGCTGGGTGGTCTTTGTGTACCGCTGGGAACGTC
GCCCAGACCGACGGGCATAGCTTCAGCTCAAGCACGCCCAG

(SEQ ID: No. 51)

Forward Primer: CGAGGACCCCACTACCTTCT

(SEQ ID: No. 52)

Reverse Primer: CCGCTCTTGGGCCAAGT

(SEQ ID: No. 53)

Probe: CAGGCATCGCGCACAT

plate controls—are wells that include the house-keeping probe without nucleic acid sample.

Puromycin Sequence

(SEQ ID: No. 54)

ATGACCGAGTACAAGCCCACGGTGCGCCTCGCCACCCGCGACGACGTCCC
CCGGGCCGTACGCACCCTCGCCGCCGCGTTCGCCGACTACCCCGCCACGC
GCCACACCGTCGACCCGGACCGCCACATCGAGCGGGTCACCGAGCTGCAA
GAACTCTTCCTCACGCGCGTCGGGCTCGACATCGGCAAGGTGTGGGTCGC
GGACGACGGCGCCGCGGTGGCGGTCTGGACCACGCCGGAGAGCGTCGAAG
CGGGGGCGGTGTTCGCCGAGATCGGCCCGCGCATGGCCGAGTTGAGCGGT
TCCCGGCTGGCCGCGCAGCAACAGATGGAAGGCCTCCTGGCGCCGCACCG
GCCCAAGGAGCCCGCGTGGTTCCTGGCCACCGTCGGCGTCTCGCCCGACC
ACCAGGGCAAGGGTCTGGGCAGCGCCGTCGTGCTCCCCGGAGTGGAGGCG
GCCGAGCGCGCCGGGGTGCCCGCCTTCCTGGAGACCTCCGCGCCCCGCAA
CCTCCCCTTCTACGAGCGGCTCGGCTTCACCGTCACCGCCGACGTCGAGT
GCCCGAAGGACCGCGCGACCTGGTGCATGACCCGCAAGCCCGGTGCCTGA

(SEQ ID NO.: 55)

Forward Primer: GCGGTGTTCGCCGAGAT

(SEQ ID NO.: 56)

Reverse Primer: GAGGCCTTCCATCTGTTGCT

(SEQ ID NO.: 57)

Probe: GCGGTGTTCGCCGAGAT
RIP7-rtTA

(SEQ ID NO.: 58)

ATGTCTAGATTAGATAAAAGTAAAGTGATTAACAGCGCATTAGAGCTGCT
TAATGAGGTCGGAATCGAAGGTTTAACAACCCGTAAACTCGCCCAGAAGC
TAGGTGTAGAGCAGCCTACATTGTATTGGCATGTAAAAAATAAGCGGGCT
TTGCTCGACGCCTTAGCCATTGAGATGTTAGATAGGCACCATACTCACTT
TTGCCCTTTAGAAGGGGAAAGCTGGCAAGATTTTTTACGTAATAACGCTA
AAAGTTTTAGATGTGCTTTACTAAGTCATCGCGATGGAGCAAAAGTACAT
TTAGGTACACGGCCTACAGAAAAACAGTATGAAACTCTCGAAAATCAATT
AGCCTTTTTATGCCAACAAGGTTTTTCACTAGAGAATGCATTATATGCAC
TCAGCGCTGTGGGGCATTTTACTTTAGGTTGCGTATTGGAAGATCAAGAG
CATCAAGTCGCTAAAGAAGAAAGGGAAACACCTACTACTGATAGTATGCC
GCCATTATTACGACAAGCTATCGAATTATTTGATCACCAAGGTGCAGAGC
CAGCCTTCTTATTCGGCCTTGAATTGATCATATGCGGATTAGAAAAACAA
CTTAAATGTGAAAGTGGGTCCGCGTACAGCCGCGCGCGTACGAAAAACAA
TTACGGGTCTACCATCGAGGGCCTGCTCGATCTCCCGGACGACGACGCCC
CCGAAGAGGCGGGGCTGGCGGCTCCGCGCCTGTCCTTTCTCCCCGCGGGA
CACACGCGCAGACTGTCGACGGCCCCCCCGACCGATGTCAGCCTGGGGGA
CGAGCTCCACTTAGACGGCGAGGACGTGGCGATGGCGCATGCCGACGCGC
TAGACGATTTCGATCTGGACATGTTGGGGGACGGGGATTCCCCGGGTCCG
GGATTTACCCCCCACGACTCCGCCCCCTACGGCGCTCTGGATATGGCCGA
CTTCGAGTTTGAGCAGATGTTTACCGATGCCCTTGGAATTGACGAGTACG
GTGGGTAG

(SEQ ID NO.: 59)

Forward Primer: TGCCAACAAGGTTTTTCACTAGAGA

(SEQ ID NO.: 60)

Reverse Primer: CTCTTGATCTTCCAATACGCAACCTA

(SEQ ID NO.: 61)

Probe: CCACAGCGCTGAGTGC

recombination—The process by which offspring derive a combination of genes different from that of either parent. In higher organisms, this can occur by crossing over.

recombinant DNA—A combination of DNA molecules of different origin that are joined using recombinant DNA technologies.

RNA—on of the two main types of nucleic acid, consisting of a long, unbranched macromolecule formed from ribonucleotides, the 3′-phosphate group of each constituent ribonucleotide (except the last) being joined in 3′,5′—phosphodiester linkage to the 5′-hydroxyl group on each ribose moiety renders these phosphodiester bonds susceptible to hydrolytic attack by alkali, in contrast to those of DNA. The RNA chain has polarity, with one 5′ end and on 3′ end. Two purines, adenine and guanine, and two pyrimidines, cytosine and uracil, are the major bases usually present. In addition, minor bases may occur; transfer RNA, however, contains unusual bases in relatively large amounts. The sequence of bases carries information, whereas the sugar and phosphate groups play a structural role. RNA is fundamental to protein biosynthesis in all living cells. Oxford Dictionary of Biochemistry and Molecular Biology; p. 577.

screening reference—are probes that are run on every sample submitted to screen laboratory. The probe is one that is found in every mouse, mutant or not.

Six-2 WT

(SEQ ID NO. 62)

GGGTGAGGCTGTTGCGACGCCTCTTATTTAAAAAAAAAGGGAGGGGTGTC
TCACACTTTTTCTCTTGAAGGCTCCTTCTGTCCCCCTCTTTTCCTTTCCT
GAAAGGCACCCCCTTAAACGGTCCTCCGCCTTCCCTTCTACTCCCTTCCT
TCCCCACTTCGGTCCTCCTCTTTTCTTCGAGGGCCCCCACCCAGCCCCCT
CCTTCGGGGTCCTCCTCCTCCTCTGCTCTTTGGGCGTCCGCCCCGTCAAT
CACCGCCGTCTCGGGGCCCCAGCCCGGCTCCTCTCCGCCTCCCGGGCTCT
GGGAGTGCCTGGGGCTCCCGTCTCGGCCAACCTCCGCTCTGTGCAGAGCC
GGGGCGATCTGTCAGCGGAGCTGGCCGAGGGGGGCGGGGGTGGGAGCCGC
CCGGGCCGCCGGGGCTCGGGTTACCGGTGACTGACAGCGTCTCCATGGCG
AATAATTTGACTCGACTATTGTCTGGCGCGGGCAGGCCCCGGGTCAGATA
ACCCGACCAATCAGGGCGCGGGCCGCCGCGCCTCATGCCCGCTTAGAATA
ATATTATTAAAAAAGCTGCAAGCGAGCTAGACGGGAGGGAGAGCGAACGA
GCGAGGAGCCGGCGAGCGAGCGGCGGGCGGGCGCGGAGCATGCGGAGCGG
CGCCCCGGGCGGCCTCCGGGCTTGGGCGCGGGCGAGGCGCGCGGGCGGCG
GGGGCGCGGAGCTGCGCGGGGCCGGCGGCGGGAGCGAGGACGGATCGTTG
TGACTCAGGAGTCGCTCGGGAGCCGGCGCCTGGCCAGGGGGCCCCGCCCG
CCTGTCGGCCGGCCGGGGCCGGCGGGGAGGCGCCCATGCGGGGCCGCGAA
GCGCGGTGAGGGCGCGCGCGGGCGGGCGGGCGCGCAGCCGCCACCATGTC
CATGCTGCCCACCTTCGGCTTCACGCAGGAGCAAGTGGCGTGCGTGTGCG
AGGTGCTGCAGCAGGGCGGCAACATCGAGCGGCTGGGTCGCTTCCTGTGG
TCGCTGCCCGCCTGCGAGCACCTCCACAAGAATGAAAGCGTGCTCAAGGC
CAAGGCCGTGGTGGCCTTCCACCGGGGCAACTTCCGCGAGCTCTACAAAA
TCCTGGAGAGCCACCAGTTCTCGCCGCACAACCACGCCA

(SEQ ID NO. 63)

Forward Primer: GGGTTACCGGTGACTGACA

(SEQ ID NO. 64)

Reverse Primer: CCCGCGCCAGACAATAGT

(SEQ ID NO. 65)

Probe: CCATGGCGAATAATTT

strain—a group of organisms bred for a genotype (at least one designated genetic sequence).

strain controls—are biomatter samples submitted by a remote user 1. Strain controls are controls positive and negative sent to the screen laboratory as the remote user that discloses the genotype.

TetAKT1

(SEQ ID NO.: 66)

ATGAACGACGTAGCCATTGTGAAGGAGGGCTGGCTGCACAAACGAGGGGA
ATATATTAAAACCTGGCGGCCACGCTACTTCCTCCTCAAGAACGATGGCA
CCTTTATTGGCTACAAGGAACGGCCTCAGGATGTGGATCAGCGAGAGTCC
CCACTCAACAACTTCTCAGTGGCACAATGCCAGCTGATGAAGACAGAGCG
GCCAAGGCCCAACACCTTTATCATCCGCTGCCTGCAGTGGACCACAGTCA
TTGAGCGCACCTTCCATGTGGAAACGCCTGAGGAGCGGGAAGAATGGGCC
ACCGCCATTCAGACTGTGGCCGATGGACTCAAGAGGCAGGAAGAAGAGAC
GATGGACTTCCGATCAGGCTCACCCAGTGACAACTCAGGGGCTGAAGAGA
TGGAGGTGTCCCTGGCCAAGCCCAAGCACCGTGTGACCATGAACGAGTTT
GAGTACCTGAAACTACTGGGCAAGGGCACCTTTGGGAAAGTGATTCTGGT
GAAAGAGAAGGCCACAGGCCGCTACTATGCCATGAAGATCCTCAAGAAGG
AGGTCATCGTCGCCAAGGATGAGGTTGCCCACACGCTTACTGAGAACCGT
GTCCTGCAGAACTCTAGGCATCCCTTCCTTACGGCCCTCAAGTACTCATT
CCAGACCCACGACCGCCTCTGCTTTGTCATGGAGTATGCCAACGGGGGCG
AGCTCTTCTTCCACCTGTCTCGAGAGCGCGTGTTCTCCGAGGACCGGGCC
CGCTTCTATGGTGCGGAGATTGTGTCTGCCCTGGACTACTTGCACTCCGA
GAAGAACGTGGTGTACCGGGACCTGAAGCTGGAGAACCTCATGCTGGACA
AGGACGGGCACATCAAGATAACGGACTTCGGGCTGTGCAAGGAGGGGATC
AAGGATGGTGCCACTATGAAGACATTCTGCGGAACGCCGGAGTACCTGGC
CCCTGAGGTGCTGGAGGACAACGACTACGGCCGTGCAGTGGACTGGTGGG
GGCTGGGCGTGGTCATGTATGAGATGATGTGTGGCCGCCTGCCCTTCTAC
AACCAGGACCACGAGAAGCTGTTCGAGCTGATCCTCATGGAGGAGATCCG
CTTCCCGCGCACACTCGGCCCTGAGGCCAAGTCCCTGCTCTCCGGGCTGC
TCAAGAAGGACCCTACACAGAGGCTCGGTGGGGGCTCTGAGGATGCCAAG
GAGATCATGCAGCACCGGTTCTTTGCCAACATCGTGTGGCAGGATGTGTA
TGAGAAGAAGCTGAGCCCACCTTTCAAGCCCCAGGTCACCTCTGAGACTG
ACACCAGGTATTTCGATGAGGAGTTCACAGCTCAGATGATCACCATCACG
CCGCCTGATCAAGATGACAGCATGGAGTGTGTGGACAGTGAGCGGAGGCC
GCACTTCCCCCAGTTCTCCTACTCAGCCAGTGGCACAGCCTGA

(SEQ ID NO.: 67)

Forward Primer: GGAACGCCGGAGTACCT

(SEQ ID NO.: 68)

Reverse Primer: ACTGCACGGCCGTAGTC

(SEQ ID NO.: 69)

Probe: CTGAGGTGCTGGAGGACA
Tetp27KIP

(SEQ ID NO.: 70)

CCATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGA
GGGCGAGGGCGATGCCACCCTACGGCAAGCTGACCCTGAAGTTCATCTGC
ACCACCGGCAAGCTGCCCGTGCCCTGGCCCACCCTCGTGACCACCCTGAC
CTACGGCGTGCAGTGCTTCAGCCGCTACCCCGACCACATGAAGCAGCACG
ACTTCTTCAAGTCCGCCATGCCCGAAGGCTACGTCCAGGAGCGCACCATC
TTCTTCAAGGACGACGGCAACTACAAGACCCGCGCCGAGGTGAAGTTCGA
GGGCGACACCCTGGTGAACCGCATCGAGCTGAAGGGCATCGACTTCAAGG
AGGACGGCAACATCCTGGGGCACAAGCTGGAGTACAACTACAACAGCCAC
AACGTCTATATCATGGCCGACAAGCAGAAGAACGGCATCAAGGTGAACTT
CAAGATCCGCCACAACATCGAGGACGGCAGCGTGCAGCTCGCCGACCACT
ACCAGCAGAACACCCCCATCGGCGACGGCCCCGTGCTGCTGCCCGACAAC
CACTACCTGAGCACCCAGTCCGCCCTGAGCAAAGACCCCAACGAGAAGCG
CGATCACATGGTCCTGCTGGAGTTCGTGACCGCCGCCGGGATCACTCTCG
GCATGGACGAGCTGTACAAGTAAAG

(SEQ ID NO.: 71)

Forward Primer: CGTCGTCCTTGAAGAAGATGGT

(SEQ ID NO.: 72)

Reverse Primer: CACATGAAGCAGCACGACTT

(SEQ ID NO.: 73)

Probe: CATGCCCGAAGGCTAC

transgene—the foreign gene or DNA.

transgenic—this term describes an organism that has had genes from an organism or additional elements of it our sequence put into its genome through recombinant DNA techniques. These organisms are usually made by microinjection of DNA in the pronucleus of fertilized eggs, with the DNA integrating at random.

transgenic line—a transgenic mouse or organism strain in which the transgene is stably integrated into the germline and therefore inherited in Mendelian fashion by succeeding generation.

web site—a computer system that serves informational content over a network using the standard protocol of the World Wide Web. A web site corresponds to a particular Internet domain name such as TransnetYX.com.

wild type—the phenotype that is characteristic of most of the members of a species occurring naturally and contrasting with the phenotype of a mutant.

zygosity—This term reflect the genetic makeup of an individual. When identical alleles exist at a loci it is said to be homozygous; when alleles are different the alleles are said to be heterozygous.

### 2. OVERVIEW OF THE SYSTEMS COMPONENTS AND OPERATIONS

The present invention provides methods for genotype screening. More specifically, the present application relates to a method to rapidly screen biological samples for at least one designated genetic sequence. Various aspects of genotype screening involve: sample collection, lysing of the biological sample, isolation of a standard concentration of purified genomic nucleic acid and nucleic acid screening. Additionally, the method operating according to the features described herein can provide screening results to a remote user 1 from the screening laboratory 20 within 24 hours of receiving the biological samples.

In order to screen for a designated genetic sequence, that sequence must first be determined or identified. Only when the designated sequence is known can a test be devised to search for its existence in the biological samples provided by the remote user 1 to the screening laboratory 20.

There are a variety of ways the designated genetic sequence can be acquired by the remote user 1 or by the screening laboratory 20. For example, if the sequence of bases that makeup the designated genetic sequence is known by the remote user 1, the sequence can be directly communicated to the screening laboratory 20 via an electronic link, such as any of the electronic communication links identified herein, and particularly the communication links extending between the remote user's computer and the screening laboratory 20.

The remote user 1 can indirectly communicate the designated genetic sequence to the screening laboratory 20 by communicating a publication, journal article, a gene name, a sequence name, a line or strain name (if the designated genetic sequence is found in animals of that line or strain), or the name of a mutation having the designated genetic sequence to the screening laboratory 20. Alternatively, the remote user 1 can communicate to the screening laboratory 20 the sequence of a primer set or probe that corresponds to a target genetic sequence of the designated genetic sequence. These primer sets or probes will have previously been created or defined to indicate the presence of the designated genetic sequence.

The indirect references may provide the entire sequence. Alternatively, the screening laboratory 20 may take the information from the references or from the remote user 1 and use it to search public genetic databases such as The National Center for Biotechnology Information (NCBI), Ensembl, or The Wellcome Trust Sanger Institute database. The screening laboratory 20 can also search proprietary databases, such as the database provided by Celera Bioscience (Rockville, Md.).

Another indirect method that may be used to acquire or identify the designated genetic sequence is to use a third party who has specific knowledge of the sequence. For example, the screening laboratory 20 can receive the name of a transgenic animal line or strain from the remote user 1, then contact the company that engineers that line or strain. The company can then transmit the sequence of bases that constitute the particular genetic sequence corresponding to that line or strain back to the screening laboratory 20. These companies include such firms as Lexicon Genetics (Woodland, Tex.) or Charles River Laboratories (Wilmington, Mass.). Even further, individual researchers who have developed the line or strain, or who work with the same line or strain at another laboratory may provide the designated genetic sequence, the primer sets or the probes necessary to identify the designated genetic sequence.

If the designated genetic sequence is not known by the remote user 1 or third party and is not found in any public or private database, the screening laboratory 20 may use scientific methods. If the remote user 1 has a working genotyping assay, and they are performing PCR and separating fragments in a gel, the appropriate bands can be cut from the gel, purified and sequenced to determine the sequence of bases in that band. The company sequencing the bands can directly communicate the base sequence to the screening laboratory 20 or to the remote user 1, who in turn can communicate the base sequence to the screening laboratory 20.

Once identity of the designated genetic sequence is acquired by the screening laboratory 20 (and assuming a probe or primer set has yet to be designed), the screening laboratory 20 must then select a target genetic sequence of the designated genetic sequence for which a primer set and/or probe can be constructed. In the preferred embodiment, the sequence of the primer set and probe is determined using software such as Primer Express® (Applied Bio Systems). The target genetic sequence may be directly selected from the designated genetic sequence by the screening laboratory 20. Once selected, the base sequence corresponding to the target genetic sequence is communicated to an oligonucleotide vendor, who manufactures the probe and primer sets and transmits them to the screening laboratory 20.

The screening laboratory 20 preferably keeps a supply of probes and primer sets on hand so each future request by the remote user need not require special production of probes and primer sets.

Alternatively, a special probe or primer set may be required. In that situation, the screening laboratory 20 may not select the target genetic sequence itself, but may communicate to a third party specific areas in the designated genetic sequence that are important for mutation detection. The third party is typically an oligonucleotide vendor, who in turn will select the target genetic sequence, manufacture the probes and primer sets, and send the probes and primer sets to the screening laboratory 20.

This alternative approach is particularly beneficial for zygosity genotyping of nontransgenic samples (as shown in Example 7), which requires such special probes or primer sets. Zygosity testing includes identifying not only the presence of a designated genetic sequence but also whether that designated genetic sequence is located on both (+/+ homozygous), one (+/− heterozygous) or neither (−/− wild type) chromosome(s). The results are then determined by evaluating both pieces of information to determine zygosity. If signal is acquired solely from the mutation probe or the endogenous probe then the samples is homozygous for the mutation or homozygous for the endogenous sequence, respectively. If signal is acquired from both primer-probe combinations then the sample is heterozygous. The LIMS will establish three distinct categories to correspond with the three control samples needed (a homozygous, a heterozygous and a wild type sample).

To effectively genotype these nontransgenic samples, additional bioinformatics are needed from the remote user 1. Specifically, the screening laboratory 20 requests that the remote user 1 provide both the base sequence of the designated genetic sequence of the mutation as well as the DNA sequence of the endogenous location. The endogenous DNA sequence is disrupted if a mutation has occurred. Once the precise sequence data is acquired, two primer-probe sets are designed. The first primer-probe set determines if the sequence of the mutation is present, irrespective of the number of times it is present. The second primer-probe set determines if the endogenous DNA sequence is present. It is these two primer-probe sets that the oligonucleotide vendor designs and transmits to the screening laboratory 20.

With respect to human genotyping, a remote user 1 can contact the screening laboratory 20 and provide information for a human mutation or suspected endogenous condition of interest. This information may include the remote user's interest in wanting to know if the sample is from a human or a mouse and if it is from a human what gender is the sample. The screening laboratory 20 can acquire primers and probe that can distinguish between humans and mice. This is accomplished by identifying areas of genetic sequence in the mouse genome that are not homologous with the genetic sequence in the Homo sapiens genome. With no input from the remote user 1, the screening laboratory 20 can query a database such as Ensembl that would discriminate between the sex chromosomes in humans (X and Y). This query would yield sequence data for the Y chromosome, which is the designated genetic sequence. The screening laboratory 20 can take the designated genetic sequence, or portion thereof, and send it to a vendor indicating where to build the primer set and probe as to be informative for screening. Moreover, where there are a large number of nucleotides that are unique on the human Y chromosome, the screening laboratory 20 may send the sequence of bases to the vendor and have them build primer sets and probe anywhere inside the sequence. The remote user 1's Internet web-based account will have a field populated that represents these reagents with an identifier such as the genetic line identification 84. The remote user 1 will use the identifier (strain name or profile name) to indicate that these specific reagents are to be used on subsequent samples.

Similarly, if the remote user 1 requires SNP genotyping a remote user 1 can contact the screening laboratory 20 and provide a literature reference of the mutation which discloses the mutation name. A mutation name query of the Mouse Genome Informatics website yields links to different databases such as Ensembl and National Center for Biotechnology Information that provides sequence data. This sequence data is the designated genetic sequence. Knowing the endogenous nucleotide and the mutant nucleotide, the screening laboratory 20 can take the designated genetic sequence, or portion thereof, and send it to a vendor indicating specifically where to build the primers and probes as to be informative for screening. For example, if the designated genetic sequence is 500 nucleotides in length, the screening laboratory 20 may indicate to the reagent vendor to build a SNP assay targeting the 239^{th} nucleotide. The reagent vendor will then supply to the screening laboratory 20, the primers and probes to specifically discriminate between a nucleotide change at the 239^{th} position of the designated genetic sequence.

The remote user 1's Internet web-based account will have a field populated that represents these reagents with an identifier such as a name or number, or what is commonly referred to as the genetic line identification 84. The remote user 1 will use the genetic line identification 84 to indicate that these specific reagents are to be used on subsequent samples.

The probes and primer sets, if they are new and have not before been tested against a sample containing the designated genetic sequence, must then be tested, preferably by the screening laboratory 20. To do this, the screening laboratory 20 preferably receives both a positive and a negative strain control samples from the remote user 1 and tests them against the probes and primer sets to confirm that they can be used successfully to determine whether the designated genetic sequence can be detected. These controls include one positive and one negative control for each mutation found in the strain of interest.

If the designated genetic sequence can be detected using the probes and primer sets, the screening laboratory 20 updates the website and the order management software to provide the remote user 1 with a web-based selection for sample testing using those tested probes and primer sets. These selections among which the remote user 1 can select are one of the screening parameter selections identified below.

Alternatively, for example, if the remote user 1 or other third party communicates to the screening laboratory 20 that a particular probe or primer set has already been tested and is known to work, or if the screening laboratory 20 has already designed a probe and primer set for the designated genetic sequence (which is commonly the case for often-used strains or lines of transgenic animals) the screening laboratory 20 can immediately add a selection to the website and does not need to test controls with the probes and primer sets.

The strain controls are used to tell LIMS 24 a signal magnitude that is then associated with a positive or negative sample. In one case, the remote user 1 may send these controls together with the samples to be tested to the screening laboratory 20 in a single shipment. Alternatively, the controls may be sent separately from the samples to be tested.

The screening laboratory 20 tests the strain controls using the process described herein for testing samples. At the end of this testing process, the signal values for the strain controls are recorded into LIMS 24. The magnitude of the signal provided by the positive control indicates the expected signal level for subsequently tested samples having the designated genetic sequence. The magnitude of the signal provided by the negative control indicating the expected signal level for subsequently tested samples that do not have the designate genetic sequence.

The computer at the screening laboratory 20 is configured to compare the test results (i.e. signal levels) for every sample that it subsequently tests for that designated genetic sequence with these multiple control signal levels and, based on that determination, to decide whether that sample has or does not have the designated genetic sequence. Positive and negative strain controls for a line therefore do not need to be resubmitted for each subsequent order but can be referenced by the screening laboratory 20 computer when later samples are tested for the same designated genetic sequence.

For transgenic zygosity genotyping, additional controls (not just a positive and a negative) are required to indicate each possible variation such as: a homozygous control, a heterozygous control and a wild type control.

Upon receipt of the primers and probe from a vendor, the sample, if available, will be screened using these reagents. Once a determination is made that there is discrimination between different genetic conditions, then the reagents will be placed in the inventory. Additionally, the screening laboratory 20 will populate a data field on the order management system, allowing the remote user 1 to select this primer sets and probe combination(s) for subsequent samples. This data filed will be populated with an indicator such as a mutation name, strain name or genetic line identification that will represent these reagents or combination of reagents that will be used in subsequent samples of this strain. This allows the remote user 1 to select the indicator of the reagents and prevents the need to transfer genetic information with each order.

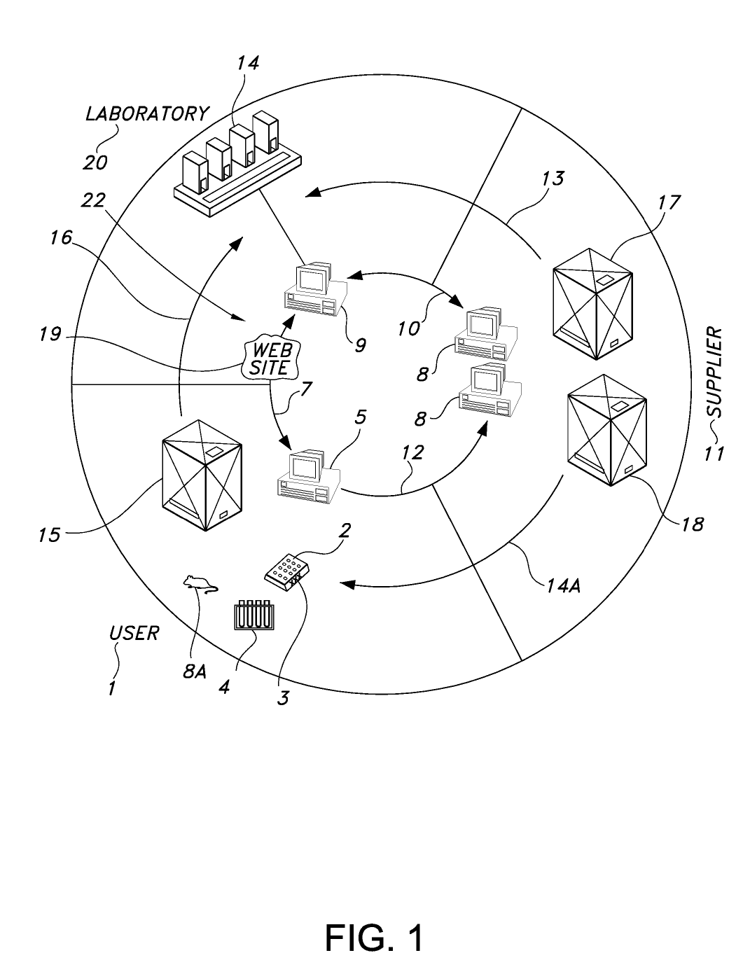

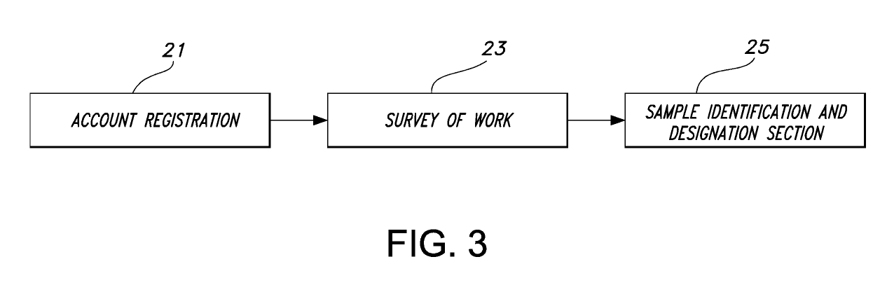

FIGS. 1-3 present an overview of certain features of the present invention. The present invention allows a remote user 1 with access to a computer 5 to order genotype screening of samples they submit to screening laboratory 20. Using the Internet or other communication link 7, the remote user 1 sends an access request from the remote user's computer 5 to a screening laboratory 20 computer 9 via an electronic communication link 7, such as the Internet. The screening laboratory 20 website 19 will transmit an access enabling response to the remote user 1 via electronic communication link 7. This response includes three distinct sections. The three sections are Account Registration 21, Survey of Work 23 and Sample Identification and Designation 25 (FIG. 3).

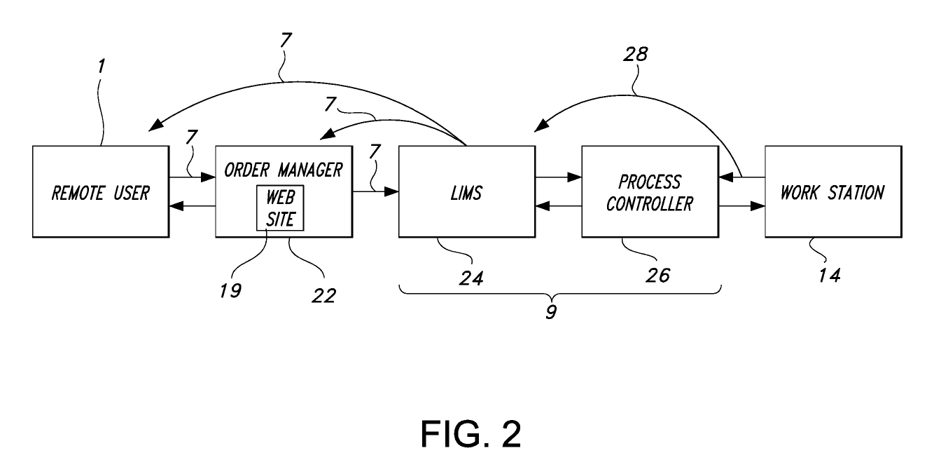

Now referring to FIG. 2, a remote user 1 can access screening laboratory 20 website 19 via communication link 7. The website 19 can be housed by an order manager 22. An order manager is a software-based order management system. In the preferred embodiment the order manager 22 is an order management system developed by “Big Fish”, a software development company in Memphis, Tenn. The order manager 22 functions to manage the placement of the order. The order received from the remote user 1 is transmitted to website 19, which reports the order to order manager 22. Manager 22 is in electronic communication via link 7 with screening laboratory 20 computer 9. Screening laboratory 20 computer 9 includes LIMS 24, which is communicatively coupled to a process controller 26.

LIMS 24 is the generic name for laboratory information management system software. The function of LIMS 24 is to be a repository for data, to control automation of a laboratory, to track samples, to chart work flow, and to provide electronic data capture. LIMS 24 can also, in another embodiment, be in direct communication with the remote user 1 via an electronic communications link 7. Any standard laboratory information management system software can configured to be used to provide these functions. Alternatively, a standard relational database management system such as Oracle (Oracle Corp., Redwood Shores, Calif.) or SQL Server (Microsoft Corp., Redmond, Wash.) either alone or in combination with a standard LIMS system can be used. In the preferred embodiment, the Nautilus® program (Thermo LabSystems, a business of Thermo Electron Corporation, Beverly, Mass.) is used.

The process controller 26 is communicatively coupled to the workstation 14. The process controller provides commands to any portions of the workstation 14 that are amenable to automation. For example, process controller 26 directs the delivery of the probes and primers to the Screening Station 95. The workstation 14 is communicatively linked 28 to LIMS 24. In this way, the workstation 14 can provide data to LIMS 24 for the formulation of the outcome report 249, and then, via link 7 to the order manager 22 or remote user 1. In an alternative embodiment, remote user 1 at remote user computer 5 can be linked 7 to the screening laboratory 20 by a direct phone line, cable or satellite connection.

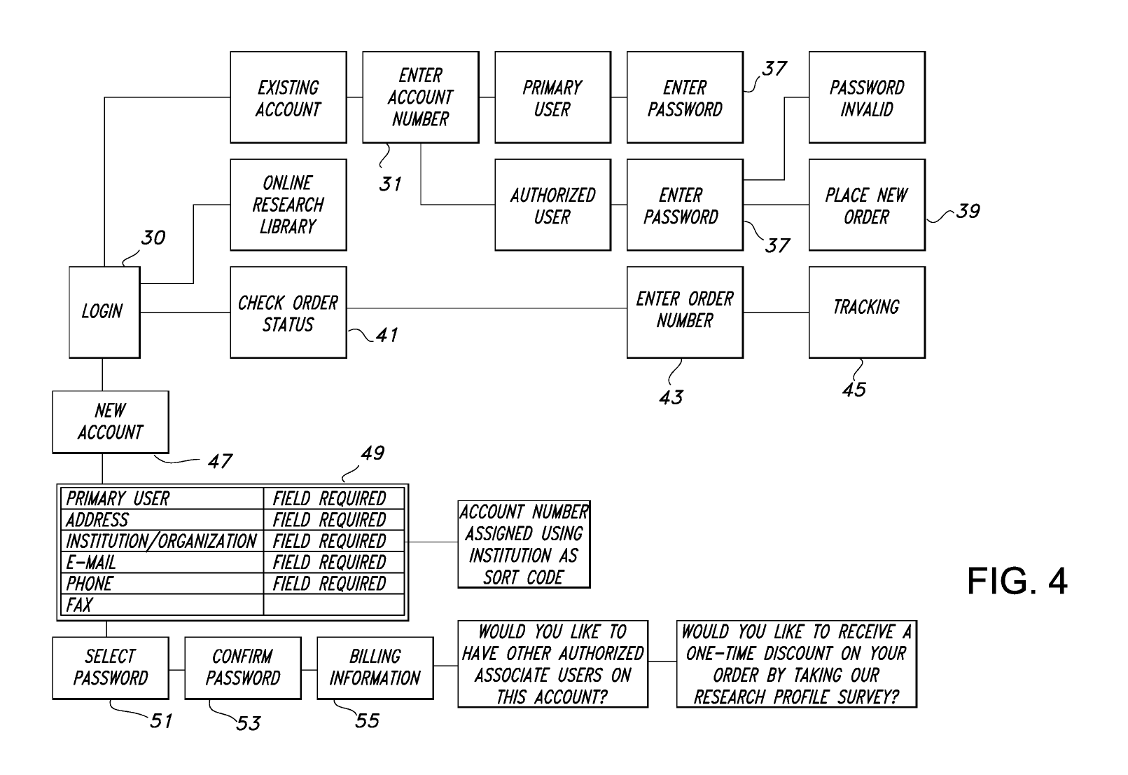

Now referring to FIG. 4, the user's Account Registration section 21 begins with logging into the system 30. A remote user 1 accesses an existing account by entering an account identification 31, which is, for example, an e-mail address. The user will then enter a password 37. If a valid password is entered, the user can place a new order 39. Alternatively, the user can check an order status 41 by providing an order number 43 and can proceed to order tracking 45. Alternatively, a new account 47 can be opened by providing an institution name, principal investigator, address, phone number, fax number, electronic mail address, billing information, and other authorized user names 49. The user can enter a password 51, confirm the password 53 and enter this billing information 55.

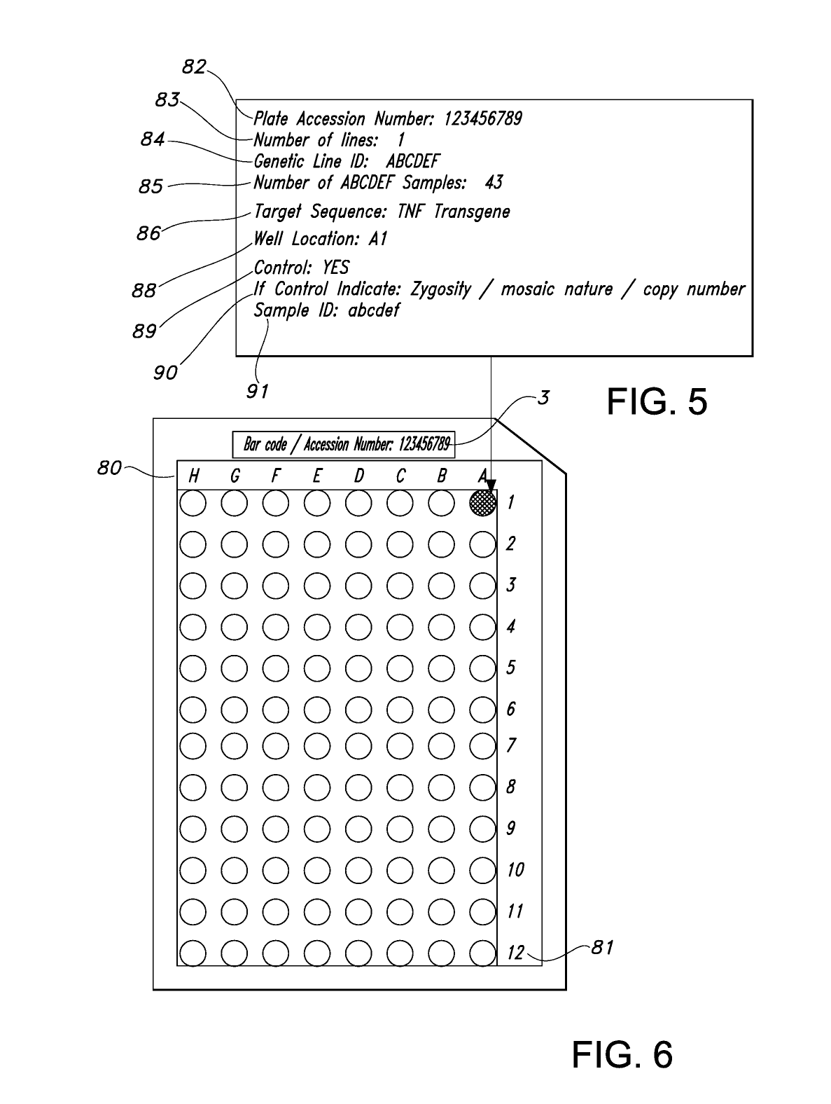

Now referring to FIGS. 5-6, once the remote user 1 submits the Survey of Work section 23 the remote user 1 will be presented with the Sample Identification and Designation section 25. In this section, the user (among other things) identifies where he will place each sample to be tested in an actual (physical) container 2 (FIG. 1) by associating each sample with a corresponding well of a virtual 96 well container displayed on the computer screen of computer 5 as described below. The Sample Identification and Designation section 25 includes 96 well container locations. The remote user 1 designates which sample was or will be placed into each well. If the remote user 1 has more than 96 samples, subsequent 96 source well containers and designations are available. With respect to FIG. 6, a 96 well source well container 2 having a barcode accession number 3 (FIG. 1) will be shown (FIG. 6) oriented in the longitudinal direction having an X axis labeled “A” to “H” (at 80) and a Y axis labeled “1” to “12” (at 81). The X and Y axes designate a well position such as “A1”.

FIGS. 5 and 6 together illustrate the Survey of Work section 23 and the Sample Identification and Designation Section 25. Referring now to FIG. 5, the remote user 1 is asked to provide: source well container 2 accession number 82, which the remote user 1 gets from the accession number 3 on the physical source well container 2 at his facility (FIG. 1) that he intends to fill (or has filled) with the samples, number of lines 83, genetic line identification 84, number of samples 85, and well location 88. The remote user 1 is also asked for any internal sample identification number 91.

For genotyping (i.e. screening to determine the presence of a designated genetic sequence) the positive strain control and the negative strain control samples are designated and deposited in wells of a microwell container. The remote user 1 indicates that a sample is a control sample at 89. This assumes, of course, that the strain controls were not earlier provided to the screening laboratory 20 as described above. If a control is deposited in source well container 2, remote user 1 can also designate the zygosity, mosaic nature and copy number of the sample.

At this point, the remote user has completed the Survey of Work section 23 and the Sample Designation section 25 of FIGS. 5-6 and is ready to transmit the screening parameter selections gathered in those sections to website 19 and thence to screening laboratory 20 computer 9.

Now referring to FIGS. 1 and 2, the remote user 1 transmits his or her order including the completed screening parameter selections to the screening laboratory 20 via link 7 such as the Internet or a direct line. The remote user 1 can transmit the selected screening parameter selections to LIMS 24 in screening laboratory 20 via electronic communications link 7. This link 7 can be direct or indirect. In the indirect route, the screening parameters are first transmitted to web site 19, wherein order manager 22 receives the order and then provides LIMS 24 with the screening parameter selections.

In a particularly preferred embodiment of the system described in the foregoing paragraphs, remote user 1 at computer 5 transmits a request for a home web page served by screening laboratory 20 web site 19 via the electronic communication link 7. Web site 19, in turn, serves a home web page to computer 5 that includes information identifying the source of the web page and including a login button. Remote user 1 at computer 5 clicks on the login button displayed on his computer screen, transmitting a signal to web site 19 requesting access to the web site. This request is transmitted over communications link 7 to web site 19, which responds with a second web page having fields for the entry of an account identifier (in the preferred embodiment an e-mail address), and a password. Remote user 1 enters the remote user 1 e-mail address and password, and transmits this information to web site 19 to gain access to the web site. Web site 19 receives this access request and compares the account identifier and password against its database of pre-existing accounts in the order manager 22 to determine whether the user is permitted to access the web site 19. If so, computer order manager 22 serves up a further web page, called an order manager web page, which includes several user selectable choices including an “order status” button for tracking previous orders and results (if any have been received), a “supply request” button for requesting supplies, and an “order” button for ordering additional tests.

To order genetic testing, user 1 clicks on the “order” button displayed on the screen of computer 5. Computer 5 transmits the user 1 request to web site 19. Web site 19 receives this request, and transmits a first ordering web page to computer 5. Computer 5, in turn, displays several fields on its computer screen, including several data entry widgets. The first of these widgets is list box including two selectable entries for requesting the speed of service. In the preferred embodiment there are two speeds of service: 24-hour service and 72 hour service. The second of these widgets is a list box providing several entries, each entry in the box corresponding to a strain for which the sample is to be tested. The third widget is a text box for entering the number of samples of the selected strain to be tested. The fourth widget is a text box for entering the accession number (typically a bar code number) of the source well container 2 in which the samples are to be placed for shipping to the screening laboratory 20.

The remote user 1 types in the number of samples to be tested. In this embodiment the samples are taken from transgenic animals, each sample typically corresponding to one animal to be tested. Typically several animals are tested to determine if they received the transgenic gene from their parents. Each strain of animal is defined by one or more designated genetic sequence. Thus, by designating the strain for which the samples are to be tested, the remote user 1 selects the one or more designated genetic sequences associated with that sequence. In the preferred embodiment, the remote user 1 can also select or deselect each individual probe and primer set that is used to screen for the designated sequences in the strain or line of the biological sample.

Once the remote user 1 has entered the number of samples to be tested, he or she then enters the name of the strain that the samples are to be tested for. Again, by selecting a strain the remote user 1 indicates the designated genetic sequence for which the samples are to be tested, since each strain is bred to have that sequence.

Once remote user 1 has selected the speed of service, the strain to be tested, and the number of samples to be tested for that strain, he enters the accession number from the source well container 2 and clicks on a button on the first ordering web page for recording this first group of samples to be tested. Computer 5, in turn, generates a revised first ordering web page, the revised page including a table entry in a table on the revised web page listing the first group of samples in tabular form, wherein each row in the table corresponds to one group of samples to be tested, identifying that group of samples by the strains for which that group of samples is to be tested, and the number of samples in that group.

This process of creating a new group of samples and identifying them by the strain for which they'll be tested, and the number of the samples, can be continued as many times as necessary until all the samples to be tested are identified in the table.

Once all of the groups of samples have been entered and listed in the table on the revised first ordering web page, the operator then selects a button identified “next” and moves to the next stage in the ordering process. Computer 5 transmits this request to web site 19, which generates a graphical image of a 96 source well container, appearing on the screen of computer 5 identical to the corresponding 96 source well container 2 that the remote user 1 is filling/has filled with samples, and transmits that image embedded in a second web page back to computer for display. The second web page includes a graphical representation of a 96 well plate, in a top view, showing the two dimensional array of all 96 wells in which the remote user 1 is to place the samples identified previously. Web site 19 calculates the respective positions of each group of samples in the well container 2. Each group is shown in the graphical representation of the well plate in a different color. All the wells in a group are shaded with the color associated with that group.

Samples of the same color from the same group are grouped together thus producing several different contiguous groups of wells, each group of wells have the same color different from the color of the adjacent groups.

The images of the wells in the web page are displayed on the computer with an initial shading to indicate that they have not been identified to a particular animal from which the sample in each well will be taken. In the preferred embodiment, each well contains a sample, such as a tissue sample, taken from an individual animal. The purpose of the testing performed on the samples in the wells is to determine the genetic characteristics of the animal from which each sample was taken. In order to relate the test results performed on each sample back to the animal from which the sample was taken, the user must make a record of the animal source of each sample (i.e. the animal from which each sample was taken).

To uniquely identify each sample in each well with an associated animal, remote user 1 selects a button on the third ordering web page. This button signals computer 9 to generate an additional web page. This web page lists each well in the well plate that was previously identified as containing a sample. Thus, if the first group of samples were 13 in number, there would be 13 entries listed in the additional web page. The web page itself is arranged as a single column of entries. Each entry in the column of entries includes a well identifier (called well location 88, above), which is a string of alphanumeric characters that uniquely identifies one well of source well container 2. A preferred well identifier for the 96 well plate is an alphabetic character followed by a numeric character. A text box is adjacent to each well identifier on the additional web page. To uniquely identify each sample in the source well container 2, the user enters alphanumeric characters in the text box that are uniquely associated with each sample. This identifier is typically a short string of consecutive alphabet or numeric characters, a practice commonly used by research facilities to identify individual animals used for testing.

Animals in a particular group of animals having (presumed) common genetic characteristics will typically be identified by tattoos, tags, or other permanent means by consecutive or sequential numbers, characters, or combinations of numbers and characters (for example “A1”, “A2”, “A3”, or “101”, “102”, 103”, or “AA”, AB”, “AC”, etc.). In a preferred embodiment, user 1 enters each animal number into the text box as a sample ID 91. Animals may also be identified by a unique combination of disfigurements such as cutting or cropping toes, tails or ears that can also be approximated to a progressive alphanumeric sequence.

To assist the remote user 1 in entering the sample ID 91 into each of the text boxes in the additional web page, a button is provided to automatically fill several consecutive text boxes based upon the alphanumeric characters typed into a few text boxes from the group. For example, if the user types in “B7” in the first text box of a group, then types in “B8” in the second text box of a group, computer 5 is configured to automatically generate consecutive alphanumeric strings to fill the remaining text boxes of the group based upon these two manually typed-in entries. In this case, computer 5 would automatically generate the alphanumeric strings “B9”, “B10”, “B11”, etc. and insert these characters sequentially into the remaining text boxes of the group in the additional web page. This process can be repeated for each subsequent group shown on the additional web page. Alternatively, the computer can be configured to automatically generate alphanumeric characters for all the groups at once and to fill the text boxes of all the groups all at once. Once the user has finished identifying all of the groups of samples and filling out all of the sample ID's 91 in the text boxes on the screen of computer 5, he clicks on a button labeled “next”. Computer 5 transmits this request to website 19, which responsively generates another web page in which the user 1 enters shipping and tracking information. This page, called the order confirmation page, includes a text box for entering a character string. This character string provides access to a web-based shipment tracking system of a commercial shipping company. In the preferred embodiment, the character string is a tracking number used by the shipping company to track the samples from the remote user 1 to the screening laboratory 20. In the preferred embodiment, the tracking number is provided to the user together with the source well container 2 and the packaging materials in which the user places the source well container 2 for shipment to the screening lab 20.

The order confirmation page also includes an invoice that lists the different tests requested by the operator in the foregoing steps on the screen of computer 5. Each test or group of tests is displayed on the screen adjacent to the price or prices for those tests. A total price of all the tests is displayed as well.

The order confirmation page has a second text box in which the remote user 1 can type the expected shipping date. The expected shipping date is the date on which remote user 1 intends to give the samples in their packaging materials to the delivery service associated with the tracking number. By providing the anticipated shipping date to the website 19 and then to the screening laboratory 20, personnel at the screening laboratory 20 can anticipate the arrival of each shipment and prepare for its arrival by pre-ordering reagents, probes and primer sets required for testing the samples in advance.

Once the operator has entered the tracking number and the expected shipping date, he clicks on a button labeled “confirm order”, which transmits the completed order, including the tracking number and expected shipping date to website 19 and order manager 22, and thence to LIMS 24.

In the preferred embodiment, once the order has been transmitted to the order manager 22, the order generates two electronic messages, which will be sent to different locations. The first message is cross-referenced in LIMS 24 with a list of stocked probes. If the probe designated by the user is not stocked, an order message is sent to a supplier 11, such as a contracted probe provider. This request can be transmitted from remote user 1 to screening laboratory 20 via any form of electronic communication, and then via a form of electronic communication 10 to suppliers' computer 8, or in the alternative, the order message can go from user 1 via any form of electronic communication link 12 to suppliers' computer 8. The supplier 11 creates the primer sets and probe based on the designated genetic sequence designated by the remote user 1 or the screening laboratory 20. The made to order probe can be referred to as the target-binding probe. This supplier 11 will then barcode and overnight ship 13 the primer sets and target-binding probes 17 to the screening laboratory 20. Once the primer sets and target-binding probes for each order for that day's screening are received by screening laboratory 20, the barcodes on the primer sets and target-binding probes are scanned into LIMS 24. The LIMS 24 records the date and time the primers and target-binding probes were received along with the quality control data provided from the probe provider.

In the preferred embodiment, the primer sets and target-binding probes are placed in workstation 14 and LIMS 24 will record the barcode of the probe and record its specific location on the deck of the workstation 14, as will be discussed in more detail with respect to the Screening Station 95. Additionally, the screening laboratory 20 and the LIMS 24 system correlates which target-binding probes will be used on which samples, as will be discussed in more detail with regard to the Screening Station 95.

The second message, in the preferred embodiment, that is generated from the order placement of the remote user 1 insures that the remote user 1 has the proper supplies to package and ship their samples. This message, sent via link 12, will define the barcode number of well container(s), shipping labels tracking number and amount of reagents needed for the user. In response to this message, supplier 11 will package 18 supplies for remote user 1 and ship 14A the supplies back to remote user 1.

Once the remote user 1 procures or receives these supplies, the remote user 1 places the appropriate samples into the source well containers 2 previously identified in the order sent to website 19, order manager 22 and LIMS 24. In other words, the remote user 1 fills each well of source well container 2 such that each well contains the same sample with the same sample ID 91 that the user previously identified in the order previously sent to website 19. Alternatively, if the user already had sufficient supplies when the user placed the order the user need not wait for a source well container 2 to be sent by a supplier, but can fill the source well container 2 when the user creates the order, or even before the order is created. What is important is that the contents of the actual 96 source well container 2 that the user fills exactly matches the description of the samples and has the same accession number as the order the user previously sent to website 19.

The samples can be obtained from prokaryotic or eukaryotic organisms. The samples may be a tissue sample from a mouse 8A, but can also come from other animals, plants and viruses. In the preferred embodiment, mouse tails or ears are snipped to provide a tissue sample. Source well container 2 is a 96 well plate or the like that receives the sample in each well of the well plate. A sufficient amount of lysis reagent can be added to cover the sample. In one embodiment, the lysis reagent is added prior to transit to the screening laboratory 20. Although, in the preferred embodiment the lysis reagent is added at the screening laboratory 20 at Lysing Station 92.

Referring now to FIG. 1, source well container 2 has an accession number 3 affixed to the side of the container. The accession number is used by LIMS 24 to track the source of source well container 2. The remote user 1 places the appropriate samples into the well locations in source well container 2 that they had previously designated while placing their order in FIG. 6. Once the samples are in the proper wells in the source well container 2 then the remote user 1 in one embodiment dispenses a predetermined amount of reconstituted lysis reagent 4 to cover the sample into each well using a pipette. The lysis reagent 4 is formulated to lyse the tissue to obtain cellular debris including genomic nucleic acid. A lysis reagent 4 can be formulated to lyse the biological sample while in transit between remote user 1 and the screening laboratory 20. The transit time is approximately 24 hours as all samples are shipped via an express delivery service, such as FedEx® (Memphis, Tenn.). The remote user 1 will add lysis reagent 4 to each well of the source well container 2. The lysis reagent 4 should completely cover the samples. Once the samples and lysis reagent 4 are in the source well container 2 the remote user 1 places a seal on the top of the source well container 2 preventing samples from leaking. The remote user 1 then places a plastic lid on the seal for transportation. The remote user 1 then places the source well container 2 into an overnight delivery service package 15. The remote user 1 will then seal the package and ship 16 to screening laboratory 20, and apply a barcode shipping label.

A biological sample can be collected in a variety of ways to facilitate rapid screening. In one aspect of the invention, the biological sample is a sample of tissue such as from a mouse biopsy. The sample of tissue can include a portion of a tail, toes and ears. The tissue sample is collected by a remote user 1 and placed in a well of a source well container 2. The microwell container is transported to the screening laboratory 20. A multi-well container as shown in FIG. 1, in the preferred embodiment, is a 96 microwell source well container 2 but can include other multi-well containers, such as Strip Racks, 24 well plates, 384 well plates and tube rack holders or the like. As described above with regard to FIG. 6, the remote user 1 operates computer 5 to enter a variety of data regarding the samples placed in the source well container. Once all of the samples in all of the wells have been identified in this manner, the remote user sends the source well container 2 containing a plurality of biological samples to a screening laboratory 20 for screening.

In another embodiment of this invention, the biological sample is a blood sample collected by nicking the animal to be tested and blotting the blood on a filter paper. The blotted filter paper is placed in individual wells of source well container 2 by the remote user 1 and transported to the screening laboratory 20. In both of these embodiments, the biological sample is disposed on an absorbent carrier.

In another embodiment, the biological sample is embryonic tissue or embryonic stem cells. A sample of embryonic tissue is placed or grown in a well of a source well container 2 by the remote user 1 and transmitted to the screening laboratory 20.

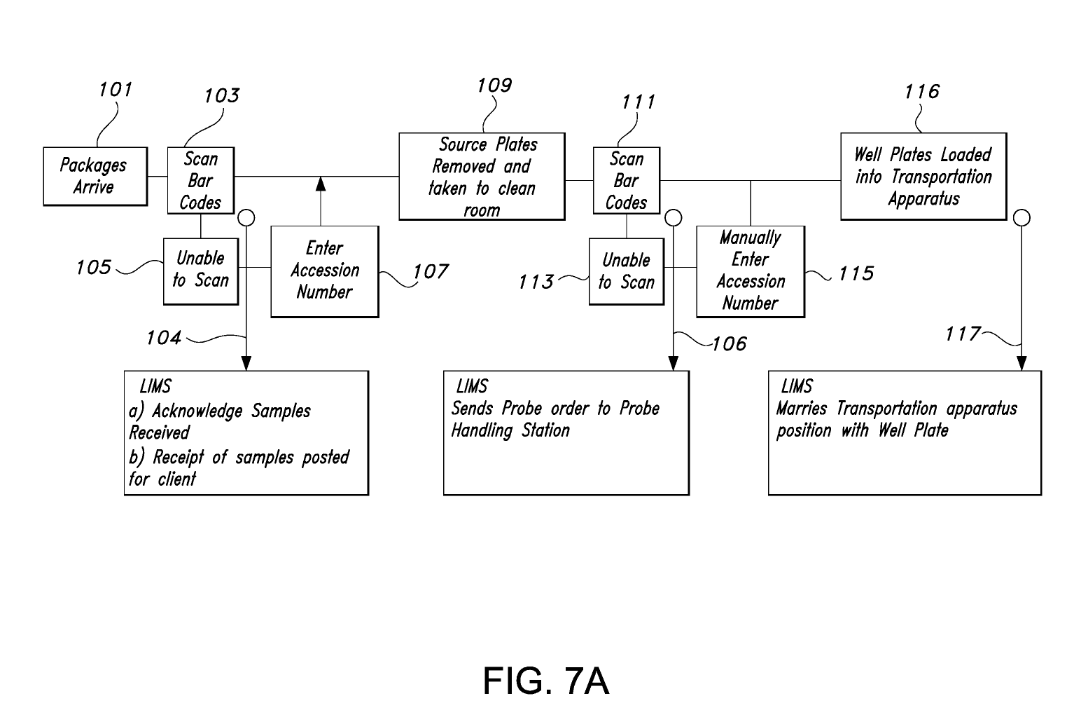

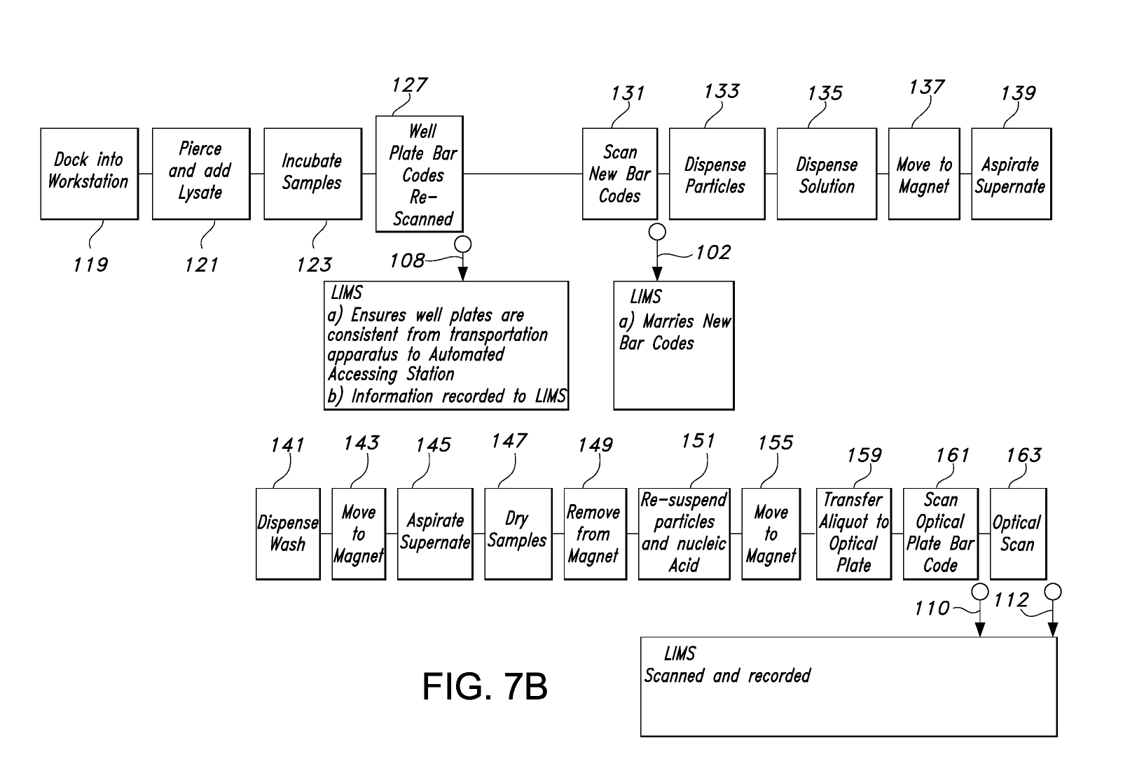

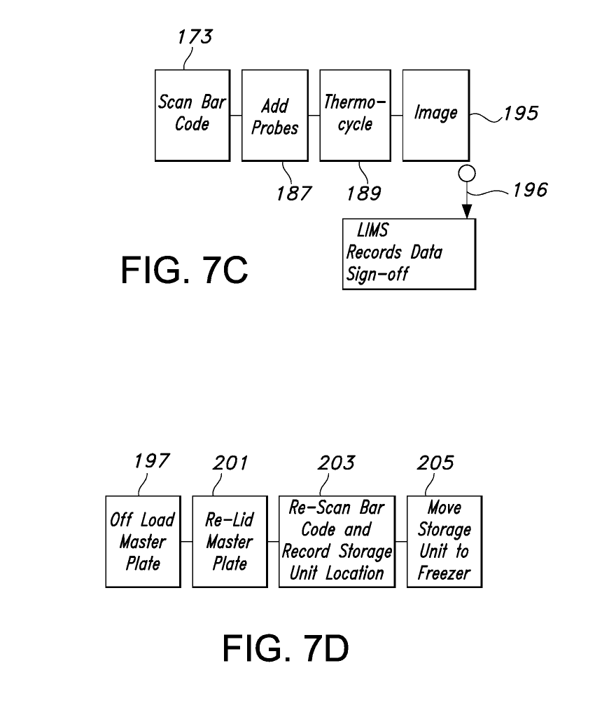

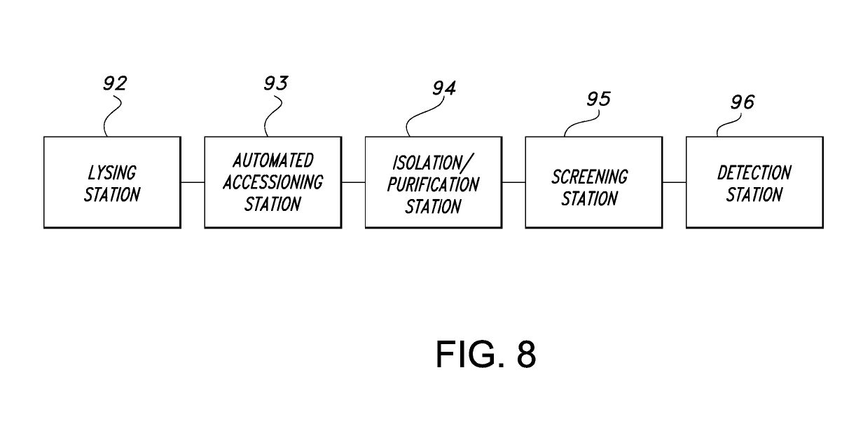

Now referring to FIG. 7A-D, the preferred embodiment of the present invention is shown. In FIG. 7A, the source well containers 2 arrive 101 at the screening laboratory 20. The tracking number of the shipping label is read with a barcode reader 103. If the shipping label is unreadable 105, the tracking numbers are manually entered 107. The scanning of the tracking number is received 104 in LIMS 24 and a received message is posted to the user's account as shown in tracking field. The source well container 2 are removed from the package and taken to a clean room 109. The source well containers 2 contain the raw biological matter and in one embodiment lysis reagent. The source well containers 2 individual barcodes are scanned by the barcode reader 111 and recorded 106 in LIMS 24 as accession numbers. LIMS 24 can send 106 a probe order to supplier 11 through the order manager 22. If the source well containers 2 individual barcodes are unable to be scanned 113, the accession numbers are entered manually 115. If the tracking number, accession number, user order and worklist properly correlate, LIMS 24 will activate (not shown) an active record number for the containers.

The source well containers 2 are loaded 116 into a transportation apparatus in a clean room. A transportation apparatus is any device that holds well containers and that can dock with the workstation. The transportation apparatus, in the preferred embodiment, includes several rigid trays stacked vertically in a housing unit that is mobile. This transportation apparatus can be moved between different automated stations, docked and the rigid trays can be removed in an automated fashion and processed on the deck of a workstation. Each rigid tray consists of nine locations for source well containers 2. Each of these nine locations per tray has a unique barcode designating its specific location inside the trays of the transportation module.

Source well container 2 accession number 3 is scanned with a barcode reader and the bar-coded source well container 2 location in the transportation apparatus trays is scanned. The barcodes of source well containers 2 are married 117 in LIMS 24 with the unique barcode locations in the transportation apparatus trays for tracking purposes. LIMS 24 records and associates each well container to this location. Once the transportation apparatus is loaded with the source well containers 2, the transportation apparatus is docked 119 into the laboratory workstation 14.

LIMS 24 will generate a worksheet for laboratory personnel (not shown). The worksheet outlines the probes and primer sets that the operator will need to prepare or gather in order to test the latest samples. The LIMS 24 worklist will generate a single file. The file format may include, but is not limited to, ASCII, XML or HTML. The file will be written into a specified directory on the network drive. The name of the file will be unique and will correlate to a run number. The extension will be unique for worklist files.

In the configuration described above, a transportation apparatus includes a housing unit provided to support several trays, each tray having nine different locations for nine source well containers 2. In an alternative embodiment, however, the housing unit can be eliminated. Instead, the source well containers 2 can be manually transported throughout the workstation in trays from functional station to functional station. In this system, operator at the laboratory loads source well containers into the trays after the source well containers 2 are received at the screening laboratory 20 and are scanned into LIMS 24 as described above for transportation to workstation 14. Alternatively, source well containers 2 can be transported individually to workstation 14 and be placed in a tray or trays that are already located at workstation 14.

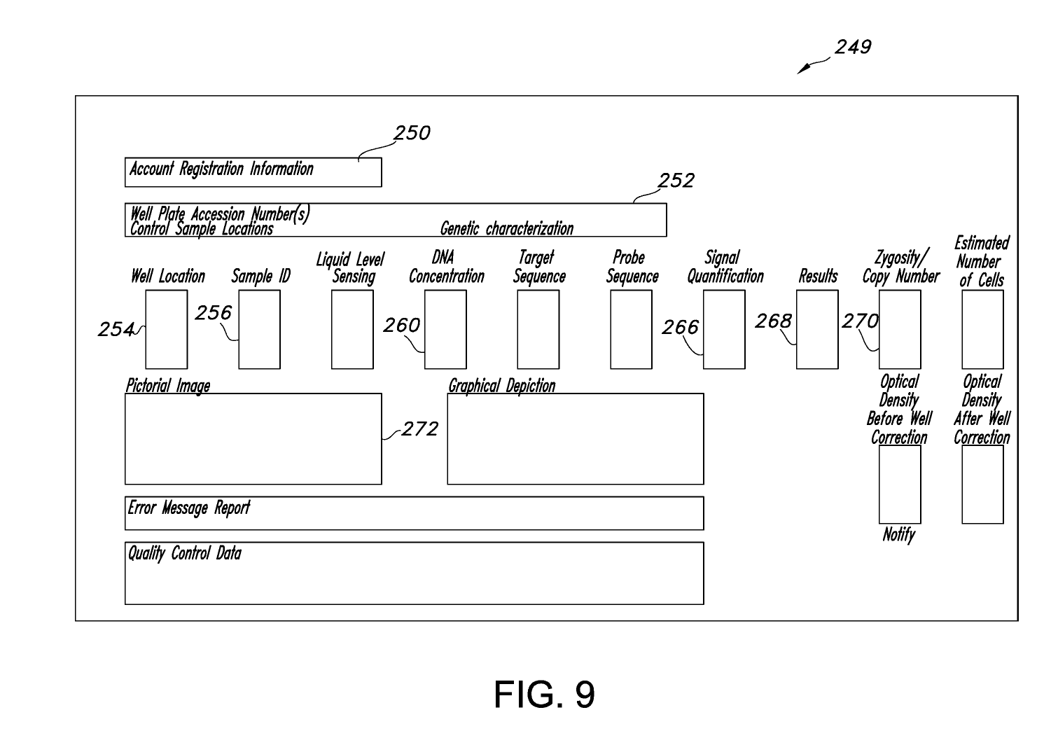

We now refer to FIG. 8, which depicts one embodiment of the workstation 14. Standard laboratory stations are logical groupings of laboratory operations. These groupings, however, do not necessarily refer to different physical stations. These logical groupings include: Lysing Station 92, Automated Accessioning Station 93, Isolation/Purification Station 94, Screening Station 95 and Detection Station 96, all of whom comprise workstation 14. The Screening Station 95 can include other screening processes such as PCR. Lysing Station 92 is an alternative step provided to lyse the samples in containers 2 in the event user 1 does not choose to lyse the samples by adding a lysis reagent before sending them to laboratory 20. The functions of the various logical stations are described below in connection with the steps shown in FIGS. 7A-D. The following description provides the preferred embodiment, although one skilled in the art could elect to conduct these methods with varying degrees of automation as required.

As mentioned above, remote user 1 need not add a lysis reagent to the samples before shipping them to screening laboratory 20. Instead, the samples may be shipped un-lysed (at room temperature) and may be lysed at laboratory 20 by piercing the cover 121 of the container 2 and treating each of the samples with a lysis reagent after docking the tray in the workstation 119 in the lysing station 92. The samples are incubated 123 to produce a lysate containing cellular debris including at least a portion of intact genomic nucleic acid.

For tissue biopsies, the lysis process in the preferred embodiment includes incubation with the lysis reagent, such as proteinase K and a Nuclei Lysing Solution (NLS) (Promega Corporation, Madison, Wis.) at 55° C. for three hours. Other lysis reagents such as sodium dodecylsulfate and proteinase K can be used. The lysis reagent is selected to not fragment the genomic nucleic acid. A sufficient amount is an amount in the wells of container 2 sufficient to cover the samples.

With respect to the blood sample collection method, a sufficient amount of a lysis reagent, such as Nuclei Lysing Solution (Promega Corporation, Madison, Wis.) is added to each well of source well containers 2 to cover the filter paper. With respect to animal embryonic tissue and embryonic stem cell screening, Nuclei Lysing Solution (Promega Corporation, Madison, Wis.) is added to each well containing the tissue/cells. The source well container 2 is treated under conditions to facilitate rapid lysis of the biological sample. In the preferred embodiment, these conditions are heating at 55° C. for three hours. Additionally, if the samples are embryonic tissue, in the preferred embodiment they are sonicated for 3-5 seconds after lysis. However, embryonic samples should not be sonicated for such a period of time to eliminate all intact genomic nucleic acid.

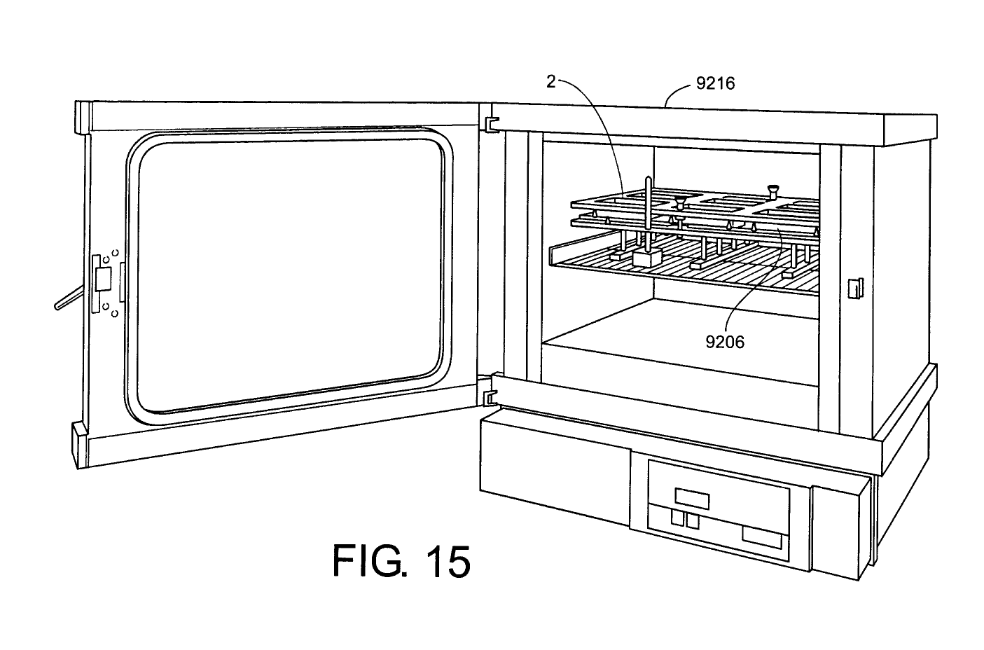

The preferred method of performing the above lysing steps at Lysing Station 92 includes loading source well containers 2 into the tray 9206 and taking the rigid tray to Lysing Station 92 to be lysed. Lysing Station 92 includes a liquid handler 9220, such as Genesis Tecan (Raleigh Durham, N.C.) or Multimeck Beckman (Indianapolis, Ind.). An example of a preferred Lysing Station 92 is shown in FIG. 14. It includes a frame 9202, on which a deck 9204 is mounted to provide a horizontal working surface, which supports tray 9206, which supports and positions up to nine source well containers 2. A material handler 9214 is fixed to frame 9202 and extends upward and across the top surface of deck 9204. A computer 9208 is coupled to material handler 9206 to direct the movement and operation of pipettes 9210. A trough or reservoir 9212 is provided on deck 9204, from which computer 9208 commands the material handler 9214 to aspirate lysis reagent into pipettes 9210 and to deposit the reagent into wells of container 2.

The operator first carries a plurality of source well containers 2 and places them on deck 9204 in one of the nine positions on the rigid tray 9206 that support and orient source well containers 2 thereby docking them 119 into the workstation 14. The operator then enters the number of wells that are filled with samples in each of the source well containers 2 into computer 9208 in combination with the location of that container with respect to tray 9206.

Knowing the location of each source well container 2 in tray 9206, and the number of wells that are filled with samples in each of these source well containers 2, computer 9208 then directs material handler 9214 to move the pipettes 9210 to each source well container 2 in turn, piercing 121 the barrier sealing mechanism and filling each of the wells of source well containers 2 containing a sample with lysis reagent. By providing the location and the number of samples, computer 9208 is configured to fill only the wells containing samples with lysis reagent and to leave the empty wells empty of lysis reagent.

Once each of the sample-containing wells has been filled with lysis reagent, the operator moves the entire tray or trays 9206 containing the samples to an oven 9216 (FIG. 15), where the samples are incubated 123 by heating for a period of about three hours at a temperature of 55° C. (described above). Once the incubation process is complete, the operator moves source well containers 2 supported on the tray or trays 9206 to Automated Accessioning Station 93.

An Automated Accessioning Station 93 provides a device to remove liquid from the source well container 2 to the primary master well container 6. The primary master well container 6 is the container in which the nucleic acid is isolated. It is preferably a 384 well plate (Fisher Scientific #NC9134044). Any commercially available automated accessioning device can perform this function such as Genesis® Tecan (Raleigh-Durham, N.C.) or Multimeck® Beckman (Indianapolis, Ind.). These devices are referred to as liquid handlers. The source well containers 2 barcode accession numbers 3 are re-scanned 127. This measurement will be recorded and posted 108 into the LIMS 24 database and reflected in the outcome report 249. Additionally, LIMS 24 ensures 108 that source well containers 2 are consistent from transportation apparatus to the Automated Accessioning Station 93. Error codes will be generated if a sufficient amount of raw testing material is not available. The liquid handler utilizes stainless steel, or the like, pipette tips that are washed between each sample transfer. Alternatively, disposable pipette tips may be used.

The nucleic acid lysate is transferred 129 to clean well containers, called primary master well containers 6. Each of the containers 6 has a scannable accession number, preferably a barcode accession number, called “barcodes” or “accession numbers” below. The barcodes of the primary master well containers 6 are scanned 131 and LIMS 24 marries 102 the barcodes for the primary master well containers 6 to the scanned barcode accession numbers 3 of the source well plates 2. The automated process accessioning continues until all of the day's pending samples are accessioned into the primary master well containers 6. The preferred method of performing the above steps at Accessioning Station 93 includes taking the rigid tray 9206 and the source well containers 2 from the incubating oven 9216 back to the same liquid handler 9220 that performs the functions of Lysing Station 92. This liquid handler 9220 is also preferably configured to function as Accessioning Station 93.

Referring now to FIG. 14, the operator returns tray 9206 to liquid handler 9220 and places tray 9206 back on deck 9204 generally in the same location it was in when the lysis reagent was inserted into each well containing a sample.

Once in that location, the operator commands computer 9208 to fetch the work list from LIMS 24 and electronically stores it in the computer memory of process controller 26. The work list includes the accession numbers of each source well container 2 that is in tray 9206, together with the probe type that should be used for each well. The work list uniquely associates the location of the well, the accession number of source well container 2 from which the well is from, the probe type that is to be used with the sample in that source well container 2, and the quantity of probe to be added to that sample.

Once computer 9208 fetches the work list, computer 9208 directs the operator to electronically scan 127 the accession numbers of all the source well containers 2 that are in rigid tray 9206 on deck 9204 of liquid handler 9220 using scanning device 9218 coupled to computer 9208. Scanning device 9218 is preferably a glyph scanner, character scanner, bar code scanner, dot matrix scanner, or RFID tag scanner, depending upon the form of the accession identifier (typically a barcode accession number 3) on source well container 2. Once source well containers 2 have been scanned 127, computer 9208 transmits 108 the accession numbers 3 to process controller 26 and thence to LIMS 24. Process controller 26 preferably includes an instrument database to which each of the computers of Lysing Station 92, Automated Accessioning Station 93, Isolation/Purification Station 94, Screening Station 95 and Detection Station 96 transmit their data in order to maintain an ongoing record of the testing process and the location of materials and samples throughout that process. The database is preferably implemented using Microsoft's SQL Server, although any relational database (e.g. Oracle), may be used.

Computer 9208 then commands material handler 9206 to transfer 129 the contents of each well (i.e. lysate) in source well containers 2 to a corresponding well in the primary master well container 6 using pipettes 9210. Computer 9208 directs the operator to scan 131 the accession numbers on the primary master well container 6. Like the accession number on source well containers 2, the accession number on the primary master well container 6 may be any electronically scannable indicia or device. Computer 9208 transmits the accession numbers to process controller 26, which sends them to LIMS 24. In this manner, LIMS 24 maintains a record of each sample and its location in each source well container 2 and in each primary master well container 6. LIMS 24 and process controller 26 correlate the accession number of each primary master well container 6 with the identity of each sample it contains, the strain for which each sample is to be tested, the designated genetic sequence or sequences that identify or indicate that strain, the probes and primer sets necessary to test for those designated genetic sequences and the results of the testing.

The tray of primary master well containers is moved by the transportation apparatus to the Isolation/Purification Station 94. In this station, the genomic nucleic acid will be isolated and purified using a separation method such as magnetic or paramagnetic particles. Purified genomic nucleic acid, substantially free of protein or chemical contamination is obtained by adding a sufficient amount of magnetic particles to each of the well containers that bind to a predefined quantity of nucleic acid. The term “magnetic” in the present specification means both magnetic and paramagnetic. The magnetic particles can range from 0.1 micron in mean diameter to 100 microns in mean diameter. The magnetic particles can be functionalized as shown by Hawkins, U.S. Pat. No. 5,705,628 at col. 3 (hereinafter '628 patent hereby incorporated by reference).

In the preferred embodiment, the magnetic particles are purchased from Promega Corporation, a measured amount of magnetically responsive particles are added 133 to the lysate mixture with or without the presence of a chaotropic salt 135. In the preferred embodiment, 13 μl amounts of 1 micron silica magnetic particles with chaotrope 113 μl (Promega Corporation, Madison, Wis.) are added to each well of the microwell container. The fixed volume of particles becomes saturated with nucleic acid and excess nucleic acid is removed. It has been observed that the resulting nucleic acid concentration between samples is very consistent. In a 50 μl pathlength read by the Genios (Tecan, Research Triangle Park, N.C.) a standard A₂₆₀ is 0.2 OD units. A standard concentration range of 0.1 to 0.3 O.D. units is disassociated from the magnetic particles to yield purified genomic nucleic acid.

Table 1 shows that with increasing amounts of magnetic particles, the nucleic acid concentration also increases.

TABLE 1

| | | Bead Volume |
|---|---|---|
| | | per |
| Average | Stdev | 150 μ1 of lysate |
| 0.7974 | 0.0072 | 27 |
| 0.8750 | 0.040 | 35 |
| 1.2328 | 0.026 | 50 |
| 1.7900 | 0.022 | 75 |

While the nucleic acid concentration is consistent between samples treated with the same protocol, several factors may increase or decrease the resulting standard concentration of genomic nucleic acid. These factors include: the binding reagent, the number of purification washes, and the solution that is used to elute the nucleic acid. The preferred binding solution for the magnetic particles obtained from Promega (Madison, Wis.) is a chaotropic salt, such as guadinium isothiocyanate. Alternatively, other binding reagents, such as 20% polyethylene glycol (PEG) 8000, 0.02% sodium azide and 2.5M sodium chloride may be used to nonspecifically bind the genomic nucleic acid to the surface chemistry of the functionalized magnetic particles. If functionalized magnetic particles are used, the preferred binding solution is PEG. The PEG or chaotropic guadinium isothiocyanate allows for the disruption of hydrogen binding of water, which causes binding of the nucleic acid to the particles. The preferred washing procedure to remove contaminants includes two chaotrope washes, after the initial chaotrope binding step, followed by four 95% ethanol washes. Aqueous solutions, or the like, are the best elution solutions. These solutions include water, saline sodium citrate (SSC) and Tris Borate EDTA (ie. 1×TBE).

The preferred device for performing the above functions of the Isolation/Purification Station 94 is a liquid handler 9402 identical in general construction to the liquid handler 9220 identified above for use as the Lysing Station 92 and the Accessioning Station 93 that has been configured to automatically transfer the various reagents and other liquids as well as the magnetic particles in the manner described below.

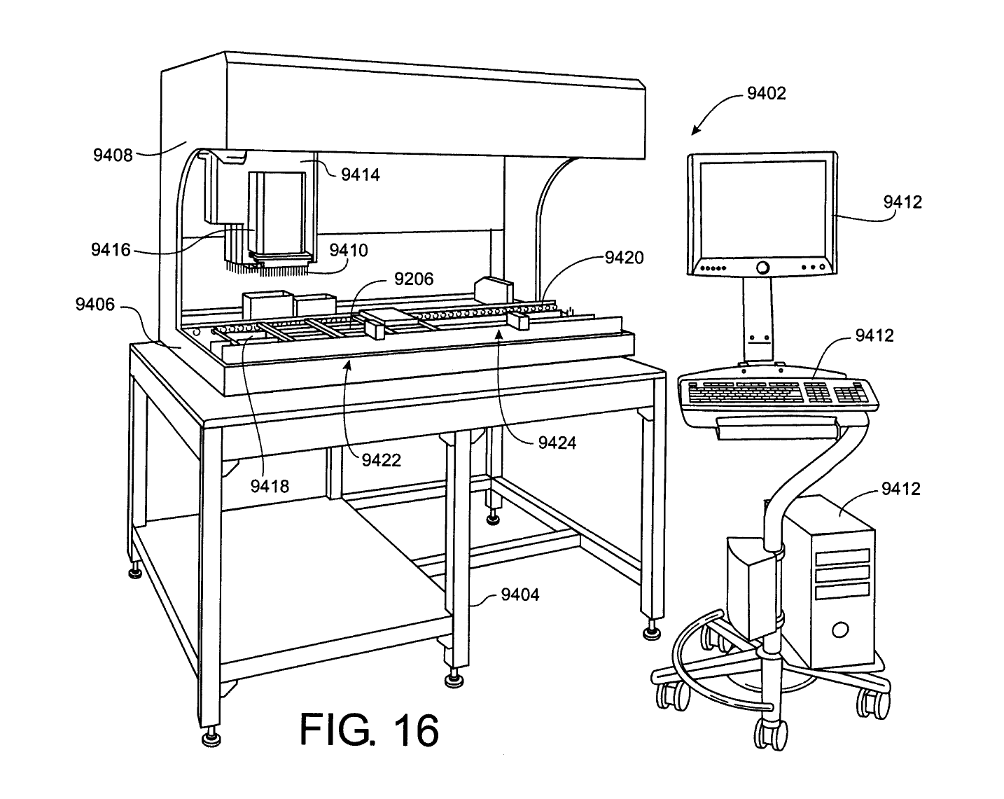

FIG. 16 illustrates a preferred embodiment of the liquid handler 9402. Handler 9402 comprises a frame 9404 on which is mounted a deck 9406, which is surmounted by material handler 9408, which supports and positions pipettes 9410 and is coupled to and controlled by computer 9412, which is in turn coupled to process controller 26 to communicate information to and from LIMS 24. Liquid handler 9402 includes a syringe pump 9414 that is coupled to and driven by computer 9412 to dispense magnetic particles via a 16×24 array of 384 pipettes 9410 simultaneously into all 384 wells of the primary master well container 6 under the command of computer 9412. Liquid handler 9402 also includes a second syringe pump 9416 that is configured to dispense a binding buffer into wells of the primary master well container 6 under computer control. The liquid handler also includes a magnet 9418 mounted in deck 9406 as well as a conveyor 9420 that is coupled to and controlled by computer 9412 to move the primary master well container 6 in tray 9206 back and forth between a first position 9422 in which the container is within the magnetic field and a second position 9424 in which the container is outside the magnetic field.

Before the functions of the Isolation and Purification Station 94 can be performed, the operator must first move the primary master well container 6 from Accessioning Station 93 to deck 9406 of liquid handler 9402 and place it in a predetermined location on the deck. Once the operator has placed the primary master well container 6, the operator starts an isolation/purification program running on computer 9412. This program drives the operations of liquid handler 9402 causing it to dispense magnetic particles 133 into all the wells of the primary master well container 6 containing lysed samples. Computer 9412 signals syringe pump 9414 to dispense the particles using pipettes 9410 into the primary master well container 6 when container 6 is in position 9424, away from the magnetic field created by magnet 9418.

Once the particles have been added, computer 9412 then directs the pipettes 9410 to add a chaotropic salt such as guadinium isothiocyanate to each of the wells to bind the genomic nucleic acid to the magnetic particles at 135. Once the chaotropic salt has been added, computer 9412 then mixes the contents of the wells by signaling the pipettes 9410 to alternately aspirate and redispense the material in each of the wells. This aspiration/redispensing process is preferably repeated three or four times to mix the contents in each well.

Once the contents of the wells have been mixed, computer 9412 pauses for two minutes to permit the particles, binding reagent, and raw biological material in the wells to incubate at room temperature in position 9424. When the two minutes have passed, computer 9412 commands the conveyor 9420 to move tray 9206 from position 9424 to position 9422, directly above magnet 9418 at 137. In this position the magnet draws the magnetic particles in each of the wells downward to the bottom of the wells of the primary master well container 6. Computer 9412 keeps tray 9206 and the primary master well container 6 over the magnet and within the magnetic field for 2-6 minutes, or until substantially all the magnetic particles are drawn to the bottom of each well and form a small pellet.

The particles drawn to the bottom of each well have genomic nucleic acid attached to their outer surface—genomic nucleic acid that the particles hold until an elution solution is placed in each well to release the genomic nucleic acid from the particles. With the particles at the bottom of each well and the wells located within the magnetic field, computer 9412 directs the pipettes to aspirate the supernatant 139.

Once the supernatant is removed, computer 9412 signals the conveyor to move the primary master well container 6 on tray 9206 to the nonmagnetic position 9424. The foregoing process of adding chaotropic salt, mixing the combination, pausing, drawing the magnetic particles down and aspirating the supernatant is repeated two more times.

Computer 9412 then directs the pipettes to introduce a wash solution (for example 70% ethanol when functionalized beads are used, or 95% ethanol (4×) when silica beads are used) to resuspend the particles 141. Computer 9412 again mixes the contents of the wells by signaling the pipettes to alternately aspirate and redispense the material in each of the wells. With the wash buffer and particles thoroughly mixed, computer 9412 again moves tray 9206 and the primary master well container 6 back over magnet 9420 in position 9422 143 and draws the magnetic particles back to the bottom of the wells. This wash process 141,143,145 is repeated three times to thoroughly cleanse the magnetic particles, and dilute and remove all supernatant.

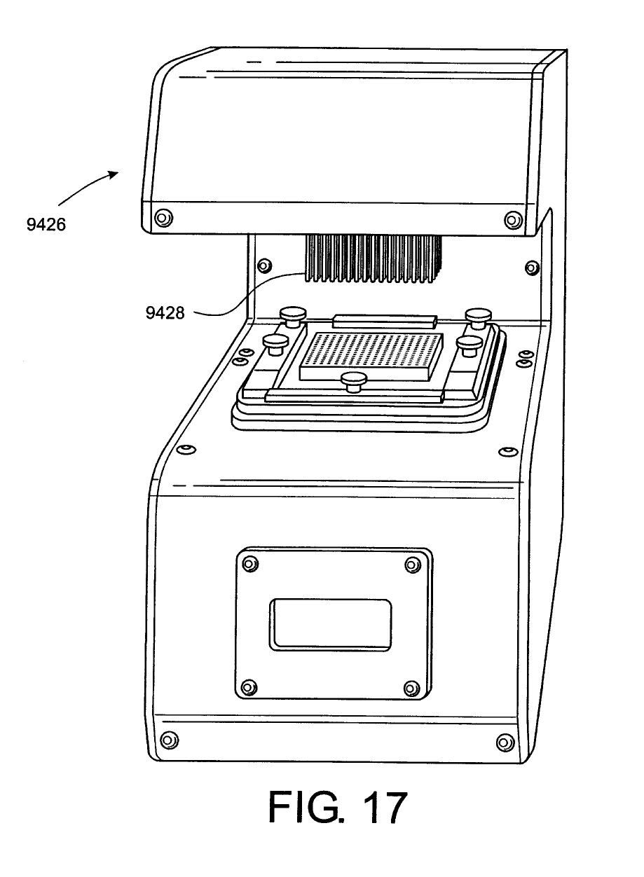

Once the particles are thoroughly washed, computer 9412 permits the magnetic particles in each well to air dry 147. In the preferred embodiment, shown in FIG. 17, the operator moves the primary master well container 6 to a dryer 9426 (an “Ultravap” dryer by Porvair Sciences, UK) having 384 tubules disposed in a 16×24 array 9428 that are configured to be simultaneously inserted into each of the wells of the primary master well container 6 and to supply warm, dry air thereto. In an alternative method, computer 9412 causes material handler 9408 to direct compressed dry nitrogen gas into each well of the primary master well container 6, drying the particles out in place while the container is in the magnetic field. Alternatively the samples can be permitted to air dry. Once the particles are completely dry, the primary master well container 6 can be subsequently moved away from the field of magnet 149.

Once the particles are almost dry, the operator returns the primary master well container 6 to the liquid handler 9402 and directs the computer 9412 to command the pipettes 9410 to fill the wells with an elution solution 151 and resuspend the particles. This elution solution is formulated to elute the bound genomic nucleic acid from the particles. An example of one such elution solution is 0.01M Tris (pH 7.4), sodium saline citrate (SSC), dimethyl sulfoxide (DMSO), sucrose (20%), 1×TBE, or formamide (100%). In the preferred embodiment, the elution solution is nuclease-free water. Nuclease free water is selected to minimize contamination and produce a standard concentration of purified genomic nucleic acid. In the preferred embodiment, the elution solution temperature is 22° C. A preferred yield is about 20 ng/μL of genomic nucleic acid is obtained.

After resuspending the genomic nucleic acid in a solution for a predetermined period of time, computer 9412 again moves tray 9206 with the primary master well container 6 via conveyor 9420 to position 9422 over magnet 9418 155. The magnet, in turn, draws the magnetic particles down to the bottom of each well. This leaves the genomic nucleic acid mixed and suspended in the elution solution. Computer 9412 then directs the pipettes to aspirate a small amount (50 μl) of purified genomic nucleic acid and to transfer 159 the small amount from each well into a corresponding well of a clean optical 384-well container that is also mounted on deck 9406. The operator scans 161 a barcode accession number on the optical container and computer 9412 transfers the scanned accession number to process controller 26, which then transfers it to LIMS 24. The operator takes this optical container to a UV spectrometer (Genios, by Tecan of Raleigh-Durham, N.C.), and directs the UV spectrometer to optically scan the optical container, by making an A₂₆₀ measurement 163. This measurement is electronically transferred 112 to LIMS 24 over a data communications link.

If another fully automated system is desired, the magnetic separator can be automated and rise from the bottom of the workstation and make contact with bottoms of all primary well containers simultaneously.

In the preferred embodiment for the biological sample, the genomic nucleic acid is not sonicated after separation from the cellular debris. The genomic nucleic acid includes at least a portion of intact nucleic acid. Unsonicated nucleic acid is recovered in the condition it is found in the lysate. Thus, if the genomic nucleic acid is intact in the lysate, it is intact (i.e., unfragmented) as attached to the particles. The sample contains at least a portion of intact genomic nucleic acid.

In certain types of samples, such as embryos, the genomic nucleic acid is substantially intact. In one embodiment, the genomic nucleic acid can be sonicated before or after separation with the magnetic particles. When the biological tissue is embryonic sonication is preferred. Sonication can be done by any conventional means such as a fixed horn instrument or plate sonicator. In the one embodiment, the genomic nucleic acid is sonicated for five seconds to produce nucleic acid fragments. Although there is a wide range of fragments from about 100 base pairs to up to 20 kilobases, the average size of the fragment is around about 500 base pairs.

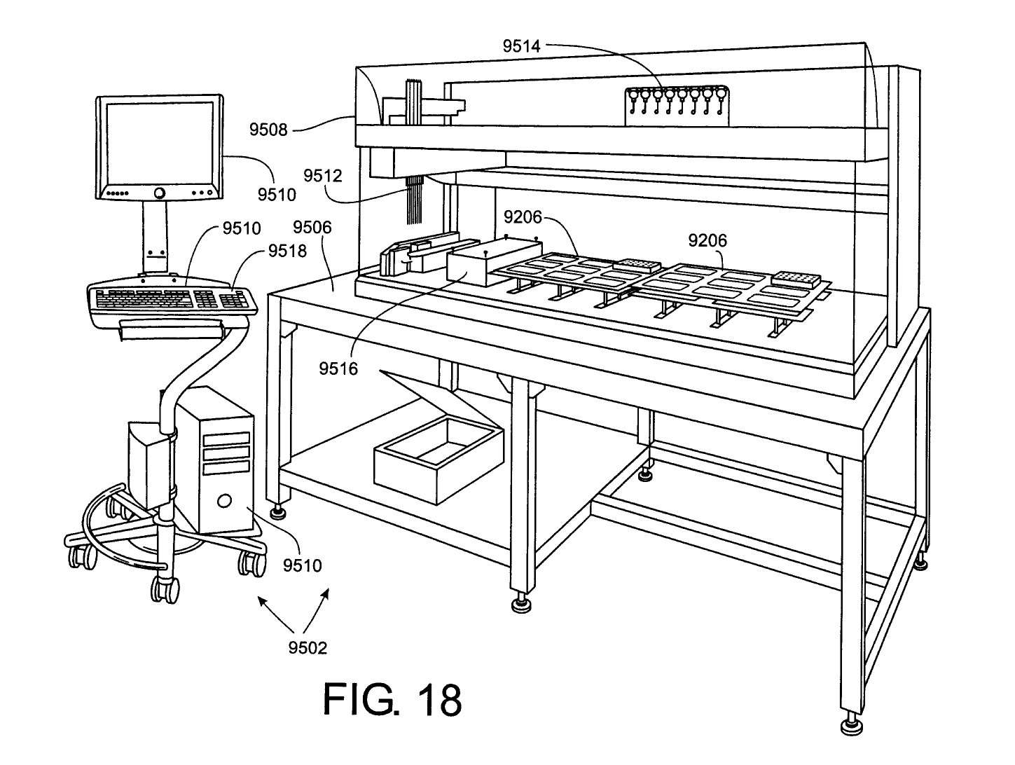

The primary master well container 6 is transported to the deck of the Screening Station 95 (FIG. 18) where its bar code is scanned 173. The operator places the container on a magnet, drawing all the magnetic particles to the bottom of the wells. The supernatant contains the purified genomic nucleic acid. LIMS 24 generates a worklist containing barcodes that list the primer/probe combinations that need to be loaded onto the deck of the machine. The primer-probe combinations are contained in barcoded tubes. An operator loads the barcoded tubes randomly into a probe box. The operator then scans the barcodes on the tubes using a Matrix scanner coupled to LIMS 24. The primer set and probe combinations in the tubes are then loaded into an ABI 384 PCR plate (Applied Biosystems, Forest City, Calif.). The genomic nucleic acid sample from each well of the primary master well container 6 is added to a corresponding well of the ABI PCR plate that contains the primer-probe combination or combinations appropriate to discern the relevant genotype 187. The ABI plate is then sealed with sealing tape and taken to the Detection Station 96 and placed in an ABI 7900. In the preferred embodiment the ABI 7900 cycles the ABI PCR plate 40 times between temperatures specified by the manufacturer. The operator can vary the number of cycles and the temperatures as desired to increase the signal provided by the samples.

FIG. 18 shows a preferred device for performing the Screening Station 95 functions. It comprises a liquid handler 9502 such as Genesis Tecan (Raleigh Durham, N.C.) or Multimeck Beckman (Indianapolis, Ind.). It includes a frame 9504, on which a deck 9506 is mounted to provide a horizontal working surface for first tray 9206 and second tray 9206. The first and second trays (as described above) can support and position nine primary master well containers 6.

Liquid handler 9502 also includes a material handler 9508 that is fixed to frame 9504 and extends upward and across the top surface of deck 9506. A computer 9510 is coupled to material handler 9508 to direct the movement and operation of pipettes 9512. Pipettes 9512 are fluidly coupled to a syringe pump 9514.

Probe block 9516 is disposed on the surface of deck 9506 and contains several tubes (not shown) each tube containing one or more combined primer sets and probes. The operator bar-codes each tube and enters the data indicative of the tube contents (the particular primer or probe in each tube, its volume and concentration) into LIMS 24, which stores the data associated with the bar code on the tube for later reference 173.

The operator places the primary master well containers 6 on deck 9506, scans the bar code accession number of the primary master well container 6, and signals computer 9510 to start transferring genomic nucleic acid, probes and primer sets.

Based upon the information provided by the remote user 1, including the samples, the strains for which the samples are to be tested, and the designated genetic sequences indicated by the strains, as well as the probes and primer sets necessary to detect those designated genetic sequences, as well as the location of each sample in the ABI PCR plate, LIMS 24 calculates a worklist that identifies for the operator which (and how many) tubes containing which probes and which primer sets must be placed in the probe block 9516 to test the samples in the primary master well container 6.

The operator first prints out this worklist, using it as a guide to identify and select particular tubes containing the proper probes and primers. The operator takes these tubes out of storage, places them in the probe block 9516 and places the probe block 9516 on the Matrix scanner.

The Matrix scanner is coupled to LIMS 24, and is configured to scan the bar codes on each tube through holes in the bottom of the probe block. The scanner passes this information to LIMS, to which it is coupled, which in turn compares the bar codes of the scanned tubes with the bar codes of the probes identified on the worklist. Only if the operator has loaded the probe block with the appropriate type and number of probes and primer sets will LIMS 24 permit the operator to proceed. In this manner, LIMS is configured to verify that the operator has inserted the appropriate and necessary tubes of probes and primer sets into the probe block.

Once LIMS 24 has verified that the proper tubes of probes and primer sets have been inserted into the probe block, it is configured to indicate to the operator that the probe block is acceptable and that the process steps at Screening Station 95 can begin.

The steps of preparing tubes of probes and primer sets, entering them into LIMS, preparing a worklist, filling a probe block and verifying the probe block, all happen prior to the time the operator takes the primary master well container 6 with its 384 wells to the deck 9506 of liquid handler 9502 and places it in position on deck 9506.

The operator places the primary master well container 6 in position on first tray 9206 located on deck 9506 of liquid handler 9502. The operator electronically scans the container with an electronic scanner 9518 coupled to computer 9510 which, in turn, is coupled to process controller 26. As described above, the scanner may be any of several types of electronic scanner but is preferably a bar code scanner.

If there are several primary master well containers 6, they are preferably carried from the liquid handler of the Isolation/Purification Station 94 to the liquid handler of the Screening Station 95 in tray 9206, which can accommodate nine separate primary master well containers 6.

The operator also places a secondary master well container 27 (preferably an ABI 384 PCR plate) in a predetermined location on the second tray 9206 located on deck 9506 adjacent to the first tray 9206. The operator electronically scans the secondary master well container 27 with the electronic scanner 9518 and stores the location and identity of the secondary master well container 27 in process controller 26 which transmits the data to LIMS 24.

If there are several primary master well containers 6 that must be transferred to secondary master well containers 27, the corresponding secondary master well containers 27 may also be taken to liquid handler 9502 in trays 9206, rather than the operator carrying each secondary master well container 27 to second tray 9206 individually.

Once the operator places at least one primary master well container 6 in first tray 9506 and at least one secondary master well container 27 in second tray 9506, the operator signals computer 9510 to begin combining the probes, primer sets, and genomic nucleic acid extracted from the samples.

Generally speaking, computer 9510 commands material handler 9508 to extract probes and primer sets from tubes in probe box 9516 and deposit them in each secondary master well container 27 in second tray 9206. Computer 9510 then commands material handler 9508 to extract the genomic nucleic acid from the wells of each primary master well container 6 in first tray 9206 and deposit the samples in wells in a corresponding secondary master well container 27. When the pipettes 9512 deposit the genomic nucleic acid samples, the probes, and the primer sets in wells in the secondary master well containers 27, computer 9510 commands material handler 9508 and pipettes 9512 to mix the samples using the aspiration/redispensing methods discussed above.

The secondary master well containers 27 receive a number of aliquots of biological sample in multiple wells of the secondary master well container. In one embodiment, an aliquot of the biological sample of the strain is dispensed into at least four wells of the secondary master well container 27. To at least two of the four wells at least one probe and primer set (e.g. SEQ ID NO. 23, 24 & 25) corresponding to at least one designated genetic sequence is added. A probe (SEQ ID NO. 21) and primer set (SEQ ID NO. 19 & 20) correspond to a reference sequence (SEQ ID NO. 18) is added to the third and fourth well. Thus, for example, if the genotype screening includes four designated genetic sequences, then four wells of the secondary master well containers 27 receive an aliquot of the biological sample and the corresponding probes and primer sets for each designated genetic sequence. Additionally, four wells receive an aliquot of the biological sample and the corresponding four probe and primer sets. This second set of wells is referred to as the replicants. The function of the replicants is quality control. Additionally, two additional wells receive aliquots of the biological sample and the housekeeping or screening reference probe/primer set.

In a simpler embodiment, the validity of the screening data can be evaluated by dispensing an aliquot of a biological sample of the strain designated by the remote user into at least two wells of a microwell container. In one well at least one probe and primer set is added corresponding to the at least one designated genetic sequence and to the other well at least one probe and primer is added corresponding to the reference sequence (SEQ ID NO. 18). The biological sample is screened and the probe signal values are compared between the probe for the designated genetic sequence and the probe for the referenced sequence.

In other embodiments, multiple probe and primer sets can be multiplexed into a single well. Furthermore, the detection of SNPs involve adding two probes to a well.

Between one and five microliters of nucleic acid and four and fifteen microliters of probes and primer sets are preferred to insure proper mixing of the samples and proper polymerization in the PCR process of the Detection Station 96 that follows.

Once the wells in the secondary master well containers 27 are filled with the appropriate purified genomic nucleic acid samples, primer sets and probes, and these materials are mixed, computer 9510 signals the operator that the screening process is complete. The plate is then sealed with optical sealing tape. The operator then moves the secondary master well containers 27 to Detection Station 96 for further processing.

In the preferred embodiment, the central component of Detection Station 96 is the ABI 7900. The secondary master well containers 27 are placed inside the ABI 7900, where they are thermocycled 189 40 times and exposed to an excitatory energy source to produce a quantifiable signal 195 from the signal molecule. More particularly, the Detection Station 96 scans the secondary master well container's 27 barcode and reports it 196 to LIMS 24.

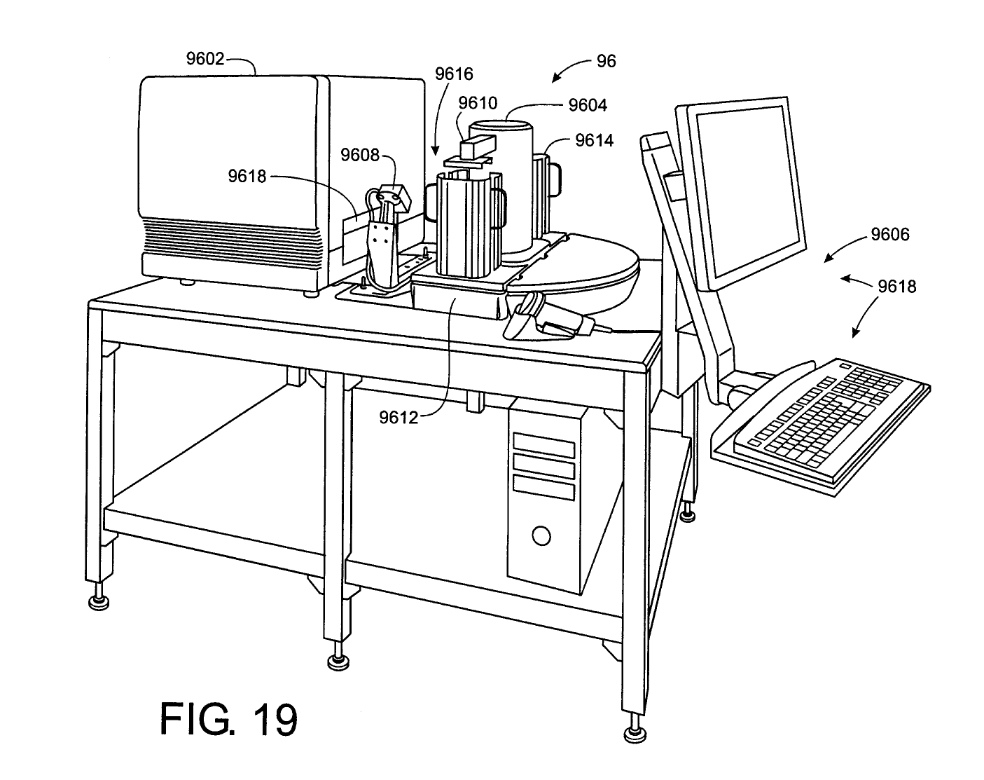

FIG. 19 illustrates a preferred device for performing the functions of Detection Station 96. It includes a PCR instrument 9602 (here shown as an ABI 7900), a material handler 9604 (here shown as a ZYmark arm), a computer 9606, and an electronic scanner 9608 (here shown as a barcode scanner).

Computer 9606 is coupled to PCR instrument 9602, material handler 9604, and process controller 26. It communicates with PCR instrument 9602 to control the insertion and removal of secondary master well containers 27 from PCR 9602 by handler 9604. Computer 9606 is also coupled to PCR instrument 9602 to process test results from the test performed by PCR instrument 9602 and to transmit those test results to process controller 26 and then to LIMS 24.

Scanner 9608 is coupled to handler 9604 to scan the accession numbers on the secondary master well containers 27, and to transmit those accession numbers to LIMS 24.

Material handler 9604 includes an arm 9610 that is commanded by computer 9606 to move between three positions: an incoming material hopper 9612, and outgoing material hopper 9614, and loading/unloading position 9616. Handler 9604 moves between these positions under the control of computer 9606, which commands this movement.

The operator first loads incoming material hopper 9612 with one or more secondary master well containers 27. The operator then operates the computer terminal 9618 of computer 9606, commanding computer 9606 to load and test the secondary master well containers 27. In response, computer 9606 commands arm 9610 to move to the incoming material hopper 9612, grasp the topmost secondary master well container 27, and to carry that container to the loading/unloading position 9616. Computer 9606 also commands PCR instrument 9602 to extend a tray (not shown) from an opening 9618 in the side of the ABI 7900, and commands arm 9610 to place the secondary master well container 27 on that tray. Scanner 9608 is configured to scan the barcode accession number on the secondary master well container 27, thereby making an electronic record of the secondary master well container 27 that is being tested. Scanner 9608 transmits this accession number to computer 9606, which later correlates the accession number with the test results provided by ABI 7900.

Once the secondary master well container 27 is placed in the tray, computer 9606 commands PCR instrument 9602 to retract the tray, and to begin testing the material in the secondary master well container 27, which is now inside PCR instrument 9602. PCR instrument 9602 signals computer 9606 when testing is complete. PCR instrument 9602 also transmits the test results to computer 9606. Computer 9606, in turn, commands PCR instrument 9602 to eject the secondary master well container 27 that has just been tested, moving it back to loading/unloading position 9616. Once the secondary master well container 27 is in this position, computer 9606 commands material handler 9604 to move arm 9610 back to the loading/unloading position 9616 and to retrieve the secondary master well container 27 that has just been tested. Computer 9606 commands arm 9610 to move the just-tested secondary master well container 27 to outgoing material hopper 9614, where it is deposited, awaiting later removal by the operator of Detection Station 96.

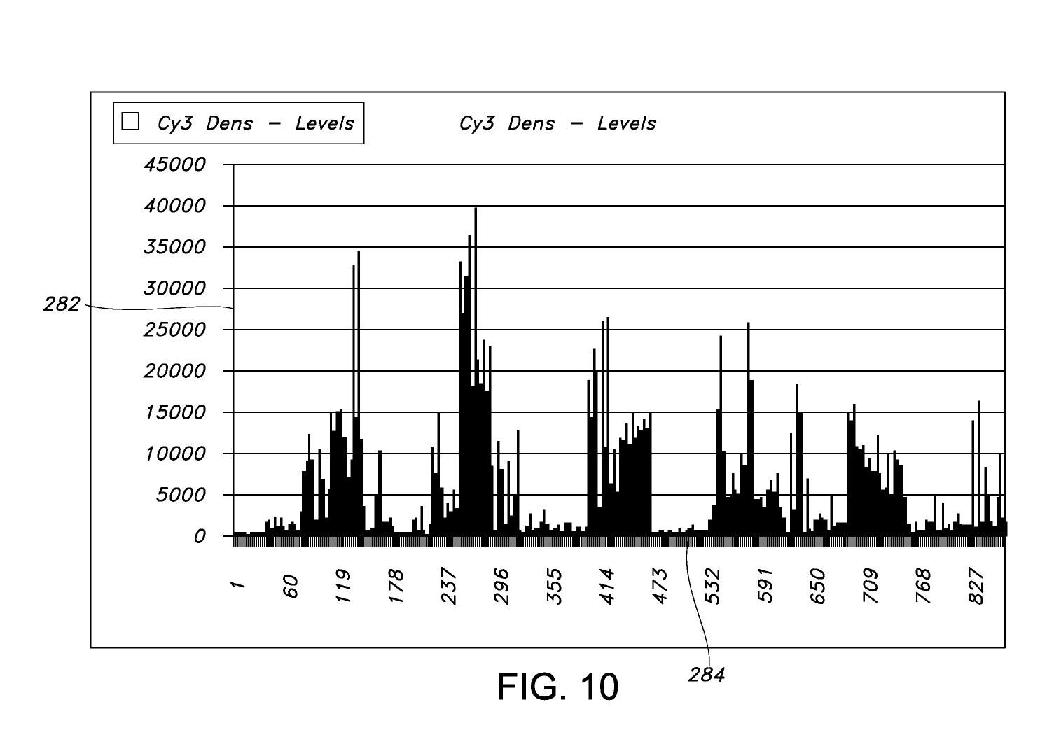

Now referring to FIG. 9, LIMS 24 now prepares the outcome report 249. Several calculations are performed before they are posted to the outcome report 249. In the preferred embodiment, such calculations include the evaluation of all replicates per sample. Calculating the relationship between the experimental quantified signal and the quantified signals of designated control may elucidate the copy number, zygosity or mosaic nature of the sample. The ratio for homozygous individuals should be twice the ratio of heterozygous individuals.

A reference sequence (SEQ ID NO. 18) and respective primer set and probe (SEQ ID NO. 19-21) is used to normalize the signal of every other probe used for that sample. The resulting value, called an RCN, is a comparison of the signal of the test probe (i.e. probes for portion of the designated genetic sequences) to the reference sequence. This control serves an additional purpose which is to evaluate the consistency of the nucleic purification system. This control will produce a magnitude of fluorescence directly proportional to the amount of starting nucleic acid, so nucleic acid concentrations can be compared. More specifically, the probe value corresponds to the designated genetic sequence is compared to the probe value of the replicant. Similarly, each value is compared to the probe value for the reference sequence to evaluate the validity of the data obtained.

For each sample, the CT values for the two wells containing the housekeeping gene, cjun, are averaged (CT_{cjun}). The RCN values are calculated by comparing the test probe (i.e. Neo or Cre) signal to the housekeeping gene signals or each of the two test probe wells (T₁ and T₂), the following equation is applied:

TABLE 2
Example of RCN Calculation
RCN₁ = 2^{-(CT}^{₁}^{- CT}^{_{cjun)}}
RCN₂ = 2^{-(CT}^{₂} ^{- CT}^{_{cjun)}}

| | | | | | Average | |
|---|---|---|---|---|---|---|
| Well | Sample Name | Detector | Task | CT | c-jun | RCN |
| C1 | Neomycin KO 1 | c-jun | Unknown | 25.37 | 25.27 | |
| D1 | Neomycin KO 1 | c-jun | Unknown | 25.17 | | |
| E1 | Neomycin KO 1 | Neo A | Unknown | 33.27 | | 0.00 |
| F1 | Neomycin KO 1 | Neo A | Unknown | 34.24 | | 0.00 |

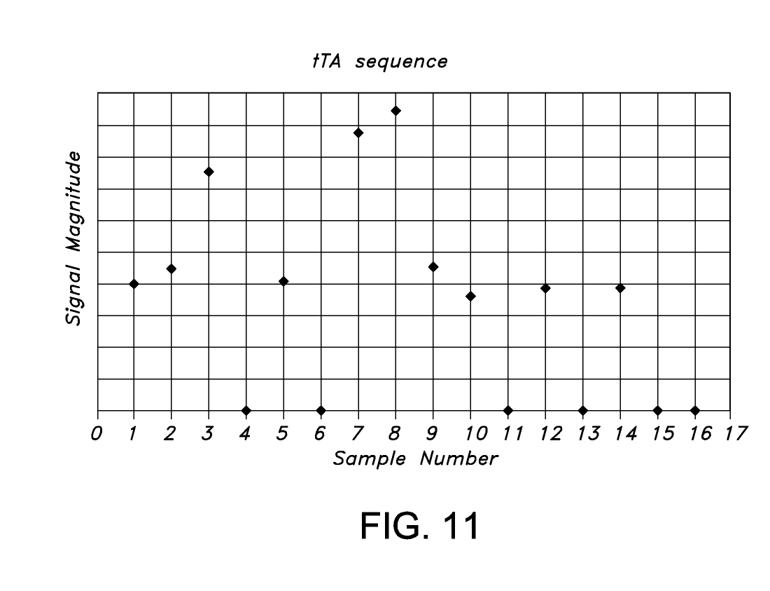

Now referring to FIG. 9, the sample outcome report 249 may include account registration 250, well plate container 2 barcode number(s) (i.e. accession numbers) 252, control sample locations 252 and genetic characterization of the designated control 252. Additionally, the outcome report 249 may include well location 254, sample identification 256, nucleic acid concentration 260, signal quantification 266, qualitative results 268, zygosity/copy number 270, quantitative analysis via comparison to designated control signal strengths allowing for copy number estimation, zygosity or mosaic nature 270. The outcome report 249 may also include a picture file (email) or pictorial representations of results 272 as shown in FIG. 10. Additionally, information gathered at the request of the remote user 1 from optimization and sequence confirmation quality control data and error messages may be included in the outcome report 249. The remote user 1 may choose to have this file electronically sent or choose to be electronically notified. Additionally, remote user 1 has the option to have a hard copy sent via the postal service or facsimile.

Once the LIMS 24 has compiled all the data for the outcome report 249, the outcome report will be sent 7 to the remote user 1. In the preferred embodiment, LIMS 24 will send the report via a remote link 7 to either the remote user 1 or the order manager 22, which can post the results on the web site 16 or via an electronic link 7. The LIMS 24 will keep results available for six months and then the results will be recorded onto a long-term storage disk and archived.

The following examples are provided by way of examples and are not intended to limit the scope of the invention.

### 8. EXAMPLES

### Example 1—Mouse Tail Genotyping

A biological sample in the form of a mouse tail biopsy is submitted via FedEx (Memphis, Tenn.) overnight delivery to the screening laboratory 20 from the remote user 1. Each sample occupies one well of a 96-well source well container 2.

The remote user 1 provides the genetic line identification 84. A line includes at least one designated genetic sequence. In the genetic line identification 84 provided by the remote user 1. The remote user 1 selects a designated genetic sequence. The genetic line identification 84 has been previously associated with the designated genetic sequence CRE (SEQ ID NO. 22); Mn1Tel (SEQ ID NO. 38) and p16 (SEQ ID NO. 50).

A lysis reagent (made of 2.5 μl of proteinase K (VWR EM-24568-3) and 147.5 μl of Nuclei Lysing Solution (Promega Corporation, Madison, Wis. A7943) per sample) is gently mixed and poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is then placed in a 55° C. oven for three hours. The well plate is then placed back on the deck of the Tecan Genesis Workstation (Research Triangle Park, N.C.). The liquid handler aspirates 50 μl of each sample and dispenses it in to a 384 well primary master well container (Fisher Scientific #NC9134044). Once all of the samples are transferred, the primary master well container is moved to the deck of the Isolation Station Purification Station 94.

One-hundred and twelve microliters of SV Lysis reagent (Promega Corporation, Madison Wis., # Z305X) a chaotropic salt are added to each sample. Next, 13 μl of magnetic particles (Promega Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container are placed on a 384 tip dryer for 11 minutes. Then the microwell container are moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 OD units is acceptable.

The primary master wellplate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog # 4326708), 1× real time PCR primer set/probe mix for a designated genetic sequence (Applied Biosystems Assays-by-Design (SM) Service 4331348) and 25% isolated DNA. In this example, the primer set as set out in SEQ ID NO. 23 and 24 and probe as set out in SEQ ID NO. 25 correspond to the designated genetic sequence CRE (SEQ ID NO. 22). Additionally, the primer set as set out in SEQ ID NO. 35 and 36 and probe as set out in SEQ ID NO. 37 correspond to the designated genetic sequence Mn1Tel (SEQ ID NO. 38). Additionally, the primer set as set out in SEQ ID NO. 51 and 52 and probe as set out in SEQ ID NO. 53 correspond to the designated genetic sequence for p16 (SEQ ID NO. 50). The Tecan Genesis added the reagents together in the ABI 7900 384 Well Optical Plate. The plate is then sealed with optical sealing tape (ABI, #4311971).

The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Tables 3 and 4. On average, these results are transmitted to the remote user 1 within twenty-four hours of receiving the biological sample at the screening laboratory 20.

TABLE 3

| | | Designated | | |
|---|---|---|---|---|
| | Sample | Genetic | | |
| Well | Named | Sequence | CT | RCN |
| 25 | 51 | Cjun | 25.722 | — |
| 49 | 51 | Cjun | 25.927 | — |
| 121 | 51 | CRE | 21.937 | 14.799 |
| 145 | 51 | CRE | 21.939 | 14.779 |
| 73 | 51 | Mn1Tel | 22.24 | 12.879 |
| 97 | 51 | Mn1Tel | 21.816 | 17.278 |
| 169 | 51 | p16 | 27.945 | 0.247 |
| 193 | 51 | p16 | 28.076 | 0.225 |
| 217 | 52 | Cjun | 26.26 | — |
| 241 | 52 | Cjun | 26.188 | — |
| 313 | 52 | CRE | 22.475 | 13.451 |
| 337 | 52 | CRE | 22.441 | 13.767 |
| 265 | 52 | Mn1Tel | 22.747 | 11.134 |
| 289 | 52 | Mn1Tel | 23.62 | 6.081 |
| 2 | 52 | p16 | 28.884 | 0.158 |
| 361 | 52 | p16 | 28.612 | 0.191 |
| 26 | 53 | Cjun | 25.919 | — |
| 50 | 53 | Cjun | 25.919 | — |
| 122 | 53 | CRE | 31.432 | 0.022 |
| 146 | 53 | CRE | 31.553 | 0.02 |
| 74 | 53 | Mn1Tel | 22.122 | 13.898 |
| 98 | 53 | Mn1Tel | 21.968 | 15.467 |
| 170 | 53 | p16 | 27.722 | 0.286 |
| 194 | 53 | p16 | 27.717 | 0.287 |
| 218 | 54 | Cjun | 25.909 | — |
| 242 | 54 | Cjun | 25.915 | — |
| 314 | 54 | CRE | 21.745 | 17.96 |
| 338 | 54 | CRE | 21.669 | 18.937 |
| 266 | 54 | Mn1Tel | 22.15 | 13.567 |
| 290 | 54 | Mn1Tel | 24.116 | 3.472 |
| 3 | 54 | p16 | 28.029 | 0.231 |
| 362 | 54 | p16 | 27.703 | 0.289 |
| 27 | 55 | Cjun | 26.729 | — |
| 51 | 55 | Cjun | 26.836 | — |
| 123 | 55 | CRE | 22.146 | 24.865 |
| 147 | 55 | CRE | 22.028 | 26.993 |
| 75 | 55 | Mn1Tel | 22.602 | 18.134 |
| 99 | 55 | Mn1Tel | 28.724 | 0.26 |
| 171 | 55 | p16 | 28.258 | 0.36 |
| 195 | 55 | p16 | 28.501 | 0.304 |
| 219 | 56 | Cjun | 27.348 | — |
| 243 | 56 | Cjun | 27.839 | — |
| 315 | 56 | CRE | 35.193 | 0.005 |
| 339 | 56 | CRE | 35.477 | 0.004 |
| 267 | 56 | Mn1Tel | 33.428 | 0.018 |
| 291 | 56 | Mn1Tel | 33.316 | 0.019 |
| 4 | 56 | p16 | 28.411 | 0.568 |
| 363 | 56 | p16 | 28.226 | 0.645 |
| 28 | 57 | Cjun | 25.569 | — |
| 52 | 57 | Cjun | 25.476 | — |
| 124 | 57 | CRE | 20.724 | 27.822 |
| 148 | 57 | CRE | 20.582 | 30.705 |
| 76 | 57 | Mn1Tel | 21.283 | 18.893 |
| 100 | 57 | Mn1Tel | 21.123 | 21.105 |
| 172 | 57 | p16 | 26.215 | 0.619 |
| 196 | 57 | p16 | 26.147 | 0.649 |
| 220 | 58 | Cjun | 25.541 | — |
| 244 | 58 | Cjun | 25.49 | — |
| 316 | 58 | CRE | 36.935 | 0 |
| 340 | 58 | CRE | 36.228 | 0.001 |
| 268 | 58 | Mn1Tel | 34.213 | 0.002 |
| 292 | 58 | Mn1Tel | 34.939 | 0.001 |
| 5 | 58 | p16 | 26.481 | 0.512 |
| 364 | 58 | p16 | 26.304 | 0.579 |
| 29 | 59 | Cjun | 25.794 | — |
| 53 | 59 | Cjun | 25.694 | — |
| 125 | 59 | CRE | 33.834 | 0.004 |
| 149 | 59 | CRE | 33.354 | 0.005 |
| 77 | 59 | Mn1Tel | 36.546 | 0.001 |
| 101 | 59 | Mn1Tel | 33.896 | 0.004 |
| 173 | 59 | p16 | 26.414 | 0.628 |
| 197 | 59 | p16 | 26.442 | 0.616 |
| 221 | 60 | Cjun | 25.998 | — |
| 245 | 60 | Cjun | 26.105 | — |
| 317 | 60 | CRE | 21.593 | 21.981 |
| 341 | 60 | CRE | 21.442 | 24.421 |
| 269 | 60 | Mn1Tel | 21.864 | 18.223 |
| 293 | 60 | Mn1Tel | 21.74 | 19.858 |
| 6 | 60 | p16 | 26.954 | 0.535 |
| 365 | 60 | p16 | 26.499 | 0.733 |
| 30 | 61 | Cjun | 24.083 | — |
| 54 | 61 | Cjun | 24.067 | — |
| 126 | 61 | CRE | 34.69 | 0.001 |
| 150 | 61 | CRE | 35.396 | 0 |
| 78 | 61 | Mn1Tel | 40 | 0 |
| 102 | 61 | Mn1Tel | 35.961 | 0 |
| 174 | 61 | p16 | 26.1 | 0.246 |
| 198 | 61 | p16 | 26.164 | 0.235 |

TABLE 4

| Sample# | Cre | | | Mn1tel | | | p16 | | |
|---|---|---|---|---|---|---|---|---|---|
| 51 | 15.89 | 15.87 | + | 12.88 | 17.28 | + | 0.25 | 0.23 | + |
| 52 | 13.45 | 13.77 | + | 11.13 | 6.08 | + | 0.16 | 0.19 | + |
| 53 | 0.02 | 0.02 | − | 13.90 | 15.47 | + | 0.29 | 0.29 | + |
| 54 | 17.96 | 18.94 | + | 13.57 | 3.47 | + | 0.23 | 0.29 | + |
| 55 | 24.87 | 26.99 | + | 18.13 | 0.26 | + | 0.36 | 0.30 | + |
| 56 | 0.01 | 0.00 | − | 0.02 | 0.02 | − | 0.57 | 0.65 | + |
| 57 | 27.82 | 30.71 | + | 18.89 | 21.11 | + | 0.62 | 0.65 | + |
| 58 | 0.00 | 0.00 | − | 0.00 | 0.00 | − | 0.51 | 0.58 | + |
| 59 | 0.00 | 0.01 | − | 0.00 | 0.00 | − | 0.63 | 0.62 | + |
| 60 | 21.98 | 24.42 | + | 18.22 | 19.86 | + | 0.54 | 0.73 | + |
| 61 | 0.00 | 0.00 | − | 0.00 | 0.00 | − | 0.25 | 0.24 | + |

### Example 2 Blood Sample Collection Method

Mouse tails are nicked with a razor blade and the resulting blood droplets are blotted on to filter paper (V&P Scientific Lint Free Blotting Media (114 mm long, 74 mm wide) #VP540D). The samples are placed in individual wells of a Nunc 96-well plate (Fisher Scientific 12-565-368). The well locations are labeled and the plates are transported shipped to the screening laboratory 20.

The remote user 1 provides the genetic line identification 84. The genetic line in this example has been previously associated by the remote user 1 with the designated genetic sequence for Mn1Tel (SEQ ID NO. 38), CRE (SEQ ID NO. 22) and MHV (SEQ ID NO. 34).

The number of samples are counted and lysis reagent is made (2.5 μl of proteinase K (VWR EM-24568-3) and 147.5 μl of Nuclei Lysing Solution (Promega Corporation, Madison Wis., A7943) per sample. The solution is gently mixed and poured into a 25 ml trough or reservoir and placed on the deck of a Tecan Genesis Workstation (Research Triangle Park, N.C.). The liquid handler dispenses 150 μl of the solution into each sample well. The well plate was then placed in a 55° C. oven for three hours.

The well plate is then placed back on the deck of the Tecan Genesis Workstation. The liquid handler aspirates 50 μl of each sample and dispenses it in to a 384 primary master well container (Fisher Scientific #NC9134044). Once all of the samples are transferred, the primary master well container is moved to the deck of the Isolation Station Purification Station 94.

One-hundred and twelve microliters of SV Lysis reagent (Promega Corporation, # Z305X) are added to each sample. Next, 13 μl of magnetic particles (Promega Corporation # A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the last ethanol wash, the well plate is placed on a 384 tip dryer for 11 minutes. Then the well plate is moved back to the deck of the Isolation Station and 155 μl of Ambion's (Houston, Tex.) nuclease free water (catalog #B9934) is added to each well. The elution solution is heated to 95°. The plate is then moved into the magnetic field and 50 μl of DNA elution is transferred to a 384 well optical storage plate (Fisher Scientific, #08-772136) for optical density analysis.

An A₂₆₀ reading of the storage plate read is performed with a Tecan Genios Spectrometer. This reading shows nucleic acid was present at the desired concentration of 0.2 O.D. units, but, a range of 0.1 to 0.5 O.D. units is acceptable.

The amount of DNA isolated from the blood is less than the DNA yield recovered from tissue. The tissue lysate has enough DNA content to saturate the binding ability of the fixed volume of beads. However, the blood lysate does not have enough DNA to saturate the binding ability of the fixed amount of beads. This is evidence by the CT (cycle threshold) values for the housekeeping probe. The housekeeping (cjun) CT values for tissue isolations are approximately 26 whereas the approximate CT for housekeeping (cjun) for the blood isolations are approximately 35. This nine cycle difference represents approximately a 512 (2ˆ9) fold difference in the amount DNA present. This non-saturated DNA yield does not present a problem for results because the housekeeping probe normalizes the results. For each sample, the CT values for the wells containing the housekeeping probe, cjun, are averaged (CT_{cjun}). The RCN (RCN₁ and RCN₂) values are calculated by comparing the test probe (i.e. Cre or MN1TEL) signal to the housekeeping gene signal average for each of the two test probe wells (CT₁ and CT₂), the following equation is applied:
RCN₁=2^{-(CT}^{₁}^{-CT}^{_{cjun}}⁾
RCN₂=2^{-(CT}^{₂}^{-CT}^{_{cjun}}⁾

The plate with the isolated DNA is moved to the deck of a Tecan Freedom Workstation; TaqMan Universal Master Mix, real time PCR primer mixture and Ambion water are placed on the deck as well. The final PCR mixture is made of 1× TaqMan Universal Master Mix (catalog # 4326708), 1× real time PCR primer mix for a designated genetic sequence (Applied Biosystems Assays-by-Design (SM) Service 4331348) and 25% isolated genomic DNA. The Tecan Genesis adds the reagents together in the ABI 7900 384 Well Optical Plate (Foster City, Calif.) catalog #4309849). The 384 well plate is then sealed with optical sealing tape (ABI, #4311971).

The samples are then placed in an Applied Biosystems SDS HT7900 (Foster City, Calif.). A standard real time PCR protocol is followed by heating the samples to 50° C. for two minutes then incubated at 95° C. for 10 minutes, followed by thermally cycling the samples 40 times between 95° C. for 15 seconds and 60° C. for one minute.

TABLE 5
Blood Samples Taken from Double KO mice
Whatman Filter Paper used to capture samples

| | | Designated | | |
|---|---|---|---|---|
| | Sample | Genetic | | Std. Dev. |
| Well | Name | Sequence | CT | CT |
| A1 | WATER | Cjun | Undetermined | |
| A2 | Blood 2 | Cjun | 35.31 | 0.587 |
| A3 | Blood 3 | MN1TEL | 33.51 | 0.061 |
| A4 | Blood 4 | CRE | 34.72 | 0.27 |
| A5 | Blood 6 | Cjun | 35.78 | 0.175 |
| A6 | Blood 7 | MN1TEL | 33.24 | 0.325 |
| A7 | Blood 8 | CRE | Undetermined | |
| A8 | Blood 10 | Cjun | 35.44 | 0.023 |
| A9 | Blood 11 | MN1TEL | 35.25 | 0.004 |
| A10 | AF 2 | Cjun | 37.25 | 0.786 |
| A11 | AF 4 | Cjun | 35.17 | 0.165 |
| B1 | WATER | Cjun | Undetermined | |
| B2 | Blood 2 | Cjun | 34.48 | 0.587 |
| B3 | Blood 3 | MN1TEL | 33.42 | 0.061 |
| B4 | Blood 4 | CRE | 34.34 | 0.27 |
| B5 | Blood 6 | Cjun | 36.03 | 0.175 |
| B6 | Blood 7 | MN1TEL | 33.7 | 0.325 |
| B7 | Blood 8 | CRE | Undetermined | |
| B8 | Blood 10 | Cjun | 35.47 | 0.023 |
| B9 | Blood 11 | MN1TEL | 35.25 | 0.004 |
| B10 | AF 2 | Cjun | 36.14 | 0.786 |
| B11 | AF 4 | Cjun | 34.94 | 0.165 |
| C1 | Blood 1 | Cjun | 35.39 | 0.218 |
| C2 | Blood 2 | MN1TEL | 34.37 | 0.281 |
| C3 | Blood 3 | CRE | Undetermined | |
| C4 | Blood 5 | Cjun | 36.35 | 0.172 |
| C5 | Blood 6 | MN1TEL | 34.96 | 0.634 |
| C6 | Blood 7 | CRE | 37.76 | 0.556 |
| C7 | Blood 9 | Cjun | 33.61 | 0.069 |
| C8 | Blood 10 | MN1TEL | 34.3 | 0.734 |
| C9 | Blood 11 | CRE | 32.9 | 0.6 |
| C10 | AF 2 | MHV | Undetermined | |
| C11 | AF 4 | MHV | Undetermined | |
| D1 | Blood 1 | Cjun | 35.08 | 0.218 |
| D2 | Blood 2 | MN1TEL | 34.77 | 0.281 |
| D3 | Blood 3 | CRE | 39.09 | |
| D4 | Blood 5 | Cjun | 36.6 | 0.172 |
| D5 | Blood 6 | MN1TEL | 34.06 | 0.634 |
| D6 | Blood 7 | CRE | 38.55 | 0.556 |
| D7 | Blood 9 | Cjun | 33.71 | 0.069 |
| D8 | Blood 10 | MN1TEL | 33.26 | 0.734 |
| D9 | Blood 11 | CRE | 33.74 | 0.6 |
| D10 | AF 2 | MHV | Undetermined | |
| D11 | AF 4 | MHV | Undetermined | |
| E1 | Blood 1 | MN1TEL | 33.7 | 0.131 |
| E2 | Blood 2 | CRE | Undetermined | |
| E3 | Blood 4 | Cjun | 37.7 | 0.252 |
| E4 | Blood 5 | MN1TEL | 35.48 | 1.053 |
| E5 | Blood 6 | CRE | 31.84 | 0.03 |
| E6 | Blood 8 | Cjun | 34.57 | 0.13 |
| E7 | Blood 9 | MN1TEL | 32.45 | 0.111 |
| E8 | Blood 10 | CRE | Undetermined | |
| E9 | AF 1 | Cjun | 39.35 | 0.278 |
| E10 | AF 3 | Cjun | 33.75 | 0.213 |
| E11 | BF 1 | Cjun | 28.14 | 0.048 |
| F1 | Blood 1 | MN1TEL | 33.52 | 0.131 |
| F2 | Blood 2 | CRE | Undetermined | |
| F3 | Blood 4 | Cjun | 38.06 | 0.252 |
| F4 | Blood 5 | MN1TEL | 36.97 | 1.053 |
| F5 | Blood 6 | CRE | 31.88 | 0.03 |
| F6 | Blood 8 | Cjun | 34.75 | 0.13 |
| F7 | Blood 9 | MN1TEL | 32.29 | 0.111 |
| F8 | Blood 10 | CRE | Undetermined | |
| F9 | AF 1 | Cjun | 38.96 | 0.278 |
| F10 | AF 3 | Cjun | 34.05 | 0.213 |
| F11 | BF 1 | Cjun | 28.21 | 0.048 |
| G1 | Blood 1 | CRE | Undetermined | |
| G2 | Blood 3 | Cjun | 34.52 | 0.041 |
| G3 | Blood 4 | MN1TEL | 36.02 | 0.284 |
| G4 | Blood 5 | CRE | 38.12 | 0.071 |
| G5 | Blood 7 | Cjun | 34.69 | 0.387 |
| G6 | Blood 8 | MN1TEL | 33.29 | 0.302 |
| G7 | Blood 9 | CRE | 37.75 | |
| G8 | Blood 11 | Cjun | 36.57 | 0.057 |
| G9 | AF 1 | MHV | Undetermined | |
| G10 | AF 3 | MHV | Undetermined | |
| G11 | BF 1 | MHV | Undetermined | |
| H1 | Blood 1 | CRE | Undetermined | |
| H2 | Blood 3 | Cjun | 34.46 | 0.041 |
| H3 | Blood 4 | MN1TEL | 35.62 | 0.284 |
| H4 | Blood 5 | CRE | 38.02 | 0.071 |
| H5 | Blood 7 | Cjun | 35.24 | 0.387 |
| H6 | Blood 8 | MN1TEL | 33.72 | 0.302 |
| H7 | Blood 9 | CRE | Undetermined | |
| H8 | Blood 11 | Cjun | 36.65 | 0.057 |
| H9 | AF 1 | MHV | Undetermined | |
| H10 | AF 3 | MHV | Undetermined | |
| H11 | BF 1 | MHV | Undetermined | |

### Example 3 Mouse Embryonic Genotyping Protocol

Mouse embryonic tissue is submitted via FedEx (Memphis, Tenn.) overnight delivery. Each sample occupies one well of a 96-well microwell container 2. The remote user 1 provides the genetic line identification 84. The genetic line in this example has been previously associated with the designated genetic sequence Neomycin (SEQ ID NO. 42) and Six 2 WT (SEQ. ID NO. 62).

A lysis reagent is made of (2.5 μl of proteinase K (VWR EM-24568-3) and 147.5 μl of Nuclei Lysing Solution (Promega Corporation A7943) per sample). The lysis reagent is gently mixed and poured into a 25 ml trough or reservoir and placed on the deck of a Tecan (Research Triangle Park, N.C.) Genesis Workstation. The liquid handler dispensed 150 μl of solution in to each sample well in the well plate. The well plate is then placed in a 55° C. oven for three hours. Samples are sonicated with a fixed horn sonicator for 3-5 seconds, to yield a sample having at least a portion of intact genomic nucleic acids and at least a portion of nucleic acid fragments. Samples are then allowed to settle at room temperature for five minutes prior to accessioning.

The well plate is then placed back on the deck of the Tecan Genesis Workstation. The liquid handler aspirates 50 μl of each sample and dispenses it in to a 384 destination well plate (Fisher Scientific #NC9134044). Once all of the samples are transferred, the well plate is moved to the deck of the Isolation/Purification Station 94.

One-hundred and twelve microliters of SV Lysis reagent (Promega Corporation, Madison Wis., # Z305X) is added to each sample. Next, 13 μl of magnetic particles (Promega Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is them moved into the magnetic field and the supernatant is drawn off and discarded. This washing process is repeated two additional times. Next, the sample is washed four times in 130 μl of 95% ethanol as described above. After the fourth ethanol wash, the destination plate is placed on a 384 tip dryer for 11 minutes. Then the well plate is moved back to the deck of the Isolation/Purification Station 94 and 155 μl of Ambion's (Houston, Tex.) nuclease free water (catalog #B9934) is added to each well of the well plate. The elution solution is heated to 95°. The well plate is then moved into the magnetic field and 50 μl of DNA elution is transferred to a 384 well optical storage plate (Fisher Scientific, #08-772136) for optical density analysis.

An A₂₆₀ reading of the storage plate read is performed with a Tecan Genios Spectrometer. This shows that nucleic acid is present at the desired concentration of 0.2 O.D. units, but a range of 0.1 to 0.5 O.D. units is acceptable.

The plate with the isolated DNA is moved to the deck of a Tecan Freedom Workstation. The final PCR mixture is made of 1× TaqMan Universal Master Mix (catalog # 4326708). 1× real time PCR probe and primer mix for a designated genetic sequence (Applied Biosystem Assays-by-Design (SM) Service 4331348) free water and 25% isolated DNA to an ABI 7900 384 Well Optical Plate (Foster City, Calif.) catalog #4309849). The well plate is then sealed with optical sealing tape (ABI, #4311971).

The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Table 5 and 6. The designated genetic sequence is Neomycin (SEQ ID NO. 42)

TABLE 6

| | Sample | Designated Genetic | | |
|---|---|---|---|---|
| Well | Name | Sequence | CT | RCN |
| 49 | 161016 | Cjun | 25.691 | — |
| 73 | 161016 | Cjun | 25.45 | — |
| 97 | 161016 | Neomycin | 22.873 | 6.488 |
| 121 | 161016 | Neomycin | 22.387 | 9.083 |
| 145 | 161016 | Six2 WT #1 | 25.063 | 1.422 |
| 169 | 161016 | Six2 WT #1 | 25.034 | 1.451 |
| 193 | 161017 | Cjun | 25.73 | — |
| 217 | 161017 | Cjun | 25.705 | — |
| 241 | 161017 | Neomycin | 33.269 | 0.005 |
| 265 | 161017 | Neomycin | 32.837 | 0.007 |
| 289 | 161017 | Six2 WT #1 | 25.403 | 1.244 |
| 313 | 161017 | Six2 WT #1 | 25.347 | 1.293 |
| 337 | 161018 | Cjun | 25.4 | — |
| 361 | 161018 | Cjun | 25.136 | — |
| 2 | 161018 | Neomycin | 22.706 | 5.908 |
| 26 | 161018 | Neomycin | 22.59 | 6.401 |
| 50 | 161018 | Six2 WT #1 | 25.34 | 0.951 |
| 74 | 161018 | Six2 WT #1 | 25.138 | 1.094 |
| 98 | 161019 | Cjun | 25.903 | — |
| 122 | 161019 | Cjun | 25.681 | — |
| 146 | 161019 | Neomycin | 21.993 | 13.921 |
| 170 | 161019 | Neomycin | 21.81 | 15.797 |
| 194 | 161019 | Six2 WT #1 | 36.329 | 0.001 |
| 218 | 161019 | Six2 WT #1 | 36.057 | 0.001 |
| 242 | 161020 | Cjun | 25.354 | — |
| 266 | 161020 | Cjun | 25.068 | — |
| 290 | 161020 | Neomycin | 21.654 | 11.767 |
| 314 | 161020 | Neomycin | 21.519 | 12.926 |
| 338 | 161020 | Six2 WT #1 | 34.738 | 0.001 |
| 362 | 161020 | Six2 WT #1 | 35.638 | 0.001 |
| 3 | 161021 | Cjun | 26.136 | — |
| 27 | 161021 | Cjun | 26.029 | — |
| 51 | 161021 | Neomycin | 34.416 | 0.003 |
| 75 | 161021 | Neomycin | 35.935 | 0.001 |
| 99 | 161021 | Six2 WT #1 | 25.762 | 1.249 |
| 123 | 161021 | Six2 WT #1 | 25.807 | 1.21 |
| 147 | 161022 | Cjun | 25.825 | — |
| 171 | 161022 | Cjun | 25.669 | — |
| 195 | 161022 | Neomycin | 22.954 | 6.931 |
| 219 | 161022 | Neomycin | 22.88 | 7.295 |
| 243 | 161022 | Six2 WT #1 | 26.04 | 0.816 |
| 267 | 161022 | Six2 WT #1 | 25.735 | 1.008 |
| 291 | 161023 | Cjun | 25.09 | — |
| 315 | 161023 | Cjun | 25.304 | — |
| 339 | 161023 | Neomycin | 21.543 | 12.592 |
| 363 | 161023 | Neomycin | 21.422 | 13.688 |
| 4 | 161023 | Six2 WT #1 | 40 | 0 |
| 28 | 161023 | Six2 WT #1 | 34.991 | 0.001 |
| 52 | 161024 | Cjun | 25.461 | — |
| 76 | 161024 | Cjun | 25.062 | — |
| 100 | 161024 | Neomycin | 34.749 | 0.001 |
| 124 | 161024 | Neomycin | 35.415 | 0.001 |
| 148 | 161024 | Six2 WT #1 | 24.991 | 1.206 |
| 172 | 161024 | Six2 WT #1 | 24.676 | 1.501 |
| 196 | 161025 | Cjun | 26.426 | — |
| 220 | 161025 | Cjun | 26.073 | — |
| 244 | 161025 | Neomycin | 23.711 | 5.81 |
| 268 | 161025 | Neomycin | 23.539 | 6.544 |
| 292 | 161025 | Six2 WT #1 | 27.013 | 0.589 |
| 316 | 161025 | Six2 WT #1 | 26.959 | 0.612 |
| 340 | 161026 | Cjun | 25.343 | — |
| 364 | 161026 | Cjun | 25.111 | — |
| 5 | 161026 | Neomycin | 32.086 | 0.009 |
| 29 | 161026 | Neomycin | 31.224 | 0.016 |
| 53 | 161026 | Six2 WT #1 | 24.779 | 1.364 |
| 77 | 161026 | Six2 WT #1 | 24.526 | 1.626 |
| 101 | 161027 | Cjun | 25.955 | — |
| 125 | 161027 | Cjun | 25.668 | — |
| 149 | 161027 | Neomycin | 23.1 | 6.549 |
| 173 | 161027 | Neomycin | 23.125 | 6.434 |
| 197 | 161027 | Six2 WT #1 | 26.701 | 0.54 |
| 221 | 161027 | Six2 WT #1 | 26.203 | 0.762 |
| 245 | 161028 | Cjun | 25.232 | — |
| 269 | 161028 | Cjun | 25.151 | — |
| 293 | 161028 | Neomycin | 22.614 | 5.966 |
| 317 | 161028 | Neomycin | 22.635 | 5.881 |
| 341 | 161028 | Six2 WT #1 | 25.977 | 0.58 |
| 365 | 161028 | Six2 WT #1 | 25.709 | 0.698 |

### Example 4 Embryonic Stem Cell Genotyping Protocol

Mouse embryonic stem cells were grown to confluence in a 96 well source well container 2 such as a cell culture plate and was submitted via FedEx (Memphis, Tenn.) overnight delivery to the screening laboratory 20. The remote user 1 provides the genetic line identification 84. The genetic line in this example has been previously associated with the designated genetic sequence for OPN4 ES (SEQ ID NO. 46). The samples are counted and a lysis reagent is made of (2.5 μl of proteinase K(VWR EM-24568-3) and 147.5 μl of Nuclei Lysing Solution (Promega Corporation A7943) per sample). The solution is gently mixed and poured into a 25 ml trough or reservoir and placed on the deck of a Tecan (Research Triangle Park, N.C.) Genesis Workstation. The liquid handler dispenses 150 μl of solution in to each source well container 2. The samples are then incubated at room temperature for ten minutes before being transferred to a polypropylene 96 well plate. The well plate is then covered and placed in a 55° C. oven for three hours.

The source well container 2 is then placed back on the deck of the Tecan Genesis Workstation. The liquid handler aspirates 50 μl of each sample and dispenses it in to a 384 primary master well container (Fisher Scientific #NC9134044). Once all of the samples are transferred, the primary master well container 6 is moved to the deck of the Isolation/Purification Station 94.

One-hundred and twelve microliters of SV Lysis reagent (Promega Corporation, Madison, Wis., #Z305X) are added to each sample. Next, 13 μl of magnetic particles (Promega Corporation, #A220X) are added and the well components were mixed. The well plate is then moved into the magnetic field of a magnet where the magnetic particles are drawn to the bottom of each well. The supernatant is then aspirated and discarded. The well plate is moved out of the magnetic field; 95 μl of SV Lysis reagent are added to each well and mixed. The well plate is then moved into the magnetic field and the supernatant is drawn off and discarded. This washing process is two additional times. Next, the sample is washed four times in 130 μl of 95% ethanol as described above. After the fourth ethanol wash, the plate is placed on a 384 tip dryer for eleven minutes. Then the plate is moved back to the deck of the Isolation/Purification Station 94 and 155 μl of Ambion's (Houston, Tex.) nuclease free water (catalog #B9934) is added to each well. The elution solution is heated to 95°. The plate is then moved into the magnetic field and 50 μl of DNA elution was transferred to a 384 well optical storage plate (Fisher Scientific, #08-772136) for optical density analysis.

An A₂₆₀ reading of the storage plate read is performed with a Tecan Genios Spectrometer. This reading should nucleic acid be present at the desired 0.2 O.D. a range of 0.1 to 0.5 O.D. units is acceptable.

The primary master wellplate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog #4326708), 1× real time PCR primer sets/probe mix for a designated genetic sequence (Applied Biosystems Assays-by-Design (SM) Service 4331348) and 25% isolated DNA. Tecan Genesis adds the reagents together in the ABI 7900 384 Well Optical Plate. The plate is then sealed with optical sealing tape (ABI, #4311971).

The samples were then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Table 7.

TABLE 7

| | | Designated | | |
|---|---|---|---|---|
| | Sample | Genetic | | |
| Well | Name | Sequence | Reporter | Ct |
| 49 | 1 | Cjun | VIC | 31.46 |
| 73 | 1 | Cjun | VIC | 31.41 |
| 97 | 1 | OPN4ES | FAM | 29.54 |
| 121 | 1 | OPN4ES | FAM | 29.54 |
| 145 | 2 | Cjun | VIC | 30.39 |
| 169 | 2 | Cjun | VIC | 30.32 |
| 193 | 2 | OPN4ES | FAM | 28.61 |
| 217 | 2 | OPN4ES | FAM | 29.09 |
| 241 | 3 | Cjun | VIC | 31.13 |
| 265 | 3 | Cjun | VIC | 31.05 |
| 289 | 3 | OPN4ES | FAM | 29.62 |
| 313 | 3 | OPN4ES | FAM | 29.63 |
| 337 | 4 | Cjun | VIC | 31.01 |
| 361 | 4 | Cjun | VIC | 31.64 |
| 2 | 4 | OPN4ES | FAM | 29.57 |
| 26 | 4 | OPN4ES | FAM | 29.66 |
| 50 | 5 | Cjun | VIC | 31.76 |
| 74 | 5 | Cjun | VIC | 31.19 |
| 98 | 5 | OPN4ES | FAM | 30.36 |
| 122 | 5 | OPN4ES | FAM | 30.08 |
| 146 | 6 | Cjun | VIC | 30.79 |
| 170 | 6 | Cjun | VIC | 30.90 |
| 194 | 6 | OPN4ES | FAM | 29.47 |
| 218 | 6 | OPN4ES | FAM | 29.57 |
| 242 | 7 | Cjun | VIC | 33.59 |
| 266 | 7 | Cjun | VIC | 33.58 |
| 290 | 7 | OPN4ES | FAM | 32.06 |
| 314 | 7 | OPN4ES | FAM | 32.11 |
| 338 | 8 | Cjun | VIC | 32.82 |
| 362 | 8 | Cjun | VIC | 33.25 |
| 3 | 8 | OPN4ES | FAM | 31.68 |
| 27 | 8 | OPN4ES | FAM | 31.44 |
| 51 | 9 | Cjun | VIC | 32.69 |
| 75 | 9 | Cjun | VIC | 32.96 |
| 99 | 9 | OPN4ES | FAM | 31.82 |
| 123 | 9 | OPN4ES | FAM | 31.33 |
| 147 | 10 | Cjun | VIC | 32.89 |
| 171 | 10 | Cjun | VIC | 32.80 |
| 195 | 10 | OPN4ES | FAM | 31.71 |
| 219 | 10 | OPN4ES | FAM | 31.46 |
| 243 | 11 | Cjun | VIC | 33.39 |
| 267 | 11 | Cjun | VIC | 32.98 |
| 291 | 11 | OPN4ES | FAM | 31.77 |
| 315 | 11 | OPN4ES | FAM | 31.69 |
| 339 | 12 | Cjun | VIC | 33.20 |
| 363 | 12 | Cjun | VIC | 33.81 |
| 4 | 12 | OPN4ES | FAM | 31.96 |
| 28 | 12 | OPN4ES | FAM | 31.90 |
| 52 | 13 | Cjun | VIC | 32.73 |
| 76 | 13 | Cjun | VIC | 32.87 |
| 100 | 13 | OPN4ES | FAM | 31.16 |
| 124 | 13 | OPN4ES | FAM | 31.52 |
| 148 | 14 | Cjun | VIC | 32.86 |
| 172 | 14 | Cjun | VIC | 32.30 |
| 196 | 14 | OPN4ES | FAM | 30.82 |
| 220 | 14 | OPN4ES | FAM | 30.64 |
| 244 | 15 | Cjun | VIC | 33.15 |
| 268 | 15 | Cjun | VIC | 33.16 |
| 292 | 15 | OPN4ES | FAM | 31.25 |
| 316 | 15 | OPN4ES | FAM | 31.81 |
| 340 | 16 | Cjun | VIC | 32.41 |
| 364 | 16 | Cjun | VIC | 32.51 |
| 5 | 16 | OPN4ES | FAM | 30.70 |
| 29 | 16 | OPN4ES | FAM | 30.89 |
| 53 | 17 | Cjun | VIC | 33.07 |
| 77 | 17 | Cjun | VIC | 33.44 |
| 101 | 17 | OPN4ES | FAM | 31.67 |
| 125 | 17 | OPN4ES | FAM | 31.94 |
| 149 | 18 | Cjun | VIC | 32.63 |
| 173 | 18 | Cjun | VIC | 32.48 |
| 197 | 18 | OPN4ES | FAM | 30.97 |
| 221 | 18 | OPN4ES | FAM | 31.10 |
| 245 | 19 | Cjun | VIC | 34.61 |
| 269 | 19 | Cjun | VIC | 34.18 |
| 293 | 19 | OPN4ES | FAM | 32.90 |
| 317 | 19 | OPN4ES | FAM | 32.93 |
| 341 | 20 | Cjun | VIC | 34.11 |
| 365 | 20 | Cjun | VIC | 34.53 |
| 6 | 20 | OPN4ES | FAM | 32.25 |
| 30 | 20 | OPN4ES | FAM | 32.64 |
| 54 | 21 | Cjun | VIC | 33.76 |
| 78 | 21 | Cjun | VIC | 33.80 |
| 102 | 21 | OPN4ES | FAM | 31.93 |
| 126 | 21 | OPN4ES | FAM | 32.36 |
| 150 | 22 | Cjun | VIC | 33.59 |
| 174 | 22 | Cjun | VIC | 33.78 |
| 198 | 22 | OPN4ES | FAM | 32.64 |
| 222 | 22 | OPN4ES | FAM | 31.98 |
| 246 | 23 | Cjun | VIC | 34.32 |
| 270 | 23 | Cjun | VIC | 34.24 |
| 294 | 23 | OPN4ES | FAM | 32.87 |
| 318 | 23 | OPN4ES | FAM | 33.08 |
| 342 | 24 | Cjun | VIC | 34.14 |
| 366 | 24 | Cjun | VIC | 34.72 |
| 7 | 24 | OPN4ES | FAM | 33.08 |
| 31 | 24 | OPN4ES | FAM | 33.46 |
| 1 | NTC | Cjun | VIC | 36.07 |
| 25 | NTC | Cjun | VIC | 36.93 |

### Example 5 MHV (RNA Virus) Screening

Biomatter in the form of fecal samples from mice is submitted via FedEx® (Memphis, Tenn.) overnight delivery. Each sample occupies one well of a 96 source well container 2. The remote user 1 provides the genetic line identification 84. The genetic line in this example has been previously associated by the remote user 1 with the designated genetic sequence for MHV (SEQ ID NO. 34). Samples are counted and 250 μl of SV Lysis reagent (Promega Corporation, Madison Wis., # Z305X) is added to each sample well of the source well container 2. The source well container 2 is then vortexed to homogenize the samples. Next, the source well container 2 two is spun in a centrifuge for one minute.

The source well container 2 is then placed back on the deck of the Tecan Genesis Workstation® (Research Triangle Park, N.C.). Once all of the samples are transferred to the primary master well plate, the well plate is moved to the deck of the Isolation/Purification Station 94.

One hundred and twelve microliters of lysis reagent (Promega Corporation #Z305X) are added to each sample. Thirty microliters of magnetic particles (Promega Corporation A220X) are added to the wells of a 384 destination well plate (Fisher Scientific #NC9134044). The well plate is moved into a magnetic field and the packing oil supernatant is aspirated off the particle bed. The liquid handler aspirates 100 μl of each sample liquid fecal biomatter sample and dispenses it into the 384 primary master well container, mixing the samples and particles. The particles are allowed to incubate at room temperature for three minutes with a sufficient amount of chaotropic salt to cover the particles. The primary master well container is then moved into the magnetic field of a magnet where the magnetic particles are drawn to the bottom of each well. The supernatant are then aspirated and discarded. The primary master well container is then moved out of the magnetic field. Next, 150 μl of 95% ethanol is added. The primary master well container is moved into the magnetic field and the ethanol supernatant is aspirated off the bead bed. Then, the primary master well container is placed on a 384 tip dryer for one minute. Then the primary master well container is moved back to the deck of the Isolation/Purification Station 94 and 50 μl of DNase solution (Promega Corporation, Yellow Core Buffer #Z317D, MnCl₂ # Z318D and DNase # Z358A) is prepared according to Promega Technical Bulletin 328 and added to each sample and incubated at room temperature for 15 minutes. Next, 100 μl of stop buffer (Promega Corporation, DNase Stop #Z312D) is added and incubated for two minutes at room temperature. Two ethanol washes are done as described above. The primary master well container is then placed back on the dryer for two minutes. Finally, 60 μl Ambion's (Houston, Tex.) nuclease free water (catalog #B9934) is added to each well of the primary master well container. The elution solution was heated to 95° C. The primary master well container is then moved into the magnetic field and 50 μl of DNA was transferred to a 384 well optical storage plate (Fisher Scientific, #08-772136) for optical density analysis.

An A₂₆₀ reading of the storage plate read was performed with a Tecan Genios Spectrometer. This reading showed nucleic acid is present at the desired standard concentration of 0.2 O.D. units, but a range of 0.1 to 0.5 O.D. units is acceptable.

The plate with the isolated RNA was moved to the deck of a Tecan Freedom Workstation; reverse transcriptase-PCR mixture and Ambion water was placed on the deck as well as a 384 optical well plate (Applied Biosystems (Foster City, Calif.) catalog #4309849)). The reverse transcriptase-PCR mixture was made with TAQ-Man® EZ RT-PCR Kit (Applied Biosystems, catalog #N808-0236). The Tecan Genesis adds the reagents together in the ABI 7900 384 Well Optical Plate. The plate is then sealed with optical sealing tape (ABI, #4311971). The samples were incubated for two minutes at 50° C., thirty minutes at 60° C. and five minutes at 95° C. The plate was then thermocycled for twenty seconds at 94° C. and one minute at 62° C., for forty cycles. The results are shown in Table 8.

TABLE 8

| | | Designated | | |
|---|---|---|---|---|
| | Sample | Genetic | | Std. Dev. |
| Well | Name | Sequence | CT | CT |
| A1 | 1 + Full | MHV | 27.15 | 0.408 |
| A2 | 1 + ¾ | MHV | 27.64 | 0.474 |
| A3 | 1 + ½ | MHV | 28.41 | 0.226 |
| A4 | 1 + ¼ | MHV | 32.5 | 1.917 |
| A5 | Water Full | MHV | Undetermined | |
| B1 | 1 + Full | MHV | 26.57 | 0.408 |
| B2 | 1 + ¾ | MHV | 26.97 | 0.474 |
| B3 | 1 + ½ | MHV | 28.09 | 0.226 |
| B4 | 1 + ¼ | MHV | 29.79 | 1.917 |
| B5 | Water Full | MHV | Undetermined | |
| C1 | 2 + Full | MHV | 24.03 | 0.033 |
| C2 | 2 + ¾ | MHV | 24.41 | 0.385 |
| C3 | 2 + ½ | MHV | 24.86 | 0.252 |
| C4 | 2 + ¼ | MHV | 26.21 | 0.273 |
| C5 | Water ¾ | MHV | Undetermined | |
| D1 | 2 + Full | MHV | 23.98 | 0.033 |
| D2 | 2 + ¾ | MHV | 23.87 | 0.385 |
| D3 | 2 + ½ | MHV | 24.51 | 0.252 |
| D4 | 2 + ¼ | MHV | 25.83 | 0.273 |

### Example 6 Zygosity Genotyping of Nontransgenic Samples (Targeted Mutation)

Specifically, a remote user 1 can contact the screening laboratory 20 and provide a description of the mutation. This description may include information such as the endogenous gene Bgal (also known as Glb1) was disrupted with the deletion of a particular exon with a Neomycin cassette. The gene name may be used to query databases to yield literature specific for this mutation by the screening laboratory 20. The Mouse Genome Informatics (MGI) J:38620, PubMed 9063740, or Medline 97217779 databases with their respective journal numbers, yield the following literature reference: Hahn C N; del Pilar Martin M; Schroder M; Vanier M T; Hara Y; Suzuki K; Suzuki K; d'Azzo A, Generalized CNS disease and massive GM1-ganglioside accumulation in mice defective in lysosomal acid beta-galactosidase., Hum Mol Genet 1997 February; 6(2):205-11.

This reference discloses that a Neomycin cassette was inserted into exon six of the Bgal at a AatII restriction site. The screening laboratory 20 would then query a database such as Ensembl. The Ensembl gene identification number is ENSMUSG00000042315. The genomic sequence with the exons and restriction sites is identified.

The screening laboratory 20 queries a database such as Ensembl. This query yields sequence data, which is the designated genetic sequence. By knowing the endogenous bases that have been deleted, the screening laboratory 20 can take the designated genetic sequence, or portion thereof, and send it to a vendor indicating where to build the primers and probes as to be informative for screening. Moreover, if there are a large number of bases that have been deleted, the screening laboratory 20 may only send the sequence of bases that will be deleted if the mutation has occurred to the vendor and have them build primers and probe anywhere inside the sequence.

The Neomycin coding sequence, or mutation sequence, does not naturally occur in mice. The same mechanism of identifying the designated genetic sequence using the National Center for Biotechnology Information database and having a vendor build anywhere inside the sequence is used.

A biological sample in the form of a mouse tail biopsy is submitted via FedEx (Memphis, Tenn.) overnight delivery to the screening laboratory 20 from the remote user 1. Each sample occupies one well of a 96-well source well container. A lysis reagent (made of 2.5 μl of proteinase K (VWR EM-24568-3) and 147.5 μl of Nuclei Lysing Solution (Promega Corporation, Madison, Wis. A7943) per sample)) is gently mixed and poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is then placed in a 55° C. oven for three hours. The well plate is then placed back on the deck of the Tecan Genesis Workstation (Research Triangle Park, N.C.). The liquid handler aspirates 50 μl of each sample and dispenses it into a 384 well primary master well container (Fisher Scientific #NC9134044). Once all of the samples are transferred, the primary master well container is moved to the deck of the Isolation Station Purification Station 94.

One hundred and twelve microliters of SV Lysis reagent (Promega Corporation, Madison Wis., #Z305X) a chaotropic salt are added to each sample. Next, 13 μl of magnetic particles (Promega Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container are placed on a 384 tip dryer for 11 minutes. Then the microwell container are moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 OD units is acceptable.

The primary master wellplate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog #4326708), 1× real time PCR primer set/probe mix for the designated genetic sequence (Applied Biosystems Assays-by-Design (SM) Service 4331348) and 25% isolated DNA. The Tecan Genesis added the reagents together in the ABI 7900 384 Well Optical Plate. The plate is then sealed with optical sealing tape (ABI, #4311971).

The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Table 9. On average, these results are transmitted to the remote user 1 within twenty-four hours of receiving the biological sample at the screening laboratory 20.

TABLE 9

| | | | Bgal | | | | |
|---|---|---|---|---|---|---|---|
| Sample | | | WT | | | NEO | |
| Name | Bgal WT RCN | | Result | NEO RCN | | Result | Interpretation |
| 147711 | 0.005 | 0.014 | − | 28.465 | 33.608 | + | Sample is Homozygous |
| 147712 | 0.004 | 0.004 | − | 27.832 | 25.023 | + | Sample is Homozygous |
| 147713 | 0.011 | 0.011 | − | 29.842 | 22.576 | + | Sample is Homozygous |
| 147714 | 0.008 | 0.006 | − | 24.467 | 22.744 | + | Sample is Homozygous |
| 147715 | 0.001 | 0.001 | − | 23.767 | 25.853 | + | Sample is Homozygous |
| 147716 | 0.024 | 0.006 | − | 23.403 | 31.924 | + | Sample is Homozygous |
| 147717 | 0.011 | 0.012 | − | 30.323 | 27.709 | + | Sample is Homozygous |
| 147718 | 0.011 | 0.013 | − | 22.351 | 24.558 | + | Sample is Homozygous |
| 147719 | 0.009 | 0.017 | − | 26.118 | 29.585 | + | Sample is Homozygous |
| 147720 | 0.009 | 0.006 | − | 25.341 | 27.121 | + | Sample is Homozygous |
| 147721 | 0.002 | 0.002 | − | 20.551 | 23.563 | + | Sample is Homozygous |
| 147722 | 0.002 | 0.005 | − | 27.756 | 29.563 | + | Sample is Homozygous |
| 147723 | 0.005 | 0.002 | − | 24.062 | 24.874 | + | Sample is Homozygous |
| 147724 | 0.01 | 0.016 | − | 24.854 | 26.924 | + | Sample is Homozygous |
| 147725 | 0.003 | 0.004 | − | 25.518 | 27.715 | + | Sample is Homozygous |
| 147726 | 0.004 | 0.003 | − | 21.355 | 22.03 | + | Sample is Homozygous |
| 147727 | 0.004 | 0.002 | − | 21.928 | 29.168 | + | Sample is Homozygous |
| 147728 | 2.39 | 2.774 | + | 12.544 | 12.556 | + | Sample is Heterozygous |
| 147729 | 2.311 | 2.242 | + | 12.77 | 12.486 | + | Sample is Heterozygous |
| 147730 | 2.529 | 2.531 | + | 14.622 | 14.119 | + | Sample is Heterozygous |
| 147731 | 5.064 | 4.727 | + | 0.009 | 0.007 | − | Sample is Wild Type |
| 147732 | 4.934 | 5.245 | + | 0.008 | 0.007 | − | Sample is Wild Type |
| 147733 | 0.015 | 0.009 | − | 32.759 | 31.868 | + | Sample is Homozygous |
| 147734 | 4.72 | 5.425 | + | 0.003 | 0.037 | − | Sample is Wild Type |
| 147735 | 4.604 | 5.268 | + | 0.008 | 0.02 | − | Sample is Wild Type |
| 147736 | 3.338 | 3.141 | + | 18.119 | 17.679 | + | Sample is Heterozygous |
| 147737 | 4.858 | 5.23 | + | 0.01 | 0.022 | − | Sample is Wild Type |
| 147738 | 6.477 | 6.364 | + | 0.013 | 0.026 | − | Sample is Wild Type |
| 147739 | 3.898 | 3.195 | + | 16.335 | 17.008 | + | Sample is Heterozygous |
| 147740 | 5.975 | 7.19 | + | 0.018 | 0.006 | − | Sample is Wild Type |
| 147741 | 0.014 | 0.003 | − | 32.369 | 38.082 | + | Sample is Homozygous |
| 147742 | 7.463 | 7.069 | + | 0.007 | 0.006 | − | Sample is Wild Type |
| 147743 | 6.464 | 6.393 | + | 0.008 | 0.004 | − | Sample is Wild Type |
| 147744 | 6.043 | 5.761 | + | 0.001 | 0.008 | − | Sample is Wild Type |
| 147745 | 4.726 | 6.105 | + | 0.007 | 0.021 | − | Sample is Wild Type |
| 147746 | 5.739 | 5.811 | + | 0.001 | 0.051 | − | Sample is Wild Type |
| 147747 | 6.254 | 6.476 | + | 0.001 | 0.006 | − | Sample is Wild Type |
| 147748 | 3.91 | 4.671 | + | 0.005 | 0 | − | Sample is Wild Type |
| 147749 | 4.805 | 4.112 | + | 0.011 | 0 | − | Sample is Wild Type |
| 147750 | 2.608 | 2.361 | + | 14.852 | 14.503 | + | Sample is Heterozygous |
| 147751 | 2.474 | 2.25 | + | 13.137 | 14.106 | + | Sample is Heterozygous |
| 147752 | 3.951 | 4.665 | + | 0.005 | 0.004 | − | Sample is Wild Type |
| 147753 | 1.649 | 1.997 | + | 9.593 | 12.709 | + | Sample is Heterozygous |
| 147754 | 2.018 | 2.075 | + | 11.349 | 13.382 | + | Sample is Heterozygous |
| 147755 | 4.045 | 4.373 | + | 0.004 | 0.003 | − | Sample is Wild Type |
| 147756 | 4.749 | 5.414 | + | 0.001 | 0 | − | Sample is Wild Type |

### Example 7 Transgenic Zygosity Genotyping

A plurality of tissue samples of RIP7-rtTA strain of mice are deposited in wells of a microwell container 2 by a remote user 1 and transmitted to the screening laboratory 20. The screening laboratory 20 has received instruction that transgenic zygosity genotyping of the strain is required. The remote user 1 correlates the source well container 2 well location with the sample identification number on a web page provided by the screening laboratory 20, e.g. www.transnetyx.com. Additionally, the remote user 1 indicates the transgene sequence information (i.e. designated genetic sequence) or a genetic line identification 4 in the survey of work section. Once the transgene sequence information (SEQ ID NO. 58) is acquired, the primer set/probe combination is created, (SEQ ID NO. 59-61) or may have been created previously for a remote user 1. The probe/primer set combination can be created for a transgene sequence using software, such as Primer Express® (Applied Biosystems, Forest City, Calif.). The tissue samples are screened using the primer set and probe for the designated genetic sequence. The magnitude of the signal for each sample, is captured and reported to the remote user 1. A remote user 1 interprets higher magnitude signal with a transgene on more chromosomes than the initial transgenic strain. Typically a remote user 1 will keep breeding individuals together with the highest magnitude. This breeding and genotyping continues until the remote user 1 is satisfied that the transgene is present in the ‘homozygous’ condition.

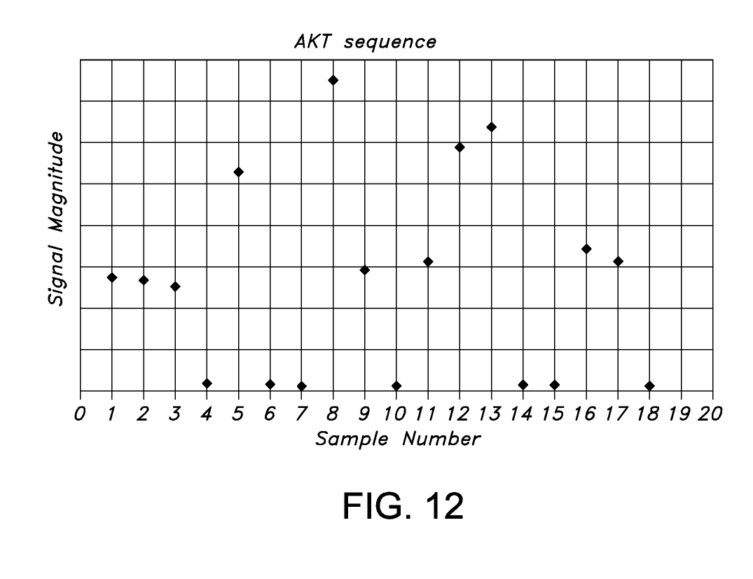

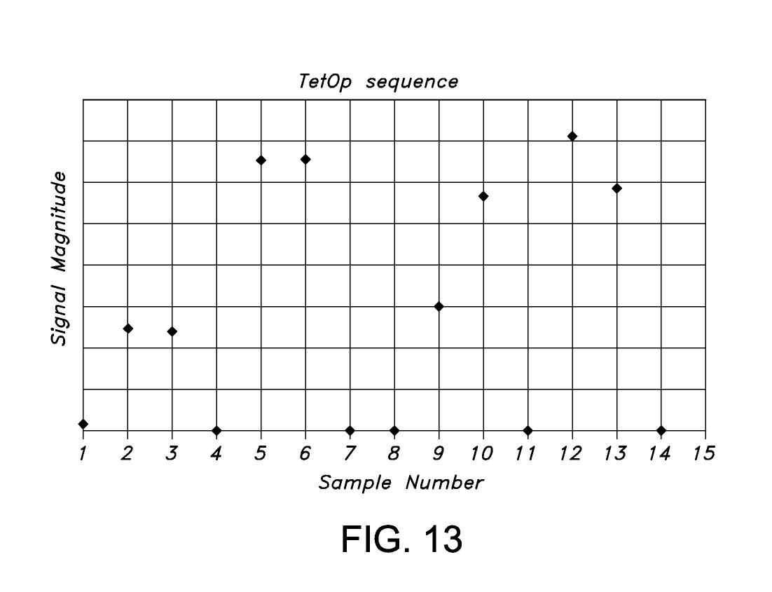

Now referring to FIGS. 11-12, the plurality of samples have been treated as described in Example 1 to obtain screening results which are shown as a graph of signal magnitude for the designated genetic sequence. The remote user 1 is provided with the graphs as shown in FIGS. 11-12 and asked to select a signal magnitude for the homozygous, heterozygous and wild type strains. In FIG. 11, the top ⅓ data points are considered homozygous samples, the middle ⅓ data points are considered heterozygous samples and the bottom ⅓ data points are considered wild type samples. The remote user 1 transmits their signal magnitude designation corresponding to the sample types to the screening laboratory 20. Then as additional RIP7-rtTA samples are received from the remote user 1 at the screening laboratory 20 in designated microwell containers and at the request of the remote user 1 for transgenic zygosity genotyping then the plurality of samples are screened according to the method described in Example 1. The remote user 1 then receives screening results as an electronic image which shows whether a sample, as designated by its well plate location and sample identification number is homozygotic (+ +); heterozygotic (+ −) or homozygotic (− −).

TABLE 10

| Well | | | | | | | | |
|---|---|---|---|---|---|---|---|---|
| plate | | | | | | | | |
| Location | Strain | Sample ID | rtTA | | AKT | | TepOp | |
| A1 | RIP7-rtTA | 1 | + | − | | | | |
| B1 | RIP7-rtTA | 2 | + | − | | | | |
| C1 | RIP7-rtTA | 3 | + | + | | | | |
| D1 | RIP7-rtTA | 4 | − | − | | | | |
| E1 | RIP7-rtTA | 5 | + | − | | | | |
| F1 | RIP7-rtTA | 6 | − | − | | | | |
| G1 | RIP7-rtTA | 7 | + | + | | | | |
| H1 | RIP7-rtTA | 8 | + | + | | | | |
| A2 | RIP7-rtTA | 9 | + | − | | | | |
| B2 | RIP7-rtTA | 10 | + | − | | | | |
| C2 | RIP7-rtTA | 11 | − | − | | | | |
| D2 | RIP7-rtTA | 12 | + | − | | | | |
| E2 | RIP7-rtTA | 13 | − | − | | | | |
| F2 | RIP7-rtTA | 14 | + | − | | | | |
| G2 | RIP7-rtTA | 15 | − | − | | | | |
| H2 | RIP7-rtTA | 16 | − | − | | | | |
| A3 | TetAKT1 | 1 | | | + | − | | |
| B3 | TetAKT1 | 2 | | | + | − | | |
| C3 | TetAKT1 | 3 | | | + | − | | |
| D3 | TetAKT1 | 4 | | | − | − | | |
| E3 | TetAKT1 | 5 | | | + | + | | |
| F3 | TetAKT1 | 6 | | | − | − | | |
| G3 | TetAKT1 | 7 | | | − | − | | |
| H3 | TetAKT1 | 8 | | | + | + | | |
| A4 | TetAKT1 | 9 | | | + | − | | |
| B4 | TetAKT1 | 10 | | | − | − | | |
| C4 | TetAKT1 | 11 | | | + | − | | |
| D4 | TetAKT1 | 12 | | | + | + | | |
| E4 | TetAKT1 | 13 | | | + | + | | |
| F5 | TetAKT1 | 14 | | | − | − | | |
| G4 | TetAKT1 | 15 | | | − | − | | |
| H4 | TetAKT1 | 16 | | | + | − | | |
| A5 | TetAKT1 | 17 | | | + | − | | |
| B5 | TetAKTI | 18 | | | − | − | | |
| C5 | Tetp27KIP | 1 | | | | | − | − |
| D5 | Tetp27KIP | 2 | | | | | + | − |
| E5 | Tetp27KIP | 3 | | | | | + | − |
| F5 | Tetp27KIP | 4 | | | | | − | − |
| G5 | Tetp27KIP | 5 | | | | | + | + |
| H5 | Tetp27KIP | 6 | | | | | + | + |
| A6 | Tetp27KIP | 7 | | | | | − | − |
| B6 | Tetp27KIP | 8 | | | | | − | − |
| C6 | Tetp27KIP | 9 | | | | | + | − |
| D6 | Tetp27KIP | 10 | | | | | + | + |
| E6 | Tetp27KIP | 11 | | | | | − | − |
| F6 | Tetp27KIP | 12 | | | | | + | + |
| G6 | Tetp27KIP | 13 | | | | | + | + |
| H6 | Tetp27KIP | 14 | | | | | − | − |

### Example 9 Single Nucleotide Polymorphism Genotyping

A single nucleotide polymorphism (SNP) is a mutation that affects only one base in the genetic sequence. These mutations occur naturally or can be engineered into a subject. Although, SNPs occur in both humans and mice the tissue source for this experiment was a mouse tail biopsies. Once the bioinformatics and SNP sequence information is acquired, two primers and two probes are created. The forward and reverse primers will hybridize to the genomic sequence flanking each side of the point mutation during the annealing step of the PCR reaction. Moreover, the wild type probe and the mutant probe will compete to hybridize to the DNA. The wild type probe, being perfectly homologous to the wild type genetic condition, will out compete the mutant probe on wild type DNA. Conversely, the mutant probe will out compete the wild type probe on mutated DNA that has the SNP. The two probes multiplexed with two primers discern the correct genotype in this reaction. The first probe determines if the sequence of the mutant is present by the probe being perfectly homologous to the mutant condition. The second probe determines if the endogenous DNA sequence is present. The second probe is perfectly homologous to the endogenous sequence. The two primers and probes are run on the individual samples at the same time. The probes compete for the DNA and a genotype is discernable. The results are then determined by evaluating both pieces of information to determine mutants from nonmutant individuals. Mutations that differ at two or more bases can also be genotyped using this method.

Specifically, a remote user 1 contacts the screening laboratory 20 and provides a mouse vendor stock number. The screening laboratory 20 can then use this number to query a vendor's database, which yields a description. This particular description states that the mutant Apc^{Min} allele has a T to A transversion at nucleotide 2549. This point mutation changes codon 850 to a pre-mature stop codon. The Ensembl database is then queried for the transcript sequence which has an Ensembl Transcript Identification number of ENSMUST00000079362. This is the designated genetic sequence. The 850th codon is identified.

(SEQ ID NO. 74)

GAGCTTCGGGCGAAGGCCCGGGAGCAGCGGACCGAGGCTGGCGCGAT
GCTGTTCCCGGGGAGCGCAGTCGGCTACCGTTGAGGAAGGTGGAGTGAGG
AGTGGCCCTTCCAGCGCCCCCTATGTACGCCTTCCTGCGCTCGGGGCCGG
TCGCCGCGTTGCCCGCCTCCGTACCGCCCGTGACTCTCGGGGCCCGGAGC
TCCGGCGGCGGCCGGGGTCGAGTCCCGGGGGAGGGGAGGCGCCCGGGCGG
CGCCCGAGCTTGCGGCCGCGGAGCGAGCGTCTGGCAGGTCCAAGGGTAGC
CAAGGATGGCTGCAGCTTCATATGATCAGTTGTTAAAGCAAGTTGAGGCA
CTGAAGATGGAGAACTCAAATCTTCGACAAGAGCTAGAAGATAATTCCAA
TCATCTTACAAAACTGGAAACTGAGGCATCTAATATGAAGGAAGTACTTA
AGCAGCTACAGGGAAGTATTGAAGATGAGACTATGACTTCTGGACAGATT
GACTTACTAGAGCGTCTTAAAGAATTTAACTTAGATAGTAATTTCCCCGG
AGTGAAACTACGCTCAAAAATGTCCCTTCGCTCCTACGGAAGTCGGGAAG
GATCTGTATCCAGCCGTTCAGGAGAATGCAGTCCTGTCCCCATGGGGTCA
TTCCCAAGAAGAACATTTGTAAATGGAAGCAGAGAGAGTACTGGGTATCT
AGAAGAGCTTGAAAAAGAAAGATCATTACTCCTTGCTGATCTTGACAAAG
AAGAGAAGGAAAAGGACTGGTATTATGCTCAACTTCAGAACCTCACAAAA
AGAATAGATAGCCTGCCTTTAACTGAAAATTTTTCCTTACAGACAGACAT
GACAAGACGGCAGCTGGAGTATGAAGCAAGGCAGATCAGGGCTGCAATGG
AGGAGCAGCTTGGCACCTGCCAGGACATGGAGAAGCGTGCACAGCGAAGA
ATAGCCAGGATCCAGCAAATAGAAAAGGACATACTGCGCGTGCGCCAGCT
TTTACAGTCCCAGGCGGCGGAAGCGGAGAGGTCATCTCAGAGCAGGCATG
ATGCTGCCTCCCATGAAGCTGGCCGGCAGCACGAAGGCCACGGAGTGGCA
GAAAGCAACACCGCAGCCTCCAGTAGTGGTCAGAGTCCAGCTACACGTGT
GGATCACGAAACAGCCAGTGTTTTGAGTTCTAGCGGCACGCACTCTGCTC
CTCGAAGGTTGACAAGTCATCTGGGGACAAAGGTGGAAATGGTGTATTCC
TTGTTGTCAATGCTTGGTACTCATGATAAGGACGATATGTCACGAACTTT
GCTAGCTATGTCCAGCTCCCAAGACAGCTGTATATCCATGCGGCAGTCTG
GATGTCTTCCTCTCCTCATCCAGCTTTTACATGGCAATGACAAAGACTCT
GTATTGTTGGGAAATTCCCGGGGCAGTAAAGAGGCTCGGGCCAGGGCCAG
TGCAGCACTCCACAACATCATTCACTCACAGCCTGATGACAAGAGAGGCA
GGCGTGAAATCCGAGTCCTTCATCTTTTGGAACAGATACGAGCTTACTGT
GAAACCTGTTGGGAGTGGCAGGAAGCCCACGAACAAGGCATGGACCAGGA
CAAAAACCCAATGCCAGCTCCTGTTGAGCATCAGATCTGTCCTGCTGTGT
GTGTTCTAATGAAGCTTTCATTTGATGAAGAGCATAGGCATGCAATGAAT
GAACTTGGGGGACTGCAGGCCATTGCAGAGTTATTGCAGGTGGACTGTGA
GATGTATGGGCTTACTAATGACCACTACAGTGTTACTTTAAGACGGTATG
CTGGAATGGCTTTGACAAACTTGACCTTTGGAGATGTTGCCAACAAGGCT
ACGCTGTGTTCTATGAAAGGCTGCATGAGAGCACTTGTGGCCCAGTTAAA
ATCTGAGAGTGAAGACTTACAGCAGGTTATTGCAAGTGTTTTGAGGAATT
TGTCTTGGCGAGCAGATGTAAATAGCAAAAAGACGTTGAGAGAAGTTGGA
AGTGTGAAAGCATTGATGGAATGTGCTTTGGAAGTTAAAAAGGAATCAAC
CCTCAAAAGCGTTTTGAGTGCCTTATGGAACCTGTCTGCACACTGCACTG
AGAATAAGGCTGACATCTGTGCTGTGGATGGAGCACTGGCATTTCTGGTT
GGCACCCTCACTTACCGGAGCCAGACAAATACTTTAGCCATTATTGAAAG
TGGAGGTGGGATATTACGGAATGTGTCCAGCTTGATAGCTACAAACGAAG
ACCACAGGCAAATCCTAAGAGAGAACAATTGCCTACAAACTTTATTACAG
CACTTGAAATCTCACAGCTTGACAATAGTCAGTAATGCATGTGGAACTTT
GTGGAATCTCTCAGCAAGAAATCCTAAAGACCAGGAAGCCTTGTGGGACA
TGGGGGCAGTGAGCATGCTCAAGAACCTCATTCATTCCAAGCACAAAATG
ATTGCCATGGGAAGTGCAGCAGCTTTAAGGAATCTCATGGCAAACAGACC
TGCAAAGTATAAGGATGCCAATATCATGTCTCCCGGCTCAAGTCTGCCAT
CCCTTCACGTTAGGAAACAGAAAGCTCTAGAAGCTGAGCTAGATGCTCAG
CATTTATCAGAAACCTTCGACAACATTGACAACCTAAGTCCCAAGGCCTC
TCACCGGAGTAAGCAGAGACACAAGCAGAATCTTTATGGTGACTATGCTT
TTGACGCCAATCGACATGATGATAGTAGGTCAGACAATTTCAATACTGGA
AACATGACTGTTCTTTCACCATATTTAAATACTACGGTATTGCCCAGCTC
TTCTTCCTCAAGGGGAAGTTTAGACAGTTCTCGTTCTGAGAAAGACAGAA
GTTTGGAGAGAGAGCGAGGTATTGGCCTCAGTGCTTACCATCCAACAACA
GAAAATGCAGGAACCTCATCAAAACGAGGTCTGCAGATCACTACCACTGC
AGCCCAGATAGCCAAAGTTATGGAAGAAGTATCAGCCATTCATACCTCCC
AGGACGACAGAAGTTCTGCTTCTACCACCGAGTTCCATTGTGTGGCAGAC
GACAGGAGTGCGGCACGAAGAAGCTCTGCCTCCCACACACACTCAAACAC
ATACAACTTCACTAAGTCGGAAAATTCAAATAGGACATGCTCTATGCCTT
ATGCCAAAGTGGAATATAAACGATCTTCAAATGACAGTTTAAATAGTGTC
ACTAGTAGTGATGGATATGGTAAAAGAGGCCAAATGAAACCCTCAGTTGA
ATCCTATTCTGAAGATGATGAAAGTAAATTTTGCAGTTATGGTCAGTATC
CAGCTGACCTAGCCCATAAGATACACAGTGCAAATCATATGGATGATAAT
GATGGAGAACTGGATACACCAATAAATTACAGTCTTAAATATTCAGATGA
GCAGTTGAACTCAGGAAGGCAGAGTCCCTCACAGAATGAAAGGTGGGCAA
GACCAAAGCATGTGATAGAAGATGAAATAAAGCAAAACGAGCAAAGACAA
GCAAGAAGCCAGAACACCAGTTATCCTGTCTATTCTGAGAATACCGATGA
CAAACACCTCAAATTCCAACCACATTTTGGACAACAAGAATGTGTTTCCC
CATATAGGTCAAGGGGAACCAGTGGTTCAGAAACAAATCGAATGGGTTCT
AGTCATGCAATTAATCAAAATGTAAACCAGTCTCTGTGTCAGGAAGATGA
TTATGAAGATGATAAACCTACCAACTACAGTGAACGTTATTCTGAGGAAG
AACAACATGAAGAAGAAGAAGAGAGACCGACAAATTATAGCATAAAATAT
AATGAAGAGAAACATCATGTGGATCAGCCTATTGATTATAGTTTAAAATA
TGCCACTGACATTTCTTCCTCACAAAAACCATCATTTTCATTCTCAAAGA
ATTCATCAGCACAAAGCACTAAACCTGAACATCTCTCTCCAAGCAGCGAG
AATACAGCTGTACCTCCATCTAATGCCAAAAGGCAGAATCAGCTGCGTCC
AAGTTCAGCACAAAGAAATGGCCAGACTCAAAAAGGCACTACTTGCAAAG
TCCCCTCCATCAACCAAGAAACAATACAGACTTACTGCGTAGAAGACACC
CCAATATGTTTTTCAAGGTGCAGTTCATTATCATCACTGTCATCAGCTGA
CGATGAAATAGGATGTGATCAGACAACACAGGAAGCAGATTCTGCTAATA
CTCTGCAGACAGCAGAAGTAAAAGAGAATGATGTAACTCGGTCAGCTGAA
GATCCTGCAACTGAAGTTCCAGCAGTGTCCCAGAATGCTAGAGCCAAACC
CAGCCGACTCCAGGCTTCTGGCTTATCTTCAGAATCAACCAGGCATAATA
AAGCTGTTGAGTTTTCTTCAGGAGCCAAGTCTCCCTCCAAAAGTGGTGCT
CAGACACCCAAAAGTCCCCCAGAACACTATGTCCAGGAGACTCCGCTCGT
ATTCAGCAGGTGTACTTCTGTCAGCTCCCTTGACAGTTTTGAGAGTCGCT
CCATTGCCAGCTCTGTTCAGAGTGAGCCATGTAGTGGAATGGTGAGTGGC
ATCATAAGCCCCAGTGACCTTCCAGATAGTCCTGGGCAGACCATGCCACC
AAGCAGAAGCAAAACCCCTCCACCTCCTCCACAGACAGTGCAGGCCAAGA
GAGAGGTGCCAAAAAGTAAAGTCCCTGCTGCTGAGAAGAGAGAGAGTGGG
CCTAAGCAGACTGCTGTAAATGCTGCCGTGCAGAGGGTGCAGGTCCTTCC
AGACGTGGATACTTTGTTACACTTCGCCACAGAAAGTACTCCAGACGGGT
TTTCTTGTTCCTCCAGCCTAAGTGCTCTGAGCCTGGATGAGCCATTTATA
CAGAAAGATGTAGAATTAAGAATCATGCCTCCAGTTCAGGAAAACGACAA
TGGGAATGAAACTGAATCAGAACAGCCTGAGGAATCAAATGAAAACCAGG
ATAAAGAGGTAGAAAAGCCTGACTCTGAAAAAGACTTATTAGATGATTCT
GATGACGATGATATTGAAATATTAGAAGAATGTATTATTTCAGCCATGCC
AACAAAGTCATCACGCAAAGCCAAAAAACTAGCCCAGACTGCTTCAAAAT
TACCTCCACCTGTGGCAAGGAAACCAAGTCAGCTACCTGTGTATAAACTT
CTGCCAGCACAGAATAGGCTGCAGGCACAAAAACATGTTAGCTTTACACC
AGGGGATGATGTGCCCCGGGTGTACTGTGTAGAAGGGACACCTATAAACT
TTTCCACAGCAACGTCTCTAAGTGATCTGACAATAGAGTCCCCTCCAAAT
GAATTGGCTACTGGAGATGGGGTCAGAGCGGGTATACAGTCAGGTGAATT
TGAAAAACGAGATACCATTCCTACAGAAGGCAGAAGTACAGATGATGCTC
AGCGAGGAAAAATCTCATCTATAGTTACACCAGACCTGGATGACAACAAA
GCAGAGGAAGGAGATATTCTTGCAGAATGTATCAATTCTGCTATGCCCAA
AGGAAAAAGCCACAAGCCTTTCCGAGTGAAAAAGATAATGGACCAAGTCC
AACAAGCATCCTCGACTTCATCTGGAGCTAACAAAAATCAAGTAGACACT
AAGAAAAAGAAGCCTACTTCACCAGTAAAGCCCATGCCACAAAATACTGA
ATATAGAACGCGTGTGAGAAAGAATACAGACTCAAAAGTTAATGTAAATA
CTGAAGAAACTTTCTCAGACAACAAAGACTCAAAGAAACCAAGCTTACAA
ACCAATGCCAAGGCCTTCAATGAAAAGCTACCTAACAATGAAGACAGAGT
GCGGGGGAGCTTCGCCTTGGACTCACCGCATCACTACACCCCTATTGAGG
GGACGCCGTACTGCTTTTCCCGAAATGACTCCTTGAGTTCTCTGGATTTT
GATGATGACGATGTTGACCTTTCCAGGGAAAAGGCCGAGTTAAGAAAGGG
CAAAGAAAGCAAGGATTCCGAAGCCAAAGTTACCTGCCGCCCAGAACCAA
ACTCAAGCCAGCAGGCAGCTAGTAAGTCACAAGCCAGTATAAAACATCCA
GCAAACAGAGCACAGTCCAAACCAGTGCTGCAGAAACAGCCCACTTTCCC
CCAGTCCTCCAAAGACGGACCAGATAGAGGGGCAGCAACTGACGAAAAAC
TGCAGAATTTTGCTATTGAAAATACTCCAGTTTGCTTTTCTCGAAATTCC
TCTCTGAGTTCCCTTAGTGACATTGACCAGGAAAACAACAATAACAAAGA
AAGTGAACCAATCAAAGAAGCTGAACCTGCCAACTCACAAGGAGAGCCCA
GTAAGCCTCAGGCATCCGGGTATGCTCCCAAGTCCTTCCACGTCGAAGAC
ACCCCTGTCTGTTTCTCAAGAAACAGCTCTCTCAGTTCTCTTAGCATTGA
CTCTGAGGACGACCTGTTACAGGAGTGTATAAGTTCTGCCATGCCAAAAA
AGAAAAGGCCTTCAAGACTCAAGAGTGAGAGCGAAAAGCAGAGCCCTAGA
AAAGTGGGTGGCATATTAGCTGAAGACCTGACGCTTGATTTGAAAGATCT
ACAGAGGCCAGATTCAGAACACGCTTTCTCCCCCGACTCAGAAAATTTTG
ACTGGAAAGCTATTCAGGAAGGCGCAAACTCCATAGTAAGTAGTTTGCAC
CAAGCTGCTGCAGCCGCCGCGTGCTTATCTAGACAAGCGTCATCCGACTC
AGATTCCATTCTGTCACTAAAGTCCGGCATTTCTCTGGGATCGCCTTTTC
ATCTTACACCTGATCAAGAGGAAAAGCCATTCACAAGCAATAAAGGCCCA
AGAATTCTCAAACCTGGAGAGAAAAGCACATTAGAAGCAAAAAAAATAGA
ATCTGAAAACAAAGGAATCAAAGGCGGGAAAAAGGTTTATAAAAGCTTGA
TTACGGGAAAGATTCGCTCCAATTCAGAAATTTCCAGCCAAATGAAACAA
CCCCTCCCGACAAACATGCCTTCAATCTCAAGAGGCAGGACGATGATTCA
CATCCCAGGGCTTCGGAATAGCTCCTCTAGTACAAGCCCTGTCTCTAAGA
AAGGCCCACCCCTCAAGACTCCAGCCTCTAAAAGCCCCAGTGAAGGGCCG
GGAGCTACCACTTCTCCTCGAGGAACTAAGCCAGCAGGAAAGTCAGAGCT
TAGCCCTATCACCAGGCAAACTTCCCAAATCAGTGGGTCAAATAAGGGGT
CTTCTAGATCAGGATCTAGAGACTCCACTCCCTCAAGACCTACACAGCAA
CCATTAAGTAGGCCAATGCAGTCTCCAGGGCGAAACTCAATTTCCCCTGG
TAGAAATGGAATAAGCCCTCCTAACAAACTGTCTCAGCTGCCCAGAACAT
CATCTCCCAGTACTGCTTCAACTAAGTCCTCCGGTTCTGGGAAAATGTCA
TATACATCCCCAGGTAGACAGCTGAGCCAACAAAATCTTACCAAACAAGC
AAGTTTATCCAAGAATGCCAGCAGTATCCCCAGAAGTGAGTCGGCATCTA
AAGGACTGAATCAGATGAGTAACGGCAATGGGTCAAATAAAAAGGTAGAA
CTTTCTAGAATGTCTTCAACTAAATCAAGTGGAAGTGAATCAGACAGATC
AGAAAGGCCTGCATTAGTACGCCAGTCTACTTTCATCAAAGAAGCCCCAA
GCCCAACCCTGAGGAGGAAACTGGAGGAATCTGCCTCATTTGAATCCCTT
TCTCCATCTTCTAGACCAGATTCTCCCACCAGGTCGCAGGCACAGACCCC
AGTTTTAAGCCCTTCCCTTCCTGATATGTCTCTGTCCACACATCCATCTG
TTCAGGCAGGTGGGTGGCGAAAGCTCCCGCCTAATCTCAGCCCCACTATC
GAGTATAATGACGGAAGGCCCACAAAACGGCATGATATTGCACGCTCCCA
TTCTGAAAGTCCTTCCAGACTACCAATCAACCGGGCGGGAACCTGGAAGC
GTGAACACAGCAAACATTCCTCGTCCCTTCCTCGAGTGAGTACTTGGAGA
AGAACTGGAAGCTCATCTTCTATTCTTTCTGCTTCATCAGAGTCCAGTGA
AAAAGCAAAAAGTGAGGATGAAAGGCATGTGAGCTCCATGCCAGCACCCA
GACAGATGAAGGAAAACCAGGTGCCCACCAAAGGAACATGGAGGAAAATC
AAGGAAAGTGACATTTCTCCCACAGGCATGGCTTCTCAGAGCGCTTCCTC
AGGTGCTGCCAGTGGTGCTGAATCCAAGCCTCTGATCTATCAGATGGCAC
CTCCTGTCTCTAAAACAGAGGATGTTTGGGTGAGAATTGAGGACTGCCCC
ATTAACAACCCTAGATCTGGACGGTCCCCCACAGGCAACACCCCCCCAGT
GATTGACAGTGTTTCAGAGAAGGGAAGTTCAAGCATTAAAGATTCAAAAG
ACACCCATGGGAAACAGAGTGTGGGCAGTGGCAGTCCTGTGCAAACCGTG
GGTCTGGAAACCCGCCTCAACTCCTTTGTTCAGGTAGAGGCCCCAGAACA
GAAAGGAACTGAGGCAAAACCAGGACAGAGTAACCCAGTCTCTATAGCAG
AGACTGCTGAGACGTGTATAGCAGAGCGTACCCCTTTCAGTTCCAGTAGC
TCCAGCAAGCACAGCTCACCTAGCGGGACTGTTGCTGCCAGAGTGACACC
TTTTAATTACAACCCTAGCCCTAGGAAGAGCAGCGCAGACAGCACTTCAG
CCCGGCCGTCTCAGATCCCTACGCCAGTGAGCACCAACACGAAGAAGAGA
GATTCGAAGACTGACAGCACAGAATCCAGTGGAGCCCAAAGTCCTAAACG
CCATTCCGGGTCTTACCTCGTGACGTCTGTTTAA

This large designated genetic sequence can be truncated for easier data handling. The smaller designated genetic sequence is a subset of nucleotides of the larger designated genetic sequence. The smaller designated genetic sequence contains the informative locations and nucleotides for the assay to be designed. The smaller designated genetic sequence is:

(SEQ ID NO. 9)

TATCATGTCTCCCGGCTCAAGTCTGCCATCCCTTCACGTTAGGAAACA
GAAAGCTCTAGAAGCTGAGCTAGATGCTCAGCATTTATCAGAAACCTTCG
ACAACATTGACAACCTAAGTCCCAAGGCCTCTCACCGGAGTAAGCAGAGA
CACAAGCAGAATCTTTATGGTGACTATGCTTTTGACGCCAATCGACATGA
TGATAGTAGGTCAGACAATTTCAATACTGGAAACATGACTGTTCTTTCAC
CATATTTAAATACTACGGTATTGCCCAGCTCTTCTTCCTCAAGGGGAAGT
TTAGACAGTTCTCGTTCTGAGAAAGACAGAAGTTTGGAGAGAGAGCGAGG
TATTGGCCTCAGTGCTTACCATCCAACAACAGAAAATGCAGGAACCTCAT
CAAAACGAGGTCTGCAGATCACTACCACTGCAGCCCAGATAGCCAAAGTT
ATGGAAGAAGTATCAGCCATTCATACCTCCCAGGACGACAGAAGTTCTGC
TTCTACCACCGAGTTCCATTGTGTGGCAGACGACAGGAGTGCGGCACGAA
GAAGCTCTGCCTNNNNNNNNNNNNNNNNNNNNNNNNNCTTCACTAAGTCG
GAAAATTCAAATAGGACATGCTCTATGCCTTATGCCAAAGTGGAATATAA
ACGATCTTCAAATGACAGTTTAAATAGTGTCACTAGTA

Upon identification of the designated genetic sequence two other software programs are utilized. The first of these programs is a blast program that identifies homologies between the designated genetic sequence and the endogenous genome of the mouse, as well as other species. The blast software can be found at http://www.ncbi.nlm.nih.gov/BLAST/.

The second of these programs is repeat masking program, such as Repeat Master Web Server found at http://www.repeatmasker.org/cgi-bin/WEBRepeatMasker. This program identifies areas in the designated genetic sequence that are highly repetitive, making them less than ideal locations to build a primer or probe. If such areas are found in the designated genetic sequence they are masked by replacing the normal nucleotide designation A, C, G or T with the letter N or X.

Applied Biosystem's FileBuilder software program is then utilized to generate a SNP assay. The FileBuilder software allows the screening laboratory 20 to identify the location inside the designated genetic sequence that is informative. The transversion, of T to an A in the mutant condition is targeted. In this designated genetic sequence this would correspond to a target location of the 333rd nucleotide. The FileBuilder software file with the 333rd nucleotide designated as the target, is electronically transmitted to Applied Biosystems to generate an Assays-by-Design order. Applied Biosystems will use a software program, such as Primer Express® or Taq Pipe, to identify primer and probe sequences that will detect this genetic condition. The software generates the following primers and probe.

(SEQ ID NO. 10)

Forward Primer: GGGAAGTTTAGACAGTTCTCGTTCT

(SEQ ID NO. 11)

Reverse Primer: GTAAGCACTGAGGCCAATACCT

(SEQ ID NO. 12)

Probe 1: CTCTCTCCAAACTTC

(SEQ ID NO. 13)

Probe 2: TCTCTCTCCTAACTTC

The primers and probes will hybridized or anneal the following areas in the designated genetic sequence.

(SEQ ID NO.9)

TATCATGTCTCCCGGCTCAAGTCTGCCATCCCTTCACGTTAGGAAACAGA
AAGCTCTAGAAGCTGAGCTAGATGCTCAGCATTTATCAGAAACCTTCGAC
AACATTGACAACCTAAGTCCCAAGGCCTCTCACCGGAGTAAGCAGAGACA
CAAGCAGAATCTTTATGGTGACTATGCTTTTGACGCCAATCGACATGATG
ATAGTAGGTCAGACAATTTCAATACTGGAAACATGACTGTTCTTTCACCA
TATTTAAATACTACGGTATTGCCCAGCTCTTCTTCCTCAAGGGGAAGTTT
AGACAGTTCTCGTTCTGAGAAAGACAGAAGTTGGAGAGAGAGCGAGGT
ATTGGCCTCAGTGCTTACCATCCAACAACAGAAAATGCAGGAACCTCATC
AAAACGAGGTCTGCAGATCACTACCACTGCAGCCCAGATAGCCAAAGTTA
TGGAAGAAGTATCAGCCATTCATACCTCCCAGGACGACAGAAGTTCTGCT
TCTACCACCGAGTTCCATTGTGTGGCAGACGACAGGAGTGCGGCACGAAG
AAGCTCTGCCTNNNNNNNNNNNNNNNNNNNNNNNNNCTTCACTAAGTCGG
AAAATTCAAATAGGACATGCTCTATGCCTTATGCCAAAGTGGAATATAAA
CGATCTTCAAATGACAGTTTAAATAGTGTCACTAGTA

The genomic DNA nucleotides from the forward primer to the end of the reverse primer and all the bases in between, whether they hybridized to primer probe are not, are known as the target genetic sequence. For Apc^{Min} the target genetic sequence is:

(SEQ ID NO. 9)

TATCATGTCTCCCGGCTCAAGTCTGCCATCCCTTCACGTTAGGAAACA
GAAAGCTCTAGAAGCTGAGCTAGATGCTCAGCATTTATCAGAAACCTTCG
ACAACATTGACAACCTAAGTCCCAAGGCCTCTCACCGGAGTAAGCAGAGA
CACAAGCAGAATCTTTATGGTGACTATGCTTTTGACGCCAATCGACATGA
TGATAGTAGGTCAGACAATTTCAATACTGGAAACATGACTGTTCTTTCAC
CATATTTAAATACTACGGTATTGCCCAGCTCTTCTTCCTCAAGGGGAAGT
TTAGACAGTTCTCGTTCTGAGAAAGACAGAAGTTGGAGAGAGAGCGAG
GTATTGGCCTCAGTGCTTACCATCCAACAACAGAAAATGCAGGAACCTCA
TCAAAACGAGGTCTGCAGATCACTACCACTGCAGCCCAGATAGCCAAAGT
TATGGAAGAAGTATCAGCCATTCATACCTCCCAGGACGACAGAAGTTCTG
CTTCTACCACCGAGTTCCATTGTGTGGCAGACGACAGGAGTGCGGCACGA
AGAAGCTCTGCCTNNNNNNNNNNNNNNNNNNNNNNNNNCTTCACTAAGTC
GGAAAATTCAAATAGGACATGCTCTATGCCTTATGCCAAAGTGGAATATA
AACGATCTTCAAATGACAGTTTAAATAGTGTCACTAGTA

A vendor, such as Applied Biosystems, will synthesize these Real-Time primer and probe sequences and send them to the screening laboratory 20. One fluorescent probe will be perfectly homologous to the endogenous condition, while the other probe labeled with a different fluorescence will be perfectly homologous to the mutant condition.

A biological sample in the form of a mouse tail biopsy is submitted via FedEx (Memphis, Tenn.) overnight delivery to the screening laboratory 20 from the remote user 1. Each sample occupies one well of a 96 well source well container. A lysis reagent (made of 2.5 μl of proteinase K (VWR EM-24568-3) and 147.5 μl of Nuclei Lysing Solution (Promega Corporation, Madison, Wis. A 7943) per sample)) is gently mixed and poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is then placed in a 55° C. oven for three hours. The well plate is then placed back on the deck of the Tecan Genesis Workstation (Research Triangle Park, N.C.). The liquid handler aspirates 50 μl of each sample and dispenses it in to a 384 well primary master well container (Fisher Scientific #NC9134044). Once all of the samples are transferred, the primary master well container is moved to the deck of the Isolation Purification Station 94.

One hundred and twelve microliters of SV Lysis reagent (Promega Corporation, Madison Wis., #Z305X) a chaotropic salt are added to each sample. Next, 13 μl of magnetic particles (Promega Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container are placed on a 384 tip dryer for 11 minutes. Then the microwell container are moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 units is acceptable.

The primary master wellplate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog #4326708), 1× real time PCR primer set/probe mix for the designated genetic sequence (Applied Biosystems Assays-by-Design (SM) Service 4331348) and 25% isolated DNA. The Tecan Genesis added the reagents together in the ABI 7900 384 Well Optical Plate. The plate is then sealed with optical sealing tape (ABI, #4311971). The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Table 11. On average, these results are transmitted to the remote user 1 within twenty-four hours of receiving the biological sample at the screening laboratory 20.

TABLE 11
APC MIN

| | APC MIN | | | APC MIN | | |
|---|---|---|---|---|---|---|
| | Positives for | | | Negatives for | | |
| | the mutation | | | the mutation | | |
| | MIN | 1.0 | | MIN | 0.0 | |
| | MAX | 7.5 | | MAX | 0.7 | |
| T5009 | 5.9 | 6.6 | + | 0.6 | 0.4 | − |
| | 5.7 | 6.0 | + | 0.5 | 0.4 | − |
| | 5.7 | 6.1 | + | 0.4 | 0.4 | − |
| | 6.2 | 5.9 | + | 0.0 | 0.0 | − |
| | 5.8 | 6.1 | + | 0.0 | 0.0 | − |
| | 5.5 | 6.0 | + | 0.5 | 0.4 | − |
| | 7.0 | 6.1 | + | 0.4 | 0.5 | − |
| | 6.1 | 6.3 | + | 0.5 | 0.5 | − |
| | 6.9 | 5.4 | + | 0.4 | 0.4 | − |
| | 7.0 | 6.1 | + | 0.5 | 0.6 | − |
| | | | | 0.5 | 0.6 | − |
| | | | | 0.4 | 0.5 | − |

In this example the relative signal for a positive individual with the mutant probe verses the endogenous probe fall in the range of 1.0 and 7.5.

### Example 10 Human Genotyping of Endogenous Sequences

The remote user 1 provides the genetic line identification 84. The genetic line in this example has been previously associated by the remote user 1 with the designated genetic sequence for Human TTTY8 (SEQ ID NO. 26).

A biological sample in the form of human tissue and mouse tissue is submitted via FedEx (Memphis, Tenn.) overnight delivery to the screening laboratory 20 from the remote user 1. Each sample occupies one well of a 96 well source well container. A lysis reagent (made of 2.5 μl of proteinase K (VWR EM-24568-3) and 147.5 μl of Nuclei Lysing Solution (Promega Corporation, Madison, Wis. A7943) per sample)) is gently mixed and poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is then placed in a 55° C. oven for three hours. The well plate is then placed back on the deck of the Tecan Genesis Workstation (Research Triangle Park, N.C.). The liquid handler aspirated 50 μl of each sample and dispenses it into a 384 well primary master well container (Fisher Scientific #NC9134044). Once all of the samples are transferred, the primary master well container is moved to the deck of the Isolation Station Purification Station 94.

One hundred and twelve microliters of SV Lysis reagent (Promega Corporation, Madison Wis., #Z305X) a chaotropic salt are added to each sample. Next, 13 μl of magnetic particles (Promega Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and is discarded. This washing process is repeated two additional times. Next, the samples are washed four times in 130 μl of 95% ethanol as described above. After the fourth ethanol wash, the microwell container is placed on a 384 tip dryer for 11 minutes. Then the microwell container is moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 O.D. units is acceptable.

The primary master well plate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog #4326708), 1× real time PCR primer set/probe mix for the designated genetic sequence (Applied Biosystems Assays-by-Design (SM) Service 4331348) and 25% isolated DNA. The Tecan Genesis added the reagents together in the ABI 7900 384 Well Optical Plate. The plate is then sealed with optical sealing tape (ABI, #4311971).

The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Table 12. On average, these results are transmitted to the remote user 1 within twenty-four hours of receiving the biological sample at the screening laboratory 20.

TABLE 12

| | | | | | HumanTTTY8 | |
|---|---|---|---|---|---|---|
| Sample Name | HumanTTTY8 RCN | | | | Result | Interpretation |
| Human - Not | 1.26 | 1.22 | 1.36 | 1.39 | + | Sample is Positive |
| Sonicated | | | | | | |
| Human - | 1.33 | 1.52 | 1.37 | 1.32 | + | Sample is Positive |
| Sonicated | | | | | | |
| Mouse DNA | N/A | N/A | N/A | N/A | − | Sample is Negative for both |
| | | | | | | HumanTTTY8 and HS0277190 |

### Example 11 Genotyping of Mouse Bone Marrow

Specifically, a remote user 1 can contacted the screening laboratory 20 and provide the Jackson Laboratory stock number, PCR genotyping protocol and the Alox-5 mutation description. The description disclosed that a pgk-neomycin cassette was inserted into the mutant sequence. However, this particular mutant model contains more than one pgk-neomycin mutation therefore a specific junction site must be targeted in order to discriminate this neomycin mutation from other neomycin mutations. Unfortunately, none of these pieces of information yielded the specific location (junction site) and nucleotide sequence of the mutation. A third party source was identified that had a working PCR fragment analysis genotyping protocol. The mutant band and the wild type band were cut from the gel. The pieces of gel were sent to a sequencing company to be purified and sequenced. Subsequently, the third party sent the remote user 1 the sequence data, who in turn forwarded it to the screening laboratory 20. The sequence data that was provided is the designated genetic sequence for the mutant.

(SEQ ID NO. 1)

TGCCCAGCGGTCCTATCTAGAGGTCATTCTCTCCACAGAGCGAGTCAA
GAACCACTGGCAGGAAGACCTCATGTTTGGCTACCAGTTCCTGAATGGCT
GCAACCCAGTAATTCTACCGGGTAGGGGAGGCGCTTTTCCCAAGGCAGTC
TGGAGCATGCGCTTTAGCAGCCCCGCTGGCACTTGGCGCTACACAAGTGG
CCTCTGGCCTCGCACACATTCCACATCCACCGGTAGCGCCAACCGGCTCC
GTTCTTTGGTGGCCCCTTCGCGCCACCTTCTACTCCTCCCCTAGTCAGGA
AGTTCCCCCCCGCCCCGCAGCTCGCGTCGTGCAGGACGTGACAAATGGAA
GTAGCACGTCTCACTAGTCTCGTGCAGATGGACAGCACCGCTGAGCAATG
GAAGCGGGTAGGCCTTTGGGGCAGCGGCCAATAGCAGCTTTGCTCCTTCG
CTTTCTGGGCTCAGAGGCTGGGAAGGGGTGGGTCCGGGGGCGGGCTCAGG
G

Knowing the gene name, Alox-5, from the mutation description provided from the remote user, the screening laboratory 20 can query the Ensembl database to provide the endogenous DNA sequence. The Ensembl gene identification number is ENSMUSG00000025701. This query yields sequence data, which is the designated genetic sequence for the endogenous condition.

(SEQ ID NO. 75)

GTTCCAGACAGTCCCACAGGTGCAGATTAGGAGTCGCCCACTCGGGCC
ACTACTTTCTAAGGCTGGTTCCCAGTACCACTAACCATTTCCCCCAAGTT
TGCTCCCACCCCGCGCCTCCCAGTACCTTGCCCAGAGAGAGAAGGTTTAC
TCATTTTGTGAAGAAACCAACATTCAAGTTTCCTTGGGGTCCACCGTGGA
GCTACAGGTACTCTCCTTGTGGGCTTCAGACCCCCTGCTCTAAGTGTAAC
TATTGCATACGCTGTGTGCTGCAACTGAATGAGGACACAGGTAGTTCTTG
AGCTGACAGTGAGGGACACTGAAACAGGCAAGAAGCCATAAAGATGGAAG
AAATAAGGGATTAATTGACTAATGAAAACAAAAATGCATGAGGGATAAAA
GCTAGGTAGAGATGGGGCAGAGGAACAAGGGCTTCAGCCTATCAGAGATC
ACTGTCCCTCCAGTGCCACAGGAGAGAAGGATGCGTTGGAAGGTGGGGTC
CTGGGACTGGCCAGAGACAGGGGCGGAGCCAGCGCCTGAAGGCAGGGCCG
GTCGCAGGGTGGAGCCAGACCCAAGCGCAAGGCTGGCCCGCTGCTGGCCA
CTGTGGCAGGGAGCTGCCGCGAGTGACAGGGTCAAGAAGTTGGTGGGCTG
CCACGCCGAGCTTCGCGGGCTCCTGCTCCCACACCAGCAGCACTCACTTG
CCCGGAGTCATGCCCTCCTACACGGTCACCGTGGCCACCGGCAGCCAGTG
GTTCGCGGGCACCGACGACTACATCTACCTCAGCCTCATTGGCTCTGCGG
GCTGTAGCGAGAAGCATCTGCTGGACAAGGCATTCTACAATGACTTCGAA
CGGGGCGCGGTGAGAATGCGCGCTTGGGACCGACGGCTGGCAGCAAAGAG
CGGGAGGGCGGCGGGGCAGGACAGGCAGGCACCTGAGAACTGTCTGTCCC
AGCGCGCTCGTGACCCTATGTGAGCGCATCTGGGGATCTGATGCAGCGCC
AGAGTCGGTGCATGCCAGGGCAAGCGAGGCACCCTAGACTTCCTGATGAC
TCGGGTATCTTAAGGGACAAATGACTTCCAATGTGGGGGACTGATGTGGC
TGGCCTCTTATGTGAGAGATGGCACAAACTTTCACCAACAGGACCCAGAA
TTTGTGAGAGGCCCCTTCACCTCGGGAGTTCTGAGGTCCTAGTGCCCCAG
AGTCCAGTACCACTGCAAAGCATAGAAAGCCCTGTTCGACAGCTAGCCAT
TTTGTGTAGACAGTGTAAGTCCAGGGTAAGTCAGAGATCCAGGATCGGGA
GTCAACTTGGGCATAATCACTCTTCTATCCCTACTGTGGACCTTCGTTTA
CCAAACTAAAAATTGGTTAGGTTTAATCTTACCCATGAGTCATGAGGGAG
CCCAGTCCCATTTGGGGGCTAGGAATGAGTCCAGGGATTGCCCCCCACAC
TTAGGTACCCTACATTTCCTGCACTCAGTCCCAGTGGAGGAATGTAAAGG
AAGGAAGGTCTGGCCCGAGCAGCCTTTGGAAAGACTGTCAGCAGGGTGCG
TGCAAGAGGACACTTCCTCCCTGAGTATTAGCTTCTGAAGGAAAAAAGGA
AGAAAGATTTCCTTTCTTCCCTCCTAATACAGACTTTGGAGTCTGGCAGC
CCCACAGGCACTGTGGGAGGCCATTCCGTTTGGATGCTGCTGGTGTGTAA
AGTCTAGCCCCATGACTTTCTCTTAACACAGGCAGGTCATGGTTTTCAGT
GACCCACTAAGTGCCTAGTACATCAGACTTGCTCAGTAAGTGAGTCTGTA
ATAGACAGGCACGCTGCAGACCCTTGGGGTGGGGGTGGGGGGTTCCTCTT
CCTTCTACTACCTCCAGGTCTAAACTAGGCTTAGGTTTGATTTTACCAGG
CTGCCTTCTTATCATTTAGTTTACTATTGAGTCGACCCACATGAACTTGC
TTAGAATTAACCATTTGTGATAAGCACAATGACAATTTCATATGCTTCCA
CTAGATAGATCTGTTCAGCCTAAACCAAGGAAATGATAGTTAAGCCCTTT
TCTGGGTCCCAAACTCTAAGCACGACGCTTACATTCCTATCCTGGGACCT
GGTACTTTGCCCTGATTAGCAAGCTTCTAACCAGGCTTGAACAAGCAAGC
GTGGGTGTTGACCTCAGAATGGCATCTTTTGCTCAGTTTCACCAGAGCTG
GCCAGAGAATGGTGCTGCCAAAGACCAAGCATGGCATCCGTTTGGGATGC
TGACACCCTGTGCAACCTCCAAAGCACTTGTGTTTATTTTCAGTAACTGG
GGCTTCTCCCAGTATACAGGGGGAGGAAGAGAGGACAGAATGCTTCCTCT
TTATAAATGGACTACAGGGGGCCTTCTCCACAAATCTAGCTATCAGTGGG
TTCAGTCTAGGTGCAGCACAGGACACCTTATGTGTCATTTCCTCCAGGAG
GAGAATGGCAATGTGGCCATCATAAATGACTGGACAGGAAGTAAGAGGCC
TGTCCTGTTCATCATTTGCCTTTCTGTCCTGCCTCCCAACCTGAAAAGTC
ATTCAGGTGACATTAATTTAACACCTTAGCAAGAACCCCAGAGGCAAATT
TCAGGAGAGATTTGCATACATATTTCCATTGTGGGGAGGGAACCACAGCT
CAAGTGAACTGCTGTCTTCTGGCTGGATGCAAAAAGAGCCTTAAAAAAAG
AAAAAGAAAACAGCCTTTGAGAAAGTTCCTGTTGATGCAAAGTTACTCCA
TACTTTGTCTTGCACAGTCCAGAGCCACTTCCTCATTCTGTGGCCAGTGT
ATCTTTAAAGTCCAGATGTCCCTTTTGGGTAAAGGTAGAAAAGAAACCTT
AAGGGAAGTGTTAGCTAAGAAGATAAGGTTATCCCACTGTTCTTAGAAAA
GTGCCACATTGTTTCCATGAATAGCAGCCACCGTGAAGGCCGGCCAGCCA
CTTCCAGCACCCCTTCCTAATGTACGACCGAATAATGGTCAGTGCTAACC
TGTTTTAATACATTCCACTATTGCCCATCCCCCATGATTCCCACATGAGT
TCTGTGTAGCGATTCAGGTCAGGAGCCTTTGCAGATGGGATTCTCCTGAG
TGTCAGGTGTCAGGCATCAGGGTTTCCCTGTTGAGAACCCAGACGGAACA
GAATGGCTTTGTTCCATAAGCTAGTCCTACGTGGCACAGCTCTAACAAAC
CTTGAAATCTTGAGTGCACATTAGTTGGCTACTTTAACTGCTCAACAGTA
CTTTGAAAAGCTAAGGATAGGCTGTCCTGCTGAGTATATGGTTCTTGAAC
ATATTCAGTACATGTAAATTCATCTTACAGGTAATACGCTTCTTAAATAC
ATCTAACAAATATTCTTATATTTATAGAGAGTAAATTCTATAGTACCTCT
TCATGAGGAAGAGAATACTGATCGGGAACAAACATTTCTTTCTTCATCAG
CCTTCATATACTAGTTCTCTCTCACTATCCATCTCCCCTACCTCTGCCCT
TCGTCCTCCTCTTCTTCCTTCCTTTATCCCCCAATGGTCAATTTCTCTCC
TGCTGCTCACCCCCCAATCTCTCCTTCCTTTTCTACTTTAATGCCCCTTA
CCTCTCTCCTTCAGCTTCTCCTAACTGCAACCTCACTTTCCCACTTCGGT
CCCTACATCCTCTTGACTACTGCTCCCAGTGTTCTCATTCTCTGCAACTT
TTGCTCTTCTTATTGCTGCCTTTGGTCTCTCATTCCCTCTGCCCCAGTCT
CTCTTTTGCCCTCCAGCCTCCCACCCCCTTCTCTGCTCTACAGCCTCTTC
CTCCTCCCACCAGTTTCTCATACCCCGTCCAAGATGGACAGCCAAGTTGG
CCATGCGGTCGCTCCAGAAGAGATCCATTTTCTCTTCCACCCATCCACAG
AGAGGTGATATAACTGAAGGTGGACAATGCTTGAGGAATATAGAGTTGTC
CTCTGGCAGAGATGGAAGTGTAAATGAATAGTTGAGTGTTTATAAAATGG
TGATAGAGAGCTCAGAGGCTGATCAACACCACTTTGCTGAGATATGCATG
TACCACATGATGATGTTTGTGTCACAACAGTCCACGTATATGATGGTGTC
CTTGTAAGATTATGACAGAGCTGAAATTTCATTTTTCCTGGAATATTCAG
TATGTTTAAACACATAAATACTTATTATATGTTACCTAATGTATTCAATA
CAGCAATAGTCAGTACATATATATTACTTAGGAGCAATTGGCTATACCAT
GTAGCCTGGGTGTGCAGTATGATATATATCTATGTTCATGTAAATATACA
CTATGACAGAATTGCCTTATACTTCATTTTCTAGAATGTACCCCTGTCAC
TGATGTATGATTGTGTTTGGATACTTTAAAACTAAATATACAACAGAGTT
GTCCTGACTTGGGCTAATGAACTACATTCTTCTTGTGTCTCTGACCAATG
CAGTGGCATCTCTGTCTTGAGAGCTGTGTTCTAGCCATGTCCTCCTCTGT
4GTGGATTCCCTAAATTGAGAATATTGTTCTGCCAACTCCCTGTATCTGA
AATCTCCTGCCCACTCCTCTCTGCTCTCTGTACCTCTGAGGTGGTCAGTC
ACTCACTAACACATCGATACCTGTTCTCTGAGTCTCAGATACACCTGTAA
AATGGAGTGACTGTCCACATCAACATTTACATGTGCAGCATTCCCTGAGT
ACCTGGATCAAGCCCCCTTTATAGTACCCACCATAAAGAACTCAGTATGT
CAAGGACATAGACAACAAATGAAGTGTCGTCCCAACACACTCAACCATAA
ACAAGGACAGAAAAGGTCAGCACATTTGGTGGCTCCACATGCCCTTCTGA
GGCCTTCTTTTTGTGAGACACCACCTATCACCCTGTATTTTCCTAGAGGG
AAGCCTCTCTCTGCTTTCTGTGATCTTGTTCTAAATACACTCTGGAACTC
TAGACCCACAAACAACATACTTTATCTCTAAGACTTCCGGGGTTCTAACT
GAACCCTTCAACTCTTGTTTATCTCCTGTCACATCCAGGCCTGAGGGCAG
AGCAGCAAGGTCTCTGGGTTGACAAGAGATAAACCAGACTACAGGCCTGA
CCTCCATCTTCTTTGGAAGATCCTACTGATTAAGTCCCTCTAAGATTGCA
CAATGGTCTATTTCAGTGCAGTTCTGAGGCCCCCAGCCAGGTGGAGTTAA
GGACCCATTTCCTGTGACTATACTATAGGTGTGCTAAAGGGTCAGCCTCC
CCTCCCCCAGAAAAGCTACTGTTTTTGTATGTCTGTCTTTTGTTTGTTTT
GTTTGTTTGCTTGCTTGCTTGCTTGCTTGCTTGCTTTGTTTGGTTTGGTT
TAATTTAGTTTGGTTGGATTTTTTTGAGACAGGATTTCTCTGTCTAGCCC
TGGCTGTCTTAGAATTACAGCTCTGTAGACCATGCCGCCCTCACACTCAC
AGAGATTCATCTGTCTCTGCCTCCCAAGTGCTGGGATTCAAGGCATGCAC
CACCACCACCCAGCTGCTCAGTGTCTTTCTTACAGGTCACCAGTTGTAGC
TGCATAGATGGCCTCAAAGTACATGGCTTTCTCCTCGGATCTTTCTCATT
ACTTTAGAATGGGTGTTTTCTCTTCATTCACTCCTGATGTCATCTTTCCA
ACCCTCAAGACCGTCCTCTCTAGTCTCTCAATGCCACAGGAAAACCACTT
CCTCTGAAGCAGCTGCTCTGGGGCTGGGTCCAGGCATTCTTGGTGGCTGC
CCTTTGGTCTTGACCACAGGCTGACTTCCCTCTCCTCTGCATGCACTATA
AGTCCCACCCACTGTCCCTTGTCCTTACAAGAGTTCTCCATCCACCCAGT
CCAGGGCTTAAACACATAGAGTGTTTCTCTCACTCCACGGGCACTTGGAG
CTATGAATGAATTGCAGTGCAAAGGGTTGGCTCAGGCTGGTCGTAGCTAG
TTCTAATATATATATATATACCTAGTGGACTATGCCCTTGGAGAAGGTAG
CTACTCAAGTGTCCCGCCACTCTGATTTCTCAATGATGCTGCAAGCCAGC
TGTTATCTCTTCTTAAAAGATGAAGAAACACAGCTGGGGTGATACTCAGG
CAGTCAAGTGTTCACCATGCAAGCACAAGGACCCAAGTTTGATCCTTAGA
CCCCATATTTTTTAAAAGCCAGTAATCCTGGGGCTACATTATAATTCTAG
TCCTGAGACAAAGGAGACAGGTGGATCTCTGAGGTTTTCTGGCTAGCCAG
CCTAGCCTAGTTAGTCAGTTCTAGGCCAGTAAGAGACATTGATTTGGTGT
AGGGAGTGATAGTGCCTGAGGAATGACATCCAAGGTTGCCCACTGTCCTA
CACACACACACACACACACACACACACACACACACACACACACACATCTC
CTCATATGTACATGTGTGAGACTGAACAATATTCCATTGCATTCATATAC
ACTATATTCTCTGTACTCATTCATATGAATACTCATTCATATATCTTTAG
CCAAGATTCTTAAGGAATCTCTTCATTAATTTAGGTAGACTCACCTCCTG
CAAGACCCTGGACCCTAACTTTACACAGATGCGGCTCCAGTGACACAGGA
ATGTCTTCAGCTCAAAAACTGATCACATAGAAATAGAGACAAGAGTCAGT
CAGTAGCATCCAGGAAGGAGTAGGGGTGGTGGTGTTGAGAGGAGGCACCA
AAATAAAGTTAGATAGGAGACATATGCTTTAATTTTTCATGGCACAGTAC
AGTGACAATAACTCATAATGATTTTTGTATATTTCAAAGTCTGAAGAGAA
AATTTTTAATGTTTCTTAGATGAGTATTTGAGGTAACAGAAATGTCAATT
ACCCTGGTTTGCCAGTGTTTTTTCTATACTTATATTGAGAGGTCATATTG
TACTTCACAAACTTATGATTATCATCAGCTATAAAAATTAATTATATAGA
AAGAAAAACTCCCTTGTCCCATTTTAGATAAGTCAAGCTTAGGAATTAAA
TTAGCAGAAGCCAAAAATAATAGGAGAAAAGTGGACAAATTTGCCTAATG
CAGCAATGTGTGTCATATGTTACTTAAGACCAAAAGAAGACTCACAAAGG
CAGTGGGAGTCCACAGCTCACACACTAAATTGTATGTAAAACATGTGATA
AAGAGAGTTGGGCTAGGGAAGTCAGATGGAGTGACTGTGGCTGTTTGAGA
TGGCCAACCGGGTCTAACTGTACAGGTTTCTGCTTTCCTTCTAAGGAAGA
CCTCTTCACACGAAGGCAGCCATTAGAATGATCTTGAACTGCCATTTTCT
ATCACTTAACTCAAGTATCTGCTGTGCTAGAGCAGCACGTTGAGGAAGGA
GGGTTGTCCACAGCATCAGTATTCAGGCCCAGGAGGCAGTGGTGGTTGAT
TGTCCTGTTTTCCTGCCCAGGTGGACTCCTACGATGTCACCGTGGATGAG
GAGCTGGGCGAGATCTACCTAGTCAAAATTGAGAAGCGCAAATACTGGCT
CCATGATGACTGGTACCTGAAGTACATCACACTGAAGACACCCCACGGGG
ACTACATCGAGTTCCCATGTTACCGCTGGATCACAGGCGAGGGCGAGATT
GTCCTGAGGGATGGACGTGGTAAGCTGCTCGGACCCCTGACACTCGTAGG
CTTCCTGGAAACTAGAAAGTCTCCCCTTCTCAGAGAGCTCTATTTCTGGG
CGTGAGAATTCCCTCTTGGGTAGAGCACACCTCTGTATCTTGTTCCCTCA
TGGGAGATGAGATCTTCCTCTGCTCTAACGAGCTCTAGAGTGGAGCTAAT
AGCCTGAGGAGTCATACCAACTATGGCTTCCCTCTGCACACTGTCCTGGA
AAGGTGTGGAAACAGGCCACTAGGTAGTGAGGGCCTCTTAACAGGCACCC
GTGACAAGAATCCTGCAAATGGGGAGCGCTGCTAGTTAGCACATGACCTC
TTAAGCCAAATTCTCTGGCTGTCCTCTCTAAGCCAGGGGAGTGGGGAGGT
GTTCAGGGCCATAGGACCCCTCCTGAAGGCCTGGACGCTAAAGGTTTGGA
CTAAGATTGCCAATATAATGCCAGCTCTAGGGGCCAGAGGCAAGCTGGAG
TTCACTACCCCCTGTTTTACTTGTTTGCTTTGAAACAAAGCCTCAGTTTC
ACTACATAGCTCAGGCTGGCCTTGAACTTTCAGTTCTGCTGATTTAGCCT
CCCAAGATCCTAGATATGCAGGGTCCAACTTGGTACTTCCATAAGAGGCC
ATTCTTTTTCAGACCCCACTCCCACAGAGGGTCAGAGAAGGAAGATGTCA
AAGATCAGCTGTTTGTTGTTTGCACTCCCCCCTCACCTCCCTCTGAGTGA
CACCCCTCACCCCCTCTGAGTAACACCATTTGCACACTAGCTGCTTATAT
GAATGGGTCAAACTAAGCCTTGGCAAACTTCTATCTTGTAGTCGTAACCC
TTGACTTCGCTTTCAGTCTGCCGACAGCCACTGCAGTGAGGATGACTGCT
GATGCTTATGACAGCAATGAGCATCTCTCAGATAAGGATCTTCTGCCGTT
GTTCATCTTCACAGTTGGGAGGGGCACACAAGATGAGATTGATAATTAAA
CCACCACCATTTTGATAGAAAAATAAGACAGAGGGGAAGAAGAAGGGGAA
GAAGAGGAAGAAAGAGGAGGAGAAAGAAGGAGGAAGAAGGAGGAGGAAGG
AGGAGGAAGGAGGAGGAAGGAGGAGGAAGGAGGAGGAAGGAGGAGAAGGA
GGAGGAGGAGGAAGAGGAAGAAGAAGAAGAAGAAGAAGAAGAAGAAGAAG
AAGAAGAAGAAGAAGAAGAAGAAGAAGAAGAAGAAGAAGAAGGAAGAAGA
AGAAGAAGAAGAAGAAGAAGAAGAAGAAGAAGAAGAAGAAGAAGAAGAAG
AAGAAGGAAGAAGAAGAAGAAGGAAGAAGAAGGAAGAAGAAGAAGAAGAA
GAAGAAGAAGAAGAAGAAGAAGAAGAAGAAGAAGAAGAAGAAGAAGAAGA
AGAAGAAGAAGAAGGAAGAGGAAGAGGAAGAGGAAGAGGAAGAGGAGGAA
GAAGAAGAAGAAGAAGAAGAAGAAGAAGAAGAAGAAGAAGAAGAAGAAGA
AGAAGAAGAAGAAGAAGAAGAAGAAGAAGAAGAAGAAGAAGAAGAAGAAG
AAGAAGAAGAAGAAGAAGAAGACCCTCTAGGGTCCTTTGGCTCACTGTCC
TTGATGTTTAACTTGACTTACACCAAATTCTGGGAAACCTTTTGACTTGG
ATACTTGGGTTTAGAAAGATTTTTATCACCCCTCCCCCAAGCTCAGTGGG
AACCCCCGCCTCCACTCACCCACTCACCCACAGATCATTTAAGTCATCCT
GTGGCTAGAACAAGGAAGGGTGCTTCTCTTAACCCAGGGTTTAGCAGGCC
TTAGAATCTATTTTCTGTCTGCTCCACCCAAGATCTCAGTGGGGAAATCT
TTCCTGCCTACAGAGTCTGTAGCTTTGCACAGCAGCATGCTTGATAACGC
AGGCAGATTGGAATGATTTGAGGAGGTGTTGAGAGAGGGACATCAAGAAG
GTGTGAAAATGTCCAGAGGGGCCACTTCTGTATGTAGGGTCGGCAGCTTG
GCCAATCCTGATGAGCTGGGAGAGAAGAAGAGTTTGAGAACAATGTAGTA
AGTGGCATTTTAAAGATAACCTCTATCTTATGAGTCAATGCAGAGCTGTG
ACCTCAGTAGAGAAGTGGACATCCCTGGAGATGAGGAAGAGCAGAATGGA
GAAGACATAACAGCCCCACCCTCACCACACACACCTCCCCTCTGCTCTGT
CCCCAGAGCTAAATCTAATGGGAAGTCCATTGATGATATCTATTCTTATC
AGCTTTATTGACAAGATAAAATGGAAGGGGGGTCTTCAGAGACACAATCT
GGCTGACAACTTCCATTAACCATGGCTGGTAACCGACCATCATAGCTTTA
ATCAGTCATTTAATTTGGCTAGTGATCATGTAGGTCTAGAACCTTGGAAG
GGATCTGCCAAGTACTTGGTCTGTGTCCTGTGTGCCATTAACTCAATGGA
CTGCAAGCTTCACCTTGAAGATGGTTCTTCTTCATGGCCTTCATCTCTGC
ATAACACCCCATTTTTAAGGAGTCTCACAACATGAGGGACTCAGGGTAGC
CTCATTTCTTACATAGTAGCTCACTTCTTTTTTCCTTCTTCTCTTTTTAT
TAGATATTTTCTTTATTTACATTTCAAATGCTATCCCAAAAGTCCCCTAT
ACTCTCCCCCCCCCCGCCCTGCTCCCCTACCCACCCACTCCCACTTCTTG
GCGCTGGCGTTCCCCTGTACTGAGGCATATAAAGTTTGCAAGACCAAGGG
GCCTCTCCTCCCAATGATGACCAGCTAGGCCATCTTCTGCTACATATGCA
GCTAGAGACACGAGCTTTGGCGGTATTGGTTAGTTCATATTGTTGTTCCT
CCTATAGGGTTGCAGACCCCTTGGTAGCTCACTTCTAAGAGCAAATATTC
CAAGAAACAGAGAATAGATGCTGCCAGGATCCAGGCCTGTGCCTGGAACA
AGTATAGTGTAACTCCACCACCCAGCCTTGAGGGGAAGAACACCAAAATA
TCTCAATTTAAAGAATGCCAGAGGATTTGGGACCATAATTAACCTACATG
GCACAACAACCAACGCCTTGAACAATGTGGCAAGAAGAGGGCCCATTCCA
CAAGTGACCCCATTTCCCACCACTGCTGGGGGCCTGCTTAGCCTTAGAGG
AGTGAGCTGAGGTGGGACACAGAGTTGGATGAAGGCCAAAGACTGATGAT
CTCTGGCCTTGAAACCCTCTCTCTTGAAACTCAGTACAGTTAGTACTATC
AATAACAAGTTCAAAGCAGTATTTCATATTCATTATAAGTTCAAAGCAGC
AGTTTTTACATGGCCTGGTCTTATCATAGTGCACACCTGTGGCTTCATCC
TTGGACCTGAATGTATTTCCTTGAACACAAATTTGTCTCAAGCCTTATTT
CTCTTTTTAGTGTAGTTATGAAAGCTCACTGTATGTCTTATTATGCAGCC
TTTTCTTGCTATTATGAGGAGTGGGTACATTCTATTCTTGATTCTAACTT
TATTATATCTTTCTGGCAAAACAACTTTCTTGGAACCTTCTTCCTATAAT
TATTTAAGTTGAATCATGAGTTCTATATAAAATATAAAATCATTAATTCA
TTTAAACAGCATTAATTCATAAAAGTCCATCTTCATGTTGATCTGCAGGA
AATTTGCCCAATAGCATGCACTGATGCCCAGAAGTTGTTAGACTGTATAA
TAGTGATATGAGTGACATATGATTAGCAAGAAGCCATTCTGGGATGAGTC
TTTTGGGACTTCACCTCCCAGAGCTCACCCATCGCAAACCATGCAAAAAA
ACCAGTGAGCCATCTCCAGGAAAGTCATCCAATAGCCTTAGCCCCTCCTA
GATATCTTCTGACAAGCCACATGATGGCCTACATCTCATATTTTACCCCT
TCACCTCAGTAATCATAAAAGTGATAGGACTATCAATGTGGTCCCAGAAG
CTATAGCAGATGTGTGTACAAATGGTTCATTCCAGAGCTCATGCGAGCCC
CTGGGGATGGGTTCTGCCATACTCAGTTCAGAAGCAGCTGAATCGGTCAT
CCTGCAGCCTGGGAAGCCCCATCCATTGTAAGCCGGAGTCTCATAACTGG
GGAGACTGAGTCAGTCTCTTGAGTCAACTTGTTTTTAGTTCCTTGGAACT
CTATTCATACCAAACGCAGGAAAAAAAAAAAAAGAATCGTCTTAGAGTCA
AGATGTCCAAGGAATATTCTGACCATCTAAGGCATTTAGCAAAGAAAATG
TCAAATGTTCACAACAGTGTCTAGCACAGTCAGTGACAACAGGCATTGCC
ACAGTTTAGACTCACCTCAACCTCGGGGCTATTGATCCTGCTCTGTTGTC
TTGATCCTCCCCTTTGGGATCACTTCTATACTCGAGGCTGGACATACAGT
GACCTAGAGAGCTCTGGGGTTTTCTGAGAACTGAGGGGAATGGATCTGCT
GTAAGGGAAACGAGAACTGAGAGTTGGTGCCTAAGAACCATTCACGGCGC
CTTGAAACAATCATCTTGTGGTAGCTAAGCCTAATGAAATAAAAAAAATG
ACTTGTAATCTTTCATGTTTTTTATTTAACTTTTCCATCAATGTTAATAT
GTTGTTCAAAATATAAATGTTTAGTGCAATTAAATATTTAACTCAGGTAG
CTAGGGAGTATAGTATACTACAGTACACTGCAGTACAGAATAATACAGTA
TATATTATAATCACCTTTTCAGATTCTTACTTGCGACCCTGTTTGCTGAA
TTTACCTTAGATTACAGTATAGCTGATTACCATTTCATCTGTGCTGAATG
CTCTGAGAAAGGGCCTTTAGCAACTTGTCAAAGTTCCTGAAATGGCAGAT
GGCATGACAGGCCAAGTGACAAAATGCTGTGACTCTTAGGCCTGCAGGCA
AGCAGGAAACCCTTGAGGTTATGGGTTACCCATTATGGGGGTAAGACATA
ATAACCATTATTCTCAAATATTAGACACATAAGAAGACCAATGCTTTGTA
GAATGCTTTACAATGTTTTTACATTAACATTCTCCTAAGATCCCAAGAAG
TAGGCGTTATCATCTATATTTTATATGAGGCAGTCAAGGTAGCTTGCTCT
GGGTCACATGACTTTAAACCACAGATCCCAAGTCCATGCCTGCTGTATCT
CTTTTACATACCCCCACAGCCTAGATGAGCACATTTGTTTTAGAGTAGAG
CCTGTCTTCTGTGTAAATAGCACAAGGGACTGGACAGTGTCTTGCATATT
GGGACTTACAGCATGCATTTCTCCATGAAGCAACCAAATGGACTTAAAAG
CATGAGCCATGAGTCCTGCTGTTTTCTCTGGCGGTATGAAAGTCAGCTGC
TCTTTCACCTCAATATCTCAAAATAACAGTTATTGGATCACTCTAAAGTG
GCTTATTAATATCCACCAAAGCCTGTCATGAGGTGTATACTTATAATCCC
AGCACTTGGAAGGCAGAAGTAAGAAAATCGGGAGTTCACAGCCAGCTTTG
GGTAAATAGAAAGTTCCAGGTCAGCCGAGTTATGTGAGATCTTGTCTCAA
ACAAACAAACAAAAAACAAAAAATCTAAACAAAACAAGGGTATCTAATAA
ACCCCACAAAATACCTCTCAGCTTGTCTTGGCAGACAGGTCTAGACCAAG
AGCCCCAGGATTCCTGCATAAGCAGCATGCTGAAGGCTAATGGAAAAGGC
TGGGAAAACCTGACCACGGGGCATTGTGTGGACATTGTTGCTCTTCCTCA
GGGTGCTCTCATATTTGTCCTTCCTCTGCAGCAAAATTGGCCCGAGATGA
CCAAATTCACATCCTCAAGCAGCACAGACGTAAAGAACTGGAGGCACGGC
AAAAACAGTATCGGTGAGTTGTGGCATGGACCAAGTGGCCGTGAGTAGCC
CCTTCTGGAGGAGGACCATGGTAGGAGACAAGCTTGCCAATGTCACATGG
ACCATATTCTTACCTCCGCCTTCAGGCAGTCAGAGTGAAGGGGGTCAAAG
AAAAGACTCCTTGGGGAGAATGGGTCCAAGAGAGTGAGTCGCCTTTCCCA
TTATTGTATGGGCTGCCTCAACTATGTATGTCTTCATGTAATATTTTAAC
TATAAAAGTGATACATAATCTACAACAGCATCAAAAAGTCACTAGACGTG
ATCAAGAAAAAAGGGTCCAAATTATTCAAATTAAACATAAGGATGTAATC
CTAGCACTACTAGAAAGATGGTGGTGGTGACAAAGAGCAGCCAGCCTGGA
ATACAGAGCCCAGCAGTAGAAACAACCCCAAAAGGTGAAAAGCCATTACC
AGCTCCCAAAAGTTGACCTCTGACCTCCACACACAGTGTTGTGTGTGCAT
GCCTGAACATAGATGCACACAGACACCCACATACATACCACAAACACAAT
ACAATATTTTTAAGAAAATAGAGAATCTGAATAAGCCAATCCCATGCAAT
ATAATTTAATTAACAATTTTCAGATGTCCTATAAATAAAAGCTTGGATAG
CTTCATGAGTAAATTCTAGCAAACATTAAGGAATTAATATCAATCTTATA
TATATATTCCCACAAATAGGTGAAAAGGCAATATTTTCCAACATATCGTA
TGGTACCAATGTTGTACAAAAATTAGGCAAAGACATCATTAAAAAATAAA
GCAATACACAAATACCTCACAAAAATACAACTGCAATTATTTTCTACAAA
ATATTAGCAAGCCAAGTTCATATGGTATTAACAAATAAGATTTTTCCCTA
GGAATACAGTGCTTTTGAACATTCAAAACTTTGTTAATCTAATATATATG
TAAAAAATGAACCAAAGTACTTATACACATATATAAAATATATAATATAG
ATACACAAAAAGGAATTTGAGAAAACTCAACATTCTTTCATAATGAAACA
TTCAAAATACTGGAATTGAAGAGAATGTCCTCAACACAATACAGAACGTC
TATGGAAAACTCCCAAGACAAAGTGCTTTTCCCCAAGATCTAGAAATGAT
AGGACACCCACTTGTGCTGCTTCTGTTCGATATTTCACTGAAGGGTCTAT
TTGGTCAGCTAGGAAAGAAGAAGAAAATGTTTTACACAATCACCTTTCAT
AGATGACACAGTCTTTTATATAACTAATAAATTCACCAAAATTTAATTAT
TTATGAAAAACTAATAAGTTAGCAAGGTTATGAGATAGAAACTAAATCAA
AAAATTGATATATTATAACAAAAGAAGAAAAAAATCTACTTACAGTAAGT
TCAAAATCAATAGAATATTTATTAGGATGTTTAACAAAAGCATTGTAAGA
CTAGTGACAAAACCCTAGAACACAGTTGAAAAGAATAAAGAAAATCTAAG
TAAGTCGAAAATATCTCATGTCCCCTTAGCTTTGCTAAAGTGGCAATCCT
TCTCCTGGGAATGACGGATGATGGAGATCCAATGCAATCGCTGTTAAAAT
CTCAGCTAGTGTTTTTTAAAGCAAGATTGATAAGATGGTCCTCAAATCCA
TATGGAAATGTATGGAAAGGCATACAAAGTTACCAGACCAATCTTGAAAA
TAGGAAAACTAAGTTAGAGAAGCTATATTTCCCAACATCCAAATTTCTGC
ATAATCATAGTATTCGGACGAAATGTAGCCGTATCATGAGCAATGACATA
GACTTCCAAGTCCAGGAAAAAAAATCCCTTATACTTACAGTGAATTGAAT
GTGGTGGGATCAGCAAGACAAATCTGTATGTAAAGAATAACCTTTTAAAT
AAATGGAGCCAAGGAAACTAGATAGGCAAAGTACAAACAACCATGACATT
GTGCACACATGTACAAAGTACATGATGTTGGATCCTTACCTAACACCAAA
TTGATTACCTAAAAGGTGGAATTAAAACCATATAATCCTGTGAAAATACA
AAAGTAAACATCCATGGCTTTGAGTTGGCAAAGGATTCTGAGACAGAACA
CCACATCACACATAGCAAGAGAAAAACGGATGACTTGGCATCACCAAAGT
TTCAGGCTTTTATGTTTCAAAGAACCCCATGAAGAAAATGAAAGACTGTT
GGGGAAGTGTTTGCCTAATGAGCAGAAAGAGCTGAGTCCAACCCCAGAAT
CCACATTTAAAAAAAAAAAAAACAAAAAACAGAAAACTGTGGGAGCTGCA
GAGACCACCCAGCAAATAAAATGTCTGCCACACAAGATGGAGGACCTAAG
TTTCAATCTTTGGCATCCACTTAAAAGCCCAGAGCTACAGTGCATGTCTA
TCACCCCAGCACTGTTGGAGTAGAGACAGGTGGAATATCAGGGCTCACTG
GTCAGCCAGTCTAGCTAAATCAGTGAATCCAGGTTCAGTAAAGAGATCCA
GTCTGAAGAAATAAAGTGGAGAGTGACTGAAGACACCCAACATCAGCCTC
TGTCCTCTACATTCACACACACACACACACACACACACACACACACACAC
ATGCACACGCACACACATACACAATCAGACATAGTAATGTATATGTGTGA
TCATAGTGCTGAGGATGAAGAGACAGGTAGATCCCTGAGGCTCTCTAACC
AGCCATCCTAGCCTACTTGGTGAGCACCTATCTAGAGAGAAAGCATATTT
CAAAAACCAGGTGGATGGCTCCTAAAATATGACAGCTGAGTTGTCCTCTG
GCCTCCGCATGAATGGATACACTTGTGCACACAAACATACCTACACACAA
AGAGCCCACAGAATGAGAAAGAATATCTGCAGAACAGTTATGTCCAAACT
ATATAAAGAATTCTTAAAACTCCAGAAAGAAATAATACAATTTTATTTTT
TATGAAGTTATTTACTTACTTTACATCCCAATCACATCTTCCCCTCCCTT
TTCTCCTCCCAGTTCCTCTCTCTCATCTTCTCTCCCCACCCCTCTCTCCT
ACTCCCCTTCTTCTCAGAAAAGGGCAGGCCTCCAATGGAAATTAATCAGC
TTTGGCATATCAAGTTGCAATAAGATTAAACCCAACTTCTCCTATAGAGG
CTAGACAAGGCAGCCCAGTTAGGGGAAAGGGATCCAAAGGCAGGCAGTGT
AGTCAGAGACAGCCAATGCTCCCACTTTAGTAGTCTCATATGAAGACCCA
GCTGCGCAACTATTACATGTGTGCAGAGGGCCTACATCTATCCTATGTGT
GCTCTCTGGTTGGCATTTCAGTCTCTATGAGTCCTTATGGGCCCAGGTTA
GTTGAGTCTATGGGTTTTGTTGTAGTGTCCTTAACTCTTTAGGCTCCTAC
AATCCTTCCTTCCCATCTTCTGCAGGATTTCCCAAGCTCCACTTAATGTT
TGTCTGTGGGTCTCTGTATCAGTGTCAATCAGTTGCTGGGTGAACTCCCT
CTGATGACAGTTATGTAGGATCCTGTCTAAAAGTATATCAGAATATCATG
AATAGTGTTGGGGGGGTCCCTCTCATGGCATGGGTCTCAAGTGGGCCAAT
CATTGGTTGGCTCTTCCTTCAAATTCTGCTCAAAAATACAATTTTAAAAG
GGGTAAGAACTCAAAGTCCAAAGAATGGACATTTCTCCAAGAAAAACATA
TAACTGGGTAAACACATAAAAAGTCAGGTGCTACATTAGTTGTCTGGGAA
ATGCTAATTCAAGCCCAGTACAGCAGCAAGAATCAAAAAGTCATATTATT
ACAAATTTTGGTGAAATTACAACACTCATATACCACTGTTGAGAATGTTA
AATGGTGCATCTGCTTTGGAAAATGGCTTAGTCATTCCCCAAACTAACAA
ATAGAGTAATTATGTTGCCCAGCCACCGCACTCTTGATTATGTACCTATT
AAAAAGGAAGTCATATGTTCTGATGGTTACTGCTCACTGCCATCTAAACT
GGAGAAGCACATGCAGGTCAGTGTGCCTGCAAGCTCCAGGGAGGATTAAG
TGAGTGAGAAAGACCCAACTTGAATGTGAGTGGCTCCATCCTACAACCCT
AGAGTCCTAGATCTAACATAGGGAGAAAGGGGGAAAGCAAGGACCTCTGG
CACTCCCCTTTCTCCACTTCGTGGACCACCATGATGTGAATGGCTCTATT
ACACCTCACCCATCCCACCAGAATGGACAAGATCTCTCAATCTGAACAAA
ACATAACTTTCTACCCTGAAGTTGTATTGAAGTCACAGTGACAAAAAAGA
CACCTAATACATATGTCTACTCTAAAATTTCTATCTGCATGATTGCTGTT
CTAATAACCGCACTAACACAAAGATGCGTACAACCTAAATGTCTATTAAT
GGACGAATGGATAAAACGTCATATATCTGTGTAATTTGATGTGGATTCCA
GGGGTCAGACGCCACTCATCAGGCTTGGTGGTGGCACCTTTACCTGATGG
CCATCTTGTCAGTCCTACTATATAGTCTTTAAAAGCTTTTGAAGTTTGTT
AGTGGGAGGAACATATAAAAGTTTGAATCTTTGGGCTAGAGAAACCCAAT
AATGCTTCAAACAGAGCTTACTGCACCATCTTGGTGGGGCTTCAGAAGGC
TGAGGTGCCAAGAGAAGCACAGAGAGTGAAGACCTGCTTCATGAGCTTCA
GTGAGGAATCAGGACTCTGCTGGTATTGGGCTGTAGGCCATCATGTGGTA
TTCTGGCATGAGATCTGGCTTCACTCTACCCGTGGCCTAAGAACTCGAAT
GAGGCTTAATTAGTGAATAATAGATTAATATGTTTGGCAGCAAAAATGTG
AAGACACGATAGCACCTAGCATGTGCTACAGCTACTGTCTGCTGCTCTTA
CCCAGGTGTCCAATGAGAGAGATTCCTTGAGTCTTAGAAGAGGCTTTGGA
GTTTTGAATGAGCATTCCAAAGACTCTGGAACTTTTAAAGCTTTGAGGGC
TTTTAGAGATGAACAAAACTTATTTTCCATTATAAGATTGGCATGAAGCT
ACAGAGAATAGGATGGGAGTTTATGGCTTAAATTTGTTTGAATGTCAAAC
TGACAAGGATGCTGGACTTGTGATGGCTAATCTTGTCAACTTGACAGGAT
GTAGAATTATCTAGGAGACAAATCTCTGGGTGTGTCTGTGAGGGAGATTT
CAGATCGGGTGAATTAAGGTGGTAATTCACCCTCTGTGTGTTTCCCCACC
AAGTGAACCACAGTAAACCTTCTACTAGGAAGTTGCTTTTATCAAATGCT
GTCACAATAAAGATAGAAATAAATAATACCTAAACTAGCATAGCAGCCAA
ACATAGACTAGTGTCTTTAAAGATTACCCTTATCTGAACATTCCTCATAA
AATGAATGATACAATTAATGACATGACATTTATGACTGGCTTGTTTTACT
TAACATGATAGTTTCTAGGCTCATGCATGTTGTAGCAGCTATTAGTATTT
CATTCACTATCTGTAGAAAGAAAAGACTCCCAAAAATTGTCCTCTGACCT
TCACACACATGTCATAACAGGTGCACAGAGAGAGAGAGAGAGAGAGAGAG
AATTCATATAGAGGAAAAGAACCACATGATCAGCATTGCCATGGCGATAC
CTTACTTCCCAATCTTAGATATTATCTATTGTTCTCATCACTGTGACAAA
ATGCTTGAAAACAGCAACTAAAATGAGATTGTTTCACCTTGGACTTTGGT
TTGTTTGCTGTTTTGTATTTTTGTACAGTCTGAGAGAAGACATAGAGGCA
AAGTAGGAGGAGCTGATGACATTGAGCCCATAGTCGGGGAACAGAGAGAG
ATGAAGTCTGGTACTCTACTTGCTTTCTCCTTTGTATTTAGTCTAGGACC
CCATCTCATGGAACGGTACTATTTACATTTGGGGTGGGTCTTCCCATGTC
AGTTAATGGCACTTTAGCAACTACCTCACAAGCATGCTGGAGGTTTATCA
CCCAAATGATTCTAGGTCCCATCAAGCTAACTGTCAACATTAGCCACCAT
TGTTAGCTTTTTCTTAGATTGAAAAGTACCACCGGGCCTGGTGGCGCATG
CCTTTAATCCCAGTACTCAGGAGGCAGAGGCAGGCAGATTTCTGAGTTCG
AGGCCAGCCTGGTCTACAAAGTGAGTTCCAGGACAGCCATGGCTATACAG
AGAAACCCTGTCTCGAAAAAACCAAACCAAACCAAAACAAACAAAAAACA
AAAAAACAAACAAACAAACAAAAACCAGAAAAGTATCTGAGTTAAAATAA
CTTATAGGAAATGTTTATTTTAGATCATTGTTTTGGAAGATTGGGTTTAC
AGCCAGGAGGACCTATGGTTTTGCCTTGTGTCAAAGAAGCACACCATTTA
CAGTCATTGTGGTAGAACACAAAGCTACTTAATCTCACAGCAGCTGGAAG
ACAGACAAAAAGGCCCATGGTCCAACATTCCTTCAATGTGTGTCAAGTGA
CTGAACTTCTGGCTACCATTTCTCAGGAAGACCATTAGCAGGAGTCAAGC
TTTGCCTTTATTATCCAAAGTATAGATGTTGCCTAGGGGCAGAGGGGCCA
GTAATAGCAATGATGACTAAAGATATGTGTTCCTTTGGGGAAATCATAAA
AATGTTCAAATATTGACTATGGAGATGGTTACCCCTATGTGATAAGGGTA
AAAAAGACTTTAATTATTGACTATCATTGGGTAAATTTTATGGTATAAAA
ATTATATGCCTATAAATCTGTAAAAATGTTTAATTAATTCAAATTGTAAT
ATAGACTTCAACATAGCAACAAAATCTATACAACATATAGAGAACAGAAG
AGCAAATCATGATAACCAGGCTCAGCAAAACCTCTTAGGTATGACACCAA
GCACACACAGCACAAGGACAATCGCCACACTGGACTTTGTCAAAACATAA
ATCTTCTATACTTCACTGAACTCCACCAAAACAGTAAAATGATAAAATGG
GCCAACGTATGCAGACTGCATCCAGCAAGAGGCTTAAATTCAAAATATAT
AGAGAACACTAACAAAAGGTAAAAAGACAGGTTATAACAAATGCTGCCAA
TGAGGGATGGAGGTCAAACCCCAGCACTCTGGTAAGGTCAAAAAGCCACA
GCCATTCCGGGAGACATATCAGTTCTTCCTCAGAATTATAGATAGTGTCA
CCTCGTGACCCAGCAGCACTTCTTATCACAAGTGTTTCCAAAAATAATAC
AACCAGAATTAGGGGTGTAGCTCATGGCAGAGCACTTACCTAGTATATAG
AAGAGCCAGGGACTCAGTGCACTCAGTGCACACCCCTGTACTGCAATATT
AACATTAATAACTAAGCCATGTGTCTACACCAAATACTTGTATGCAAATA
TTCGTGGCAACCGTCTTCCTTGGGGTTTCTATTGGTGTAATAAGACACCA
TGACCAAACGAAACTTGGGAAGGGAAGGGTTTATTTCACTTCATACTGCC
AGGTAATATCTACCAGTGAGCCACTGAGACCAATATAGTTGATCATACAA
TGAAGGCAAGATGCCAGAATCGCATGCAGCTGTCCCATAACTCTGGATTC
TGTTGCTAAGGGAAACATGTGGCAGTGGAGCTATGACCAATAACAAAGAA
GTGACATCACATCCCAGGGGTGGCTGGGCTTGGTGCCGTGCCTATTGTCC
TAGATGATTTGGGAGGCTAAACAGAAGAATCATTTAGTCCCCGATGTTCA
AGACCAACCTATTTCAAAAAGAAAATTCAGTATGTCAATGCTAAGAGTAA
GGGTTGAGGGTACTCTCACTGATGTGATAGATAGCTAGAGAGATGTTTAG
GGTCTGAAAGGGAGCAGGGAAGAAAGAGAGAGAGGGAGAAAGAGAAAAAC
AGGCAGAAAGACAGACAGGTTGAGAGAGATAGAGACAAATGCATATGTCT
ACATACCCTAGTTCACAGTTTAAAAGTTAAAAACTCTTGCGCCATCCCTG
CATGGTGTAAGAGCATAACCTGATTATACTGTAGTCAGGCCAAAAATTCA
AAAATTCAATGTGTATGCAAGCCTTTCCGCTCAGCACCCTATGATGTAGT
TTGGAACTTCTGTACTTTTACAGGGCATAGATCATTGTCCCAGAGTCTGC
CTTCCCTTCCATCCGGCTGAGGAAATCTTCAGCTCTCTTCTCCATTTCAA
CTCCCTGTGACAAAGCCCTTTATAGAGTTCTACAGGACTTCTAGTCTGTG
GAGTGGGTTAGGACAAAACTCCAGAGTGAGTCAATTTGTTGTCAGCCACT
ATTATCTTAGTGTGGTTTGGTGCACACCTAATTAGACTTCTTACCCATTA
CACCTAAGAGATCACAATCTAGCCTGATCTGGAAGCCGTTTACTTCATCA
CAACCTGGGTCTTGTGAAGAGGATGAGAAGGGCACTGTGAAATATCTTGT
CCCTTTCTTAAGCCCTATTTTACTCCCTCCTTCCAAGCCGCTGCTCTCTT
CAGCAGCCCGCCTCCCACCCATGCCCCATTTCCGGTCAGAAAACACTGAA
CTTTGCACTGTCTGCACAATTGTGAAGTTCCCCTTTCACATACACTTTCA
GGCTTCCAGATTATGGAAGCTGAGCAAGAGAATACTTCCAGGAATGCTGC
TGGCTTGACTCAGTAGCCCATCTGGGAAAGGGCACAGAAAGGGCCCAGGT
CAGAGCTCCGTGGCCCCTGGCCCTCACTCGCTGCACTGTCAGTGAGTATG
ACACAGTATACTGTGGTCTGAGGGCTCTCACATCCCTGACCTAATACGTC
AGTATCTTCAGTTTTCAATCATCATTATGCTCTGAAGACCAGCCATTGAC
ACAACTCGACAGCCTTTCATTTTAATTCCTGCATAGTTAAATGCATCACA
CATTTTTGGTTTTGTGTTCTGAAAACTGAGCGCAGGACCTATTAAATGGT
AAGCAAGCAAACACTCTATCCCCAGCTCCGGAGGCATCATTCTTGGGTAC
CACTTTGCTATATTCATTTGGCTACCTGCTGATGAACACATGGGTACCTC
CTAGTGCTTTCAGACTGAGCCACAGTGAGCTTTCTGCTAACGTGAGAGAA
TTACTCTGGGGAACATGTCTAGATGAAGATCTGTGAGATGACCAAGTGTG
TGCGTCATCTGACTTCTCTAGATCTTGGTAATTGTTTGCCCAGCTGTCCA
GACACTTTCCACCCTTAGCTGGTGTTGCTGTAAGATGCCATTCCCCTGCA
TCACCAACCCATTCGACATGAGACATTTTTGTTCTCGACTGTCTAATGGG
TAGGATTGTGTCTCATTTCTTCGTCATAAGATGCTACATATTCATGGATT
ACACATAAGCACTACAGGCTGAGCCAATGACTCCCCTACTGGTGAACATT
CAGCTCGATTCCCTACTTCTGTTTGTTTTGAATATTACAGAGAAGCAACT
TGTATGCATGATATATAAGTCATTTAATCATTGGCAGATCTGTCTAGAGA
ACATTAATCACTTGTCTCAAAGCAGCTCTCATCTCACATAAAATGAGATA
ATGTATTGTGACACAAATATGAATGTCCAGGCCTGGCTGGCTTGGGTGTT
CTGCATGCTGAATTTCCACACAGAAGCAATTGCATAAACATCCATACCCA
CAGGATAAGAGAGAGCATTAAACCAAAGCATTTTTTCCAGCACATCAGTG
GGAACAAGAGTTGCATGAGCTACATCCAATTGGGGAAAATCTGCTGCAGG
TGTGAGAGTTACCTACCTGGTGGTAGTCTTAGCTTTGGGGTGAGAGTTAC
CTGGTGGTAGTCTCAGCTTTGGGGTTAGTAAGGAAGCTGCTGGTGTGTTT
ATACGGTCCAAAGATTTCACCTGGTATTTGGAAGGATAAAAAGTCAGACA
TAGAGGCAGACAATGATTGGCTATTAAAGAGACAAAAGGTGACCCAAGAT
AACTTTTCTCTAGATCTGCACACCAACTCTTTATAATAACAAAGTTCCCA
TAAGCTGATCAACCTTGTAAAACCTTTAGAGCAAGTGCAGCTTTCTTTTC
TAAGAACATTATTAGTTCACAGTTGTGTCGGGCAAGTGTCACATTTTGCG
CGTGCAGAAATGTTGTTTTATGTCCTCTGATGCTAAGAAGGCTCATTCTG
AGGAACTGATAAACAGGAGGCGGAGCAGCCAGGAGAGCAGTCTCCAGAAG
AGAGAAGAGGGTAAAGAAGAGGATGTCTTTCAGAAGACATCCTTTATTTA
TAGCAGTTTTACTGGCTATCATGATCTCAATATGACCTTTCTTTCTCCCT
CTGCATCTGACAAAACATATACATATAACATAAAATGGTAATATTATCCC
AAAAGTTAAAACAATTAAACACAGAGCCTAAAGGAGAAAAAGGTAATGGG
TGAAGGAAGCAGGCAGCTAGCCAGCCAAAAAGTCTTTCTAAAAAAAAAAA
ATATGTATAATTCTATTTGTCAATTAAATGAATGAAAGCATTAAAATAAT
TAAATTAAAATAACTATTTTAATCTTTTCATAATTTAATTAAAATATGAT
TATATCATTTCTCTCCTCTCCTAACTCCTCCCCTTCCAATGTCTACACAT
CCCCCAATTCACCGCCTCCTCTTTTATCTAACCCTTGTTGGCAAATATAC
ATAAAAACCAGTTAACATACAAATACAACCTGCTGAGCCCCTGTCCCCTA
AGTGTTGCCTGCATGTGTATGTTTCTAGGGCTTGGGATTAGATGACAAAC
CAAGCCAGGCATCTCTGGGTGAGACTGACTCTCCTCTCCACAGCTGTTAA
CTGCTTGTAGCTCTTCAACTAGGAGTGGGACCCCTGAGATTTTTCCCCAG
TCTGTGTTGGAATGACAATTATTTTGGAATTATTTGGGTTCTTTTTTGGC
AGCCATGTTGTGGGCGTAGCTTTTCTGTCATAGCTAGAAGAGACTATCAC
ACAGCAGACCTTCTAATTTTCTAGTTCTTATGATCTTTCTGACTCCTCTT
CTTCGATGCTTTCTGAGACTCAGGTGCTGGAGTTGTGTGGTAGATACATC
CCCTGGGTACCACACAACCATTTCTTCTCTTCATTTTGACTAGATGTGGC
TTTCTGTAACGGTCTCCTTCTGCTGCAAAAAGAAGTTCCTTTGATGAGGG
TTGAAAGTTACACTTGCTTGTGGGTGTCTGGATAAGTATTCAGAATTCCA
TTAAGAATTATTCTGGTTAGAGAAATTGGCAGTAGTACAATCTATGACCT
CCCTAGCCAAAAGCAGTTGGCTAAGTTTCCAGGGCCAAGAAGGATGTCTT
TCCTGTTAAGCAGGCTTTGAGTCCAAGTAGACAGTTGTTGGTTACTGCCA
AGATATAGGCACCACTACTGCCCTGTAGGGATATCTTGCCATGATGGTCA
TTGTGGTTTATATTCAATGACAGCTGGCTAAGACTATTGACTCTTTCCTT
CCCTGCATATGAAACACAGCTTATCACTTTCTGGTCCTATGAAAGCTGGT
CCTCGAGGGACTCTTTTGGTTCCTTTCCAGCTCAAATCCTCCACATTTTA
TGTGTGGAGTGCATGGTGTCCTCAGCAATAAGGACTTGCTTTTAACTTCT
GGGAGGCAATCAAGGACAAGCAATAGCCTATATTGTTTCAAGAGCTCTCT
TGGATTTCCCTGATCAAAAGCTCAAAGGGGGCTTGTCATGCATGGTACCG
AACTTTTTGTTAGCTACGCTTGGCTCTTGTTGGAGCGAATCATCCCTCCA
TATGTTGTAATTTTTTATATTGCATGTTTTCTCTTGTTTTTGAGAAATTG
AGAAATTTCCCACAGGGTATTAATATATTATTATATTGGCATTACATATA
GTGATTCACTGTGAGGCTTCCATGGTCATAAAGACTGCACACTATTCAAC
ATTACGTATATGATGTTGGCTGAAGAGAGAAGGTACTATTCTGCATTTGT
AATTTAGAAGGTTGGATTCTCTGGATGATTTTTTTCTTTTTTTTTAAGCC
TAAGTATACTTTAATTTGGTACTGATATAGTAGTTCATAAAGATGATACC
ACTATGGTGTAGAACTCAAAGTTATGATATTAAGTTCAGGAAGCGAATGT
ATGTAATTCATTGCAAATTTGCAATGCTTGATTACACATGTTCAAGATTC
AACACGGAGAGGCTTCTGATACTACGGTGGCACCAGCAGGGAAGTCACAA
GTTCAGACTGTCTGAGACATGCAATAAGGTTCTTTCTTAAAGAAAACAAA
ATGTGGAAAAACAGTCTTGAAATTTTTCACAGCTCTTTCTTCCTTTCAAT
TTTCTTGTGCAGAAATTTGTGTGATGATAAGAATCAGAATGTTGCTCTGC
ATTATCTAGTAATACTAACTATCTTGTGGAGTGATCCTGTGCATTCACCA
GAATGCAAATACTGCTTCCTTACTAAATCACTCGGAGCCACTGATTCCCA
TCAGCTAGAAACAAACCTGAGCAACAGAGTCAGTGGGCAGAACACCATAA
TCCTCCTAAGAGCATGGAGTTAGTACTCTTCGCATACATCCCTTTATTTA
TTATTTATGTATTTATTTTCGGGGAAAGGGATAGCATTTTATTTTTTAAT
CTTCTTATATCTGTTGAGGCCCGTTTTGTGACCAATTATATGGTCAATTT
TGGAGAAGGTACCATGAGGTGCTGAAAAGAAGGTATATTCTTTTGTTTTA
GGATGAAGTAGTCTATAAATATCTGTTAAGTTCATTTGGTTCATAACTTC
TGTTAGTTTCACTGTGTCTCTGTTTGGTTTCTGTTTCTATGATCTGTCCA
TTGATGAGAGTGGGGTGGCGAAGTCTCCCACTACTATTGTGTGCAGTACA
ATGTGTACTTTGAGTTTTACTAACGTTTCTTTTATGAATGTGGATGCCCT
TGCATTTCGAGAATAGATGTTCAGAATTGAGAGTTTATCTTGGTAGATTT
TTCCTTTGATGTGTATGAAGTGTCCTTCCTTATCTTTTTAAATAACTTTT
GGTTGAAAGTGAATTTTATTCAATATTAGAATGGCTACTCCAGCTTGTTT
CTTGGGACCATTTGCTTGGAAAATTGTTTTCCAGCCCTTTACTCTGAGGT
AATGTTTTAGCCATTGAAGTGCATTTCCTGTAGGCAGCAAAATTCTGGGT
TCTGTTTATGTATCCAGTCTGTTATTCTATGTCTTTTTATTTGGGAATCT
GGTCCATCAATGTTAAGATATTGAGGAATAATGATTGTTACTTCCTGTTA
TTTTTGTTGTTAGAGGTAGAATTATGATTGTGTGACTATCTTCTATTGGG
TTTGTTGAAAGAAGATTACTTTCTTGCTTTTTCTATGGTATAGTTTTCCT
CCTTGTTTTGGTGTTTTCCATCTATTATCCTTTGTAGGGCTGGATTTGCG
AAAAGATATTGTGTAAATTTGGTTTTGTCATGGAATATCTCGTTTTCTCC
ATCTATGATAATTGAGAGTTTTGCTGGGTATAGTAGCCTGGGCTGGCATT
TGTGTTCTCTTAGGGTCTGTATGACATGTGCCCAGGATCTTCTAGCTTTC
ATAGTCTCTGGTGAGAAGTCTGGTATAATTTTGATAAGTCTGCCTTTATA
TGTTACTTGACCTTTTTCCATTACTGCTTTTAATATTCTTTCTTTGTTTA
GTGCATTTGGTGTTTTGATTATTATGTGATGGGAGGAATTTCTTTTCTGG
TCCCCTCTATTTGGAGCTCTGTAGGCTTCTTGTATGTTCATGGGGATCTC
TCTCTTTAGGTTAGGGAAGTTTTCTTCTATAATTTTGTTGAAGATATTTA
CTGGCCCTTTAAGTTGAAAATCTTCACTCTCTTCTATACCTGTCATCCTT
TGGTTTGGTCTTCTCATTGTGTCCTGGATTTCCTGGATGTTTTTGGTTAG
GAGCTTTTTGCTTTTTGCATTTTCTTTGCGTGTTGTGTCAATGTTTTCTA
TGGTATCTTCTGCACCTGAGATTCTCTCTTCTATCTCTTGTATTCTGTTG
GTGATGCTTGCATCTATGACTCCTGATATCTTTCCAATGTTTTCTAACTT
CAGGGTTGTCTCCCTTTGTGATTTCTTTATTGTTTCTAGTTCCATTTTTA
GATCCTGGGTGGTTTTGTTCATTCCCTTCACCTGTTTGATTGTGTTTTCC
CATAATTCTTTAAGGGATTTTTGTGTTTCTTCTTTAAGGGCTTCTACTTG
TTTACCCATGTTCTCCTGTATTTTTTAAGGGAGTTATTTACGTCCTTCTT
AAAGTCCTCTATCATCATCATGAGAAGTGATTTAAGGTCCAAATCTTTCT
TTTCCAGTGTGATGGTGTATCCAGGACTTGCTATGTTGGAGCAGTGGGTT
CTGATTATGCTAAATAACCTTGGTTTCTGTTGCTTACGTTCTTATGCTTG
CCTCTTGCTATCTGATTATCTCTAGAGCTACATGCCCTCACTGTATCTGA
CTGGAGCCTGTCTTTCCTATGATCCTGGTTGTGTCAAAACTCCTTAGAGT
TTGGCTGTCTCTGTGGTCCTGTGATTCTGGGATCTTGTGATCCTGAGATC
CTGGATGTGTCAGAGCTCCTGGGAGTCAAGCTGCCTCTGGGACCCTGAAG
ATCCTGGTGTGACCAAGCTCCTGAGATTCTGTGATCCTGTGATCCTAGGC
ATGTTAGAGAGCCTGGGAGTTGAGCTTTCTCTGGGTGTTGTGGGGCTGGC
TCTCATGTTTTCTCTTATATGTAGATATTAGATTTTAAGCTTTCCATATG
TGTGTTTCATTTAAAATAGCCACAGAGGTTGGGTAGCTTTTATGAGACCA
GAGAGAGGAAGGAAACTCCCCCCAAAAGAGACATAGAATATAGTGTTATG
GGTAATCGTAAGGGAAACTAAAATGGGAGGTTTAAATGGGGAGAGGATGG
GAATGTGTTACAAAGGAGAAATTATGGGAGGGACAACTAATACTAAAAGC
CTTTTGACTAGCCATATGGAAACTCACAACTCTCAAAGCTTCCTAAAATG
TATAAAATGGAATTACCGTATAATGGAAGAGACAATGCCCCAACTAGACA
TCATATGCTAGCAAGTAAAACTGCCAGTATCAGGAATGGGTTACATCTTG
TTGTGTCCCTGATCAAAGGTGACCCACAGACAGTCCCCCATAAACAATAT
AGGCTATTGTCTATTATTAGTTATCCTTCAGAACCTGACAGAAAATGAGT
GTTGTGTCCGGCCAGCAGACCACAACCTGGGTTCTAGCCTGGAAAGGCAT
TTTGGAAACCTGGAAGAGAAGAGGGGCTAGGTGGCGAGAGAAAAGAATGT
AGCCAAGACAGTTACTCTGATCAAGGCTCAAATTTTATTGTTGCGACACT
AGTTATGAAGGAAGGGGGAGGGGACCCGATTCCCGCCGAATAATCTCTGG
TCCAGTAGAAAGGTGCACGTGTGTGGCTCCGCAGGTTCCAGCAGTGGGCG
TGGCAGAACGAATGAGCAGGAAGCTCCACCCCTGAGCAAGCAGGTTTCAG
GCTAGGGGAGGGGAGACTACAAATGAGGACATCACTTACTTATGTCATCA
AACATGAGAAAATTAACCTGGTGCCCAACCCGAAGCTTTAGCCATACTGA
CAAGCACTCACAATGATGAAAGGTGCTATATATACATGTGACCAGATGAG
CAACAGCATCATCATTCTTTCCCAGCTACAAACCCTGAGACCTATAACTG
TGTCCTGCCTTGCAAGATATACTGGTACAATAATGGCACAAACGTTATGG
AAAAACCAACCACTTCCTAAAAATCACATTTAAAGCTTATTCCATGAGAT
GAAACCCATATCCAGAAACTATTAATGAGGCCACAAACTTGAAACAAGAT
GAGGCATGGACCCTAGGGGAACAACTACTACTGCTATCCTGCTAACGGAG
CATGCCACGCCTAAAGACATACTGCCATACCCATAAGCTAATATGTATGA
CAAGCCCCATCAGAGAAGTTTCTTCTTGGAGTAGATGAGAATTAGCACAG
AAACACACACACTCCCAAATGGAGGATGGGCAGACAATGTCAGTTCTTGA
AATGCTCAATCCTAAATGGAGTGTCTTTGTCAAACCCCTTTCCTCCAGCC
TTGGGGATCTAGGCAGACGTGGCAGAAAGAGCTGAAGGTGGTGGATGACT
CCGAAAATACAGTGTCTTTCAGACACAAAGGGGTTGATGGACATTGAAAC
TCACAGAGACATTAACACTATGTACAAGACTTTGCAAATTCAAGCTAGAT
AAATCTCAGTACCGAGAGAAGTGGGCACAAGTCCCACCCTCAACCAGGAT
GCTATTTAAAATTGATACCTGCTCAGAGAGGGAAAATCCATTTTCTCCAA
TGGAGTGACACTGGGTATATATCAACTGTACTGCAGGGCAGGGCTCATGC
TCAAGGGTAGTTGGTCAACACAACATGGGCTCCGTAGTTCTCTTTGGGGG
AGGGTGTGTCTCTTTTTGTATTGTTTATATTTTTCTGAGAGAGAGAGAGT
AAAAGTGTGGATTTTGGGTGGGTAGGAAGGTATAGAAAACTTAGGAGTCG
AGGAAGTGAAAGATATGGTGAAAATATGTTGTATGAAAATTTCAAATAAT
TTTTAAAATGTAATAAAAAGAAAACAGCCTTATTCCAAAAGAAAAAAAAA
TCCATAAACCCTTTACAGTGTGTAACACTTTAAGCCTGAGAAATTTATTA
AATTTTCTGATTTGGCATTCAAGCATCTAGAGAGTTTTCAACCATAGCTC
TTCTGCATTTCCTTTGGAGCTTTACTGCCCACTTGTGTTACATTGCCTCT
GGATTGCAGAAAGTCAAGAGCACTTCATGGACCAAAGATGGCACTTAGAT
AGTTCATTATGGAATGAATACACTTTGTCCTTTAAGAGCCTGATCAGTTC
TCATGCTGATTGTAATCTTCTCCTATTTCTTTGTTGAAGTCATACTTTAT
CAATCATGTCTTTATGAGGTCAGATATCTTTTTTACAGTAAATTGCACCA
TCTACACCTCCTTAAGCTCTCTCCTTCCAAACCAGATGTCTTTGCTGCCT
CTTCCTTATTTCTGTGTAGTTGATCCTTGTGGCCTTCCTCCATGGAGTGT
GAGGTTTTTGTTCCCCCACCCCCACCCCCACCACCTGGCCTATTTATGTC
TGTGGGTCTGGAGGCCTTTGGCTTCTGTGGGTTGACTTGTCTGTATGTGT
CATTCAGTGGAAGGGACATGCCAGTAGCAAGCTAGCTGGCAAACTGATCC
ACTCTCATCTGCAAGGCTCTCATCTGCAAAGGAGGTAACGGAGAGCCCCT
TGGTGTCATATCTTGAGAAAGTTCTTTCTGAGCTGACAAAACAATTTCTA
AACTCTGCAGAAAATTTGTATGGACATTTGGGGCAGGTGAAATGGCTCAG
CTAGTAAAAGCACTCGCCGTGCAAGCCAATGACCTGAGTCTTTCCTCTAC
TTTCCTGACTTGCTGCAGGAGAGAACCAACTCCCAAAGCAGTCCTCACAG
GGAGGTCCTGTCTGATCAAGAAGCTGCTTTAGGAACCTGTTTCTTAGTTT
TAAGAAACTAAGCAGCCTGGAGAGCTGCAAGTGCACCTGCTGAAACCGTG
CCTGAGGTACTGACAGCACAGTGCCTCGGGTACTGACAGCACAGTGCCTC
AGGGACTGACAGCACAGTGCCTCAGGGACTGACAGCACAGTGCCTCAGGG
ACTGACAGCACAGTGCCTCAGGGACTGACAGCACAGTGCCTCAGGGACTG
ACAGCACAGTGCCTCGGATACTGACAACACAGTGCCTCAGGTACTGACAG
CACAGTGCCTCAGGTACTGACAGCACAGTGCCTCAGGTACTGGCAGCACA
GTGCCTCAGGTACTGGCAGCACAGTGCCTCAGGTACTGGCAGCACAGTGC
CTCAGGGACTGACAGCACAGTGCCTCGGATACTGACAAAGGTGCTTTCTC
TCTTCCGGCTGCAAACAAGCCCAGTCTTCCTACTAACTCCCAAAGGTCAG
GGACACTTCGAATTGTGGCCCACTTCTTATCAACTCTTGCCTGAATTCTG
GATCAAAGTACCTCATATAGCACCTAGCCCTTCCGGGAAAACACATAAGT
AAAGACTCCTCTTTGTTCCTCATTCTTCCTTCAACAGCAATCTGGGATTG
AGTCCCCACTATGGTGGTCAGCAATATTGAAGGATCTGTTTACCTGAAAC
ACATCTCAATGTTGTTTTTGTGTTATTTTTGTTTTCTTTTGTTTCTGAGA
TAGAGTCTCACTGTAGAGCCTTAGCTGGCCTGGAAGTAGCTCTGTAGAGC
AGGCTGGCCTGAGACTCACAGAGACCCGTCTGGCTCTGCCTCTTGAGTGC
TGGGATTAAAGTGCACCACTTGGATGGCTCTGCCTATCTATTTTTAAACC
AGAACTCAAAGCTATGCACACAACTACACTCACATGTCCTCACACATATC
ATGTACACACAAGTTAACAATAAATATAATTTTAAAAATAAAATAATAAA
ATGTGAATGCTATTGTTGGAAACTAAGAAAGGGGGGCCTGGGGAAGAGGG
GGGAAAGGGAAAACCCACACCCCACCAGAGTTTTGCCTATTCTCTGGTCT
GTCAGGCGTGGGAGAGCTGCTATCTACCTTCCACTCATCCCTGGGTGGGC
ATTCAAGCCTCTGACCCGCTCTTCGAGGGGGCTGCAAGGGGCAGCTCTAC
CTGGGATTCCCGGAGCTANNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNCCAATTCCCCACATGCTATCCCTGG
TACACATGTTGCCTGGTACACTGGATCAATGAAAACTTTAGCTCCACTCA
ACCCTTTGAAGGCATTGCATCCTTTGAACTGCCAAGCATGAAGAGAAGAA
CATTTATGTCATTGCTGATAGTTGACTGGTGGGTGGTCATACAGTGGAGT
CCCTCAATAACTGGCTTGCTCGTGGATGTGTATTGGAGACCCCTCTATTC
ATTTTTCCAAGTAAACAACATTGTACACTATTCTTATTTTTATGCTTATT
AATGTACTTGTAAAGGTCACAGAAACAATAAAAATGGAAGGTTTTCATAA
AGCTCGCTGAGGAGGGTCTAGAAGTTAGACGGATCTCTGATAGACAGTGG
GACCACAAATAGAATTTTTCAATAAAAGGCAAACATCCAGCTTTTTCCTC
ACACTCTTGACCTGAAAAAGCCATAAAGGCCTGGAGAGTATTTCTCCCAC
CAGTAGGGCAGAAGGGCCCCCTGCTGGTGGACTGGCCAACAAATCGAAAG
CCTGCTCAGGATATTTACCCTGAGACAAGGACCTATCAATCAGCCCTCTA
CTCTTTGTTCAAACTGATGAACCTTAAACTGGGGGAAGCACGTTCCTCAA
AGACCCAGAAACCCCCAATTATGGAGGAGAGACTTGATTTCCGACAATGC
TCTTGGACCTATAATATTTCAAACTCCACAAACTCATCAAAGTTTCAATC
AGTCCATCAGTCCCATTTGTACACTCAGTTTCTTTCCAGGGATGTTCATT
TCTAAGAGGGTCCCTGGGCTAGCCAGACACCAAGGAGAAGACGAGGAGTC
TGCACCAGTGGGAGCACAAGGTCATGTTAAAGTGCTGAAGACTTCTTGAT
GCCCAAAACCTGCATCCCTAAAGGGAGCTTTAATGGCAAGGAAAATCGGG
AATATCCATGAAAACTGAGCTCAGGGTGAATATTTCAGTGTTTAAGCAAA
ACTGATTTATTTTCTCTGTCCCAAATGAATGAACATGATTTTGTTTTCAT
TAGTTTGTTCTCAGAGAATGTTCAGTAAGTGTTCAATGCATGAAGGCTGT
AAGAATTTAAACGTGCTCACTGGAGTTGGTGTCTGCTTCTCTCCAGAGGG
AACAACTTTGGTTGTGGCTACCTCTAACCGGGCTGGAGGCCAGATGTTAT
AGGGGCATTCCCTCAGTGGTCTGCACCCAATGTTCTTTCTCACTTCCTTT
AATGCCCTCTGATTCCACCGTGAAAACTATCTCCTTCCCCAACCACCACA
CACAGAATCAGGATGTGCATTGACCAAGCTGGCAGGACATTTGACACTGG
CTGAGTGTGGCATAGGAGCCTTAAGCCCTCTTCCTTTGACTGTCTAGACA
GACCCCTCTTGGGGGCTGGGGCTGTTCAAAGTCACCTGTATTAAGTGAGG
TCTCTAGGATCTCCCTCGCTCCTTTCTATTCATGAGTGCACATCTGTTCC
ACTCACTAACTCTTTGGTATAAGCCAGTCTCATAGCTCTACTGTTGTGAT
CATGCCATGGCACAAATGTTTGCCATTCAGCATTGGAAAGATTCCTGGCA
CTTCCACTTATAGTGCATTGAGCTTATAATGGGAAATAGACCACCATATA
CAACTTTGTCTGCAACATGGCAGCCTCAGCTCAGGGGATAGCCAAAAGGG
CCACCTCTAACATAGTGTTTTCACTTTTCTCATGCTAGATGGATGGAGTG
GAACCCCGGCTTCCCTTTGAGTATTGATGCCAAATGCCACAAGGATCTGC
CCCGAGATATCCAGTTTGATAGTGAAAAAGGAGTGGACTTTGTTCTGAAC
TACTCAAAAGCGTAAGTTTTAAGAACTTCAGGGTATGAGTGGCTCATCTG
ATAGCTGGGGATGGGAGAGTGTTAGGACTTCAGGACCACAAAGAAAGAGC
ACACCATTCCACCTCAAGTGTCTGGACCAGGAACAGCTGTACTCTTGATC
AGAAGGGCAGCCATACACCAAGACAGACATTGTATTTGCTTATTCTAACT
CAGGTAGTGTCTGTATTCTCCCCATTGACTGGGATCCAATTCACGGCCTT
AATGAGCCAACCTGAACAGAATTGGTACACAAGAAATGAAGGAAGGGGAA
AAGGGTCACTGATCCAGGTCATCAAATGCCTAGAATACAGCAATGGACCT
TGGTGTAAATAAAGATTTCGTTGGGGAAGGGGAAATTAAAGCATTTCCAT
AGAAGTCCAAGGTTATTTGGGGAGGGATAAAAAATGAGTTTATTCCTTGT
CTGAAGGCAAGTTGTAGAGCATCTGATAGAAAAGACTCGTATTTGTTCTT
TCTTATAGTAGGAAAGGCCTTTTTTATAGGACTTGGGCTGGGGGAGCAGT
TCAGCTGAACACCATTCCCCATGCGTTTGCTCTACCCAGGCCAGGAGGTA
GTCTCTCATCGAACAGTTGCAAGATGACTTATATCTAAACAGCCTTGACC
CAAAAGTCTTCTAGCACCTACAGCCCCGTGCTTTCAGCTAAAAGCAGAGG
CTGTTCTTACTTACAACACTCCATCTCCACTTCCAGTTCACTCCTGTGGC
CAGTCCATCAGAAGATGGAGTCCAGACTGATGCCAAGAGGCAGGTTCCAC
AGTAACAGTGACTCAGATGACACTGTTGGTCCCATGACTTAGATTCTTGT
TCATTTGTCTTAAGGACCCCATGAAGTAGGCATATGTGTTTATCACTGTT
CTGGTGCTGTGAAGAGACACTAAGACCAAAGCAATTCTTATAAAAGAAAG
CATTTAAGTGGGAGCTTGCTCACAGTTCCAGAGGTTAAGTTCATTATCAT
CCTGGTGGGGAGCACGGTGGCACACACGATGCTGGAGAAGTAGCTCAGAA
CTTTACATGCTGAACCACAGGCAGACACAGAGAAAGACCTGGATCAGGTG
TAGGCTTTTGAAACCTCAAAGCCCAACCCCAGTTTCCTCCAACAAGGACA
CATCTATTCCAACAAGGCAACATCTCCTAATCCTTTTAATCCTTTCAAAT
AGGCCCATTCCCTGATGACTAAGCATTCAAATATAGGAGCCTATGGGGGC
CATCCAAGCACCATAGGACATATTGTCCTCTCTGCAGTTGAGGAACCGAG
AAGGAATAAGAAGGTTGAGGACCTTGCCTAAAGTCCTTTGCTAATAAACA
ACAGAGACACTCAACTCTGGGTTACATGTCTGCACTAGAGTTGCTGCAGG
ATGCCAGCACAACCATGGATACTCTGTCAAAGAGTAGACTCTTTCAGCAC
CAGACCAAGGTTAGCAAGGACACCTTTGATGTTATACTTTGACTCTTAGA
GAACTTTTATACTCATACAAACCTAGAAAACCCTAAACTCTTGAAGCCCA
ACTGAGCCTTATACTCCCCCAGGAACTTTTAGCACTTCCCAGCACACAGC
ACATCCACAGACAAGTCTCAGAGAGAAGGACTTGGCTACCTACACACCCA
ACAGGCAGCCAGGGCTGAGGACCACAAATCAGTCTGCTCCATCTGGGTGT
TTTCACAGCTATTGTTGGTCATTTCTAGGTAAGGAAATCAAGAGTCCAGT
GGGTTAAGATAAAGTAATTTACATATATATATTCCCATACATAAATAGAT
GATTTATATATAAACTAACTCTGGTGACCTGAATGAGAAATGACCCTGTA
AGCTTGAGTATATGAACACTTAGTACCCAGTTGGTGCTGGCTTTAGAGGC
ATGAAGGATGCAAGAATGAAGGGATTGTGGAGTCTTCCCCCATGGTTTTA
GAAAGCCACTAAGGCAAGGCACTGTGTGGTACGGATGTCCCTGTGTGAAG
ACCACAAGAGCAAGAGGCTCTGAGAGGCTATTGATCAAGCTATACAAGTG
TGGCCTTGGCTGCAGTGGAGACACCAAGAGATTGGAGGTGTCAGAGCTGA
GAGATACATACATAGGAGAGCTGCTCACATAGATTGGAATCAGCCAAAGA
GAGAAATATATGTTGTAGTCAGCAAAGTTGGAAGGGAAGAGCCATTTAAG
CCCTTTGACAATAGGCATAGGGCTAAAGGATTTGGAGTGTGCCCTGCTGG
GTTTCAGTCTTGCTTTCATCCAGTATTTCCTCTCTATTCCTGCTTTCCTC
TCTTGTGGAACGGTAATGTATATTCTTTGTGGAAGTATGTAGTTTTTTTT
TTTAAATGGGGGTTATAATTAAGAGATTGCCTAGAGTCTCAGAAGAGACT
TTAACACAGAACTGAGCTTGCAAGACTCTGGGGACATCTGCAGTTGGACT
AAAAGATTTTGAATTATGATATGGCACAAACCTACAGGGAACCAGGAGTT
GAGTGTGGTTTAAGTGAGAAATGTCTATAGAACTGGCGTAGGTAATATTT
GGTGCTCTATTTGGGAAAGTTTAGGACGGGCAGCCTTGCTGCAGAAAGAA
TGAACTAGGGGCAGGTTTTTAAAGTTTAAAGTCTTGCCCCATTTATCATT
TGTGCTCTGCAAAGGAATGAATGAACACATGCAGAGGTGGGATTCAGAGA
CATTCCAGAGGGCATAGGCCTGGTCTCTTACACAGCTGGGGTGCAGTAGG
AAGAGCGTCTGCCACAGGATAAGTTCTATCCTGGTCCCACAAGGCCAGCA
CCTACCTCTTGGAACTGCCTGGGCTATGAGGAGGTTCCCAAGTATGAAGT
AAAAACACTCTGCGATGGCATAGTGGGTGGGCAAGGTGACCTGTCCCCTG
GTTTGCAGGATGGAGAACCTGTTCATCAACCGCTTCATGCACATGTTCCA
GTCTTCCTGGCACGACTTTGCTGACTTTGAGAAAATCTTCGTCAAAATCA
GCAACACTATATCTGGTAAGAGGGTTCTGTGGACGGGAGAGTTTATTTTT
CTGTCTGAGACTTGCAAACATCCAACCCATGCACCCCCTCGACATCATCT
CACCAGAAGTGTCAAGGCCAATGGGACTTTTTCTTTACATACATAAATCA
TTTGTGGCTCTTCTGCTCAGACCCGAGGCAGGACTGGGCTACATCAACCT
AGCATTGGAGGCTGAAATGATGGCTGAGCAATTCTGCTCTTGCAAAAGAC
TCAAGTGCAGTTTCAAACATCCACACTGGGTGGCTCACAACCACCACTAA
CTTCAGCTACAGGGCATCCAAAGTCTCTGGTCTCTGGAGACACATGTGCA
TNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NTTACACACACACACACACACACACACACACACACACACTTAATAGTAGT
AAAAACAAAATTTACAGCAATGGGGTCCTTAAGACCAAAGTTCATTTAGT
TGGAGCCCTGGCACTGTCCTGGACAGTAATAGATGGAGAGTCATGACAGC
CATCACTCTTCCAAGAGGCAGGAATGGTTAAAGATGCTGACCTCAGAACA
AGGGATCAACATTCTCCCCTCTCTTTTCATGCTGGGCGGAAGGACCGGAA
ACCTTGAGAAGAGCAAGTGCCTGGAAATCTTACTGTTCACTAAGGCATAG
CTCTAGGAAAACTAGCTCTATAGCCTGACTCAGCAATGAGTAGGGACACA
GCAAGTAGGGACATGGCAAGTGAGAAGATCCCGATACCTACAAAGATTTG
TCCTCCTGCAGGATCGTGGGATCCTGCCCAGCGGTCCTATCTAGAGGTCA
TTCTCTCCACAGAGCGAGTCAAGAACCACTGGCAGGAAGACCTCATGTTT
GGCTACCAGTTCCTGAATGGCTGCAACCCAGTACTCATCAAGCGCTGCAC
AGCGTTGCCCCCGAAGCTCCCAGTGACCACAGAGATGGTGGAGTGCAGCC
TAGAGCGGCAGCTCAGTTTAGAACAGGAAGTACAGGTAGGCCAGCTTGCT
GAAGACCTAGTCTTTCCACAGTAGCCACTTCCTGCTGTCCAATGCTTGCT
GGTCCCCAGACTTTTACTGGCTTGTCTGGTTGATTGATTGTGTGAAATCA
GTAGTTTCTTCATTTTTCTGGCTGCAGCATCCAGGAGGATCTTGGGCACT
GTGCAAGCCACATGCAGTCACAGGAGGGGCTGCATCTACACAATCCTCAC
ATCTGCAAACACAGTGTCAGTGTTGTGTTCAATGTCCATATCAATAATTA
CTTGACATTCCCTGTGATAGGTGTGTTCTACACATAGTCACCCAACTGTC
ATCTGCTTTGCAAAGGTGAGGAAACTAGAGAAAGAAGTAGACTTTGTGTA
GGTAGAACTTACTGCTAATTAGAAACATAGGCACTACCTGTCCCTCACAC
ACTTTCCCAAGCCAGTCTTTCCTGACTTTCACTTGCAGGCTCAAGCCCTC
ATCCCCAGTTCCAAGGCTGATCTTGTCTTGAGTCGGATAGAGTTTAAACC
AAATCGGCTTGCATCTGATGGAATCTTCCAGGCATTACCGTGCTAGATTT
TATTTTGCTTGCTGAGAGTCCCAACAACCCTCAGCATACTCCCATCTCCC
CATTGCAGCTAGCACCACTGGGCCTCCATCTCCAGCTGTTCATGTAACCT
CCATTGCCTCTGTTCACCCTGTCTAGGTACCCAGCCCACATGGAGACTCC
AAACACATACCATTCAGGCCTAACTTCCTGCCTACATGACTTTGGGGGTT
ACTTAACCAACTGAAGTTGGGGTCCTGATCCACATGGAAAGTCCTTCCCC
ACTCATCAGGACCTTCATGGGATCTGACATATTTCTAGGCATGCAGACAG
CCAGCAACAACAACTCCTTACACCCACCCCATGCACAGAAATAAGCATGT
CTTAGAACTAAGAGGGACCTAAACCTCTGCAATTGCCCCTAGCTACCCAG
AAGTAGGACCTTGCCTGTTCTCCAAGACAAGCCCCCTACCAACTCCAGGG
GAGGTGGCTTATGGGTTATCTTCACTTTGAGCTGACCCATCAAACACATG
ATAGTGATGATGTTCTGCTGTGGTTTAGATCATGAGGCAATAATCTAACA
ATCTAACAGCCACTCCTGATTAGACTCAGACTGACTACCTTGTCTCTTAT
ACTGTCACCAACTAACACACCAGAACAGTTCACAGGGCCCTCCATCATTC
TTGACACCTTTCTTTGACAGCCTCAGTGACATGTTAAACACCAAGCCCAC
GAGTTGCTTTGTTTACAGTATTGAGAAGTGGCCCCAGAGCCTCACATGTC
TTAGAAAGCCCTCTTCCACTGAGCTATATTACCAACCCCTAAATATTTTT
TAAAAATATTTTCCAGTGAAATTTGGTTTTGGAACATGATTGGAAGAACC
TTTCAAGGACCCTTTTCCCCCCATATATGCAGAGAGTAGCCCCCCCCCCC
CATCTCTTCCTTTCCAGGAGAACCCTGTCCTGGATGACAATGGCTAGTTT
GCCCAGTGTTGAACTTGATCTAAATGAAGTCCTCCAGCTGCATCTGCATG
TCTGACTTCTCCTCTCTCACTCTCAGAGTGGTCTTTCCGCTTCTTTCCTG
AGCATTAAGCAGCTTTCAGGCACAATGAACCTGAGTCAGTTGTGGCCAGT
CTTTCATTTTAGCTTTGAAAATAATTAGAAAGCAGAAAATAAATATGTAA
GCCAGCTTTGGAGCTGGAGAACCAGACCCATGCATTCTGAATTCCATTTG
TACCCAAAGCTTTAGGGTAAAAGCCTGGGTTCCCGAGGCCCAGATAGACT
AGGTTTGGCCACCCAGCCCCACACACAAACAAAAGAGATAGGTAGAAAGG
GAAAAAACAGAGATTTAATCAATATGGACACACAGGGGGAAGAGATAATG
AGGTCCAGTGACCCCCTAAATCCATCTTGGGGCACTGTCATGAGTTTTAA
GTTTAAATAGAAGAAGTTCTGGCCTAGGGGACAAGATGTGGCTAAATAAG
AAGTCCCAGTCTTTTTGACCTATTTTTTGATACTGCAAGTATCAAACAAT
AGCAAGTCTTCATTACTGATTGAACATACGGATCTAGTTTCCACAAGATT
ACTTCTGCCCCAGGCTCTGTAGTATTGCTATCCTGGTACAAGTGTGTGTG
TGTGTGTGTGTGTGTATCTGTCTGTCTGTCCTGGTAGGTGTTACCTGTTT
ACCTGTTTCTGGGACACAAGACTGTGACATATTTTAAATTTTTGCAGCAA
ATATTTTGGTTCAAATAAACCTCTGAACCATGAAGGAGGAAGCTCAGAAC
TTCTGTTCTATATGATACATGAATGGATCACTGCAGAAACATTCCTGTGT
TGAATGTAAATGTAAAATGTATTATGTTAATATACTGCAGGAGAGAATGA
GGGCTTACCTTTGTACCCTTGGCCTTGAAGAGAGGAAAGTAGGTTAGACA
GACTCTTCCAGTGACCAGCAGGCTGACTTGCAGAGTCTGCAGGGGTGTGA
CAAAGGCAGGAGTAGCAGGCTGTCGTCCTGCATTTAGTTACTACGTGGTC
ACATGAAGAATCGTCCACTCTTACTAAAACAGTTTCTAGTGTTTGTTACA
CACCAAGTTACAGGGAAGGTGCGTGCATGTGGGTGTGGTGCACATGTCCT
GAAGACCGTCAGTCACATCCCACCCCTGGCCTGGCTCCCGGGTCCGTTTC
TGCTTCAAGTAGCTTGAATATATTTGGCTGCCTCCTGCAGGAAGGGAACA
TTTTCATCGTTGATTACGAGCTACTGGATGGCATCGATGCTAACAAAACT
GACCCCTGTACACACCAGTTCCTGGCTGCCCCCATCTGCCTGCTATATAA
GAACCTAGCCAACAAGATTGTTCCCATTGCCATCCAGGTAGGTGGCTGCT
CAGCCCCAGCCCTTCTGTACGACTTGTACCTCTAGGGCTATGTGTGTGTG
TGTGTGTGGTGGGGGTTCTGGGGCAGCACCATCCACTCCACTGAGAGGCA
CCAGAAGCTCTCACTCCAGCTTTCCCTGGGGTGGGGGAGAAAAATGAGCT
ACTGGGGGTCGCTGCTGCCATGAGAAGGCTGTCCACACCACGGCAGACAG
TGCCTTTCCCAGGTACCTTTGAGACATTCCCTACTGAGGCTAAGCACTGG
GGAGGGCTTGTCCTGCAGTGACCTGCTTTTCCTCCTACCACCTCCTGACG
ACTGTTTCCAAATTGACATGCCCACACAAGCCAGATGGGTGAAGCTAGGG
GTGAAGTTGCAAACAGAAGCCACTGAGCTGTGTCGGAGACCTCACCAGTC
CTGGAACATTGTCTTCCATACAAGTAGATTCTTGCAGCCCCGAGGACAGT
GGAGGGCCACGGGAGCAGTCTCATGGGGATGGAAAGTCTGTGACACTGGG
TAATGGGAATAATATCAGTGATATGCTGGTGTCTACACAGTCTAAAAGCC
TTTGCCTGGGAAAGCTTAGAAATCATGAAAACTGCTGTATGTTAAGTGCC
TAAGAACAGACACTATGCTTGAGCTTGGCCAAAAGACCAAGAAGCGGTAA
AACTGTGACACATTTTAAATTTTCCAGCAACTGTATTAGTTTCAAATAGA
CCTCTGAACCAGAGGAGAAGTAGTTTAGAGCTTTTGTTCTGTTCAATATG
ATTCCCGAATGGGTCACTGCAGAAACATCTCTGTGTTGACTGTAGATATA
AAATACCTTCTGTTGTCTCAGACCTGGAGGAGGGACTTGGCACATAAAGA
AGCGTGTATATCGCTGGCATCCTAAATATTCTGGATCCTGGAGCCTATGA
TCATTGGGTCTCACACAGCATCTGTCTCCATTTGTGTAACTGTCACCTTA
CTGCTCTCAACCATCTTACCCATGCAAGGAGACTAAGGTACAGGTGCTCA
TAGTTAGCCAGCCACGGAGCTGAAATTCAAGCCAGCTGTCTCAAGCCTGA
AGTTTAACTTATGTTTATAATCCCAGGCTGTCGGTTCAAGGACAGCCTGG
GCTACAGAGTGGTGCCCAGTGTCAATTTGTTTAACTAATTAATCAAATCA
GTTAGCTGCATATCTATACCCCCACCCCTCTGCACTCCAGGAGTCCTGAG
CCTATTATCAGAGAAAGCTCTATGTGTAAGAAGCTGAAGCATCAAATGAA
GGCACAGGGGATGTGGGAAATCACCACTGAAAGATGCCAGGAGAGGGATG
GATTACCACTGACAGGTGTTCTGAAATAGCATGTCCCAGACATGTATTTA
TTAGAGCCAGAGGCCTGGCTCAAATCTCAGCTCTTTCCATTGTTAGCAGG
AATTAGGCATTGGACAAGCCACAGGACATGGTTAACCCTTTCTCTATAGC
TGCCCAGGAGAATTAATGCACCCCAGGGCAGTGGTAGCTGCAGTTACTGG
GACAACCCTTGAACAGGGCCCACAGCTGCTGTAACAGATGGATTCTAGGC
TGGGCTGTAAGAAGGCCCATGGGTCTCCGTGTTGAGGCTGGCAAGGCCAG
GACAATTGAAAGCAGTGCTGAGAGGTTCTCTGCCAAGACTGAACTGAACC
AGCCTTTTTGAGGATCAACAGAGCAACATGCTAGAGGATGGAGATCATGG
TTCTGGCTTGGGTGTGAGGCTGCAGACAGACAGAACATAGGAGAGGTTGA
GGGGATGGGACCGTGTCCAAAGCAGGAAACACTACAGCACTGTACACCTT
CAAAGAGGACTGCATCTGCTCTCAGCTGACTTGGCCCTTCCCAGGGCTAG
TACCAAAGCAGAAAAGCAAAACATGGAATATGGATGATATCAGCTCATTT
ATGCAAAAATGAATGCAAGAACACAGCATGCCACAAACCATATGCTTTAC
ATGGGTAGACATTGGTGTTGGCTTGGTGAGACACAGGACACAGGGTCACC
ACGCTGCATACAGAACAGGTTGGAGATAGTAAGGATAGGTCTAAGTTCCT
CATAATAAGGTTCTGTTTATGAAAAGTAAACACAAATACACACATATGCA
AAGACCTAGGGAAGCAGCGACCTCTTGTGGCTGGACAAGAGGTACATTCT
CACTAGTTCTTGGACTCTTCTGTAAATGCTGGAGTAGTTTACAATAATAA
TCATATATTATTTTCTACTCAGAAACAAAAGATGGCCTCCTTCAAAAGCA
TTTAAAATAATGCAAGCACGTTGTCCTGGGCATATTCTAGAAGAGCTGGG
ACAAGGACAAGTCTGGAGGGGTGGGAGGAGGTGTGGCATGCTCTGTATAA
AAGCTCACCATGGGAGCCCCTGTGTGGCATGGTCAGACAGAGAGGAAGGA
ACTGCAGGGTGATGGAGTTACAGCAGGCAAAGCAACAGAGCATCCCTACT
TAGCTCCATCCAGACCTCTGTGTGGCCTGGAGAGAGGCTGAGTCACTCAG
AACCTCACACTTTATCTCTTTGCAAAATAGGAAGGATGAGGAAGAACTGT
TGGTCAGGCTGTGGTGAGGATTTTGTGATGACACATACGTCAAACCTTAC
GACACAGCAGAAAGTCTCTGGACTCAGTGCCTGAGGATACCTGAGTAGGA
CAGGTGGCCCGTGTGCTCTAGGAAGCAGGCCACAGGGTGGCTGTCTGTGG
GCCACACAGCTATGGAAACAGAGCGATCACTGTGAGGAGCAGTCCTCTGA
GAACCTTGCTCACTAGGACCCTCTGGAAACGGCAAGCCTGTTGTCCTCTG
AGGACACATGTGCTGACTTCTGACTTAAACCTGATCACTTAAGGAATCTG
AAATTCTCTCCTAAAGGTAGAGGACAAATCCATTAGCGAACAAGTGGGAA
GGATACTGTGGGGGTCAGGAGTGTCTCCCTGGCCTCCTTGCTCATCCTCA
GGCGGTAGGGGAGAGCTGCTTCCCTAGTAGTCCTGCCACCTCCCCCACAG
CACAGTGGCTTAGGAAGCTTAGGACGCAGTACTCCTTGCTCTGGTGTTCT
CGAGACGCACTTAATCCAAACCAAGACCCGGAGTAAATTCTTAGTGTCCA
TAACCCAAAGACTTAGAAGAAGAGTAAAAGAATGAAACCTGCCAGGACTA
TTTCAGAATGGTTTTGTGTTGTAGAAAAGTGGGTGAGGCAAGGAGCTAAA
GGGGTCTGCAACCCTATAGGTGGAACAACAATATGAACTAACCAGTACCC
CCGGAGCTCGTGTCTCTAGCTGCATATGTAGCAGAAGACGGCCTAGTCGG
CCATCATTGGGAAGAGAGGCCCCTTGGTCTTGCAAACTTTATATGCCTCA
GTACAGGGGAATGCCAGGGCCAAGAAGTGGGAGTGGGGGGGGGAGTGGGG
GGAGGGTCTGGGGGACTTTTGGGATAGCATTTGAAATGTAAATGAAGAAA
ATACCTAATTAAAAAATAAATAAATAAAATTCTCAAAGATTAAAAAAGAA
AGAAAGAAAGAAAAGTAAAGAAAAGAAAAGTGGGTGAGGTAAATATTTTA
CCCTGGGGTTACGTATTTCTTTTTTCTTTTAAAAAATATGGCTGACTAAA
AAAAAAAAAATGTAATTTAAAAAAAGGTGGCCTCCTAGAAATTTGAATTT
GTGCTGCTTGCATTATATTTCTATTGGCTAGTGCTACTTTGGATGGAGAG
ACAAATCAGAAAGGTGCTTATAACCTCTCTAATGAAAACAACTTAAGGAA
GGGAGAGTTATTTTGGTGCTCAGTTTAAGGGAATACAGTCCATCGCAGAG
GGTGAGGCATGGCTGTAGAAATAGGAAGTGGTTGGTTCCATTCATAATCA
GGAAGCAAATAGACAGGAAGTGGGGCCAGTATGTGGAACCTCCAAGCCCA
AGTCTCCTGCTCCTAAAAGTTCCAAATCTTCCAAAACAGCACTGCCAGCT
AGCGATCAAGTGTTCAAACACACGAGCCTACCGGGACATCACATTTAAAC
CGCAAAACCATCTTCCAGATTAGTTAGGGGGGTGCAGCTAACTCTGCTCA
GGGGCCTTATCTTTCTGGCTTTAAAGAAATGAGTTTTTAAAGAACAGATA
ACAAATTTCTTTACATTTTAGAGAAAAATCACATGACCCAAGCAAGGAAA
CTGGAAATGCAAACGCAAGCTGGGCCCCTGGGTACCTGGTTTCAGAGTTA
GGGCTCAGAGCAGGGCTCCAGCAGCAAATCCTGATTTGCAGCTGGAGCAA
AAGGGAACTGGGCAGCATGCAGCCCCAGCCTTGGTCCAGAAGGAGCACGG
CACTCAAGGTCAGGGTGTCTGTCAGGGAGGAGGATGGAGGGCACTGCTGA
GCAGAAGTGGTAAGCTGGAGGATGCCAGGCTCTGGGGAGGGCACAGGTCA
CCGACCCTTGCAGCTGCTCGCTCCTTCCCCGAGGTCTGTACCACTGTCCT
CAAGTTACAGACAAGGAACCTACAGCTCACAAACAGGTCAAAAGACATGT
CACTAGGGCCCTCCACCACTCCCCACGTGCTTTTTGTGATCTGAAAGTCA
TGGCCGGTTGATGTTTGCTTTCTTGTGGAACTTCCCCAGAGTTCTCCCAC
AGCTCCCAATGAGAAATAAAGTGGAGCGTTCTCTTTCTCTAATGCACCAG
CTCAACCAAACCCCTGGAGAGAGTAACCCAATCTTCCTCCCTACGGATTC
AAAGTACGACTGGCTTTTGGCCAAAATCTGGGTGCGTTCCAGTGACTTCC
ACGTCCATCAAACGATCACCCACCTTCTGCGCACGCATCTGGTGTCTGAG
GTGTTTGGTATCGCCATGTACCGCCAGCTGCCTGCTGTGCATCCCCTTTT
CAAGGTACAACCAGCCAGGGCTCCACCTACAAGGAAAGATTATCTAGGAG
AGTAGCTGGCATCCCAGGGTGTGTGCGAGTGGAGTGGTGATAGCTAGGAG
TAGGTTGCAAAGAGGGGCCTAGGACAGACATTCAAGGGCCAAGTCACAGG
AGCCTGTTACAACCCTGCTGTCCAGAAAGCAGAGTTCAAAAGGGCAGTTA
AGTCATTTTCCTTCTCTTCCCAAATGCTGCCAGCACTTGATTGTTGGGTC
AACTACAGGCTAAAAAAAATTACTGCTGCTAAGCCAGGTGGTGGTGGTGG
CAGCACTTGTCTTTAATGCCAGTACTTGGGAGGCAGAAGCAGGCAGATCT
CTGAGTTCAAGGCTAGCCTGGTCTACAGAGTGAGTTCTGGTACAGCCAGG
GCTATATACCTAGAGGAACTCTTTGTCTAGAAAAAAAAATAAAAATAAAA
AAAAATAAAACAAAAAAGAAGAGAGCTGGTGCTTTGGTCTCTTCGTTGTA
AATTAGTTCAAAGCAGGACGTCTGCCTTAGAGACTGATAACCAGGATCTG
TTCCTAGTGTAGTAGGAGACAAGCGCCAGGGCCCTTACTCCTCAGCATAA
CAGTGGTGGCCACAGGCCTCCTATAGCTACAGGGCAGGAAGGTCTACACA
GAGCCCTTTCACTTCCTCTCTGGGGCCAGAGTGACCCCCGCACTGTGGGC
TGGCCTGGCCTGGGCTGGGAGTGCTGAGTGAGCATGGAGACCCTCAGTGA
GATTTCTCCTCCATCCAGCTGCTGGTAGCCCATGTGAGGTTCACCATTGC
TATCAACACTAAGGCCCGGGAACAGCTTATCTGCGAGTATGGCCTTTTTG
ACAAGGTGAGTGCCCTCTCTTCATGTGGAGCCTGGACAAGCTCTGCCTTG
TGGCTCCATCCCTATCTGAGGTGTGAAAAGTGTGGGAAACTCTGGTCCTT
CAACCAACACCACAGCCAGCTCTCCCACTCTGTGTTCTCTGTCACCTTTT
TATGTATCCTGTCCAGTTTCAGTGTCTGAGCCTCCTCCACACAAGCCACT
AACCTCTACCTTACAAAACATCTGTTATTTCTCCCATACTACCATATCCC
TGTCACCCTGCCTGTGGCCTTCGAGGTATTTGAACAGAACTGCCCAATAA
TTGCCTACCCATGTCCTTGATGTTCCCAGTTCTTCAAAGCTGATTGGATG
CCCAAAGCTATGTTCTGCACAAAATCCTGCTCTCTCCGTTTGCAGCTATA
CAGTGTCCGGTGCACGTCACCTCATCGTGGGAGTGGGAGCGTGGGCCTAC
TTAAAGCTGAACAGCTCTCCCACTCCACCTCACAGGACAGACTGGCCTCT
CTATACCCAGCCACCCACTGGCCTCCAAGTCCCTCACAGATGAATCTACA
GTATGGATATGGACAGCTTTTGGGGAAAGCAACCAAACTCAGGCTCTCTG
CTCTCTTACCAGGCCAATGCCACCGGGGGTGGAGGGCACGTGCAGATGGT
GCAGAGGGCTGTCCAGGATCTGACCTATTCCTCCCTGTGTTTCCCGGAGG
CCATCAAGGCCCGGGGCATGGACAGCACGGAAGACATCCCCTTCTACTTC
TATCGTGATGATGGACTGCTCGTGTGGGAGGCTATCCAGTCGTGAGTGTG
ACTGGGTTCTGTGGGAAGGGGAAACCCTAAAGAAGAGTATGATGAGGCAA
GCTTGCTCCTGGGTTGGCTGCTGATGGAGGGAAGGGGCAGAGTCCGACAT
TGAGAACTGATGGGACTGGGAGAGGGACCTGCTGGATACGCACTCCTGAT
AGCCCCCTGACCCAGGTTCACAATGGAGGTGGTGAGCATCTACTATGAGA
ACGACCAGGTGGTGGAGGAGGACCAGGAACTGCAGGACTTCGTGAAGGAT
GTTTACGTGTACGGCATGCGGGGCAAAAAGGCCTCAGGTAGGCTACAGGG
CAAGTGTGCATCTCCAGGTCATGAGGAACAGAAGGCAGGTGACTCTGCTC
TCGGGTACCCACCAGTCTCAGAGCGTCCCTCGGAGCATCCAGCCTCCCTT
TCTCTGAGCATCTTGCTAAGTGTGTGTGGGGAGCTAAGATAGAGGCAGAG
GTGGGTGTCCCTCACAGAGATGAGCTCACGGTCGGTGGTCGGTGGCTCTG
TCTTAGGTTTCCCCAAGTCCATCAAGAGCAGGGAGAAGCTGTCCGAGTAC
CTGACGGTGGTGATCTTCACGGCCTCTGCCCAGCATGCAGCTGTAAACTT
CGGCCAGGTAGGCCAGGCTAGCCCTTTCTGGGGGAGAGTCTTAGGTACCT
CACAGTGGGAACCACCTCCAATTCCACACCCCTGGCCCAGGCTCTTGCCC
TACTGGTCCTCCCATCTCCATGCAGCCTCTGAGCCTGGAACCAGCAGCAG
CAGGCATGAGGCACCCACCAGGCTGGGGCAGTTGGAAGTTCTGGAAAAGG
AGGGAAAGGGTCTGAGGAGGGAGGTCTGGGGACCCTGGAAGAAAGGCAAG
GTAGGTAACAAAGGAGGGGGACACTAGTGGTATGGTGACATGTCAAAGGT
GTGGCCTGGGAAACCAGGAGAGGAGAGGGCCAAGACAGGTGCAGTGGGGA
CATCAGAGAGGTGGATGGTGTCCACAGGGCGGGGTGGAGCCTGCTGAGCT
CCCTATGAACCAAGGAAAAGCAGGCCTGTGGATCCGAGGTGGGCAGCCAC
TGGGCTTCTTGGGCACCCACGCTGTTGATGTTGATGTTGCTCTCACAGTA
TGACTGGTGCTCCTGGATCCCCAACGCTCCTCCAACTATGCGGGCCCCAC
CACCCACGGCCAAGGGTGTGGTCACCATCGAGCAGATCGTGGATACTCTA
CCAGACCGTGGCCGATCATGTTGGCATCTAGGTGCAGTGTGGGCCTTGAG
CCAGTTTCAAGAAAATGAGGTGAGACCAGGCACTGTTGGAAACACCGTAG
ATCACTCTAGTTTTAACCCATTCTCCAGCGCACGGCTTTGGGGCTCTGAC
TCAAGCTAGAAACCTGCAGTCAGAAATCCTGATTTCTAAGGTGGAGCTAT
TAGGAGTTGGGGGTGGGGTTACTCCACCCCAGGTCCTCAGCGTGGGTCTG
AGCCCTGGGCAGGATGGCAGTGGGAGGCACAGGCTCTGCTCGGAGTCACC
AGAGGGGTGTGTGTGTGTGTGTGTGTGTGTGTGTGTGTGTGTGTGTGTGT
GTGCTATCACCCTGTACCCCATACTCAGTTGATAAATCTATTCCACATGG
TTTCCTGACTCCCCACAAGGAGAATTAGAGCTTCCTAGTTTCACTTAGGT
ATCCTTCACAGCTATGCTAAGTGCAGGTCTTGGTGGAATCAGCCCTTTGC
AATGTCTCAGCGCTGTTCCTATCAAAGTACCTTCAGCTTGTCCCAGACCT
GCTAATGTGCTGGCTCTATGCTCAGTAGGACCAGAGGAGTTGTTCCTAGG
AGACTAATTTCCTGGTCAAAGGATGTCAAGTCTGACTTAGCTTCTCCAAA
CTACACCCCAGATCTCCCACCCCCTTCCAGCCACTGAACACTTTGCAGAC
ATTGGTGTAAGCACATTCCCCTGCAGGATGGCCACCTCTCCATGCCCAGG
ACCCCTGCCGGTCTCACGCCTTAAAAGGAAGATGAGAAAGACAAAGGGCA
GGCCAGCAGGACCCTGTGGCCACAGAACTTCAGAGCTGGGAGCTTCCAGG
TTCCCTTGCACCTTACCTTGTCTAAAAAAAAACACCTGGCAACAAGGAAG
TAACTTCCTTTAAGATCTCCAGCCCTTAGCTTTTCATAGGGCAAAGGAGG
TAGCATTTGCATAATCTAAGTTTTAAAAAAGAAACAAACTTAATTTGCAT
ATTTATGAGATCTAAGTGTGATATGTGTATGCATTGGTGCTGCTCAGTTA
TGGCATCTGATCTATCACCTCAAACAAGTTACTTCCTTGTGTCCTTGTGT
AACATTCAGACTCTTCTAGTTTAGAGATATACTACAGCATTAACTATTTA
CCCTACCATGCTGTAGAACACTGCTTAGGCCTCCTTTGAATATACTAATT
TGCCATTTTTCCTCCTCTGTGGTAACTTCTTTGAGATGACCAGGCAGGAC
TGCCTTACTGTTTTAGATAGGGTCTCACTATGTAGCCCTGGCTGGCCTGG
AACTCTCAGAGACCTGCTAGCGTTTGCCTCCCAAATGCTGAGATTAAAGA
CATGTATGTGCCACTGCACCTGTTGAGACAGCAGTTTTTAGTATCCTGTA
TATGGTATTTGCATCTGCTTCTTTCCCAGTTCATCTAAGTTCCAAAGACA
GGATTTTGTATGGCCACGTAGTGTTCCATCGAGCATTCTCAGTGGGTAGC
AAAGGTCAAAGGTGATAAGAGTAGGTCCTCCTCCTTCCCCCCCCCTCCCC
CCAAAGGAAGCCTCTTAGAGTTAGACAAGGCCTATCAGTCTATAAGGTAC
CCTCTTAAACTCTTTCCATGTCTGTCGCAGCTGTTTCTAGGCATGTACCC
AGAGGAGCATTTCATTGAGAAGCCAGTGAAGGAAGCCATGATCCGATTCC
GCAAGAACCTGGAGGCCATCGTCAGCGTGATCGCCGAGCGCAATAAGAAC
AAAAAGCTCCCCTACTACTACCTGTCACCAGACAGGATTCCCAACAGCGT
AGCCATCTAAGGCCTTGCCTCCCTACCCAGCAGCTCTCTGGGAAGGCCAG
TGGCTTTATTAGCCAGATCCCAGCTTGCCTGGCAGGCTCTGGGTCGATCT
TCCTGCAGCTGGTGCCTCTTCCAAGCTCGAAGTGCTGCTCTTGGGCCTAG
GTGGTCTGGTTGAAACTGAAGGCTGTTGTAGGATGGGGAGACATCACAGA
GCCTCAGCATGTGCTACTTCTTCAGTGGACACAGTTGAGGAACCTCCCAG
GCAGGGCAGAGATGTGCAGCTGTGTCCCCCAGCCCAGCTCAGTGCCTCGT
CACTCGGTAGCATCAGAATAAGTGACAACTGTTCTGGCTGGGTCAGGGGT
ACTTTATTCTATTTATGCTTCCTCCAATTGCTTGCATAGAGTAGGTGCTT
AGAGAAAGTTCTTGGATTAAGAGTTTGTTATAAAATAAACTTCATTTAAA
ACAGGTGTCATACCACATGCTGAGGTCCAGTCACCCCCACTCCCACCCCC
ACCAAAATCACTGTTCTCTTTCGATCAACAATAAAAAAGCTGGTCTACTA
CCTCCTCCAACTGACAAGGTCTTTGCCCCCCACTCCATACCAGTGGCCCT
TTCTGCTTTTGTAGACAATACAGGTATTCTAAATTAAACACACAAATCTA
AAGATCTGAATTTATGCTATGATTCATATATGAGAACACAT

The screening laboratory 20 having both the endogenous DNA sequence, and the mutation DNA sequence can compare these elements to reveal the junction site in the mutant DNA sequence. These sequences are compared using a software program, such as Fasta Two Sequence Compare found at http://fasta.bioch.virginia.edu/fasta_www/cgi/search_form2.cgi. These alignment show the screening laboratory 20 that the junction site where the mutation is inserted occurs at eight 108th nucleotide of the mutant designated genetic sequence.

Upon identification of the mutant designated genetic sequence and the junction site, two other software programs are utilized. The first of these programs is a blast program that identifies homologies between the designated genetic sequence and the endogenous genome of the mouse, as well as other species. The blast software can be found at http://www.ncbi.nlm.nih.gov/BLAST/.

The second of these programs is repeat masking program, such as Repeat Master Web Server found at http://www.repeatmasker.org/cgi-bin/WEBRepeatMasker. This program identifies areas in the designated genetic sequence that are highly repetitive, making them less than ideal locations to build a primer probe. If such areas are found in the designated genetic sequence they are masked by replacing the normal nucleotide designation A, C, G or T with the letter N or X.

Applied Biosystem's FileBuilder software program is then utilized to generate a gene expression assay. The FileBuilder software allows the screening laboratory 20 to identify the location inside the designated genetic sequence that is informative. The insertion of the pgk-neomycin cassette is at the 108^{th} nucleotide of the mutant designated genetic sequence. The FileBuilder software file with the 108th nucleotide designated as the target, is electronically transmitted to Applied Biosystems to generate Assays-by-Design order. Applied Biosystems will use a software program to identify primer and probe sequences that will detect this genetic condition. The software generates the following primers and probe.

| | |
|---|---|
| Forward Primer Seq.: | |
| TTGGCTACCAGTTCCTGAATGG | (SEQ ID NO. 2) |
| Reverse Primer Seq.: | |
| CAGACTGCCTTGGGAAAAGC | (SEQ ID NO. 3) |
| Probe: | |
| CTGCAACCCAGTAATTC | (SEQ ID NO. 4) |

The primers and probes will hybridized or anneal the following areas in the designated genetic sequence.

(SEQ ID NO. 1)

TGCCCAGCGGTCCTATCTAGAGGTCATTCTCTCCACAGAGCGAGTCAA
GAACCACTGGCAGGAAGACCTCATGTTTGGCTACCAGTTCCTGAATGGCT
GCAACCCAGTAATTCTACCGGGTAGGGGAGGCGCTTTTCCCAAGGCAGTC
TGGAGCATGCGCTTTAGCAGCCCCGCTGGCACTTGGCGCTACACAAGTGG
CCTCTGGCCTCGCACACATTCCACATCCACCGGTAGCGCCAACCGGCTCC
GTTCTTTGGTGGCCCCTTCGCGCCACCTTCTACTCCTCCCCTAGTCAGGA
AGTTCCCCCCCGCCCCGCAGCTCGCGTCGTGCAGGACGTGACAAATGGAA
GTAGCACGTCTCACTAGTCTCGTGCAGATGGACAGCACCGCTGAGCAATG
GAAGCGGGTAGGCCTTTGGGGCAGCGGCCAATAGCAGCTTTGCTCCTTCG
CTTTCTGGGCTCAGAGGCTGGGAAGGGGTGGGTCCGGGGGCGGGCTCAGG
G

The genomic DNA nucleotides from the forward primer to the end of the reverse primer and all the bases in between, whether they hybridized to primer probe are not, are known as the target genetic sequence.

(SEQ ID NO. 1)

TGCCCAGCGGTCCTATCTAGAGGTCATTCTCTCCACAGAGCGAGTCAA
GAACCACTGGCAGGAAGACCTCATGTTTGGCTACCAGTTCCTGAATGGCT
GCAACCCAGTAATTCTACCGGGTAGGGGAGGCGCTTTTCCCAAGGCAGTC
TGGAGCATGCGCTTTAGCAGCCCCGCTGGCACTTGGCGCTACACAAGTGG
CCTCTGGCCTCGCACACATTCCACATCCACCGGTAGCGCCAACCGGCTCC
GTTCTTTGGTGGCCCCTTCGCGCCACCTTCTACTCCTCCCCTAGTCAGGA
AGTTCCCCCCCGCCCCGCAGCTCGCGTCGTGCAGGACGTGACAAATGGAA
GTAGCACGTCTCACTAGTCTCGTGCAGATGGACAGCACCGCTGAGCAATG
GAAGCGGGTAGGCCTTTGGGGCAGCGGCCAATAGCAGCTTTGCTCCTTCG
CTTTCTGGGCTCAGAGGCTGGGAAGGGGTGGGTCCGGGGGCGGGCTCAGG
G

A vendor, such as Applied Biosystems, will synthesize these Real-Time primer and probe sequences and send them to the screening laboratory 20.

This large endogenous designated genetic sequence can be truncated for easier data handling. The smaller designated genetic sequence is a subset of nucleotides of the larger designated genetic sequence. The smaller designated genetic sequence contains the informative locations and nucleotides for the assay to be designed. The smaller designated genetic sequence contains the site where the endogenous DNA is disrupted by the pgk-neomycin insert. The 62nd nucleotide is where the disruption occurs in the endogenous DNA.

(SEQ ID NO. 5)

AAGAACCACTGGCAGGAAGACCTCATGTTTGGCTACCAGTTCCTGAAT
GGCTGCAACCCAGTACTCATCAAGCGCTGCACAGCGTTGCCCCCGAAGCT
CCCAGTGACCACAGAGATGGTGGAGTGCAGCCTAGAGCGGCAGCTCAGTT
TAGAACA

Upon identification of the designated genetic sequence and junction site, two other software programs are utilized. The first of these programs is a blast program that identifies homologies between the designated genetic sequence and the endogenous genome of the mouse, as well as other species. The blast software can be found at http://www.ncbi.nlm.nih.gov/BLAST/.

The second of these programs is repeat masking program, such as Repeat Master Web Server found at http://www.repeatmasker.org/cgi-bin/WEBRepeatMasker. This program identifies areas in the designated genetic sequence that are highly repetitive, making them less than ideal locations to build a primer probe. If such areas are found in the designated genetic sequence they are masked by replacing the normal nucleotide designation A, C, G or T with the letter N or X.

Applied Biosystem's FileBuilder software program is then utilized to generate a gene expression assay. The FileBuilder software allows the screening laboratory 20 to identify the location inside the designated genetic sequence that is informative. The insertion of the neomycin cassette in the designated genetic sequence would correspond to a target location of the 62nd nucleotide. The FileBuilder software file with the 62nd nucleotide designated as the target, is electronically transmitted to Applied Biosystems to generate Assays-by-Design order. Applied Biosystems will use a software program to identify primer and probe sequences that will detect this genetic condition. The software generates the following primers and probe.

| | |
|---|---|
| Forward Primer Seq.: | |
| TTGGCTACCAGTTCCTGAATGG | (SEQ ID NO. 6) |
| Reverse Primer Seq.: | |
| CTGTGGTCACTGGGAGCTT | (SEQ ID NO. 7) |
| Probe: | |
| CTGCAACCCAGTACTCAT | (SEQ ID NO. 8) |

The primers and probes will hybridized or anneal the following areas in the designated genetic sequence.

(SEQ ID NO. 5)

AAGAACCACTGGCAGGAAGACCTCATGTTTGGCTACCAGTTCCTGA
ATGGCTGCAACCCAGTACTCATCAAGCGCTGCACAGCGTTGCCCCCGAAG
CTCCCAGTGACCACAGAGATGGTGGAGTGCAGCCTAGAGCGGCAGCTCAG
TTTAGAACA

The genomic DNA nucleotides from the forward primer to the end of the reverse primer and all the bases in between, whether they hybridized to primer probe are not, are known as the target genetic sequence.

(SEQ ID NO. 5)

AAGAACCACTGGCAGGAAGACCTCATGTTTGGCTACCAGTTCCTGA
ATGGCTGCAACCCAGTACTCATCAAGCGCTGCACAGCGTTGCCCCCGAAG
CTCCCAGTGACCACAGAGATGGTGGAGTGCAGCCTAGAGCGGCAGCTCAG
TTTAGAACA

A vendor, such as Applied Biosystems, will synthesize these Real-Time primer and probe sequences and send them to the screening laboratory 20.

A biological sample in the form of a mouse bone marrow is submitted via FedEx (Memphis, Tenn.) overnight delivery to the screening laboratory 20 from the remote user 1. Each sample occupies one well of a 96 well source well container. A lysis reagent (made of 2.5 μl of Nuclei Lysing Solution (Promega Corporation, Madison, Wis. A7943) per sample)) is gently mixed and poured into a 25 ml trough or reservoir and is placed on the deck of a Tecan Genesis Workstation (Research Triangle Park, N.C.). The liquid handler dispenses 150 μl of the lysis reagent into each sample well of the source well container 2. The well plate is then placed in a 55° C. oven for three hours. The well plate is then placed back on the deck of the Tecan Genesis Workstation (Research Triangle Park, N.C.). The liquid handler aspirates 50 μl of each sample and dispenses it into a 384 well primary master well container (Fisher Scientific #NC9134044). Once all of the samples are transferred, the primary master well container is moved to the deck of the Isolation Purification Station 94.

One hundred and twelve microliters of SV Lysis reagent (Promega Corporation, Madison Wis., #Z305X) a chaotropic salt are added to each sample. Next, 13 μl of magnetic particles (Promegal Corporation, #A220X) are added and the well components are mixed. The well plate is then moved into the magnetic field of a magnet where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container is placed on a 384 tip dryer for 11 minutes. Then the microwell container is moved back to the deck of the Isolation Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 O.D. units is acceptable.

The primary master well plate with the isolated DNA is moved to the deck of a Tecan Freedom Workstation. The TaqMan Universal Master Mix, real time PCR primer mixture and Ambion water are placed on the deck as well. The final PCR mixture is made of 1× TaqMan Universal Master Mix (catalog #4326708), 1× real time PCR primer set/probe mix for the designated genetic sequence (Applied Biosystems Assays-by-Design (SM) Service 4331348) and 25% isolated DNA. The Tecan Genesis added the reagents together in the ABI 7900 384 Well Optical Plate. The plate is then sealed with optical sealing tape (ABI, #4311971).

The samples are then placed in an Applied Biosystems SDS HT7900. A standard real time PCR protocol is followed by heating the samples to 50° C. for two minutes then incubated at 95° C. for 10 minutes, followed by thermally cycling the samples 40 times between 95° C. for 15 seconds and 60° C. for one minute. The results are shown in Tables 2 and 3. On average, these results are transmitted to the remote user 1 within twenty-four hours of receiving the biological sample at the screening laboratory 20.

TABLE 13

| | | | | | | Alox5 | |
|---|---|---|---|---|---|---|---|
| Sample | | | Alox5 KO | | | WT | |
| Name | Alox5 KO RCN | | Result | Alox5 WT RCN | | Result | Interpretation |
| 149198 | 0.032 | 0.018 | − | 9.397 | 9.634 | + | Sample is Wild Type |
| 149199 | 0.072 | 0.03 | − | 9.912 | 8.196 | + | Sample is Wild Type |
| 149200 | 0.015 | 0.025 | − | 7.513 | 9.054 | + | Sample is Wild Type |
| 149201 | 0.041 | 0.09 | − | 11.946 | 15.737 | + | Sample is Wild Type |
| 149202 | 0.03 | 0.037 | − | 7.897 | 6.941 | + | Sample is Wild Type |
| 149203 | 0.044 | 0.013 | − | 10.855 | 13.577 | + | Sample is Wild Type |
| 149204 | 0.032 | 0.131 | − | 9.29 | 10.966 | + | Sample is Wild Type |
| 149205 | 2.693 | 2.901 | + | 0.028 | 0.155 | − | Sample is Homozygous |
| 149206 | 2.753 | 2.702 | + | 0.011 | 0.376 | − | Sample is Homozygous |
| 149207 | 3.027 | 3.424 | + | 0.303 | 0.254 | − | Sample is Homozygous |

Although the present invention has been described and illustrated with respect to preferred embodiments and a preferred user thereof, it is not to be so limited since modifications and changes can be made therein which are within the full scope of the invention.

I claim:

1. A method to screen a biological sample for at least one designated genetic sequence within 24 hours of receiving the biological sample at a screening laboratory:
a) acquiring the identity of at least one designated genetic sequence for a plurality of samples;
b) acquiring positive and negative controls for said designated genetic sequence;
c) obtaining means to determine the presence of said designated genetic sequence;
d) receiving said biological sample from a remote user at a screening laboratory;
e) reporting screening result to the remote user within 24 hours of receiving said sample at said screening laboratory.

2. The method of claim 1 wherein said biological sample is murine.

3. The method of claim 1 wherein said biological sample is obtained from a human.

4. The method of claim 3 wherein said designated genetic sequence is SEQ ID NO. 26.

5. The method of claim 4 wherein said means to determine the presence of said designated genetic sequence is forward primer set out as SEQ ID NO. 27, reverse primer set out as SEQ ID NO. 28 and probe set out as SEQ ID NO. 29.


SEQUENCE LISTING (75 sequences)

SEQ ID NO: 1 - DNA, 499 bp, Mus sp.
tgcccagcggtcctatctagaggtcattctctccacagagcgagtcaagaaccactggcaggaagacctcatgtttggctaccagttcctgaatggctgcaacccagtaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggg

SEQ ID NO: 2 - DNA, 22 bp, Mus sp.
  misc_feature (1)..(22): Forward Primer
ttggctaccagttcctgaatgg

SEQ ID NO: 3 - DNA, 20 bp, Mus sp.
  misc_feature (1)..(20): Reverse Primer
cagactgccttgggaaaagc

SEQ ID NO: 4 - DNA, 17 bp, Mus sp.
  misc_feature (1)..(17): Probe
ctgcaacccagtaattc

SEQ ID NO: 5 - DNA, 155 bp, Mus sp.
aagaaccactggcaggaagacctcatgtttggctaccagttcctgaatggctgcaacccagtactcatcaagcgctgcacagcgttgcccccgaagctcccagtgaccacagagatggtggagtgcagcctagagcggcagctcagtttagaaca

SEQ ID NO: 6 - DNA, 22 bp, Mus sp.
  misc_feature (1)..(22): Forward Primer
ttggctaccagttcctgaatgg

SEQ ID NO: 7 - DNA, 19 bp, Mus sp.
  misc_feature (1)..(19): Reverse Primer
ctgtggtcactgggagctt

SEQ ID NO: 8 - DNA, 18 bp, Mus sp.
  misc_feature (1)..(18): Probe
ctgcaacccagtactcat

SEQ ID NO: 9 - DNA, 686 bp, Mus sp.
  misc_feature (561)..(585): n is a, c, g, or t
tatcatgtctcccggctcaagtctgccatcccttcacgttaggaaacagaaagctctagaagctgagctagatgctcagcatttatcagaaaccttcgacaacattgacaacctaagtcccaaggcctctcaccggagtaagcagagacacaagcagaatctttatggtgactatgcttttgacgccaatcgacatgatgatagtaggtcagacaatttcaatactggaaacatgactgttctttcaccatatttaaatactacggtattgcccagctcttcttcctcaaggggaagtttagacagttctcgttctgagaaagacagaagttaggagagagagcgaggtattggcctcagtgcttaccatccaacaacagaaaatgcaggaacctcatcaaaacgaggtctgcagatcactaccactgcagcccagatagccaaagttatggaagaagtatcagccattcatacctcccaggacgacagaagttctgcttctaccaccgagttccattgtgtggcagacgacaggagtgcggcacgaagaagctctgcctnnnnnnnnnnnnnnnnnnnnnnnnncttcactaagtcggaaaattcaaataggacatgctctatgccttatgccaaagtggaatataaacgatcttcaaatgacagtttaaatagtgtcactagta

SEQ ID NO: 10 - DNA, 25 bp, Mus sp.
  misc_feature (1)..(25): Forward Primer
gggaagtttagacagttctcgttct

SEQ ID NO: 11 - DNA, 22 bp, Mus sp.
  misc_feature (1)..(22): Reverse Primer
gtaagcactgaggccaatacct

SEQ ID NO: 12 - DNA, 15 bp, Mus sp.
  misc_feature (1)..(15): Probe 1
ctctctccaaacttc

SEQ ID NO: 13 - DNA, 16 bp, Mus sp.
  misc_feature (1)..(16): Probe 2
tctctctcctaacttc

SEQ ID NO: 14 - DNA, 181 bp, Mus sp.
gttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacag

SEQ ID NO: 15 - DNA, 18 bp, Mus sp.
  misc_feature (1)..(18): Forward Primer
caccgcttccgctaccat

SEQ ID NO: 16 - DNA, 18 bp, Mus sp.
  misc_feature (1)..(18): Reverse Primer
gctccgtcggtggtgaag

SEQ ID NO: 17 - DNA, 20 bp, Mus sp.
  misc_feature (1)..(20): Probe
ctgggtaatgacgtcattct

SEQ ID NO: 18 - DNA, 2205 bp, Mus sp.
gaccggtaacaagtggccgggagcgaacttttgcaaatctcttctgcgccttaaggctgccaccgagactgtaaagaaaagggagaagaggaacctatactcataccagttcgcacaggcggctgaagttgggcgagcgctagccgcggctgcctagcgtccccctccccctcacagcggaggaggggacagttgtcggaggccgggcggcagagcccgatcgcgggcttccaccgagaattccgtgacgactggtcagcaccgccggagagccgctgttgctgggactggtctgcgggctccaaggaaccgctgctccccgagagcgctccgtgagtgaccgcgacttttcaaagctcggcatcgcgcgggagcctaccaacgtgagtgctagcggagtcttaaccctgcgctccctggagcgaactggggaggagggctcagggggaagcactgccgtctggagcgcacgctcctaaacaaactttgttacagaagcggggacgcgcgggtatccccccgcttcccggcgcgctgttgcggccccgaaacttctgcgcacagcccaggctaaccccgcgtgaagtgacggaccgttctatgactgcaaagatggaaacgaccttctacgacgatgccctcaacgcctcgttcctccagtccgagagcggtgcctacggctacagtaaccctaagatcctaaaacagagcatgaccttgaacctggccgacccggtgggcagtctgaagccgcacctccgcgccaagaactcggaccttctcacgtcgcccgacgtcgggctgctcaagctggcgtcgccggagctggagcgcctgatcatccagtccagcaatgggcacatcaccactacaccgacccccacccagttcttgtgccccaagaacgtgaccgacgagcaggagggcttcgccgagggcttcgtgcgcgccctggctgaactgcatagccagaacacgcttcccagtgtcacctccgcggcacagccggtcagcggggcgggcatggtggctcccgcggtggcctcagtagcaggcgctggcggcggtggtggctacagcgccagcctgcacagtgagcctccggtctacgccaacctcagcaacttcaacccgggtgcgctgagcagcggcggtggggcgccctcctatggcgcggccgggctggcctttccctcgcagccgcagcagcagcagcagccgcctcagccgccgcaccacttgccccaacagatcccggtgcagcacccgcggctgcaagccctgaaggaagagccgcagaccgtgccggagatgccgggagagacgccgcccctgtcccctatcgacatggagtctcaggagcggatcaaggcagagaggaagcgcatgaggaaccgcattgccgcctccaagtgccggaaaaggaagctggagcggatcgctcggctagaggaaaaagtgaaaaccttgaaagcgcaaaactccgagctggcatccacggccaacatgctcagggaacaggtggcacagcttaagcagaaagtcatgaaccacgttaacagtgggtgccaactcatgctaacgcagcagttgcaaacgttttgagaacagactgtcagggctgaggggcaatggaagaaaaaaaataacagagacaaacttgagaacttgactggttgcgacagagaaaaaaaaagtgtccgagtactgaagccaagggtacacaagatggactgggttgcgacctgacggcgcccccagtgtgctggagtgggaaggacgtggcgcgcctggctttggcgtggagccagagagcagcggcctattggccggcagactttgcggacgggctgtgcccgcgcgcgaccagaacgatggacttttcgttaacattgaccaagaactgcatggacctaacattcgatctcattcagtattaaaggggggtgggaggggttacaaactgcaatagagactgtagattgcttctgtagtgctccttaacacaaagcagggagggctgggaaggggggggaggcttgtaagtgccaggctagactgcagatgaactcccctggcctgcctctctcaactgtgtatgtacatatatatttttttttaatttgatgaaagctgattactgtcaataaacagcttcctgcctttgtaagttattccatgtttgtttgtttgggtgtcctgccc

SEQ ID NO: 19 - DNA, 22 bp, Mus sp.
  misc_feature (1)..(22): Forward Primer
gagtgctagcggagtcttaacc

SEQ ID NO: 20 - DNA, 18 bp, Mus sp.
  misc_feature (1)..(18): Reverse Primer
ctccagacggcagtgctt

SEQ ID NO: 21 - DNA, 18 bp, Mus sp.
  misc_feature (1)..(18): Probe
aagcactgccgtctggag

SEQ ID NO: 22 - DNA, 1091 bp, Mus sp.
atgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgtaaatgattgctataattatttgatat

SEQ ID NO: 23 - DNA, 27 bp, Mus sp.
  misc_feature (1)..(27): Forward Primer
ttaatccatattggcagaacgaaaacg

SEQ ID NO: 24 - DNA, 22 bp, Mus sp.
  misc_feature (1)..(22): Reverse Primer
caggctaagtgccttctctaca

SEQ ID NO: 25 - DNA, 15 bp, Mus sp.
  misc_feature (1)..(15): Probe
cctgcggtgctaacc

SEQ ID NO: 26 - DNA, 205 bp, Homo sapiens
aaagaagagcagcacgtcatacccaagaccaacatctctcagtgtttcacgctaacccaaggagagacactagcagtcttctctgcaggaccccttgaatttacattgaattccatccccagccgagcaggtgcttaaagtcaacaggggacactccattttcttggaatttcattctggcaaagagggtgtgagcagcaataag

SEQ ID NO: 27 - DNA, 25 bp, Homo sapiens
  misc_feature (1)..(25): Forward Primer
gcaggaccccttgaatttacattga

SEQ ID NO: 28 - DNA, 20 bp, Homo sapiens
  misc_feature (1)..(20): Reverse Primer
tggagtgtcccctgttgact

SEQ ID NO: 29 - DNA, 16 bp, Homo sapiens
  misc_feature (1)..(16): Probe
ccgagcaggtgcttaa

SEQ ID NO: 30 - DNA, 1026 bp, Mus sp.
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 31 - DNA, 24 bp, Mus sp.
  misc_feature (1)..(24): Forward Promer
cgcaaggaatcggtcaatacacta

SEQ ID NO: 32 - DNA, 24 bp, Mus sp.
  misc_feature (1)..(24): Reverse Primer
cacagtttgccagtgatacacatg

SEQ ID NO: 33 - DNA, 16 bp, Mus sp.
  misc_feature (1)..(16): Probe
catggcgtgatttcat

SEQ ID NO: 34 - DNA, 31357 bp, Mus sp.
tataagagtgattggcgtccgtacgtaccctctcaactctaaaactcttgtagtttaaatctaatctaaactttataaacggcacttcctgcgtgtccatgcccgcgggcctggtcttgtcatagtgctgacatttgtagttccttgactttcgttctctgccagtgacgtgtccattcggcgccagcagcccacccataggttgcataatggcaaagatgggcaaatacggtctcggcttcaaatgggccccagaatttccatggatgcttccgaacgcatcggagaagttgggtaaccctgagaggtcagaggaggatgggttttgcccctctgctgcgcaagaaccgaaagttaaaggaaaaactttggttaatcacgtgagggtgaattgtagccggcttccagctttggaatgctgtgttcagtctgccataatccgtgatatttttgtagatgaggatccccagaaggtggaggcctcaactatgatggcattgcagttcggtagtgccgtcttggttaagccatccaagcgcttgtctattcaggcatggactaatttgggtgtgcttcccaaaacagctgccatggggttgttcaagcgcgtctgcctgtgtaacaccagggagtgctcttgtgacgcccacgtggcctttcacctttttacggtccaacccgatggtgtatgcctgggtaatggccgttttataggctggttcgttccagtcacagccataccggagtatgcgaagcagtggttgcaaccctggtccatccttcttcgtaagggtggtaacaaagggtctgtgacatccggccacttccgccgcgctgttaccatgcctgtgtatgactttaatgtagaggatgcttgtgaggaggttcatcttaacccgaagggtaagtactcctgcaaggcgtatgctcttcttaagggctatcgcggtgttaagcccatcctgtttgtggaccagtatggttgcgactatactggatgtctcgccaagggtcttgaggactatggcgatctcaccttgagtgagatgaaggagttgttccctgtgtggcgtgactccttggatagtgaagtccttgtggcttggcacgttgatcgagatcctcgggctgctatgcgtctgcagactcttgctactgtacgttgcattgattatgtgggccaaccgaccgaggatgtggtggatggagatgtggtagtgcgtgagcctgctcatcttctcgcagccaatgccattgttaaaagactcccccgtttggtggagactatgctgtatacggattcgtccgttacagaattctgttataaaaccaagctgtgtgaatgcggttttatcacgcagtttggctatgtggattgttgtggtgacacctgcgattttcgtgggtgggttgccggcaatatgatggatggctttccatgtccagggtgtaccaaaaattatatgccctgggaattggaggcccagtcatcaggtgttataccagaaggaggtgttctattcactcagagcactgatacagtgaatcgtgagtcctttaagctctacggtcatgctgttgtgccttttggttctgctgtgtattggagcccttgcccaggtatgtggcttccagtaatttggtcttctgttaagtcatactctggtttgacttatacaggagtagttggttgtaaggcaattgttcaagagacagacgctatatgtcgttctctgtatatggattatgtccagcacaagtgtggcaatctcgagcagagagctatccttggattggacgatgtctatcatagacagttgcttgtgaataggggtgactatagtctcctccttgagaatgtggatttgtttgttaagcggcgcgctgaatttgcttgcaaattcgccacctgtggagatggtcttgtacccctcctactagatggtttagtgccccgcagttattatttgattaagagtggtcaagctttcacctctatgatggttaattttagccatgaggtgactgacatgtgtatggacatggctttattgttcatgcatgatgttaaagtggccactaagtatgttaagaaggttactggcaaactggccgtgcgctttaaagcgttgggtgtagccgttgtcagaaaaattactgaatggtttgatttagccgtggacattgctgctagtgccgctggatggctttgctaccagctggtaaatggcttatttgcagtggccaatggtgttataacctttgtacaggaggtgcctgagcttgtcaagaattttgttgacaagttcaaggcatttttcaaggttttgatcgactctatgtcggtttctatcttgtctggacttactgttgtcaagactgcctcaaatagggtgtgtcttgctggcagtaaggtttatgaagttgtgcagaaatctttgtctgcatatgttatgcctgtgggttgcagtgaagccacttgtttggtgggtgagattgaacctgcagtttttgaagatgatgttgttgatgtggttaaagccccattaacatatcaaggctgttgtaagccacccacttctttcgagaagatttgtattgtggataaattgtatatggccaagtgtggtgatcaattttaccctgtggttgttgataacgacactgttggcgtgttagatcagtgctggaggtttccctgtgcgggcaagaaagtcgagtttaacgacaagcccaaagtcaggaagataccctccacccgtaagattaagatcaccttcgcactggatgcgacctttgatagtgttctttcgaaggcgtgttcagagtttgaagttgataaagatgttacattggatgagctgcttgatgttgtgcttgacgcagttgagagtacgctcagcccttgtaaggagcatgatgtgataggcacaaaagtttgtgctttacttgataggttggcaggagattatgtctatctttttgatgagggaggcgatgaagtgatcgccccgaggatgtattgttccttttctgctcctgatgatgaagactgcgttgcagcggatgttgtagatgcagatgaaaaccaagatgatgatgctgaagactcagcagtccttgtcgctgatacccaagaagaggacggcgttgccaaggggcaggttgaggcggattcggaaatttgcgttgcgcatactggtagtcaagaagaattggctgagcctgatgctgtcggatctcaaactcccatcgcctctgctgaggaaaccgaagtcggagaggcaagcgacagggaagggattgctgaggcgaaggcaactgtgtgtgctgatgctgtagatgcctgccccgatcaagtggaggcatttgaaattgaaaaggttgaagactctatcttggatgagcttcaaactgaacttaatgcgccagcggacaagacctatgaggatgtcttggcattcgatgccgtatgctcagaggcgttgtctgcattctatgctgtgccgagtgatgagacgcactttaaagtgtgtggattctattcgcctgctatagagcgcactaattgttggctgcgttctactttgatagtaatgcagagtctacctttggaatttaaagacttggagatgcaaaagctctggttgtcttacaaggccggctatgaccaatgctttgtggacaaactagttaagagcgtgcccaagtctattatccttccacaaggtggttatgtggcagattttgcctatttctttctaagccagtgtagctttaaagcttatgctaactggcgttgtttagagtgtgacatggagttaaagcttcaaggcttggacgccatgtttttctatggggacgttgtgtctcatatgtgcaagtgtggtaatagcatgaccttgttgtctgcagatataccctacactttgcattttggagtgcgagatgataagttttgcgctttttacacgccaagaaaggtctttagggctgcttgtgcggtagatgttaatgattgtcactctatggctgtagtagagggcaagcaaattgatggtaaagtggttaccaaatttattggtgacaaatttgattttatggtgggttacgggatgacatttagtatgtctccttttgaactcgcccagttatatggttcatgtataacaccaaatgtttgttttgttaaaggagatgttataaaggttgttcgcttagttaatgctgaagtcattgttaaccctgctaatgggcgtatggctcatggtgcaggtgttgcaggtgctatagctgaaaaggcgggcagtgcttttattaaagaaacctccgatatggtgaaggctcagggcgtttgccaggttggtgaatgctatgaatctgccggtggtaagttatgtaaaaaggtgcttaacattgtagggccagatgcgcgagggcatggcaagcaatgctattcacttttagagcgtgcttatcagcatattaataagtgtgacaatgttgtcactactttaatttcggctggtatatttagtgtgcctactgatgtctccctaacttacttacttggtgtagtgacaaagaatgtcattcttgtcagtaacaaccaggatgattttgatgtgatagagaagtgtcaggtgacctccgttgctggtaccaaagcgctatcacttcaattggccaaaaatttgtgccgtgatgtaaagtttgtgacgaatgcatgtagttcgctttttagtgaatcttgctttgtctcaagctatgatgtgttgcaggaagttgaagcgctgcgacatgatatacaattggatgatgatgctcgtgtctttgtgcaggctaatatggactgtctgcccacagactggcgtctcgttaacaaatttgatagtgttgatggtgttagaaccattaagtattttgaatgcccgggcgggatttttgtatccagccagggcaaaaagtttggttatgttcagaatggttcatttaaggaggcgagtgttagccaaataagggctttactcgctaataaggttgatgtcttgtgtactgttgatggtgttaacttccgctcctgctgcgtagcagagggtgaagtttttggcaagacattaggttcagtcttttgtgatggcataaatgtcaccaaagttaggtgtagtgccatttacaagggtaaggttttctttcagtacagtgatttgtccgaggcagatcttgtggctgttaaagatgcctttggttttgatgaaccacaactgctgaagtactacactatgcttggcatgtgtaagtggtcagtagttgtttgtggcaattattttgctttcaagcagtcaaataataattgctatataaatgtggcatgtttaatgctgcaacacttgagtttaaagtttcctaagtggcaatggcaagaggcttggaacgagttccgctctggtaaaccactaaggtttgtgtccttggtattagcaaagggcagctttaaatttaatgaaccttctgattctatcgattttatgcgtgtggtgctacgtgaagcagatttgagtggtgccacgtgcaatttggaatttgtttgtaaatgtggtgtgaagcaagagcagcgcaaaggtgttgacgctgttatgcattttggtacgttggataaaggtgatcttgtcaggggttataatatcgcatgtacgtgcggtagtaaacttgtgcattgcacccaatttaacgtaccatttttaatttgctccaacacaccagagggtaggaaactgcccgacgatgttgttgcagctaatatttttactggtggtagtgtgggccattacacgcatgtgaaatgtaaacccaagtaccagctttatgatgcttgtaatgttaataaggtttcggaggctaagggtaattttaccgattgcctctaccttaaaaatttaaagcaaactttttcgtctgtgctgacgactttttatttagatgatgtaaagtgtgtggagtataagccagatttatcgcagtattactgtgagtctggtaaatattatacaaaacccattattaaggcccaatttagaacatttgagaaggttgatggtgtctataccaactttaaattggtgggacatagtattgctgaaaaactcaatgctaagctgggatttgattgtaattctccctttgtggagtataaaattacagagtggccaacagctactggagatgtggtgttggctagtgatgatttgtatgtaagtcggtactcaagcgggtgcattacttttggtaaaccggttgtctggcttggccatgaggaagcatcgctgaaatctctcacatattttaatagacctagtgtcgtttgtgaaaataaatttaatgtgttgcccgttgatgtcagtgaacccacggacaaggggcctgtgcctgctgcagtccttgttaccggcgtccctggagctgatgcgtcagctggtgccggtattgccaaggagcaaaaagcctgtgcttctgctagtgtggaggatcaggttgttacggaggttcgtcaagagccatctgtttcagctgctgatgtcaaagaggttaaattgaatggtgttaaaaagcctgttaaggtggaaggtagtgtggttgttaatgatcccactagcgaaaccaaagttgttaaaagtttgtctattgttgatgtctatgatatgttcctgacagggtgtaagtatgtggtttggactgctaatgagttgtctcgactagtaaattcaccgactgttagggagtatgtgaagtggggtaagggaaagattgtaacacccgctaagttgttgttgttaagagatgagaagcaagagttcgtagcgccaaaagtagtcaaggcgaaagctattgcctgctattgtgctgtgaagtggtttctcctctattgttttagttggataaagtttaatactgataataaggttatatacaccacagaagtagcttcaaagcttactttcaagttgtgctgtttggcctttaagaatgccttacagacgtttaattggagcgttgtgtctaggggctttttcctagttgcaacggtctttttattatggtttaactttttgtatgctaatgttattttgagtgacttctatttgcctaatattgggcctctccctacgtttgtgggacagatagttgcgtggtttaagactacatttggtgtgtcaaccatctgtgatttctaccaggtgacggatttgggctatagaagttcgttttgtaatggaagtatggtatgtgaactatgcttctcaggttttgatatgctggacaactatgatgctataaatgttgttcaacacgttgtagataggcgtttgtcctttgactatattagcctatttaaattagtagttgagcttgtaatcggctactctctttatactgtgtgcttctacccactgtttgtccttattggaatgcagttgttgaccacatggttgcctgaattctttatgctggagactatgcattggagtgctcgtttgtttgtgtttgttgccaatatgcttccagcttttacgttactgcgattttacatcgtggtgacagctatgtataaggtctattgtctttgtagacatgttatgtatggatgtagtaagcctggttgcttgttttgttataagagaaaccgtagtgtccgtgttaagtgtagcaccgttgttggtggttcactacgctattacgatgtaatggctaacggcggcacaggtttctgtacaaagcaccagtggaactgtcttaattgcaattcctggaaaccaggcaatacattcataactcatgaagcagcggcggacctctctaaggagttgaaacgccctgtgaatccaacagattctgcttattactcggtcacagaggttaagcaggttggttgttccatgcgtttgttctacgagagagatggacagcgtgtttatgatgatgttaatgctagtttgtttgtggacatgaatggtctgctgcattctaaagttaaaggtgtgcctgaaacgcatgttgtggttgttgagaatgaagctgataaagctggttttctcggcgccgcagtgttttatgcacaatcgctctacagacctatgttgatggtggaaaagaaattaataactaccgccaacactggtttgtctgttagtcgaactatgtttgacctttatgtagattcattgctgaacgtcctcgacgtggatcgcaagagtctaacaagttttgtaaatgctgcgcacaactctctaaaggagggtgttcagcttgaacaagttatggatacctttattggctgtgcccgacgtaagtgtgctatagattctgatgttgaaaccaagtctattaccaagtccgtcatgtcggcagtaaatgctggcgttgattttacggatgagagttgtaataacttggtgcctacctatgttaaaagtgacactatcgttgcagccgatttgggtgttcttattcagaataatgctaagcatgtacaggctaatgttgctaaagccgctaatgtggcttgcatttggtctgtggatgcttttaaccagctatctgctgacttacagcataggctgcgaaaagcatgttcaaaaactggcttgaagattaagcttacttataataagcaggaggcaaatgttcctattttaactacaccgttctctcttaaagggggcgctgtttttagtagaatgttacaatggttgtttgttgctaatttgatttgtttcattgtgttgtgggcccttatgccaacatatgcagtgcacaaatcggatatgcagttgcctttatatgccagttttaaagttatagataatggtgtgctaagggatgtgtctgttactgacgcatgcttcgcaaacaaatttaatcaatttgatcaatggtatgagtctacttttggtcttgcttattaccgcaactctaaggcttgtcctgttgtggttgctgtaatagatcaagacattggccataccttatttaatgttcctaccacagttttaagatatggatttcatgtgttgcattttataacccatgcatttgctactgatagcgtgcagtgttacacgccacatatgcaaatcccctatgataatttctatgctagtggttgcgtgttgtcatccctctgtactatgcttgcgcatgcagatggaaccccgcatccttattgttatacagggggtgttatgcacaatgcctctctgtatagttctttggctcctcatgtccgttataacctggctagttcaaatggttatatacgttttcccgaagtggttagtgaaggcattgtgcgtgttgtgcgcactcgctctatgacctactgcagggttggtttatgtgaggaggccgaggagggtatctgctttaattttaatcgttcatgggtattgaacaacccgtattatagggccatgcctggaactttttgtggtaggaatgcttttgatttaatacatcaagttttaggaggattagtgcggcctattgatttctttgccttaacggcgagttcagtggctggtgctatccttgcaattattgtcgttttggctttctattatttaataaagcttaaacgtgcctttggtgactacactagtgttgtggttatcaatgtaattgtgtggtgtataaattttctgatgctttttgtgtttcaggtttatcccacattgtcttgtttatatgcttgtttttatttctacacaacgctttatttcccttcggagataagtgttgttatgcatttgcaatggcttgtcatgtatggtgctattatgcccttgtggttttgcattatttacgtggcagtcgttgtttcaaaccatgcattgtggttgttctcttactgccgcaaaattggtaccgaggttcgtagtgacggcacatttgaggaaatggcccttactacctttatgattactaaagaatcttattgtaagttgaaaaattctgtttctgatgttgcttttaacaggtacttgagtctttataacaagtatcgttattttagtggcaaaatggatactgccgcttatagagaggctgcctgttcacaactggcaaaggcaatggaaacatttaaccataataatggtaatgatgttctctatcagcctccaaccgcctctgttactacatcatttttacagtctggtatagtgaagatggtgtcgcccacctctaaagtggagccttgtattgttagtgttacttatggtaacatgacacttaatgggttgtggttggatgataaagtttattgcccaagacatgttatctgttcttcagctgacatgacagaccctgattatcctaatttgctttgtagagtgacatcaagtgatttttgtgttatgtctggtcgtatgagccttactgtaatgtcttatcaaatgcagggctgccaacttgttttgactgttacactgcaaaatcctaacacgcctaagtattccttcggtgttgttaagcctggtgagacatttactgtactggctgcatacaatggcagacctcaaggagccttccatgttacgcttcgtagtagccataccataaagggctcctttctatgtggatcctgcggttctgtaggatatgttttaactggcgatagtgtacgatttgtttatatgcatcagctagagttgagtactggttgtcataccggtactgactttagtgggaacttttatggtccctatagagatgcgcaagttgtacaattgcctgttcaggattatacgcagactgttaatgttgtagcttggctttatgctgctatttttaacagatgcaactggtttgtgcaaagtgatagttgttccctggaggagtttaatgtttgggctatgaccaatggttttagctcaatcaaagccgatcttgtcttggatgcgcttgcttctatgacaggcgttacagttgaacaggtgttggccgctattaagaggctgcattctggattccagggcaaacaaattttaggtagttgtgtgcttgaagatgagctgacaccaagtgatgtttatcaacaactagctggtgtcaagctacagtcaaagcgcacaagagttataaaaggtacatgttgctggatattggcttcaacgtttttgttctgtagcattatctcagcatttgtaaaatggactatgtttatgtatgttactacccatatgttgggagtgacattgtgtgcactttgttttgtaagctttgctatgttgttgatcaagcataagcatttgtatttaactatgtatattatgcctgtgttatgcacactgttttacaccaactatttggttgtgtacaaacagagttttagaggtctagcttatgcttggctttcacactttgtccctgctgtagattatacatatatggatgaagttttatatggtgttgtgttgctagtagctatggtgtttgttaccatgcgtagcataaaccacgacgtcttttctattatgttcttggttggtagacttgtcagcctggtatccatgtggtattttggagccaatttagaggaagaggtactattgttcctcacatccctatttggcacgtacacatggactactatgttgtcattggctaccgctaaggttattgctaaatggttggctgtgaatgtcttgtacttcacagacgtaccgcaaattaaattagttcttttgagctacttgtgtattggttatgtgtgttgttgttattggggaatcttgtcactccttaatagcatttttaggatgccattgggcgtctacaattataaaatctccgttcaggagttacgttatatgaatgctaatggcttgcgcccacctagaaatagttttgaggccctgatgcttaattttaagctgttgggaattggtggtgtgccagtcattgaagtatctcaaattcaatcaagattgacggatgttaaatgtgctaatgttgtgttgcttaattgcctccagcacttgcatattgcatctaattctaagttgtggcagtattgtagtactttgcacaatgaaatactggctacatctgatttgagcgtggccttcgataagttggctcagctcttagttgttttatttgctaatccagcagcagtggatagcaagtgccttgcaagtattgaagaagtgagcgatgattacgttcgcgacaatactgtcttgcaagccttacagagtgaatttgttaatatggctagcttcgttgagtatgaacttgctaagaagaatctagatgaggctaaggctagcggctctgccaatcaacagcagattaagcagctagagaaggcgtgtaatattgctaagtcagcatatgagcgcgacagagctgttgctcgtaagctggaacgtatggctgatttagctcttacaaacatgtataaagaagctagaattaatgataagaagagtaaggtagtgtctgcattgcaaaccatgctctttagtatggtgcgtaagctagataaccaagctcttaattctattttagataatgcagttaagggttgtgtacctttgaatgcaataccatcattgacttcgaacactctgactataatagtgccagataagcaggtttttgatcaggttgtggataatgtgtatgtcacctatgctgggaatgtatggcatatacagtttattcaagatgctgatggtgctgttaaacaattgaatgagatagatgttaattcaacctggcctctagtcattgctgcaaataggcataatgaagtgtctactgttgttttgcagaacaatgagttgatgcctcagaagttgagaactcaggttgtcaatagtggctcagatatgaattgtaatactcctacccagtgttactataatactactggcacgggtaagattgtgtatgctatacttagtgactgtgatggtctcaagtacactaagatagtaaaagaagatggaaattgtgttgttttggaattggatcctccctgtaagttttctgttcaggatgtgaagggccttaaaattaagtacctttactttgtgaaggggtgtaatacactggctagaggctgggttgtaggcaccttatcctcgacagtgagattgcaggcgggtacggcaactgagtatgcctccaactctgcaatactgtcgctgtgtgcgttttctgtagatcctaagaaaacgtacttggattatataaaacagggtggagttcccgttactaattgtgttaagatgttatgtgaccatgctggcactggtatggccattactattaagccggaggcaaccactaatcaggattcttatggtggtgcttccgtttgtatatattgccgctcgcgtgttgaacatccagatgttgatggattgtgcaaattacgcggcaagtttgtccaagtgcccttaggcataaaagatcctgtgtcatatgtgttgacgcatgatgtttgtcaggtttgtggcttttggcgagatggtagctgttcctgtgtaggcacaggctcccagtttcagtcaaaagacacgaactttttaaacgggttcggggtacaagtgtaaatgcccgtcttgtaccctgtgccagtggcttggacactgatgttcaattaagggcatttgacatttgtaatgctaatcgagctggcattggtttgtattataaagtgaattgctgccgcttccagcgtgtagatgaggacggcaacaagttggataagttctttgttgttaaaagaactaatttagaagtgtataataaggagaaagaatgctatgagttgacaaaagaatgcggtgttgtggctgaacacgagttcttcacatttgatgtggagggaagtcgggtaccacacatagtccgtaaagatctttcaaagtttactatgttagatctttgctatgcattgcgtcattttgaccgcaatgattgttcaactcttaaggaaattctccttacatatgctgagtgtgaagagtcctacttccaaaagaaggactggtatgattttgttgagaatcctgatataattaatgtgtataaaaagcttggtcctatatttaatagagccctgcttaacactgccaagtttgcagacgcattagtggaggcaggcttagtaggtgttttaacacttgataatcaagatttatatggtcaatggtatgactttggagattttgtcaagacagtacctggttgtggtgttgccgtggcagactcttattattcatatatgatgccaatgctgactatgtgtcatgcgttggatagtgagttgtttgttaatggtacttatagggagtttgaccttgttcagtatgattttactgatttcaagctagagctcttcactaagtattttaagcattggagtatgacctaccacccgaacacctgtgagtgcgaggatgacaggtgcattattcattgcgccaattttaatatactttttagtatggtcttacctaagacctgttttgggcctcttgttaggcagatatttgtggatggtgttcctttcgttgtgtcgatcggttaccattataaagaattaggtgttgttatgaatatggatgtggatacacatcgttatcgcttgtctcttaaggacttgcttttgtatgctgcagaccctgcccttcatgtggcgtctgctagtgcactgcttgatttgcgcacatgttgttttagcgttgcagctattacaagtggcgtaaaatttcaaacagttaaacctggaaattttaatcaggatttttatgagtttattttgagtaaaggcctgcttaaagaggggagctccgttgatttgaagcacttcttctttacgcaggatggtaatgctgctattactgattataattattacaagtataatctacccaccatggtggatattaagcagttgttgtttgttttagaagttgttaataagtattttgagatctatgagggtgggtgtatacccgcaacacaggtcattgttaataattatgataagagtgctggctatccatttaataaatttggaaaggccaggctctattatgaggcattatcatttgaggagcaggatgaaatttatgcgtataccaaacgcaatgtcctgccgaccctaactcaaatgaatcttaaatatgctattagtgctaagaatagggcccgcaccgttgctggtgtctctattctcagtactatgactggcagaatgtttcatcaaaagtgtctaaagagtatagcagctactcgcggtgttcctgtagttataggcaccacgaagttctatggcggttgggatgatatgttacgccgccttattaaagatgttgatagtcctgtactcatgggttgggactatcctaaatgtgatcgtgctatgccaaacatactgcgtattgttagtagtttggtgctagcccgtaaacatgattcgtgctgttcgcatacggatagattctatcgtcttgcgaacgagtgcgcccaagttttgagtgaaattgttatgtgtggtggttgttattatgttaaaccaggtggcactagtagtggggatgcaaccactgcttttgctaattctgtgtttaacatttgtcaagctgtttccgccaatgtatgctcgcttatggcatgcaatggacacaaaattgaagatttgagtatacgcgagttacaaaagcgcctatactctaatgtctatcgtgcggaccatgttgaccccgcatttgttagtgagtattatgagtttttaaataagcattttagtatgatgattttgagtgatgatggtgttgtgtgttataattcagagtttgcgtccaagggttatattgctaatataagtgcctttcaacaggtattatattatcaaaataatgtgtttatgtctgaggccaaatgttgggtagaaacagacatcgaaaagggaccgcatgaattttgttctcaacatacaatgctagtcaagatggatggtgatgaagtctaccttccataccctgatccttcgagaatcttaggagcaggctgttttgttgatgatttattaaagactgatagcgttctcttgatagagcgtttcgtaagtcttgcaattgatgcttatcctttagtataccatgagaacccagagtatcaaaatgtgttccgggtatatttagaatatataaagaagctgtacaatgatctcggtaatcagatcctggacagctacagtgttattttaagtacttgtgatggtcaaaagtttactgatgagaccttttacaagaacatgtatttaagaagtgcagtgctgcaaagcgttggtgcctgcgttgtctgtagttctcaaacatcattacgttgtggcagttgcatacgcaagcctttgctgtgttgcaaatgcgcctatgatcatgttatgtccactgatcataaatatgtcctgagtgtgtcaccatatgtgtgtaattcaccgggatgtgatgtaaatgatgttaccaaattgtatttaggtggtatgtcatattattgtgaggaccataaaccacagtattcattcaaattggtgatgaatggtatggtttttggtttatataaacaatcttgtactggttcgccctacatagaggattttaataaaatagctagttgcaaatggacagaagtcgatgattatgtgctagctaatgaatgcaccgaacgccttaaattgtttgccgcagaaacgcagaaggccacagaagaggcctttaagcaatgttatgcgtcagcaacgatccgtgagatcgtgagcgatcgggagttaattttatcttgggaaattggtaaagtgagaccaccacttaataaaaattatgtttttactggctaccattttactaataatggtaagacagttttaggtgagtatgtttttgataagagtgagttgactaatggtgtgtactatcgcgccacaaccacttataagttatctgtaggtgatgtgttcattttaacatcacacgcagtgtctagtttaagtgctcctacattagtaccgcaggagaattatactagcattcgttttgctagtgtttatagtgtgcctgagacgtttcagaataatgtgcctaattatcagcacattggaatgaagcgctattgtactgtacagggaccgcctggtactggtaagtcccatctagccattgggctagctgtttattattgtacagcgcgcgtggtgtataccgctgctagccatgctgcagttgacgcgctgtgtgaaaaggcacataaatttttaaatattaatgactgcacgcgtattgttcctgcaaaggtgcgtgtagattgttatgataaatttaaggtcaatgacaccactcgcaagtatgtgtttactacaataaatgcattacctgagttggtgactgacattattgtcgttgatgaagttagtatgcttaccaactatgagctgtctgttattaacagtcgtgttagtgctaagcattatgtgtatattggagaccctgcgcagttacctgcaccacgtgtgctactgaataagggaactctagaacctagatattttaattccgttaccaagctaatgtgttgtttgggtccagatattttcttgggcacctgttatagatgccctaaggagattgtggatacggtgtcagccttggtttataataataagctgaaggctaaaaatgataatagctccatgtgctttaaggtttattataagggccagactacacatgagagttctagtgctgttaatatgcagcaaatacatttaattagtaagtttttaaaggcaaaccccagttggagtaacgccgtatttattagtccttataatagtcagaactatgttgctaagagagtcttgggattacaaacccagacagtagactcagcgcagggttctgaatatgattttgttatttattcacagactgcggaaacagcgcattctgtcaatgtaaatagattcaatgttgctattacacgtgctaagaagggtattctctgtgtcatgagtagtatgcaattatttgagtctcttaattttactacactgacgttggataagattaacaatccacgattacagtgtactacaaatttgtttaaggattgtagcaggagctatgtaggatatcacccagcccatgcaccatcctttttggcagttgatgacaaatataaggtaggcggtgatttagccgtttgccttaatgttgctgattctgctgtcacttattcgcggcttatatcactcatgggattcaagcttgacttgacccttgatggttattgtaagctgtttataactagagatgaagctatcaaacgtgttagagcctgggttggcttcgatgcagaaggtgcccatgcgatacgtgatagcattgggacaaatttcccattacaattaggcttttcgactggaattgattttgttgtcgaagccactggaatgtttgctgagagagatggttatgtctttaaaaaggcagccgcacgagctcctcctggcgaacaatttaaacaccttatcccacttatgtcaagagggcagaaatgggatgtggttcgaattagaatagtacaaatgttgtcagaccacctagcggatttggcagacagtgttgtacttgtgacgtgggctgccagctttgagctcacatgtttgcgatatttcgctaaagttggaagagaagttgtgtgtagtgtctgcaccaagcgtgcgacatgttttaattctagaactggatactatggatgctggcgacatagttattcctgtgattacctgtacaacccactaatagttgacattcaacagtggggatatacaggatctttaactagcaatcatgatcctatttgcagcgtgcataagggtgctcatgttgcatcatctgatgctatcatgacccggtgtctagctgttcatgattgcttttgtaagtctgttaattggaatttagaataccccattatttcaaatgaggtcagtgttaatacctcctgcaggttattgcagcgcgtaatgtttagggctgcgatgctatgcaataggtatgatgtgtgttatgacattggcaaccctaaaggtcttgcctgtgtcaaaggatatgattttaagttttatgatgcctcccctgttgttaagtctgttaaacagtttgtttataaatacgaggcacataaagatcaatttttagatggtttgtgtatgttttggaactgcaatgtggataagtatccagcgaatgcagttgtgtgtaggtttgacacgcgtgtgttgaacaaattaaatctccctggctgtaatggtggcagtttgtatgttaacaaacatgcattccacaccagtccctttacccgggctgccttcgagaatttgaagcctatgcctttcttttattattcagatacgccctgtgtgtatatggaaggcatggaatctaagcaggtcgattatgtcccattgagaagcgctacatgcatcacaagatgcaatttaggtggcgctgtttgtttaaaacatgctgaggagtatcgtgagtaccttgagtcttacaatacggcaaccacagcgggttttactttttgggtctataagacttttgatttttataacctttggaatacttttactaggctccaaagtttagaaaatgtagtgtataatttggtcaatgctggacactttgatggccgggcgggtgaactgccttgtgctgttataggtgagaaagtcattgccaagattcaaaatgaggatgtcgtggtctttaaaaataacacgccattccccactaatgtggctgtcgaattatttgctaagcgcagtattcggccccaccccgagcttaagctctttagaaatttgaatattgacgtgtgctggagtcacgtcctttgggattatgctaaggatagtgtgttttgcagttcgacgtataaggtctgcaaatacacagatttacagtgcattgaaagcttgaatgtactttttgatggtcgtgataatggtgctcttgaagcttttaagaagtgccggaatggcgtctacattaacacgacaaaaattaaaagtctgtcgatgattaaaggcccacaacgtgccgatttgaatggcgtagttgtggagaaagttggagattctgatgtggaattttggtttgctgtgcgtaaagacggtgacgatgttatcttcagccgtacagggagccttgaaccgagccattaccggagcccacaaggtaatccgggtggtaatcgcgtgggtgatctcagcggtaatgaagctctagcgcgtggcactatctttactcaaagcagattattatcttctttcacacctcgatcagagatggagaaagattttatggatttagatgatgatgtgttcattgcaaaatatagtttacaggactacgcgtttgaacacgttgtttatggtagttttaaccagaagattattggaggtttgcatttgcttattggcttagcccgtaggcagcaaaaatccaatctggtaattcaagagttcgtgacatacgactctagcattcattcgtactttatcactgacgagaacagtggtagtagtaagagtgtgtgcactgttattgatttattgttagatgattttgtggacattgtaaagtccctgaatctaaagtgtgtgagtaaggttgttaatgttaatgttgattttaaagatttccagtttatgttgtggtgcaatgaggagaaggtcatgactttctatcctcgtttgcaggctgctgctgactggaaacctggttatgttatgcctgtcttatataagtatttggaatcgcctctggaaagagtaaacctctggaattatggcaagccgattactttacctacaggatgtatgatgaatgttgctaagtatactcaattatgtcaatatttgagcactacaacattagcagttccggctaatatgcgtgtcttacaccttggtgccggttcggataagggtgttgcccctgggtctgcagttcttaggcagtggctaccagcgggaagtattcttgtagataatgatgtgaatccatttgtgagtgacagtgtcgcctcatattatggaaattgtataaccttaccctttgattgtcagtgggatctgataatttctgatatgtacgaccctcttactaagaacattggggagtacaacgtgagtaaagatggattctttacttacctctgtcatttaattcgtgacaagttggctctgggtggcagtgttgccataaaaataacagagttttcttggaacgctgagttatatagtttaatggggaagtttgcgttctggacaatcttttgcaccaacgtaaacgcctcttcaagtgaaggatttttgattggcataaattggttgaataagacccgtaccgaaattgacggtaaaaccatgcatgccaattatctgttttggagaaatagtacaatgtggaatggaggggcttacagtctctttgacatgagtaagttccctttgaaagcggctggtacggctgttgttagccttaaaccagaccaaataaatgacttagtcctctccttgattgagaagggcaagttattagtgcgtgatacacgcaaagaagtttttgttggcgatagcctagtaaatgtcaaataaatctatacttgtcgtggctgtgaaaatggcctttgctgacaagcctaatcatttcataaactttcccctggcccaatttagtggctttatgggtaagtatttaaagctacagtctcaacttgtggaaatgggtttagactgtaaattacagaaggcaccacatgttagtattaccctgcttgatattaaagcagaccaatacaaacaggtggaatttgcaatacaagaaataatagatgatctggcggcatatgagggagatattgtctttgacaaccctcacatgcttggcagatgccttgttcttgatgttagaggatttgaagagttgcatgaagatattgttgaaattctccgcagaaggggttgcacggcagatcaatccagacactggattccgcactgcactgtggcccaatttgacgaagaaagagaaacaaaaggaatgcaattctatcataaagaacccttctacctcaagcataacaacctattaacggatgctgggcttgagctcgtgaagataggttcttccaaaatagatgggttttattgtagtgaactgagtgtttggtgtggtgagaggctttgttataagcctccaacacccaaattcagtgatatatttggctattgctgcatagataaaatacgtggtgatttagaaataggagacctaccgcaggatgatgaggaagcgtgggccgagctaagttaccactatcaaagaaacacctacttcttcagacatgtgcacgataatagcatctattttcgtaccgtgtgtagaatgaagggttgtatgtgttgatttgtttttacactattagtgtaataagcttattattttgttgaaaagggcaggatgtgcatagctatggctcctcgcacactgcttttgctgatttgatgtcagctggtgtttgggttcaatgaacctcttaacatcgtttcacatttaaatgatgactggtttctatttggtgacagtcgttctgactgtacctatgtagaaaataacggtcatcctaaattagattggcttgacctcgacccaaagttgtgtaattcaggaaagatttccgcaaagagtggtaactctctctttaggagttttcacttcactgatttttacaattatacgggtgagggagaccaaattgtattttatgaaggagttaattttagtcccagccatggctttaaatgcctggctcatggagataataaaagatggatgggcaataaagctcgattttatgcccgagtgtatgagaagatggcccaatataggagcctatcgtttgttaatgtgtcttatgcctatggaggtaatgcaaagcccgcctccatttgcaaagacaatactttaacactcaataaccccaccttcatatcgaaggagtctaattatgttgattattactatgagagtgaggctaatttcacactagaaggttgtgatgaatttatagtaccgctctgtggttttaatggccattccaagggcagctcttcggatgctgccaataaatattatactgactctcagagttactataatatggatattggtgtcttatatgggttcaattcgaccttggatgttggcaacactgctaaggatccgggtcttgatctcacttgcaggtatcttgcattgactcctggtaattataaggctgtgtccttagaatatttgttaagcttaccctcaaaggctatttgcctccataagacaaagcgctttatgcctgtgcaggtagttgactcaaggtggagtagcatccgccagtcagacaatatgaccgctgcagcctgtcagctgccatattgtttctttcgcaacacatctgcgaattatagtggtggcacacatgatgcgcaccatggtgattttcatttcaggcagttattgtctggtttgttatataatgtttcctgtattgcccagcagggtgcatttctttataataatgttagttcctcttggccagcctatgggtacggtcattgtccaacggcagctaacattggttatatggcacctgtttgtatctatgaccctctcccggtcatactgctaggtgtgttattgggtatagctgtgttgattattgtgtttttgatgttttattttatgacggatagcggtgttagattgcatgaggcataatctaaacatgctgttcgtgtttattctatttttgccctcttgtttagggtatattggtgattttagatgtatccagcttgtgaattcaaacggtgctaatgttagtgctccaagcattagcactgagaccgttgaagtttcacaaggcctggggacatattatgtgttagatcgagtttatttaaatgccacattattgcttactggttactacccggtcgatggttctaagtttagaaacctcgctcttacgggaactaactcagttagcttgtcgtggtttcaaccaccctatttaagtcagtttaatgatggcatatttgcgaaggtgcagaaccttaagacaagtacgccatcaggtgcaactgcatattttcctactatagttataggtagtttgtttggctatacttcctataccgttgtaatagagccatataatggtgttataatggcctcagtgtgccagtataccatttgtctgttaccttacactgattgtaagcctaacactaatggtaataagcttatagggttttggcacacggatgtaaaacccccaatttgtgtgttaaagcgaaatttcacgcttaatgttaatgctgatgcattttattttcatttttaccaacatggtggtactttttatgcgtactatgcggataaaccctccgctactacgtttttgtttagtgtatatattggcgatattttaacacagtattatgtgttacctttcatctgcaacccaacagctggtagcacttttgctccgcgctattgggttacacctttggttaagcgccaatatttgtttaatttcaaccagaagggtgtcattactagtgctgttgattgtgctagtagttataccagtgaaataaaatgtaagacccagagcatgttacctagcactggtgtctatgagttatccggttatacggtccaaccagttggagttgtataccggcgtgttgctaacctcccagcttgtaatatagaggagtggcttactgctaggtcagtcccctcccctctcaactgggagcgtaagacttttcagaattgtaattttaatttaagcagcctgttacgttatgttcaggctgagagtttgttttgtaataatatcgatgcttccaaagtgtatggcaggtgctttggtagtatttcagttgataagtttgctgtaccccgaagtaggcaagttgatttacagcttggtaactctggatttctgcagactgctaattataagattgatacagctgccacttcgtgtcagctgcattacaccttgcctaagaataatgtcaccataaacaaccataacccctcgtcttggaataggaggtatggctttaatgatgctggcgtctttggcaaaaaccaacatgacgttgtttacgctcagcaatgttttactgtaagatctagttattgcccgtgtgctcaaccggacatagttagcccttgcactactcagactaagcctaagtctgcttttgttaatgtgggtgaccattgtgaaggcttaggtgttttagaagataattgtggcaatgctgatccacataagggttgtatctgtgccaacaattcatttattggatggtcacatgatacctgccttgttaatgatcgctgccaaatttttgctaatatattgttaaatggcattaatagtggtaccacatgttccacagatttgcagttgcctaatactgaagtggttactggcatttgtgtcaaatatgacctctacggtattactggacaaggtgtttttaaagaggttaaggctgactattataatagctggcaaacccttctgtatgatgttaatggtaatttgaatggttttcgtgatcttaccactaacaagacttatacgataaggagctgttatagtggccgtgtttctgctgcatttcataaagatgcacccgaaccggctctgctctatcgtaatataaattgtagctatgtttttagcaataatatttcccgtgaggagaacccacttaattactttgatagttatttgggttgtgttgttaatgctgataaccgcacggatgaggcgcttcctaattgtgatctccgtatgggtgctggcttatgcgttgattattcaaaatcacgcagggctgaccgatcagtttctactggctatcggttaactacatttgagccatacactccgatgttagttaatgatagtgtccaatccgttgatggattatatgagatgcaaataccaaccaattttactattgggcaccatgaggagttcattcaaactagatctccaaaggtgactatagattgtgctgcatttgtctgtggtgataacactgcatgcaggcagcagttggttgagtatggctctttctgtgttaatgttaatgccattcttaatgaggttaataacctcttggataatatgcaactacaagttgctagtgcattaatgcagggtgttactataagctcgagactgccagacggcatctcaggccctatagatgacattaattttagtcctctacttggatgcataggttcaacatgtgctgaagacggcaatggacctagtgcaatccgagggcgttctgctatagaggatttgttatttgacaaggtcaaattatctgatgttggctttgtcgaggcttataataattgcaccggtggtcaagaagttcgtgacctcctttgtgtacaatcttttaatggcatcaaagtattacctcctgtgttgtcagagagtcagatctctggctacacaaccggtgctactgcggcagctatgttcccaccgtggtcagcagctgccggtgtgccatttagtttaagtgttcaatatagaattaatggtttaggtgtcactatgaatgtgcttagtgagaaccaaaagatgattgctagtgcttttaacaatgcgctgggtgctatccaggatgggtttgatgcaaccaattctgctttaggtaagatccagtccgttgttaatgcaaatgctgaagcactcaataacttactaaatcaactttctaacaggtttggtgctattagtgcttctttacaagaaattctaactcggcttgaggctgtagaagcaaaagcccagatagatcgtcttattaatggcaggttaactgcacttaatgcgtatatatccaagcaacttagtgatagtacgcttattaaagttagtgctgctcaggccatagaaaaggtcaatgagtgcgttaagagccaaaccacgcgtattaatttctgtggcaatggtaatcatatattatctcttgtccagaatgcgccttatggcttatattttatacacttcagctatgtgccaatatcctttacaaccgcaaatgtgagtcctggactttgcatttctggtgatagaggattagcacctaaagctggatattttgttcaagatgatggagaatggaagttcacaggcagttcatattactaccctgaacccattacagataaaaacagtgtcattatgagtagttgcgcagtaaactacacaaaggcacctgaagttttcttgaacacttcaatacctaatccacccgactttaaggaggagttagataaatggtttaagaatcagacgtctattgcgcctgatttatctctcgatttcgagaagttaaatgttactttgctggacctgacgtatgagatgaacaggattcaggatgcaattaagaagttaaatgagagctacatcaacctcaaggaagttggcacatatgaaatgtatgtgaaatggccttggtatgtttggttgctaattggattagctggtgtagctgtttgtgtgttgttattctttatatgttgctgcacaggttgtggctcatgttgttttaagaagtgtggaaattgttgtgatgagtatggaggacaccaggacagtattgtgatacataatatttcctctcatgaggattgactatcacagcctctcctggaaagacagaaaatctaaacaatttatagcattctcattgctacctggccccgtaagaggcagtcatagctatggccgtgttggtcctaaggctacattggctgctgtctttattggtccatttattgtagcatgtatgctaggcattggcctagtttatttattgcaattgcaagttcaaatttttcatgttaaggataccatacgtgtgactggcaagccagccactgtgtcttatactacaagtacaccagtaacaccgagcgcgacgacgctcgatggtactacgtatactttaattagacccactagctcttatacaagagtttatcttggtactccaagaggttttgattatagtacatttgggcctaagaccctagattatgttactaatctaaacctcatcttaattctggtcgtccatatacttttaaggcattgtccaggcatatgagaccaacagccacatggatttggcatgtgagtgatgcatggttacgccgcacgcgggactttggtgtcattcgcctagaagatttttgttttcaatttaattatagccaaccccgagttggttattgtagagttcctttaaaggcttggtgtagcaaccagggtaaatttgcagcgcagtttaccctaaaaagttgcgaaaaaccaggtcacgaaaaatttattactagcttcacggcctacggcagaactgtccaacaggccgttagcaagttagtagaagaagctgttgattttattctttttagggccacgcagctcgaaagaaatgtttaatttattccttacagacacagtatggtatgtggggcagattatttttatattcgcagtgtgtttgatggtcaccataattgtggttgccttccttgcgtctatcaaactttgtattcaactttgcggtttatgtaatactttggtgctgtccccttctatttatttgtatgataggagtaagcagctttataagtattataatgaagaaatgagactgcccctattagaggtggatgatatctaatctaaacattatgagtagtactactcaggccccagagcccgtctatcaatggacggccgacgaggcagttcaattccttaaggaatggaacttctcgttgggcattatactactctttattactatcatactacagttcggttacacgagccgtagcatgtttatttatgttgtgaaaatgataatcttgtggttaatgtggccactgactattgttttgtgtattttcaattgcgtgtatgcgctaaataatgtgtatcttggattttctatagtgtttactatagtgtccattgtaatctggattatgtattttgttaatagcataaggttgtttatcaggactggtagctggtggagcttcaaccccgaaacaaacaaccttatgtgtatagatatgaaaggtaccgtgtatgttagacccattattgaggattaccatacactaacagccactattattcgtggccacctctacatgcaaggtgttaagctaggcaccggtttctctttgtctgacttgcccgcttatgttacagttgctaaggtgtcacacctttgcacttataagcgcgcattcttagacaaggtagacggtgttagcggttttgctgtttatgtgaagtccaaggtcggaaattaccgactgccctcaaacaaaccgagtggcgcggacaccgcattgttgagaatctaatctaaactttaaggatgtcttttgttcctgggcaagaaaatgccggtggcagaagctcctctgtaaaccgcgctggtaatggaatcctcaagaagaccacttgggctgaccaaaccgagcgtggaccaaataatcaaaatagaggcagaaggaatcagccaaagcagactgcaactactcaacccaactccgggagtgtggttccccattactcctggttttctggcattacccagttccaaaagggaaaggagtttcagtttgcagaaggacaaggagtgcctattgccaatggaatccccgcttcagagcaaaagggatattggtatagacacaaccgccgttcttttaaaacacctgatgggcagcagaagcaattactgcccagatggtatttttactatcttggcacagggccccatgctggagccagttatggagacagcattgaaggtgtcttctgggttgcaaacagccaagcggacaccaatacccgctctgatattgtcgaaagggacccaagcagtcatgaggctattcctactaggtttgcgcccggcacggtattgcctcagggcttttatgttgaaggctctggaaggtctgcacctgctagccgatctggttcgcggtcacaatcccgtgggccaaataatcgcgctagaagcagttccaaccagcgccagcctgcctctactgtaaaacctgatatggccgaagaaattgctgctcttgttttggctaagctcggtaaagatgccggccagcccaagcaagtaacgaagcaaagtgccaaagaagtcaggcagaaaattttaaacaagcctcgccaaaagaggactccaaacaagcagtgcccagtgcagcagtgttttggaaagagaggccccaatcagaattttggaggctctgaaatgttaaaacttggaactagtgatccacagttccccattcttgcagagttggctccaacagttggtgccttcttctttggatctaaattagaattggtcaaaaagaattctggtggtgctgatgaacccaccaaagatgtgtatgagctgcaatattcaggtgcagttagatttgatagtactctacctggttttgagactatcatgaaagtgttgaatgagaatttgaatgcctaccagaaggatggtggtgcagatgtggtgagcccaaagccccaaagaaaagggcgtagacaggctcaggaaaagaaagatgaagtagataatgtaagcgttgcaaagcccaaaagctctgtgcagcgaaatgtaagtagagaattaaccccagaggatagaagtctgttggctcagatccttgatgatggcgtagtgccagatgggttagaagatgactctaatgtgtaaagagaatgaatcctatgtcggcgctcggtggtaacccctcgcgagaaagtcgggataggacactctctatcagaatggatgtcttgctgtcataacagatagagaaggttgtggcagaccctgtatcaattagttgaaagagattgcaaaatagagaatgtgtgagagaagttagcaaggtcctacgtctaaccataagaacggcgataggcgccccctgggaagagctcacatcagggtactattcctgcaatgccctagtaaatgaatgaagttgatcatggccaattggaagaatcacaaaaaaaaaaaaaaaaaaaaaaa

SEQ ID NO: 35 - DNA, 25 bp, Mus sp.
  misc_feature (1)..(25): Forward Primer
tgaacccaccaaagatgtgtatgag

SEQ ID NO: 36 - DNA, 25 bp, Mus sp.
  misc_feature (1)..(25): Reverse Primer
ccatccttctggtaggcattcaaat

SEQ ID NO: 37 - DNA, 16 bp, Mus sp.
  misc_feature (1)..(16): Probe
ctgcacctgaatattg

SEQ ID NO: 38 - DNA, 416 bp, Mus sp.
ggcagctgctgctccgaggcggtcaagagcgccatgagcaccattgacctggactcgctgatggcagagcacagcgctgcctggtacatgcccgctgacaaggccctggtggacagcgcggacgacgacaagacgttggcgccctgggagaaggccaaaccccagaaccccaacagcaaagaaggcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcacc

SEQ ID NO: 39 - DNA, 20 bp, Mus sp.
  misc_feature (1)..(20): Forward Primer
aaaccccagaaccccaacag

SEQ ID NO: 40 - DNA, 23 bp, Mus sp.
  misc_feature (1)..(23): Reverse Primer
tcatccctgctccagtaaattgg

SEQ ID NO: 41 - DNA, 16 bp, Mus sp.
  misc_feature (1)..(16): Probe
ctgcaagccttctttg

SEQ ID NO: 42 - DNA, 792 bp, Mus sp.
cattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctg

SEQ ID NO: 43 - DNA, 15 bp, Mus sp.
  misc_feature (1)..(15): Forward Primer
gggcgcccggttctt

SEQ ID NO: 44 - DNA, 21 bp, Mus sp.
  misc_feature (1)..(21): Reverse Primer
cctcgtcctgcagttcattca

SEQ ID NO: 45 - DNA, 15 bp, Mus sp.
  misc_feature (1)..(15): Probe
acctgtccggtgccc

SEQ ID NO: 46 - DNA, 1288 bp, Mus sp.
  misc_feature (301)..(421): n is a, c, g, or t
  misc_feature (671)..(702): n is a, c, g, or t
  misc_feature (920)..(954): n is a, c, g, or t
ttaaagctcatgcctagacctgatgctatagaaggtgtgctcctcgcttctctgccaatcttaaggtgccctggatggagctgggtgacgtgtttacccttgtagtctgtcctgtctatatgcatggatatgcacagtgcccttgacccaaccctgccaaccaggcacctgcagaaggtgtagatgaccgtcagattgcccagcatccctgtgagtcccaccagcaggatcaccgtgcctagggtatagtgagcatggtctgggacatcgactgtggggaaggggacccaggcagcagccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcccatagaagaaagtgcaagtcttccaaaatttaaccccacgcccatatatgtgtggatactgagcttctaagagggagtgaaaggctcagatggcctgctggaggttaacaggacaaatgcgtgcctgcaggacagagcacagcttgggtgaccttaaggaatgagtagagccaggtcctgggtactgccctcccaacgaatggataccccacagcaagcctccaaggagaacttgcaacccctgtnnnnnnnnnnnnnnnnnnnnnnnnnnnnnnnnaaacgagggaggagaactttccactagaaagagagtttaggttcccccaggctgctgggaggccatttcccccatgaggttagtacacagggactaaggatagctcccagggagaggcaggagtctgcccaatgtcctgcccagcatcccactctggcctgtacaagtccagaagcctagggcatgcctttccccctaggatactcccccaggggatnnnnnnnnnnnnnnnnnnnnnnnnnnnnnnnnnnngaagagcaggtcagcccctgcctttctggttctccagtggtctctgccaacaaagacattgcctgtgccctcttgtctcagccactgtgtagagaaagcttagagaacttcagtgacgctcaaggtccttcgtctaagctcagaccttttctatctccctgttaaaacaagggtggggacaggagtctctgtgtacacacatgctccccaaacttaccgtggggctaacagagagaagctgggctcttacggagacgttctgagtgccgttccaaatgccttgcagggcaggactggttgtgaagctgggatcctgagttaagcttgacaagac

SEQ ID NO: 47 - DNA, 25 bp, Mus sp.
  misc_feature (1)..(25): Forward Primer
tgggtgaccttaaggaatgagtaga

SEQ ID NO: 48 - DNA, 20 bp, Mus sp.
  misc_feature (1)..(20): Reverse Primer
gttctccttggaggcttgct

SEQ ID NO: 49 - DNA, 16 bp, Mus sp.
  misc_feature (1)..(16): Probe
ctgccctcccaacgaa

SEQ ID NO: 50 - DNA, 341 bp, Mus sp.
gtgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatagcttcagctcaagcacgcccag

SEQ ID NO: 51 - DNA, 20 bp, Mus sp.
  misc_feature (1)..(20): Forward Primer
cgaggaccccactaccttct

SEQ ID NO: 52 - DNA, 17 bp, Mus sp.
  misc_feature (1)..(17): Reverse Primer
ccgctcttgggccaagt

SEQ ID NO: 53 - DNA, 16 bp, Mus sp.
  misc_feature (1)..(16): Probe
caggcatcgcgcacat

SEQ ID NO: 54 - DNA, 600 bp, Mus sp.
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctga

SEQ ID NO: 55 - DNA, 17 bp, Mus sp.
  misc_feature (1)..(17): Forward Primer
gcggtgttcgccgagat

SEQ ID NO: 56 - DNA, 20 bp, Mus sp.
  misc_feature (1)..(20): Reverse Primer
gaggccttccatctgttgct

SEQ ID NO: 57 - DNA, 17 bp, Mus sp.
  misc_feature (1)..(17): Probe
gcggtgttcgccgagat

SEQ ID NO: 58 - DNA, 1008 bp, Mus sp.
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 59 - DNA, 25 bp, Mus sp.
  misc_feature (1)..(25): Forward Primer
tgccaacaaggtttttcactagaga

SEQ ID NO: 60 - DNA, 26 bp, Mus sp.
  misc_feature (1)..(26): Reverse Primer
ctcttgatcttccaatacgcaaccta

SEQ ID NO: 61 - DNA, 16 bp, Mus sp.
  misc_feature (1)..(16): Probe
ccacagcgctgagtgc

SEQ ID NO: 62 - DNA, 1139 bp, Mus sp.
gggtgaggctgttgcgacgcctcttatttaaaaaaaaagggaggggtgtctcacactttttctcttgaaggctccttctgtccccctcttttcctttcctgaaaggcacccccttaaacggtcctccgccttcccttctactcccttccttccccacttcggtcctcctcttttcttcgagggcccccacccagccccctccttcggggtcctcctcctcctctgctctttgggcgtccgccccgtcaatcaccgccgtctcggggccccagcccggctcctctccgcctcccgggctctgggagtgcctggggctcccgtctcggccaacctccgctctgtgcagagccggggcgatctgtcagcggagctggccgaggggggcgggggtgggagccgcccgggccgccggggctcgggttaccggtgactgacagcgtctccatggcgaataatttgactcgactattgtctggcgcgggcaggccccgggtcagataacccgaccaatcagggcgcgggccgccgcgcctcatgcccgcttagaataatattattaaaaaagctgcaagcgagctagacgggagggagagcgaacgagcgaggagccggcgagcgagcggcgggcgggcgcggagcatgcggagcggcgccccgggcggcctccgggcttgggcgcgggcgaggcgcgcgggcggcgggggcgcggagctgcgcggggccggcggcgggagcgaggacggatcgttgtgactcaggagtcgctcgggagccggcgcctggccagggggccccgcccgcctgtcggccggccggggccggcggggaggcgcccatgcggggccgcgaagcgcggtgagggcgcgcgcgggcgggcgggcgcgcagccgccaccatgtccatgctgcccaccttcggcttcacgcaggagcaagtggcgtgcgtgtgcgaggtgctgcagcagggcggcaacatcgagcggctgggtcgcttcctgtggtcgctgcccgcctgcgagcacctccacaagaatgaaagcgtgctcaaggccaaggccgtggtggccttccaccggggcaacttccgcgagctctacaaaatcctggagagccaccagttctcgccgcacaaccacgcca

SEQ ID NO: 63 - DNA, 19 bp, Mus sp.
  misc_feature (1)..(19): Forward Primer
gggttaccggtgactgaca

SEQ ID NO: 64 - DNA, 18 bp, Mus sp.
  misc_feature (1)..(18): Reverse Primer
cccgcgccagacaatagt

SEQ ID NO: 65 - DNA, 16 bp, Mus sp.
  misc_feature (1)..(16): Probe
ccatggcgaataattt

SEQ ID NO: 66 - DNA, 1443 bp, Mus sp.
atgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctga

SEQ ID NO: 67 - DNA, 17 bp, Mus sp.
  misc_feature (1)..(17): Forward Primer
ggaacgccggagtacct

SEQ ID NO: 68 - DNA, 17 bp, Mus sp.
  misc_feature (1)..(17): Reverse Primer
actgcacggccgtagtc

SEQ ID NO: 69 - DNA, 18 bp, Mus sp.
  misc_feature (1)..(18): Probe
ctgaggtgctggaggaca

SEQ ID NO: 70 - DNA, 725 bp, Mus sp.
ccatggtgagcaagggcgaggagctgttcaccggggtggtgcccatcctggtcgagctggacggcgacgtaaacggccacaagttcagcgtgtccggcgagggcgagggcgatgccaccctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaag

SEQ ID NO: 71 - DNA, 22 bp, Mus sp.
  misc_feature (1)..(22): Forward Primer
cgtcgtccttgaagaagatggt

SEQ ID NO: 72 - DNA, 20 bp, Mus sp.
  misc_feature (1)..(20): Reverse Primer
cacatgaagcagcacgactt

SEQ ID NO: 73 - DNA, 16 bp, Mus sp.
  misc_feature (1)..(16): Probe
catgcccgaaggctac

SEQ ID NO: 74 - DNA, 8831 bp, Mus sp.
gagcttcgggcgaaggcccgggagcagcggaccgaggctggcgcgatgctgttcccggggagcgcagtcggctaccgttgaggaaggtggagtgaggagtggcccttccagcgccccctatgtacgccttcctgcgctcggggccggtcgccgcgttgcccgcctccgtaccgcccgtgactctcggggcccggagctccggcggcggccggggtcgagtcccgggggaggggaggcgcccgggcggcgcccgagcttgcggccgcggagcgagcgtctggcaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaagcagctacagggaagtattgaagatgagactatgacttctggacagattgacttactagagcgtcttaaagaatttaacttagatagtaatttccccggagtgaaactacgctcaaaaatgtcccttcgctcctacggaagtcgggaaggatctgtatccagccgttcaggagaatgcagtcctgtccccatggggtcattcccaagaagaacatttgtaaatggaagcagagagagtactgggtatctagaagagcttgaaaaagaaagatcattactccttgctgatcttgacaaagaagagaaggaaaaggactggtattatgctcaacttcagaacctcacaaaaagaatagatagcctgcctttaactgaaaatttttccttacagacagacatgacaagacggcagctggagtatgaagcaaggcagatcagggctgcaatggaggagcagcttggcacctgccaggacatggagaagcgtgcacagcgaagaatagccaggatccagcaaatagaaaaggacatactgcgcgtgcgccagcttttacagtcccaggcggcggaagcggagaggtcatctcagagcaggcatgatgctgcctcccatgaagctggccggcagcacgaaggccacggagtggcagaaagcaacaccgcagcctccagtagtggtcagagtccagctacacgtgtggatcacgaaacagccagtgttttgagttctagcggcacgcactctgctcctcgaaggttgacaagtcatctggggacaaaggtggaaatggtgtattccttgttgtcaatgcttggtactcatgataaggacgatatgtcacgaactttgctagctatgtccagctcccaagacagctgtatatccatgcggcagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgagcttactgtgaaacctgttgggagtggcaggaagcccacgaacaaggcatggaccaggacaaaaacccaatgccagctcctgttgagcatcagatctgtcctgctgtgtgtgttctaatgaagctttcatttgatgaagagcataggcatgcaatgaatgaacttgggggactgcaggccattgcagagttattgcaggtggactgtgagatgtatgggcttactaatgaccactacagtgttactttaagacggtatgctggaatggctttgacaaacttgacctttggagatgttgccaacaaggctacgctgtgttctatgaaaggctgcatgagagcacttgtggcccagttaaaatctgagagtgaagacttacagcaggttattgcaagtgttttgaggaatttgtcttggcgagcagatgtaaatagcaaaaagacgttgagagaagttggaagtgtgaaagcattgatggaatgtgctttggaagttaaaaaggaatcaaccctcaaaagcgttttgagtgccttatggaacctgtctgcacactgcactgagaataaggctgacatctgtgctgtggatggagcactggcatttctggttggcaccctcacttaccggagccagacaaatactttagccattattgaaagtggaggtgggatattacggaatgtgtccagcttgatagctacaaacgaagaccacaggcaaatcctaagagagaacaattgcctacaaactttattacagcacttgaaatctcacagcttgacaatagtcagtaatgcatgtggaactttgtggaatctctcagcaagaaatcctaaagaccaggaagccttgtgggacatgggggcagtgagcatgctcaagaacctcattcattccaagcacaaaatgattgccatgggaagtgcagcagctttaaggaatctcatggcaaacagacctgcaaagtataaggatgccaatatcatgtctcccggctcaagtctgccatcccttcacgttaggaaacagaaagctctagaagctgagctagatgctcagcatttatcagaaaccttcgacaacattgacaacctaagtcccaaggcctctcaccggagtaagcagagacacaagcagaatctttatggtgactatgcttttgacgccaatcgacatgatgatagtaggtcagacaatttcaatactggaaacatgactgttctttcaccatatttaaatactacggtattgcccagctcttcttcctcaaggggaagtttagacagttctcgttctgagaaagacagaagtttggagagagagcgaggtattggcctcagtgcttaccatccaacaacagaaaatgcaggaacctcatcaaaacgaggtctgcagatcactaccactgcagcccagatagccaaagttatggaagaagtatcagccattcatacctcccaggacgacagaagttctgcttctaccaccgagttccattgtgtggcagacgacaggagtgcggcacgaagaagctctgcctcccacacacactcaaacacatacaacttcactaagtcggaaaattcaaataggacatgctctatgccttatgccaaagtggaatataaacgatcttcaaatgacagtttaaatagtgtcactagtagtgatggatatggtaaaagaggccaaatgaaaccctcagttgaatcctattctgaagatgatgaaagtaaattttgcagttatggtcagtatccagctgacctagcccataagatacacagtgcaaatcatatggatgataatgatggagaactggatacaccaataaattacagtcttaaatattcagatgagcagttgaactcaggaaggcagagtccctcacagaatgaaaggtgggcaagaccaaagcatgtgatagaagatgaaataaagcaaaacgagcaaagacaagcaagaagccagaacaccagttatcctgtctattctgagaataccgatgacaaacacctcaaattccaaccacattttggacaacaagaatgtgtttccccatataggtcaaggggaaccagtggttcagaaacaaatcgaatgggttctagtcatgcaattaatcaaaatgtaaaccagtctctgtgtcaggaagatgattatgaagatgataaacctaccaactacagtgaacgttattctgaggaagaacaacatgaagaagaagaagagagaccgacaaattatagcataaaatataatgaagagaaacatcatgtggatcagcctattgattatagtttaaaatatgccactgacatttcttcctcacaaaaaccatcattttcattctcaaagaattcatcagcacaaagcactaaacctgaacatctctctccaagcagcgagaatacagctgtacctccatctaatgccaaaaggcagaatcagctgcgtccaagttcagcacaaagaaatggccagactcaaaaaggcactacttgcaaagtcccctccatcaaccaagaaacaatacagacttactgcgtagaagacaccccaatatgtttttcaaggtgcagttcattatcatcactgtcatcagctgacgatgaaataggatgtgatcagacaacacaggaagcagattctgctaatactctgcagacagcagaagtaaaagagaatgatgtaactcggtcagctgaagatcctgcaactgaagttccagcagtgtcccagaatgctagagccaaacccagccgactccaggcttctggcttatcttcagaatcaaccaggcataataaagctgttgagttttcttcaggagccaagtctccctccaaaagtggtgctcagacacccaaaagtcccccagaacactatgtccaggagactccgctcgtattcagcaggtgtacttctgtcagctcccttgacagttttgagagtcgctccattgccagctctgttcagagtgagccatgtagtggaatggtgagtggcatcataagccccagtgaccttccagatagtcctgggcagaccatgccaccaagcagaagcaaaacccctccacctcctccacagacagtgcaggccaagagagaggtgccaaaaagtaaagtccctgctgctgagaagagagagagtgggcctaagcagactgctgtaaatgctgccgtgcagagggtgcaggtccttccagacgtggatactttgttacacttcgccacagaaagtactccagacgggttttcttgttcctccagcctaagtgctctgagcctggatgagccatttatacagaaagatgtagaattaagaatcatgcctccagttcaggaaaacgacaatgggaatgaaactgaatcagaacagcctgaggaatcaaatgaaaaccaggataaagaggtagaaaagcctgactctgaaaaagacttattagatgattctgatgacgatgatattgaaatattagaagaatgtattatttcagccatgccaacaaagtcatcacgcaaagccaaaaaactagcccagactgcttcaaaattacctccacctgtggcaaggaaaccaagtcagctacctgtgtataaacttctgccagcacagaataggctgcaggcacaaaaacatgttagctttacaccaggggatgatgtgccccgggtgtactgtgtagaagggacacctataaacttttccacagcaacgtctctaagtgatctgacaatagagtcccctccaaatgaattggctactggagatggggtcagagcgggtatacagtcaggtgaatttgaaaaacgagataccattcctacagaaggcagaagtacagatgatgctcagcgaggaaaaatctcatctatagttacaccagacctggatgacaacaaagcagaggaaggagatattcttgcagaatgtatcaattctgctatgcccaaaggaaaaagccacaagcctttccgagtgaaaaagataatggaccaagtccaacaagcatcctcgacttcatctggagctaacaaaaatcaagtagacactaagaaaaagaagcctacttcaccagtaaagcccatgccacaaaatactgaatatagaacgcgtgtgagaaagaatacagactcaaaagttaatgtaaatactgaagaaactttctcagacaacaaagactcaaagaaaccaagcttacaaaccaatgccaaggccttcaatgaaaagctacctaacaatgaagacagagtgcgggggagcttcgccttggactcaccgcatcactacacccctattgaggggacgccgtactgcttttcccgaaatgactccttgagttctctggattttgatgatgacgatgttgacctttccagggaaaaggccgagttaagaaagggcaaagaaagcaaggattccgaagccaaagttacctgccgcccagaaccaaactcaagccagcaggcagctagtaagtcacaagccagtataaaacatccagcaaacagagcacagtccaaaccagtgctgcagaaacagcccactttcccccagtcctccaaagacggaccagatagaggggcagcaactgacgaaaaactgcagaattttgctattgaaaatactccagtttgcttttctcgaaattcctctctgagttcccttagtgacattgaccaggaaaacaacaataacaaagaaagtgaaccaatcaaagaagctgaacctgccaactcacaaggagagcccagtaagcctcaggcatccgggtatgctcccaagtccttccacgtcgaagacacccctgtctgtttctcaagaaacagctctctcagttctcttagcattgactctgaggacgacctgttacaggagtgtataagttctgccatgccaaaaaagaaaaggccttcaagactcaagagtgagagcgaaaagcagagccctagaaaagtgggtggcatattagctgaagacctgacgcttgatttgaaagatctacagaggccagattcagaacacgctttctcccccgactcagaaaattttgactggaaagctattcaggaaggcgcaaactccatagtaagtagtttgcaccaagctgctgcagccgccgcgtgcttatctagacaagcgtcatccgactcagattccattctgtcactaaagtccggcatttctctgggatcgccttttcatcttacacctgatcaagaggaaaagccattcacaagcaataaaggcccaagaattctcaaacctggagagaaaagcacattagaagcaaaaaaaatagaatctgaaaacaaaggaatcaaaggcgggaaaaaggtttataaaagcttgattacgggaaagattcgctccaattcagaaatttccagccaaatgaaacaacccctcccgacaaacatgccttcaatctcaagaggcaggacgatgattcacatcccagggcttcggaatagctcctctagtacaagccctgtctctaagaaaggcccacccctcaagactccagcctctaaaagccccagtgaagggccgggagctaccacttctcctcgaggaactaagccagcaggaaagtcagagcttagccctatcaccaggcaaacttcccaaatcagtgggtcaaataaggggtcttctagatcaggatctagagactccactccctcaagacctacacagcaaccattaagtaggccaatgcagtctccagggcgaaactcaatttcccctggtagaaatggaataagccctcctaacaaactgtctcagctgcccagaacatcatctcccagtactgcttcaactaagtcctccggttctgggaaaatgtcatatacatccccaggtagacagctgagccaacaaaatcttaccaaacaagcaagtttatccaagaatgccagcagtatccccagaagtgagtcggcatctaaaggactgaatcagatgagtaacggcaatgggtcaaataaaaaggtagaactttctagaatgtcttcaactaaatcaagtggaagtgaatcagacagatcagaaaggcctgcattagtacgccagtctactttcatcaaagaagccccaagcccaaccctgaggaggaaactggaggaatctgcctcatttgaatccctttctccatcttctagaccagattctcccaccaggtcgcaggcacagaccccagttttaagcccttcccttcctgatatgtctctgtccacacatccatctgttcaggcaggtgggtggcgaaagctcccgcctaatctcagccccactatcgagtataatgacggaaggcccacaaaacggcatgatattgcacgctcccattctgaaagtccttccagactaccaatcaaccgggcgggaacctggaagcgtgaacacagcaaacattcctcgtcccttcctcgagtgagtacttggagaagaactggaagctcatcttctattctttctgcttcatcagagtccagtgaaaaagcaaaaagtgaggatgaaaggcatgtgagctccatgccagcacccagacagatgaaggaaaaccaggtgcccaccaaaggaacatggaggaaaatcaaggaaagtgacatttctcccacaggcatggcttctcagagcgcttcctcaggtgctgccagtggtgctgaatccaagcctctgatctatcagatggcacctcctgtctctaaaacagaggatgtttgggtgagaattgaggactgccccattaacaaccctagatctggacggtcccccacaggcaacacccccccagtgattgacagtgtttcagagaagggaagttcaagcattaaagattcaaaagacacccatgggaaacagagtgtgggcagtggcagtcctgtgcaaaccgtgggtctggaaacccgcctcaactcctttgttcaggtagaggccccagaacagaaaggaactgaggcaaaaccaggacagagtaacccagtctctatagcagagactgctgagacgtgtatagcagagcgtacccctttcagttccagtagctccagcaagcacagctcacctagcgggactgttgctgccagagtgacaccttttaattacaaccctagccctaggaagagcagcgcagacagcacttcagcccggccgtctcagatccctacgccagtgagcaccaacacgaagaagagagattcgaagactgacagcacagaatccagtggagcccaaagtcctaaacgccattccgggtcttacctcgtgacgtctgtttaa

SEQ ID NO: 75 - DNA, 52138 bp, Mus sp.
  misc_feature (32166)..(33122): n is a, c, g, or t
  misc_feature (38299)..(38398): n is a, c, g, or t
gttccagacagtcccacaggtgcagattaggagtcgcccactcgggccactactttctaaggctggttcccagtaccactaaccatttcccccaagtttgctcccaccccgcgcctcccagtaccttgcccagagagagaaggtttactcattttgtgaagaaaccaacattcaagtttccttggggtccaccgtggagctacaggtactctccttgtgggcttcagaccccctgctctaagtgtaactattgcatacgctgtgtgctgcaactgaatgaggacacaggtagttcttgagctgacagtgagggacactgaaacaggcaagaagccataaagatggaagaaataagggattaattgactaatgaaaacaaaaatgcatgagggataaaagctaggtagagatggggcagaggaacaagggcttcagcctatcagagatcactgtccctccagtgccacaggagagaaggatgcgttggaaggtggggtcctgggactggccagagacaggggcggagccagcgcctgaaggcagggccggtcgcagggtggagccagacccaagcgcaaggctggcccgctgctggccactgtggcagggagctgccgcgagtgacagggtcaagaagttggtgggctgccacgccgagcttcgcgggctcctgctcccacaccagcagcactcacttgcccggagtcatgccctcctacacggtcaccgtggccaccggcagccagtggttcgcgggcaccgacgactacatctacctcagcctcattggctctgcgggctgtagcgagaagcatctgctggacaaggcattctacaatgacttcgaacggggcgcggtgagaatgcgcgcttgggaccgacggctggcagcaaagagcgggagggcggcggggcaggacaggcaggcacctgagaactgtctgtcccagcgcgctcgtgaccctatgtgagcgcatctggggatctgatgcagcgccagagtcggtgcatgccagggcaagcgaggcaccctagacttcctgatgactcgggtatcttaagggacaaatgacttccaatgtgggggactgatgtggctggcctcttatgtgagagatggcacaaactttcaccaacaggacccagaatttgtgagaggccccttcacctcgggagttctgaggtcctagtgccccagagtccagtaccactgcaaagcatagaaagccctgttcgacagctagccattttgtgtagacagtgtaagtccagggtaagtcagagatccaggatcgggagtcaacttgggcataatcactcttctatccctactgtggaccttcgtttaccaaactaaaaattggttaggtttaatcttacccatgagtcatgagggagcccagtcccatttgggggctaggaatgagtccagggattgccccccacacttaggtaccctacatttcctgcactcagtcccagtggaggaatgtaaaggaaggaaggtctggcccgagcagcctttggaaagactgtcagcagggtgcgtgcaagaggacacttcctccctgagtattagcttctgaaggaaaaaaggaagaaagatttcctttcttccctcctaatacagactttggagtctggcagccccacaggcactgtgggaggccattccgtttggatgctgctggtgtgtaaagtctagccccatgactttctcttaacacaggcaggtcatggttttcagtgacccactaagtgcctagtacatcagacttgctcagtaagtgagtctgtaatagacaggcacgctgcagacccttggggtgggggtggggggttcctcttccttctactacctccaggtctaaactaggcttaggtttgattttaccaggctgccttcttatcatttagtttactattgagtcgacccacatgaacttgcttagaattaaccatttgtgataagcacaatgacaatttcatatgcttccactagatagatctgttcagcctaaaccaaggaaatgatagttaagcccttttctgggtcccaaactctaagcacgacgcttacattcctatcctgggacctggtactttgccctgattagcaagcttctaaccaggcttgaacaagcaagcgtgggtgttgacctcagaatggcatcttttgctcagtttcaccagagctggccagagaatggtgctgccaaagaccaagcatggcatccgtttgggatgctgacaccctgtgcaacctccaaagcacttgtgtttattttcagtaactggggcttctcccagtatacagggggaggaagagaggacagaatgcttcctctttataaatggactacagggggccttctccacaaatctagctatcagtgggttcagtctaggtgcagcacaggacaccttatgtgtcatttcctccaggaggagaatggcaatgtggccatcataaatgactggacaggaagtaagaggcctgtcctgttcatcatttgcctttctgtcctgcctcccaacctgaaaagtcattcaggtgacattaatttaacaccttagcaagaaccccagaggcaaatttcaggagagatttgcatacatatttccattgtggggagggaaccacagctcaagtgaactgctgtcttctggctggatgcaaaaagagccttaaaaaaagaaaaagaaaacagcctttgagaaagttcctgttgatgcaaagttactccatactttgtcttgcacagtccagagccacttcctcattctgtggccagtgtatctttaaagtccagatgtcccttttgggtaaaggtagaaaagaaaccttaagggaagtgttagctaagaagataaggttatcccactgttcttagaaaagtgccacattgtttccatgaatagcagccaccgtgaaggccggccagccacttccagcaccccttcctaatgtacgaccgaataatggtcagtgctaacctgttttaatacattccactattgcccatcccccatgattcccacatgagttctgtgtagcgattcaggtcaggagcctttgcagatgggattctcctgagtgtcaggtgtcaggcatcagggtttccctgttgagaacccagacggaacagaatggctttgttccataagctagtcctacgtggcacagctctaacaaaccttgaaatcttgagtgcacattagttggctactttaactgctcaacagtactttgaaaagctaaggataggctgtcctgctgagtatatggttcttgaacatattcagtacatgtaaattcatcttacaggtaatacgcttcttaaatacatctaacaaatattcttatatttatagagagtaaattctatagtacctcttcatgaggaagagaatactgatcgggaacaaacatttctttcttcatcagccttcatatactagttctctctcactatccatctcccctacctctgcccttcgtcctcctcttcttccttcctttatcccccaatggtcaatttctctcctgctgctcaccccccaatctctccttccttttctactttaatgccccttacctctctccttcagcttctcctaactgcaacctcactttcccacttcggtccctacatcctcttgactactgctcccagtgttctcattctctgcaacttttgctcttcttattgctgcctttggtctctcattccctctgccccagtctctcttttgccctccagcctcccacccccttctctgctctacagcctcttcctcctcccaccagtttctcataccccgtccaagatggacagccaagttggccatgcggtcgctccagaagagatccattttctcttccacccatccacagagaggtgatataactgaaggtggacaatgcttgaggaatatagagttgtcctctggcagagatggaagtgtaaatgaatagttgagtgtttataaaatggtgatagagagctcagaggctgatcaacaccactttgctgagatatgcatgtaccacatgatgatgtttgtgtcacaacagtccacgtatatgatggtgtccttgtaagattatgacagagctgaaatttcatttttcctggaatattcagtatgtttaaacacataaatacttattatatgttacctaatgtattcaatacagcaatagtcagtacatatatattacttaggagcaattggctataccatgtagcctgggtgtgcagtatgatatatatctatgttcatgtaaatatacactatgacagaattgccttatacttcattttctagaatgtacccctgtcactgatgtatgattgtgtttggatactttaaaactaaatatacaacagagttgtcctgacttgggctaatgaactacattcttcttgtgtctctgaccaatgcagtggcatctctgtcttgagagctgtgttctagccatgtcctcctctgtgtggattccctaaattgagaatattgttctgccaactccctgtatctgaaatctcctgcccactcctctctgctctctgtacctctgaggtggtcagtcactcactaacacatcgatacctgttctctgagtctcagatacacctgtaaaatggagtgactgtccacatcaacatttacatgtgcagcattccctgagtacctggatcaagccccctttatagtacccaccataaagaactcagtatgtcaaggacatagacaacaaatgaagtgtcgtcccaacacactcaaccataaacaaggacagaaaaggtcagcacatttggtggctccacatgcccttctgaggccttctttttgtgagacaccacctatcaccctgtattttcctagagggaagcctctctctgctttctgtgatcttgttctaaatacactctggaactctagacccacaaacaacatactttatctctaagacttccggggttctaactgaacccttcaactcttgtttatctcctgtcacatccaggcctgagggcagagcagcaaggtctctgggttgacaagagataaaccagactacaggcctgacctccatcttctttggaagatcctactgattaagtccctctaagattgcacaatggtctatttcagtgcagttctgaggcccccagccaggtggagttaaggacccatttcctgtgactatactataggtgtgctaaagggtcagcctcccctcccccagaaaagctactgtttttgtatgtctgtcttttgtttgttttgtttgtttgcttgcttgcttgcttgcttgcttgctttgtttggtttggtttaatttagtttggttggatttttttgagacaggatttctctgtctagccctggctgtcttagaattacagctctgtagaccatgccgccctcacactcacagagattcatctgtctctgcctcccaagtgctgggattcaaggcatgcaccaccaccacccagctgctcagtgtctttcttacaggtcaccagttgtagctgcatagatggcctcaaagtacatggctttctcctcggatctttctcattactttagaatgggtgttttctcttcattcactcctgatgtcatctttccaaccctcaagaccgtcctctctagtctctcaatgccacaggaaaaccacttcctctgaagcagctgctctggggctgggtccaggcattcttggtggctgccctttggtcttgaccacaggctgacttccctctcctctgcatgcactataagtcccacccactgtcccttgtccttacaagagttctccatccacccagtccagggcttaaacacatagagtgtttctctcactccacgggcacttggagctatgaatgaattgcagtgcaaagggttggctcaggctggtcgtagctagttctaatatatatatatatacctagtggactatgcccttggagaaggtagctactcaagtgtcccgccactctgatttctcaatgatgctgcaagccagctgttatctcttcttaaaagatgaagaaacacagctggggtgatactcaggcagtcaagtgttcaccatgcaagcacaaggacccaagtttgatccttagaccccatattttttaaaagccagtaatcctggggctacattataattctagtcctgagacaaaggagacaggtggatctctgaggttttctggctagccagcctagcctagttagtcagttctaggccagtaagagacattgatttggtgtagggagtgatagtgcctgaggaatgacatccaaggttgcccactgtcctacacacacacacacacacacacacacacacacacacacacacacacatctcctcatatgtacatgtgtgagactgaacaatattccattgcattcatatacactatattctctgtactcattcatatgaatactcattcatatatctttagccaagattcttaaggaatctcttcattaatttaggtagactcacctcctgcaagaccctggaccctaactttacacagatgcggctccagtgacacaggaatgtcttcagctcaaaaactgatcacatagaaatagagacaagagtcagtcagtagcatccaggaaggagtaggggtggtggtgttgagaggaggcaccaaaataaagttagataggagacatatgctttaatttttcatggcacagtacagtgacaataactcataatgatttttgtatatttcaaagtctgaagagaaaatttttaatgtttcttagatgagtatttgaggtaacagaaatgtcaattaccctggtttgccagtgttttttctatacttatattgagaggtcatattgtacttcacaaacttatgattatcatcagctataaaaattaattatatagaaagaaaaactcccttgtcccattttagataagtcaagcttaggaattaaattagcagaagccaaaaataataggagaaaagtggacaaatttgcctaatgcagcaatgtgtgtcatatgttacttaagaccaaaagaagactcacaaaggcagtgggagtccacagctcacacactaaattgtatgtaaaacatgtgataaagagagttgggctagggaagtcagatggagtgactgtggctgtttgagatggccaaccgggtctaactgtacaggtttctgctttccttctaaggaagacctcttcacacgaaggcagccattagaatgatcttgaactgccattttctatcacttaactcaagtatctgctgtgctagagcagcacgttgaggaaggagggttgtccacagcatcagtattcaggcccaggaggcagtggtggttgattgtcctgttttcctgcccaggtggactcctacgatgtcaccgtggatgaggagctgggcgagatctacctagtcaaaattgagaagcgcaaatactggctccatgatgactggtacctgaagtacatcacactgaagacaccccacggggactacatcgagttcccatgttaccgctggatcacaggcgagggcgagattgtcctgagggatggacgtggtaagctgctcggacccctgacactcgtaggcttcctggaaactagaaagtctccccttctcagagagctctatttctgggcgtgagaattccctcttgggtagagcacacctctgtatcttgttccctcatgggagatgagatcttcctctgctctaacgagctctagagtggagctaatagcctgaggagtcataccaactatggcttccctctgcacactgtcctggaaaggtgtggaaacaggccactaggtagtgagggcctcttaacaggcacccgtgacaagaatcctgcaaatggggagcgctgctagttagcacatgacctcttaagccaaattctctggctgtcctctctaagccaggggagtggggaggtgttcagggccataggacccctcctgaaggcctggacgctaaaggtttggactaagattgccaatataatgccagctctaggggccagaggcaagctggagttcactaccccctgttttacttgtttgctttgaaacaaagcctcagtttcactacatagctcaggctggccttgaactttcagttctgctgatttagcctcccaagatcctagatatgcagggtccaacttggtacttccataagaggccattctttttcagaccccactcccacagagggtcagagaaggaagatgtcaaagatcagctgtttgttgtttgcactcccccctcacctccctctgagtgacacccctcaccccctctgagtaacaccatttgcacactagctgcttatatgaatgggtcaaactaagccttggcaaacttctatcttgtagtcgtaacccttgacttcgctttcagtctgccgacagccactgcagtgaggatgactgctgatgcttatgacagcaatgagcatctctcagataaggatcttctgccgttgttcatcttcacagttgggaggggcacacaagatgagattgataattaaaccaccaccattttgatagaaaaataagacagaggggaagaagaaggggaagaagaggaagaaagaggaggagaaagaaggaggaagaaggaggaggaaggaggaggaaggaggaggaaggaggaggaaggaggaggaaggaggagaaggaggaggaggaggaagaggaagaagaagaagaagaagaagaagaagaagaagaagaagaagaagaagaagaagaagaagaagaagaagaagaaggaagaagaagaagaagaagaagaagaagaagaagaagaagaagaagaagaagaagaagaagaaggaagaagaagaagaaggaagaagaaggaagaagaagaagaagaagaagaagaagaagaagaagaagaagaagaagaagaagaagaagaagaagaagaagaagaagaaggaagaggaagaggaagaggaagaggaagaggaggaagaagaagaagaagaagaagaagaagaagaagaagaagaagaagaagaagaagaagaagaagaagaagaagaagaagaagaagaagaagaagaagaagaagaagaagaagaagaagaagaagaccctctagggtcctttggctcactgtccttgatgtttaacttgacttacaccaaattctgggaaaccttttgacttggatacttgggtttagaaagatttttatcacccctcccccaagctcagtgggaacccccgcctccactcacccactcacccacagatcatttaagtcatcctgtggctagaacaaggaagggtgcttctcttaacccagggtttagcaggccttagaatctattttctgtctgctccacccaagatctcagtggggaaatctttcctgcctacagagtctgtagctttgcacagcagcatgcttgataacgcaggcagattggaatgatttgaggaggtgttgagagagggacatcaagaaggtgtgaaaatgtccagaggggccacttctgtatgtagggtcggcagcttggccaatcctgatgagctgggagagaagaagagtttgagaacaatgtagtaagtggcattttaaagataacctctatcttatgagtcaatgcagagctgtgacctcagtagagaagtggacatccctggagatgaggaagagcagaatggagaagacataacagccccaccctcaccacacacacctcccctctgctctgtccccagagctaaatctaatgggaagtccattgatgatatctattcttatcagctttattgacaagataaaatggaaggggggtcttcagagacacaatctggctgacaacttccattaaccatggctggtaaccgaccatcatagctttaatcagtcatttaatttggctagtgatcatgtaggtctagaaccttggaagggatctgccaagtacttggtctgtgtcctgtgtgccattaactcaatggactgcaagcttcaccttgaagatggttcttcttcatggccttcatctctgcataacaccccatttttaaggagtctcacaacatgagggactcagggtagcctcatttcttacatagtagctcacttcttttttccttcttctctttttattagatattttctttatttacatttcaaatgctatcccaaaagtcccctatactctccccccccccgccctgctcccctacccacccactcccacttcttggcgctggcgttcccctgtactgaggcatataaagtttgcaagaccaaggggcctctcctcccaatgatgaccagctaggccatcttctgctacatatgcagctagagacacgagctttggcggtattggttagttcatattgttgttcctcctatagggttgcagaccccttggtagctcacttctaagagcaaatattccaagaaacagagaatagatgctgccaggatccaggcctgtgcctggaacaagtatagtgtaactccaccacccagccttgaggggaagaacaccaaaatatctcaatttaaagaatgccagaggatttgggaccataattaacctacatggcacaacaaccaacgccttgaacaatgtggcaagaagagggcccattccacaagtgaccccatttcccaccactgctgggggcctgcttagccttagaggagtgagctgaggtgggacacagagttggatgaaggccaaagactgatgatctctggccttgaaaccctctctcttgaaactcagtacagttagtactatcaataacaagttcaaagcagtatttcatattcattataagttcaaagcagcagtttttacatggcctggtcttatcatagtgcacacctgtggcttcatccttggacctgaatgtatttccttgaacacaaatttgtctcaagccttatttctctttttagtgtagttatgaaagctcactgtatgtcttattatgcagccttttcttgctattatgaggagtgggtacattctattcttgattctaactttattatatctttctggcaaaacaactttcttggaaccttcttcctataattatttaagttgaatcatgagttctatataaaatataaaatcattaattcatttaaacagcattaattcataaaagtccatcttcatgttgatctgcaggaaatttgcccaatagcatgcactgatgcccagaagttgttagactgtataatagtgatatgagtgacatatgattagcaagaagccattctgggatgagtcttttgggacttcacctcccagagctcacccatcgcaaaccatgcaaaaaaaccagtgagccatctccaggaaagtcatccaatagccttagcccctcctagatatcttctgacaagccacatgatggcctacatctcatattttaccccttcacctcagtaatcataaaagtgataggactatcaatgtggtcccagaagctatagcagatgtgtgtacaaatggttcattccagagctcatgcgagcccctggggatgggttctgccatactcagttcagaagcagctgaatcggtcatcctgcagcctgggaagccccatccattgtaagccggagtctcataactggggagactgagtcagtctcttgagtcaacttgtttttagttccttggaactctattcataccaaacgcaggaaaaaaaaaaaaagaatcgtcttagagtcaagatgtccaaggaatattctgaccatctaaggcatttagcaaagaaaatgtcaaatgttcacaacagtgtctagcacagtcagtgacaacaggcattgccacagtttagactcacctcaacctcggggctattgatcctgctctgttgtcttgatcctcccctttgggatcacttctatactcgaggctggacatacagtgacctagagagctctggggttttctgagaactgaggggaatggatctgctgtaagggaaacgagaactgagagttggtgcctaagaaccattcacggcgccttgaaacaatcatcttgtggtagctaagcctaatgaaataaaaaaaatgacttgtaatctttcatgttttttatttaacttttccatcaatgttaatatgttgttcaaaatataaatgtttagtgcaattaaatatttaactcaggtagctagggagtatagtatactacagtacactgcagtacagaataatacagtatatattataatcaccttttcagattcttacttgcgaccctgtttgctgaatttaccttagattacagtatagctgattaccatttcatctgtgctgaatgctctgagaaagggcctttagcaacttgtcaaagttcctgaaatggcagatggcatgacaggccaagtgacaaaatgctgtgactcttaggcctgcaggcaagcaggaaacccttgaggttatgggttacccattatgggggtaagacataataaccattattctcaaatattagacacataagaagaccaatgctttgtagaatgctttacaatgtttttacattaacattctcctaagatcccaagaagtaggcgttatcatctatattttatatgaggcagtcaaggtagcttgctctgggtcacatgactttaaaccacagatcccaagtccatgcctgctgtatctcttttacatacccccacagcctagatgagcacatttgttttagagtagagcctgtcttctgtgtaaatagcacaagggactggacagtgtcttgcatattgggacttacagcatgcatttctccatgaagcaaccaaatggacttaaaagcatgagccatgagtcctgctgttttctctggcggtatgaaagtcagctgctctttcacctcaatatctcaaaataacagttattggatcactctaaagtggcttattaatatccaccaaagcctgtcatgaggtgtatacttataatcccagcacttggaaggcagaagtaagaaaatcgggagttcacagccagctttgggtaaatagaaagttccaggtcagccgagttatgtgagatcttgtctcaaacaaacaaacaaaaaacaaaaaatctaaacaaaacaagggtatctaataaaccccacaaaatacctctcagcttgtcttggcagacaggtctagaccaagagccccaggattcctgcataagcagcatgctgaaggctaatggaaaaggctgggaaaacctgaccacggggcattgtgtggacattgttgctcttcctcagggtgctctcatatttgtccttcctctgcagcaaaattggcccgagatgaccaaattcacatcctcaagcagcacagacgtaaagaactggaggcacggcaaaaacagtatcggtgagttgtggcatggaccaagtggccgtgagtagccccttctggaggaggaccatggtaggagacaagcttgccaatgtcacatggaccatattcttacctccgccttcaggcagtcagagtgaagggggtcaaagaaaagactccttggggagaatgggtccaagagagtgagtcgcctttcccattattgtatgggctgcctcaactatgtatgtcttcatgtaatattttaactataaaagtgatacataatctacaacagcatcaaaaagtcactagacgtgatcaagaaaaaagggtccaaattattcaaattaaacataaggatgtaatcctagcactactagaaagatggtggtggtgacaaagagcagccagcctggaatacagagcccagcagtagaaacaaccccaaaaggtgaaaagccattaccagctcccaaaagttgacctctgacctccacacacagtgttgtgtgtgcatgcctgaacatagatgcacacagacacccacatacataccacaaacacaatacaatatttttaagaaaatagagaatctgaataagccaatcccatgcaatataatttaattaacaattttcagatgtcctataaataaaagcttggatagcttcatgagtaaattctagcaaacattaaggaattaatatcaatcttatatatatattcccacaaataggtgaaaaggcaatattttccaacatatcgtatggtaccaatgttgtacaaaaattaggcaaagacatcattaaaaaataaagcaatacacaaatacctcacaaaaatacaactgcaattattttctacaaaatattagcaagccaagttcatatggtattaacaaataagatttttccctaggaatacagtgcttttgaacattcaaaactttgttaatctaatatatatgtaaaaaatgaaccaaagtacttatacacatatataaaatatataatatagatacacaaaaaggaatttgagaaaactcaacattctttcataatgaaacattcaaaatactggaattgaagagaatgtcctcaacacaatacagaacgtctatggaaaactcccaagacaaagtgcttttccccaagatctagaaatgataggacacccacttgtgctgcttctgttcgatatttcactgaagggtctatttggtcagctaggaaagaagaagaaaatgttttacacaatcacctttcatagatgacacagtcttttatataactaataaattcaccaaaatttaattatttatgaaaaactaataagttagcaaggttatgagatagaaactaaatcaaaaaattgatatattataacaaaagaagaaaaaaatctacttacagtaagttcaaaatcaatagaatatttattaggatgtttaacaaaagcattgtaagactagtgacaaaaccctagaacacagttgaaaagaataaagaaaatctaagtaagtcgaaaatatctcatgtccccttagctttgctaaagtggcaatccttctcctgggaatgacggatgatggagatccaatgcaatcgctgttaaaatctcagctagtgttttttaaagcaagattgataagatggtcctcaaatccatatggaaatgtatggaaaggcatacaaagttaccagaccaatcttgaaaataggaaaactaagttagagaagctatatttcccaacatccaaatttctgcataatcatagtattcggacgaaatgtagccgtatcatgagcaatgacatagacttccaagtccaggaaaaaaaatcccttatacttacagtgaattgaatgtggtgggatcagcaagacaaatctgtatgtaaagaataaccttttaaataaatggagccaaggaaactagataggcaaagtacaaacaaccatgacattgtgcacacatgtacaaagtacatgatgttggatccttacctaacaccaaattgattacctaaaaggtggaattaaaaccatataatcctgtgaaaatacaaaagtaaacatccatggctttgagttggcaaaggattctgagacagaacaccacatcacacatagcaagagaaaaacggatgacttggcatcaccaaagtttcaggcttttatgtttcaaagaaccccatgaagaaaatgaaagactgttggggaagtgtttgcctaatgagcagaaagagctgagtccaaccccagaatccacatttaaaaaaaaaaaaaacaaaaaacagaaaactgtgggagctgcagagaccacccagcaaataaaatgtctgccacacaagatggaggacctaagtttcaatctttggcatccacttaaaagcccagagctacagtgcatgtctatcaccccagcactgttggagtagagacaggtggaatatcagggctcactggtcagccagtctagctaaatcagtgaatccaggttcagtaaagagatccagtctgaagaaataaagtggagagtgactgaagacacccaacatcagcctctgtcctctacattcacacacacacacacacacacacacacacacacacacatgcacacgcacacacatacacaatcagacatagtaatgtatatgtgtgatcatagtgctgaggatgaagagacaggtagatccctgaggctctctaaccagccatcctagcctacttggtgagcacctatctagagagaaagcatatttcaaaaaccaggtggatggctcctaaaatatgacagctgagttgtcctctggcctccgcatgaatggatacacttgtgcacacaaacatacctacacacaaagagcccacagaatgagaaagaatatctgcagaacagttatgtccaaactatataaagaattcttaaaactccagaaagaaataatacaattttattttttatgaagttatttacttactttacatcccaatcacatcttcccctcccttttctcctcccagttcctctctctcatcttctctccccacccctctctcctactccccttcttctcagaaaagggcaggcctccaatggaaattaatcagctttggcatatcaagttgcaataagattaaacccaacttctcctatagaggctagacaaggcagcccagttaggggaaagggatccaaaggcaggcagtgtagtcagagacagccaatgctcccactttagtagtctcatatgaagacccagctgcgcaactattacatgtgtgcagagggcctacatctatcctatgtgtgctctctggttggcatttcagtctctatgagtccttatgggcccaggttagttgagtctatgggttttgttgtagtgtccttaactctttaggctcctacaatccttccttcccatcttctgcaggatttcccaagctccacttaatgtttgtctgtgggtctctgtatcagtgtcaatcagttgctgggtgaactccctctgatgacagttatgtaggatcctgtctaaaagtatatcagaatatcatgaatagtgttgggggggtccctctcatggcatgggtctcaagtgggccaatcattggttggctcttccttcaaattctgctcaaaaatacaattttaaaaggggtaagaactcaaagtccaaagaatggacatttctccaagaaaaacatataactgggtaaacacataaaaagtcaggtgctacattagttgtctgggaaatgctaattcaagcccagtacagcagcaagaatcaaaaagtcatattattacaaattttggtgaaattacaacactcatataccactgttgagaatgttaaatggtgcatctgctttggaaaatggcttagtcattccccaaactaacaaatagagtaattatgttgcccagccaccgcactcttgattatgtacctattaaaaaggaagtcatatgttctgatggttactgctcactgccatctaaactggagaagcacatgcaggtcagtgtgcctgcaagctccagggaggattaagtgagtgagaaagacccaacttgaatgtgagtggctccatcctacaaccctagagtcctagatctaacatagggagaaagggggaaagcaaggacctctggcactcccctttctccacttcgtggaccaccatgatgtgaatggctctattacacctcacccatcccaccagaatggacaagatctctcaatctgaacaaaacataactttctaccctgaagttgtattgaagtcacagtgacaaaaaagacacctaatacatatgtctactctaaaatttctatctgcatgattgctgttctaataaccgcactaacacaaagatgcgtacaacctaaatgtctattaatggacgaatggataaaacgtcatatatctgtgtaatttgatgtggattccaggggtcagacgccactcatcaggcttggtggtggcacctttacctgatggccatcttgtcagtcctactatatagtctttaaaagcttttgaagtttgttagtgggaggaacatataaaagtttgaatctttgggctagagaaacccaataatgcttcaaacagagcttactgcaccatcttggtggggcttcagaaggctgaggtgccaagagaagcacagagagtgaagacctgcttcatgagcttcagtgaggaatcaggactctgctggtattgggctgtaggccatcatgtggtattctggcatgagatctggcttcactctacccgtggcctaagaactcgaatgaggcttaattagtgaataatagattaatatgtttggcagcaaaaatgtgaagacacgatagcacctagcatgtgctacagctactgtctgctgctcttacccaggtgtccaatgagagagattccttgagtcttagaagaggctttggagttttgaatgagcattccaaagactctggaacttttaaagctttgagggcttttagagatgaacaaaacttattttccattataagattggcatgaagctacagagaataggatgggagtttatggcttaaatttgtttgaatgtcaaactgacaaggatgctggacttgtgatggctaatcttgtcaacttgacaggatgtagaattatctaggagacaaatctctgggtgtgtctgtgagggagatttcagatcgggtgaattaaggtggtaattcaccctctgtgtgtttccccaccaagtgaaccacagtaaaccttctactaggaagttgcttttatcaaatgctgtcacaataaagatagaaataaataatacctaaactagcatagcagccaaacatagactagtgtctttaaagattacccttatctgaacattcctcataaaatgaatgatacaattaatgacatgacatttatgactggcttgttttacttaacatgatagtttctaggctcatgcatgttgtagcagctattagtatttcattcactatctgtagaaagaaaagactcccaaaaattgtcctctgaccttcacacacatgtcataacaggtgcacagagagagagagagagagagagagaattcatatagaggaaaagaaccacatgatcagcattgccatggcgataccttacttcccaatcttagatattatctattgttctcatcactgtgacaaaatgcttgaaaacagcaactaaaatgagattgtttcaccttggactttggtttgtttgctgttttgtatttttgtacagtctgagagaagacatagaggcaaagtaggaggagctgatgacattgagcccatagtcggggaacagagagagatgaagtctggtactctacttgctttctcctttgtatttagtctaggaccccatctcatggaacggtactatttacatttggggtgggtcttcccatgtcagttaatggcactttagcaactacctcacaagcatgctggaggtttatcacccaaatgattctaggtcccatcaagctaactgtcaacattagccaccattgttagctttttcttagattgaaaagtaccaccgggcctggtggcgcatgcctttaatcccagtactcaggaggcagaggcaggcagatttctgagttcgaggccagcctggtctacaaagtgagttccaggacagccatggctatacagagaaaccctgtctcgaaaaaaccaaaccaaaccaaaacaaacaaaaaacaaaaaaacaaacaaacaaacaaaaaccagaaaagtatctgagttaaaataacttataggaaatgtttattttagatcattgttttggaagattgggtttacagccaggaggacctatggttttgccttgtgtcaaagaagcacaccatttacagtcattgtggtagaacacaaagctacttaatctcacagcagctggaagacagacaaaaaggcccatggtccaacattccttcaatgtgtgtcaagtgactgaacttctggctaccatttctcaggaagaccattagcaggagtcaagctttgcctttattatccaaagtatagatgttgcctaggggcagaggggccagtaatagcaatgatgactaaagatatgtgttcctttggggaaatcataaaaatgttcaaatattgactatggagatggttacccctatgtgataagggtaaaaaagactttaattattgactatcattgggtaaattttatggtataaaaattatatgcctataaatctgtaaaaatgtttaattaattcaaattgtaatatagacttcaacatagcaacaaaatctatacaacatatagagaacagaagagcaaatcatgataaccaggctcagcaaaacctcttaggtatgacaccaagcacacacagcacaaggacaatcgccacactggactttgtcaaaacataaatcttctatacttcactgaactccaccaaaacagtaaaatgataaaatgggccaacgtatgcagactgcatccagcaagaggcttaaattcaaaatatatagagaacactaacaaaaggtaaaaagacaggttataacaaatgctgccaatgagggatggaggtcaaaccccagcactctggtaaggtcaaaaagccacagccattccgggagacatatcagttcttcctcagaattatagatagtgtcacctcgtgacccagcagcacttcttatcacaagtgtttccaaaaataatacaaccagaattaggggtgtagctcatggcagagcacttacctagtatatagaagagccagggactcagtgcactcagtgcacacccctgtactgcaatattaacattaataactaagccatgtgtctacaccaaatacttgtatgcaaatattcgtggcaaccgtcttccttggggtttctattggtgtaataagacaccatgaccaaacgaaacttgggaagggaagggtttatttcacttcatactgccaggtaatatctaccagtgagccactgagaccaatatagttgatcatacaatgaaggcaagatgccagaatcgcatgcagctgtcccataactctggattctgttgctaagggaaacatgtggcagtggagctatgaccaataacaaagaagtgacatcacatcccaggggtggctgggcttggtgccgtgcctattgtcctagatgatttgggaggctaaacagaagaatcatttagtccccgatgttcaagaccaacctatttcaaaaagaaaattcagtatgtcaatgctaagagtaagggttgagggtactctcactgatgtgatagatagctagagagatgtttagggtctgaaagggagcagggaagaaagagagagagggagaaagagaaaaacaggcagaaagacagacaggttgagagagatagagacaaatgcatatgtctacataccctagttcacagtttaaaagttaaaaactcttgcgccatccctgcatggtgtaagagcataacctgattatactgtagtcaggccaaaaattcaaaaattcaatgtgtatgcaagcctttccgctcagcaccctatgatgtagtttggaacttctgtacttttacagggcatagatcattgtcccagagtctgccttcccttccatccggctgaggaaatcttcagctctcttctccatttcaactccctgtgacaaagccctttatagagttctacaggacttctagtctgtggagtgggttaggacaaaactccagagtgagtcaatttgttgtcagccactattatcttagtgtggtttggtgcacacctaattagacttcttacccattacacctaagagatcacaatctagcctgatctggaagccgtttacttcatcacaacctgggtcttgtgaagaggatgagaagggcactgtgaaatatcttgtccctttcttaagccctattttactccctccttccaagccgctgctctcttcagcagcccgcctcccacccatgccccatttccggtcagaaaacactgaactttgcactgtctgcacaattgtgaagttcccctttcacatacactttcaggcttccagattatggaagctgagcaagagaatacttccaggaatgctgctggcttgactcagtagcccatctgggaaagggcacagaaagggcccaggtcagagctccgtggcccctggccctcactcgctgcactgtcagtgagtatgacacagtatactgtggtctgagggctctcacatccctgacctaatacgtcagtatcttcagttttcaatcatcattatgctctgaagaccagccattgacacaactcgacagcctttcattttaattcctgcatagttaaatgcatcacacatttttggttttgtgttctgaaaactgagcgcaggacctattaaatggtaagcaagcaaacactctatccccagctccggaggcatcattcttgggtaccactttgctatattcatttggctacctgctgatgaacacatgggtacctcctagtgctttcagactgagccacagtgagctttctgctaacgtgagagaattactctggggaacatgtctagatgaagatctgtgagatgaccaagtgtgtgcgtcatctgacttctctagatcttggtaattgtttgcccagctgtccagacactttccacccttagctggtgttgctgtaagatgccattcccctgcatcaccaacccattcgacatgagacatttttgttctcgactgtctaatgggtaggattgtgtctcatttcttcgtcataagatgctacatattcatggattacacataagcactacaggctgagccaatgactcccctactggtgaacattcagctcgattccctacttctgtttgttttgaatattacagagaagcaacttgtatgcatgatatataagtcatttaatcattggcagatctgtctagagaacattaatcacttgtctcaaagcagctctcatctcacataaaatgagataatgtattgtgacacaaatatgaatgtccaggcctggctggcttgggtgttctgcatgctgaatttccacacagaagcaattgcataaacatccatacccacaggataagagagagcattaaaccaaagcattttttccagcacatcagtgggaacaagagttgcatgagctacatccaattggggaaaatctgctgcaggtgtgagagttacctacctggtggtagtcttagctttggggtgagagttacctggtggtagtctcagctttggggttagtaaggaagctgctggtgtgtttatacggtccaaagatttcacctggtatttggaaggataaaaagtcagacatagaggcagacaatgattggctattaaagagacaaaaggtgacccaagataacttttctctagatctgcacaccaactctttataataacaaagttcccataagctgatcaaccttgtaaaacctttagagcaagtgcagctttcttttctaagaacattattagttcacagttgtgtcgggcaagtgtcacattttgcgcgtgcagaaatgttgttttatgtcctctgatgctaagaaggctcattctgaggaactgataaacaggaggcggagcagccaggagagcagtctccagaagagagaagagggtaaagaagaggatgtctttcagaagacatcctttatttatagcagttttactggctatcatgatctcaatatgacctttctttctccctctgcatctgacaaaacatatacatataacataaaatggtaatattatcccaaaagttaaaacaattaaacacagagcctaaaggagaaaaaggtaatgggtgaaggaagcaggcagctagccagccaaaaagtctttctaaaaaaaaaaaatatgtataattctatttgtcaattaaatgaatgaaagcattaaaataattaaattaaaataactattttaatcttttcataatttaattaaaatatgattatatcatttctctcctctcctaactcctccccttccaatgtctacacatcccccaattcaccgcctcctcttttatctaacccttgttggcaaatatacataaaaaccagttaacatacaaatacaacctgctgagcccctgtcccctaagtgttgcctgcatgtgtatgtttctagggcttgggattagatgacaaaccaagccaggcatctctgggtgagactgactctcctctccacagctgttaactgcttgtagctcttcaactaggagtgggacccctgagatttttccccagtctgtgttggaatgacaattattttggaattatttgggttcttttttggcagccatgttgtgggcgtagcttttctgtcatagctagaagagactatcacacagcagaccttctaattttctagttcttatgatctttctgactcctcttcttcgatgctttctgagactcaggtgctggagttgtgtggtagatacatcccctgggtaccacacaaccatttcttctcttcattttgactagatgtggctttctgtaacggtctccttctgctgcaaaaagaagttcctttgatgagggttgaaagttacacttgcttgtgggtgtctggataagtattcagaattccattaagaattattctggttagagaaattggcagtagtacaatctatgacctccctagccaaaagcagttggctaagtttccagggccaagaaggatgtctttcctgttaagcaggctttgagtccaagtagacagttgttggttactgccaagatataggcaccactactgccctgtagggatatcttgccatgatggtcattgtggtttatattcaatgacagctggctaagactattgactctttccttccctgcatatgaaacacagcttatcactttctggtcctatgaaagctggtcctcgagggactcttttggttcctttccagctcaaatcctccacattttatgtgtggagtgcatggtgtcctcagcaataaggacttgcttttaacttctgggaggcaatcaaggacaagcaatagcctatattgtttcaagagctctcttggatttccctgatcaaaagctcaaagggggcttgtcatgcatggtaccgaactttttgttagctacgcttggctcttgttggagcgaatcatccctccatatgttgtaattttttatattgcatgttttctcttgtttttgagaaattgagaaatttcccacagggtattaatatattattatattggcattacatatagtgattcactgtgaggcttccatggtcataaagactgcacactattcaacattacgtatatgatgttggctgaagagagaaggtactattctgcatttgtaatttagaaggttggattctctggatgatttttttctttttttttaagcctaagtatactttaatttggtactgatatagtagttcataaagatgataccactatggtgtagaactcaaagttatgatattaagttcaggaagcgaatgtatgtaattcattgcaaatttgcaatgcttgattacacatgttcaagattcaacacggagaggcttctgatactacggtggcaccagcagggaagtcacaagttcagactgtctgagacatgcaataaggttctttcttaaagaaaacaaaatgtggaaaaacagtcttgaaatttttcacagctctttcttcctttcaattttcttgtgcagaaatttgtgtgatgataagaatcagaatgttgctctgcattatctagtaatactaactatcttgtggagtgatcctgtgcattcaccagaatgcaaatactgcttccttactaaatcactcggagccactgattcccatcagctagaaacaaacctgagcaacagagtcagtgggcagaacaccataatcctcctaagagcatggagttagtactcttcgcatacatccctttatttattatttatgtatttattttcggggaaagggatagcattttattttttaatcttcttatatctgttgaggcccgttttgtgaccaattatatggtcaattttggagaaggtaccatgaggtgctgaaaagaaggtatattcttttgttttaggatgaagtagtctataaatatctgttaagttcatttggttcataacttctgttagtttcactgtgtctctgtttggtttctgtttctatgatctgtccattgatgagagtggggtggcgaagtctcccactactattgtgtgcagtacaatgtgtactttgagttttactaacgtttcttttatgaatgtggatgcccttgcatttcgagaatagatgttcagaattgagagtttatcttggtagatttttcctttgatgtgtatgaagtgtccttccttatctttttaaataacttttggttgaaagtgaattttattcaatattagaatggctactccagcttgtttcttgggaccatttgcttggaaaattgttttccagccctttactctgaggtaatgttttagccattgaagtgcatttcctgtaggcagcaaaattctgggttctgtttatgtatccagtctgttattctatgtctttttatttgggaatctggtccatcaatgttaagatattgaggaataatgattgttacttcctgttatttttgttgttagaggtagaattatgattgtgtgactatcttctattgggtttgttgaaagaagattactttcttgctttttctatggtatagttttcctccttgttttggtgttttccatctattatcctttgtagggctggatttgcgaaaagatattgtgtaaatttggttttgtcatggaatatctcgttttctccatctatgataattgagagttttgctgggtatagtagcctgggctggcatttgtgttctcttagggtctgtatgacatgtgcccaggatcttctagctttcatagtctctggtgagaagtctggtataattttgataagtctgcctttatatgttacttgacctttttccattactgcttttaatattctttctttgtttagtgcatttggtgttttgattattatgtgatgggaggaatttcttttctggtcccctctatttggagctctgtaggcttcttgtatgttcatggggatctctctctttaggttagggaagttttcttctataattttgttgaagatatttactggccctttaagttgaaaatcttcactctcttctatacctgtcatcctttggtttggtcttctcattgtgtcctggatttcctggatgtttttggttaggagctttttgctttttgcattttctttgcgtgttgtgtcaatgttttctatggtatcttctgcacctgagattctctcttctatctcttgtattctgttggtgatgcttgcatctatgactcctgatatctttccaatgttttctaacttcagggttgtctccctttgtgatttctttattgtttctagttccatttttagatcctgggtggttttgttcattcccttcacctgtttgattgtgttttcccataattctttaagggatttttgtgtttcttctttaagggcttctacttgtttacccatgttctcctgtattttttaagggagttatttacgtccttcttaaagtcctctatcatcatcatgagaagtgatttaaggtccaaatctttcttttccagtgtgatggtgtatccaggacttgctatgttggagcagtgggttctgattatgctaaataaccttggtttctgttgcttacgttcttatgcttgcctcttgctatctgattatctctagagctacatgccctcactgtatctgactggagcctgtctttcctatgatcctggttgtgtcaaaactccttagagtttggctgtctctgtggtcctgtgattctgggatcttgtgatcctgagatcctggatgtgtcagagctcctgggagtcaagctgcctctgggaccctgaagatcctggtgtgaccaagctcctgagattctgtgatcctgtgatcctaggcatgttagagagcctgggagttgagctttctctgggtgttgtggggctggctctcatgttttctcttatatgtagatattagattttaagctttccatatgtgtgtttcatttaaaatagccacagaggttgggtagcttttatgagaccagagagaggaaggaaactccccccaaaagagacatagaatatagtgttatgggtaatcgtaagggaaactaaaatgggaggtttaaatggggagaggatgggaatgtgttacaaaggagaaattatgggagggacaactaatactaaaagccttttgactagccatatggaaactcacaactctcaaagcttcctaaaatgtataaaatggaattaccgtataatggaagagacaatgccccaactagacatcatatgctagcaagtaaaactgccagtatcaggaatgggttacatcttgttgtgtccctgatcaaaggtgacccacagacagtcccccataaacaatataggctattgtctattattagttatccttcagaacctgacagaaaatgagtgttgtgtccggccagcagaccacaacctgggttctagcctggaaaggcattttggaaacctggaagagaagaggggctaggtggcgagagaaaagaatgtagccaagacagttactctgatcaaggctcaaattttattgttgcgacactagttatgaaggaagggggaggggacccgattcccgccgaataatctctggtccagtagaaaggtgcacgtgtgtggctccgcaggttccagcagtgggcgtggcagaacgaatgagcaggaagctccacccctgagcaagcaggtttcaggctaggggaggggagactacaaatgaggacatcacttacttatgtcatcaaacatgagaaaattaacctggtgcccaacccgaagctttagccatactgacaagcactcacaatgatgaaaggtgctatatatacatgtgaccagatgagcaacagcatcatcattctttcccagctacaaaccctgagacctataactgtgtcctgccttgcaagatatactggtacaataatggcacaaacgttatggaaaaaccaaccacttcctaaaaatcacatttaaagcttattccatgagatgaaacccatatccagaaactattaatgaggccacaaacttgaaacaagatgaggcatggaccctaggggaacaactactactgctatcctgctaacggagcatgccacgcctaaagacatactgccatacccataagctaatatgtatgacaagccccatcagagaagtttcttcttggagtagatgagaattagcacagaaacacacacactcccaaatggaggatgggcagacaatgtcagttcttgaaatgctcaatcctaaatggagtgtctttgtcaaacccctttcctccagccttggggatctaggcagacgtggcagaaagagctgaaggtggtggatgactccgaaaatacagtgtctttcagacacaaaggggttgatggacattgaaactcacagagacattaacactatgtacaagactttgcaaattcaagctagataaatctcagtaccgagagaagtgggcacaagtcccaccctcaaccaggatgctatttaaaattgatacctgctcagagagggaaaatccattttctccaatggagtgacactgggtatatatcaactgtactgcagggcagggctcatgctcaagggtagttggtcaacacaacatgggctccgtagttctctttgggggagggtgtgtctctttttgtattgtttatatttttctgagagagagagagtaaaagtgtggattttgggtgggtaggaaggtatagaaaacttaggagtcgaggaagtgaaagatatggtgaaaatatgttgtatgaaaatttcaaataatttttaaaatgtaataaaaagaaaacagccttattccaaaagaaaaaaaaatccataaaccctttacagtgtgtaacactttaagcctgagaaatttattaaattttctgatttggcattcaagcatctagagagttttcaaccatagctcttctgcatttcctttggagctttactgcccacttgtgttacattgcctctggattgcagaaagtcaagagcacttcatggaccaaagatggcacttagatagttcattatggaatgaatacactttgtcctttaagagcctgatcagttctcatgctgattgtaatcttctcctatttctttgttgaagtcatactttatcaatcatgtctttatgaggtcagatatcttttttacagtaaattgcaccatctacacctccttaagctctctccttccaaaccagatgtctttgctgcctcttccttatttctgtgtagttgatccttgtggccttcctccatggagtgtgaggtttttgttcccccacccccacccccaccacctggcctatttatgtctgtgggtctggaggcctttggcttctgtgggttgacttgtctgtatgtgtcattcagtggaagggacatgccagtagcaagctagctggcaaactgatccactctcatctgcaaggctctcatctgcaaaggaggtaacggagagccccttggtgtcatatcttgagaaagttctttctgagctgacaaaacaatttctaaactctgcagaaaatttgtatggacatttggggcaggtgaaatggctcagctagtaaaagcactcgccgtgcaagccaatgacctgagtctttcctctactttcctgacttgctgcaggagagaaccaactcccaaagcagtcctcacagggaggtcctgtctgatcaagaagctgctttaggaacctgtttcttagttttaagaaactaagcagcctggagagctgcaagtgcacctgctgaaaccgtgcctgaggtactgacagcacagtgcctcgggtactgacagcacagtgcctcagggactgacagcacagtgcctcagggactgacagcacagtgcctcagggactgacagcacagtgcctcagggactgacagcacagtgcctcagggactgacagcacagtgcctcggatactgacaacacagtgcctcaggtactgacagcacagtgcctcaggtactgacagcacagtgcctcaggtactggcagcacagtgcctcaggtactggcagcacagtgcctcaggtactggcagcacagtgcctcagggactgacagcacagtgcctcggatactgacaaaggtgctttctctcttccggctgcaaacaagcccagtcttcctactaactcccaaaggtcagggacacttcgaattgtggcccacttcttatcaactcttgcctgaattctggatcaaagtacctcatatagcacctagcccttccgggaaaacacataagtaaagactcctctttgttcctcattcttccttcaacagcaatctgggattgagtccccactatggtggtcagcaatattgaaggatctgtttacctgaaacacatctcaatgttgtttttgtgttatttttgttttcttttgtttctgagatagagtctcactgtagagccttagctggcctggaagtagctctgtagagcaggctggcctgagactcacagagacccgtctggctctgcctcttgagtgctgggattaaagtgcaccacttggatggctctgcctatctatttttaaaccagaactcaaagctatgcacacaactacactcacatgtcctcacacatatcatgtacacacaagttaacaataaatataattttaaaaataaaataataaaatgtgaatgctattgttggaaactaagaaaggggggcctggggaagaggggggaaagggaaaacccacaccccaccagagttttgcctattctctggtctgtcaggcgtgggagagctgctatctaccttccactcatccctgggtgggcattcaagcctctgacccgctcttcgagggggctgcaaggggcagctctacctgggattcccggagctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaattccccacatgctatccctggtacacatgttgcctggtacactggatcaatgaaaactttagctccactcaaccctttgaaggcattgcatcctttgaactgccaagcatgaagagaagaacatttatgtcattgctgatagttgactggtgggtggtcatacagtggagtccctcaataactggcttgctcgtggatgtgtattggagacccctctattcatttttccaagtaaacaacattgtacactattcttatttttatgcttattaatgtacttgtaaaggtcacagaaacaataaaaatggaaggttttcataaagctcgctgaggagggtctagaagttagacggatctctgatagacagtgggaccacaaatagaatttttcaataaaaggcaaacatccagctttttcctcacactcttgacctgaaaaagccataaaggcctggagagtatttctcccaccagtagggcagaagggccccctgctggtggactggccaacaaatcgaaagcctgctcaggatatttaccctgagacaaggacctatcaatcagccctctactctttgttcaaactgatgaaccttaaactgggggaagcacgttcctcaaagacccagaaacccccaattatggaggagagacttgatttccgacaatgctcttggacctataatatttcaaactccacaaactcatcaaagtttcaatcagtccatcagtcccatttgtacactcagtttctttccagggatgttcatttctaagagggtccctgggctagccagacaccaaggagaagacgaggagtctgcaccagtgggagcacaaggtcatgttaaagtgctgaagacttcttgatgcccaaaacctgcatccctaaagggagctttaatggcaaggaaaatcgggaatatccatgaaaactgagctcagggtgaatatttcagtgtttaagcaaaactgatttattttctctgtcccaaatgaatgaacatgattttgttttcattagtttgttctcagagaatgttcagtaagtgttcaatgcatgaaggctgtaagaatttaaacgtgctcactggagttggtgtctgcttctctccagagggaacaactttggttgtggctacctctaaccgggctggaggccagatgttataggggcattccctcagtggtctgcacccaatgttctttctcacttcctttaatgccctctgattccaccgtgaaaactatctccttccccaaccaccacacacagaatcaggatgtgcattgaccaagctggcaggacatttgacactggctgagtgtggcataggagccttaagccctcttcctttgactgtctagacagacccctcttgggggctggggctgttcaaagtcacctgtattaagtgaggtctctaggatctccctcgctcctttctattcatgagtgcacatctgttccactcactaactctttggtataagccagtctcatagctctactgttgtgatcatgccatggcacaaatgtttgccattcagcattggaaagattcctggcacttccacttatagtgcattgagcttataatgggaaatagaccaccatatacaactttgtctgcaacatggcagcctcagctcaggggatagccaaaagggccacctctaacatagtgttttcacttttctcatgctagatggatggagtggaaccccggcttccctttgagtattgatgccaaatgccacaaggatctgccccgagatatccagtttgatagtgaaaaaggagtggactttgttctgaactactcaaaagcgtaagttttaagaacttcagggtatgagtggctcatctgatagctggggatgggagagtgttaggacttcaggaccacaaagaaagagcacaccattccacctcaagtgtctggaccaggaacagctgtactcttgatcagaagggcagccatacaccaagacagacattgtatttgcttattctaactcaggtagtgtctgtattctccccattgactgggatccaattcacggccttaatgagccaacctgaacagaattggtacacaagaaatgaaggaaggggaaaagggtcactgatccaggtcatcaaatgcctagaatacagcaatggaccttggtgtaaataaagatttcgttggggaaggggaaattaaagcatttccatagaagtccaaggttatttggggagggataaaaaatgagtttattccttgtctgaaggcaagttgtagagcatctgatagaaaagactcgtatttgttctttcttatagtaggaaaggccttttttataggacttgggctgggggagcagttcagctgaacaccattccccatgcgtttgctctacccaggccaggaggtagtctctcatcgaacagttgcaagatgacttatatctaaacagccttgacccaaaagtcttctagcacctacagccccgtgctttcagctaaaagcagaggctgttcttacttacaacactccatctccacttccagttcactcctgtggccagtccatcagaagatggagtccagactgatgccaagaggcaggttccacagtaacagtgactcagatgacactgttggtcccatgacttagattcttgttcatttgtcttaaggaccccatgaagtaggcatatgtgtttatcactgttctggtgctgtgaagagacactaagaccaaagcaattcttataaaagaaagcatttaagtgggagcttgctcacagttccagaggttaagttcattatcatcctggtggggagcacggtggcacacacgatgctggagaagtagctcagaactttacatgctgaaccacaggcagacacagagaaagacctggatcaggtgtaggcttttgaaacctcaaagcccaaccccagtttcctccaacaaggacacatctattccaacaaggcaacatctcctaatccttttaatcctttcaaataggcccattccctgatgactaagcattcaaatataggagcctatgggggccatccaagcaccataggacatattgtcctctctgcagttgaggaaccgagaaggaataagaaggttgaggaccttgcctaaagtcctttgctaataaacaacagagacactcaactctgggttacatgtctgcactagagttgctgcaggatgccagcacaaccatggatactctgtcaaagagtagactctttcagcaccagaccaaggttagcaaggacacctttgatgttatactttgactcttagagaacttttatactcatacaaacctagaaaaccctaaactcttgaagcccaactgagccttatactcccccaggaacttttagcacttcccagcacacagcacatccacagacaagtctcagagagaaggacttggctacctacacacccaacaggcagccagggctgaggaccacaaatcagtctgctccatctgggtgttttcacagctattgttggtcatttctaggtaaggaaatcaagagtccagtgggttaagataaagtaatttacatatatatattcccatacataaatagatgatttatatataaactaactctggtgacctgaatgagaaatgaccctgtaagcttgagtatatgaacacttagtacccagttggtgctggctttagaggcatgaaggatgcaagaatgaagggattgtggagtcttcccccatggttttagaaagccactaaggcaaggcactgtgtggtacggatgtccctgtgtgaagaccacaagagcaagaggctctgagaggctattgatcaagctatacaagtgtggccttggctgcagtggagacaccaagagattggaggtgtcagagctgagagatacatacataggagagctgctcacatagattggaatcagccaaagagagaaatatatgttgtagtcagcaaagttggaagggaagagccatttaagccctttgacaataggcatagggctaaaggatttggagtgtgccctgctgggtttcagtcttgctttcatccagtatttcctctctattcctgctttcctctcttgtggaacggtaatgtatattctttgtggaagtatgtagtttttttttttaaatgggggttataattaagagattgcctagagtctcagaagagactttaacacagaactgagcttgcaagactctggggacatctgcagttggactaaaagattttgaattatgatatggcacaaacctacagggaaccaggagttgagtgtggtttaagtgagaaatgtctatagaactggcgtaggtaatatttggtgctctatttgggaaagtttaggacgggcagccttgctgcagaaagaatgaactaggggcaggtttttaaagtttaaagtcttgccccatttatcatttgtgctctgcaaaggaatgaatgaacacatgcagaggtgggattcagagacattccagagggcataggcctggtctcttacacagctggggtgcagtaggaagagcgtctgccacaggataagttctatcctggtcccacaaggccagcacctacctcttggaactgcctgggctatgaggaggttcccaagtatgaagtaaaaacactctgcgatggcatagtgggtgggcaaggtgacctgtcccctggtttgcaggatggagaacctgttcatcaaccgcttcatgcacatgttccagtcttcctggcacgactttgctgactttgagaaaatcttcgtcaaaatcagcaacactatatctggtaagagggttctgtggacgggagagtttatttttctgtctgagacttgcaaacatccaacccatgcaccccctcgacatcatctcaccagaagtgtcaaggccaatgggactttttctttacatacataaatcatttgtggctcttctgctcagacccgaggcaggactgggctacatcaacctagcattggaggctgaaatgatggctgagcaattctgctcttgcaaaagactcaagtgcagtttcaaacatccacactgggtggctcacaaccaccactaacttcagctacagggcatccaaagtctctggtctctggagacacatgtgcatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttacacacacacacacacacacacacacacacacacacttaatagtagtaaaaacaaaatttacagcaatggggtccttaagaccaaagttcatttagttggagccctggcactgtcctggacagtaatagatggagagtcatgacagccatcactcttccaagaggcaggaatggttaaagatgctgacctcagaacaagggatcaacattctcccctctcttttcatgctgggcggaaggaccggaaaccttgagaagagcaagtgcctggaaatcttactgttcactaaggcatagctctaggaaaactagctctatagcctgactcagcaatgagtagggacacagcaagtagggacatggcaagtgagaagatcccgatacctacaaagatttgtcctcctgcaggatcgtgggatcctgcccagcggtcctatctagaggtcattctctccacagagcgagtcaagaaccactggcaggaagacctcatgtttggctaccagttcctgaatggctgcaacccagtactcatcaagcgctgcacagcgttgcccccgaagctcccagtgaccacagagatggtggagtgcagcctagagcggcagctcagtttagaacaggaagtacaggtaggccagcttgctgaagacctagtctttccacagtagccacttcctgctgtccaatgcttgctggtccccagacttttactggcttgtctggttgattgattgtgtgaaatcagtagtttcttcatttttctggctgcagcatccaggaggatcttgggcactgtgcaagccacatgcagtcacaggaggggctgcatctacacaatcctcacatctgcaaacacagtgtcagtgttgtgttcaatgtccatatcaataattacttgacattccctgtgataggtgtgttctacacatagtcacccaactgtcatctgctttgcaaaggtgaggaaactagagaaagaagtagactttgtgtaggtagaacttactgctaattagaaacataggcactacctgtccctcacacactttcccaagccagtctttcctgactttcacttgcaggctcaagccctcatccccagttccaaggctgatcttgtcttgagtcggatagagtttaaaccaaatcggcttgcatctgatggaatcttccaggcattaccgtgctagattttattttgcttgctgagagtcccaacaaccctcagcatactcccatctccccattgcagctagcaccactgggcctccatctccagctgttcatgtaacctccattgcctctgttcaccctgtctaggtacccagcccacatggagactccaaacacataccattcaggcctaacttcctgcctacatgactttgggggttacttaaccaactgaagttggggtcctgatccacatggaaagtccttccccactcatcaggaccttcatgggatctgacatatttctaggcatgcagacagccagcaacaacaactccttacacccaccccatgcacagaaataagcatgtcttagaactaagagggacctaaacctctgcaattgcccctagctacccagaagtaggaccttgcctgttctccaagacaagccccctaccaactccaggggaggtggcttatgggttatcttcactttgagctgacccatcaaacacatgatagtgatgatgttctgctgtggtttagatcatgaggcaataatctaacaatctaacagccactcctgattagactcagactgactaccttgtctcttatactgtcaccaactaacacaccagaacagttcacagggccctccatcattcttgacacctttctttgacagcctcagtgacatgttaaacaccaagcccacgagttgctttgtttacagtattgagaagtggccccagagcctcacatgtcttagaaagccctcttccactgagctatattaccaacccctaaatattttttaaaaatattttccagtgaaatttggttttggaacatgattggaagaacctttcaaggacccttttccccccatatatgcagagagtagccccccccccccatctcttcctttccaggagaaccctgtcctggatgacaatggctagtttgcccagtgttgaacttgatctaaatgaagtcctccagctgcatctgcatgtctgacttctcctctctcactctcagagtggtctttccgcttctttcctgagcattaagcagctttcaggcacaatgaacctgagtcagttgtggccagtctttcattttagctttgaaaataattagaaagcagaaaataaatatgtaagccagctttggagctggagaaccagacccatgcattctgaattccatttgtacccaaagctttagggtaaaagcctgggttcccgaggcccagatagactaggtttggccacccagccccacacacaaacaaaagagataggtagaaagggaaaaaacagagatttaatcaatatggacacacagggggaagagataatgaggtccagtgaccccctaaatccatcttggggcactgtcatgagttttaagtttaaatagaagaagttctggcctaggggacaagatgtggctaaataagaagtcccagtctttttgacctattttttgatactgcaagtatcaaacaatagcaagtcttcattactgattgaacatacggatctagtttccacaagattacttctgccccaggctctgtagtattgctatcctggtacaagtgtgtgtgtgtgtgtgtgtgtgtatctgtctgtctgtcctggtaggtgttacctgtttacctgtttctgggacacaagactgtgacatattttaaatttttgcagcaaatattttggttcaaataaacctctgaaccatgaaggaggaagctcagaacttctgttctatatgatacatgaatggatcactgcagaaacattcctgtgttgaatgtaaatgtaaaatgtattatgttaatatactgcaggagagaatgagggcttacctttgtacccttggccttgaagagaggaaagtaggttagacagactcttccagtgaccagcaggctgacttgcagagtctgcaggggtgtgacaaaggcaggagtagcaggctgtcgtcctgcatttagttactacgtggtcacatgaagaatcgtccactcttactaaaacagtttctagtgtttgttacacaccaagttacagggaaggtgcgtgcatgtgggtgtggtgcacatgtcctgaagaccgtcagtcacatcccacccctggcctggctcccgggtccgtttctgcttcaagtagcttgaatatatttggctgcctcctgcaggaagggaacattttcatcgttgattacgagctactggatggcatcgatgctaacaaaactgacccctgtacacaccagttcctggctgcccccatctgcctgctatataagaacctagccaacaagattgttcccattgccatccaggtaggtggctgctcagccccagcccttctgtacgacttgtacctctagggctatgtgtgtgtgtgtgtgtggtgggggttctggggcagcaccatccactccactgagaggcaccagaagctctcactccagctttccctggggtgggggagaaaaatgagctactgggggtcgctgctgccatgagaaggctgtccacaccacggcagacagtgcctttcccaggtacctttgagacattccctactgaggctaagcactggggagggcttgtcctgcagtgacctgcttttcctcctaccacctcctgacgactgtttccaaattgacatgcccacacaagccagatgggtgaagctaggggtgaagttgcaaacagaagccactgagctgtgtcggagacctcaccagtcctggaacattgtcttccatacaagtagattcttgcagccccgaggacagtggagggccacgggagcagtctcatggggatggaaagtctgtgacactgggtaatgggaataatatcagtgatatgctggtgtctacacagtctaaaagcctttgcctgggaaagcttagaaatcatgaaaactgctgtatgttaagtgcctaagaacagacactatgcttgagcttggccaaaagaccaagaagcggtaaaactgtgacacattttaaattttccagcaactgtattagtttcaaatagacctctgaaccagaggagaagtagtttagagcttttgttctgttcaatatgattcccgaatgggtcactgcagaaacatctctgtgttgactgtagatataaaataccttctgttgtctcagacctggaggagggacttggcacataaagaagcgtgtatatcgctggcatcctaaatattctggatcctggagcctatgatcattgggtctcacacagcatctgtctccatttgtgtaactgtcaccttactgctctcaaccatcttacccatgcaaggagactaaggtacaggtgctcatagttagccagccacggagctgaaattcaagccagctgtctcaagcctgaagtttaacttatgtttataatcccaggctgtcggttcaaggacagcctgggctacagagtggtgcccagtgtcaatttgtttaactaattaatcaaatcagttagctgcatatctatacccccacccctctgcactccaggagtcctgagcctattatcagagaaagctctatgtgtaagaagctgaagcatcaaatgaaggcacaggggatgtgggaaatcaccactgaaagatgccaggagagggatggattaccactgacaggtgttctgaaatagcatgtcccagacatgtatttattagagccagaggcctggctcaaatctcagctctttccattgttagcaggaattaggcattggacaagccacaggacatggttaaccctttctctatagctgcccaggagaattaatgcaccccagggcagtggtagctgcagttactgggacaacccttgaacagggcccacagctgctgtaacagatggattctaggctgggctgtaagaaggcccatgggtctccgtgttgaggctggcaaggccaggacaattgaaagcagtgctgagaggttctctgccaagactgaactgaaccagcctttttgaggatcaacagagcaacatgctagaggatggagatcatggttctggcttgggtgtgaggctgcagacagacagaacataggagaggttgaggggatgggaccgtgtccaaagcaggaaacactacagcactgtacaccttcaaagaggactgcatctgctctcagctgacttggcccttcccagggctagtaccaaagcagaaaagcaaaacatggaatatggatgatatcagctcatttatgcaaaaatgaatgcaagaacacagcatgccacaaaccatatgctttacatgggtagacattggtgttggcttggtgagacacaggacacagggtcaccacgctgcatacagaacaggttggagatagtaaggataggtctaagttcctcataataaggttctgtttatgaaaagtaaacacaaatacacacatatgcaaagacctagggaagcagcgacctcttgtggctggacaagaggtacattctcactagttcttggactcttctgtaaatgctggagtagtttacaataataatcatatattattttctactcagaaacaaaagatggcctccttcaaaagcatttaaaataatgcaagcacgttgtcctgggcatattctagaagagctgggacaaggacaagtctggaggggtgggaggaggtgtggcatgctctgtataaaagctcaccatgggagcccctgtgtggcatggtcagacagagaggaaggaactgcagggtgatggagttacagcaggcaaagcaacagagcatccctacttagctccatccagacctctgtgtggcctggagagaggctgagtcactcagaacctcacactttatctctttgcaaaataggaaggatgaggaagaactgttggtcaggctgtggtgaggattttgtgatgacacatacgtcaaaccttacgacacagcagaaagtctctggactcagtgcctgaggatacctgagtaggacaggtggcccgtgtgctctaggaagcaggccacagggtggctgtctgtgggccacacagctatggaaacagagcgatcactgtgaggagcagtcctctgagaaccttgctcactaggaccctctggaaacggcaagcctgttgtcctctgaggacacatgtgctgacttctgacttaaacctgatcacttaaggaatctgaaattctctcctaaaggtagaggacaaatccattagcgaacaagtgggaaggatactgtgggggtcaggagtgtctccctggcctccttgctcatcctcaggcggtaggggagagctgcttccctagtagtcctgccacctcccccacagcacagtggcttaggaagcttaggacgcagtactccttgctctggtgttctcgagacgcacttaatccaaaccaagacccggagtaaattcttagtgtccataacccaaagacttagaagaagagtaaaagaatgaaacctgccaggactatttcagaatggttttgtgttgtagaaaagtgggtgaggcaaggagctaaaggggtctgcaaccctataggtggaacaacaatatgaactaaccagtacccccggagctcgtgtctctagctgcatatgtagcagaagacggcctagtcggccatcattgggaagagaggccccttggtcttgcaaactttatatgcctcagtacaggggaatgccagggccaagaagtgggagtgggggggggagtggggggagggtctgggggacttttgggatagcatttgaaatgtaaatgaagaaaatacctaattaaaaaataaataaataaaattctcaaagattaaaaaagaaagaaagaaagaaaagtaaagaaaagaaaagtgggtgaggtaaatattttaccctggggttacgtatttcttttttcttttaaaaaatatggctgactaaaaaaaaaaaaatgtaatttaaaaaaaggtggcctcctagaaatttgaatttgtgctgcttgcattatatttctattggctagtgctactttggatggagagacaaatcagaaaggtgcttataacctctctaatgaaaacaacttaaggaagggagagttattttggtgctcagtttaagggaatacagtccatcgcagagggtgaggcatggctgtagaaataggaagtggttggttccattcataatcaggaagcaaatagacaggaagtggggccagtatgtggaacctccaagcccaagtctcctgctcctaaaagttccaaatcttccaaaacagcactgccagctagcgatcaagtgttcaaacacacgagcctaccgggacatcacatttaaaccgcaaaaccatcttccagattagttaggggggtgcagctaactctgctcaggggccttatctttctggctttaaagaaatgagtttttaaagaacagataacaaatttctttacattttagagaaaaatcacatgacccaagcaaggaaactggaaatgcaaacgcaagctgggcccctgggtacctggtttcagagttagggctcagagcagggctccagcagcaaatcctgatttgcagctggagcaaaagggaactgggcagcatgcagccccagccttggtccagaaggagcacggcactcaaggtcagggtgtctgtcagggaggaggatggagggcactgctgagcagaagtggtaagctggaggatgccaggctctggggagggcacaggtcaccgacccttgcagctgctcgctccttccccgaggtctgtaccactgtcctcaagttacagacaaggaacctacagctcacaaacaggtcaaaagacatgtcactagggccctccaccactccccacgtgctttttgtgatctgaaagtcatggccggttgatgtttgctttcttgtggaacttccccagagttctcccacagctcccaatgagaaataaagtggagcgttctctttctctaatgcaccagctcaaccaaacccctggagagagtaacccaatcttcctccctacggattcaaagtacgactggcttttggccaaaatctgggtgcgttccagtgacttccacgtccatcaaacgatcacccaccttctgcgcacgcatctggtgtctgaggtgtttggtatcgccatgtaccgccagctgcctgctgtgcatccccttttcaaggtacaaccagccagggctccacctacaaggaaagattatctaggagagtagctggcatcccagggtgtgtgcgagtggagtggtgatagctaggagtaggttgcaaagaggggcctaggacagacattcaagggccaagtcacaggagcctgttacaaccctgctgtccagaaagcagagttcaaaagggcagttaagtcattttccttctcttcccaaatgctgccagcacttgattgttgggtcaactacaggctaaaaaaaattactgctgctaagccaggtggtggtggtggcagcacttgtctttaatgccagtacttgggaggcagaagcaggcagatctctgagttcaaggctagcctggtctacagagtgagttctggtacagccagggctatatacctagaggaactctttgtctagaaaaaaaaataaaaataaaaaaaaataaaacaaaaaagaagagagctggtgctttggtctcttcgttgtaaattagttcaaagcaggacgtctgccttagagactgataaccaggatctgttcctagtgtagtaggagacaagcgccagggcccttactcctcagcataacagtggtggccacaggcctcctatagctacagggcaggaaggtctacacagagccctttcacttcctctctggggccagagtgacccccgcactgtgggctggcctggcctgggctgggagtgctgagtgagcatggagaccctcagtgagatttctcctccatccagctgctggtagcccatgtgaggttcaccattgctatcaacactaaggcccgggaacagcttatctgcgagtatggcctttttgacaaggtgagtgccctctcttcatgtggagcctggacaagctctgccttgtggctccatccctatctgaggtgtgaaaagtgtgggaaactctggtccttcaaccaacaccacagccagctctcccactctgtgttctctgtcacctttttatgtatcctgtccagtttcagtgtctgagcctcctccacacaagccactaacctctaccttacaaaacatctgttatttctcccatactaccatatccctgtcaccctgcctgtggccttcgaggtatttgaacagaactgcccaataattgcctacccatgtccttgatgttcccagttcttcaaagctgattggatgcccaaagctatgttctgcacaaaatcctgctctctccgtttgcagctatacagtgtccggtgcacgtcacctcatcgtgggagtgggagcgtgggcctacttaaagctgaacagctctcccactccacctcacaggacagactggcctctctatacccagccacccactggcctccaagtccctcacagatgaatctacagtatggatatggacagcttttggggaaagcaaccaaactcaggctctctgctctcttaccaggccaatgccaccgggggtggagggcacgtgcagatggtgcagagggctgtccaggatctgacctattcctccctgtgtttcccggaggccatcaaggcccggggcatggacagcacggaagacatccccttctacttctatcgtgatgatggactgctcgtgtgggaggctatccagtcgtgagtgtgactgggttctgtgggaaggggaaaccctaaagaagagtatgatgaggcaagcttgctcctgggttggctgctgatggagggaaggggcagagtccgacattgagaactgatgggactgggagagggacctgctggatacgcactcctgatagccccctgacccaggttcacaatggaggtggtgagcatctactatgagaacgaccaggtggtggaggaggaccaggaactgcaggacttcgtgaaggatgtttacgtgtacggcatgcggggcaaaaaggcctcaggtaggctacagggcaagtgtgcatctccaggtcatgaggaacagaaggcaggtgactctgctctcgggtacccaccagtctcagagcgtccctcggagcatccagcctccctttctctgagcatcttgctaagtgtgtgtggggagctaagatagaggcagaggtgggtgtccctcacagagatgagctcacggtcggtggtcggtggctctgtcttaggtttccccaagtccatcaagagcagggagaagctgtccgagtacctgacggtggtgatcttcacggcctctgcccagcatgcagctgtaaacttcggccaggtaggccaggctagccctttctgggggagagtcttaggtacctcacagtgggaaccacctccaattccacacccctggcccaggctcttgccctactggtcctcccatctccatgcagcctctgagcctggaaccagcagcagcaggcatgaggcacccaccaggctggggcagttggaagttctggaaaaggagggaaagggtctgaggagggaggtctggggaccctggaagaaaggcaaggtaggtaacaaaggagggggacactagtggtatggtgacatgtcaaaggtgtggcctgggaaaccaggagaggagagggccaagacaggtgcagtggggacatcagagaggtggatggtgtccacagggcggggtggagcctgctgagctccctatgaaccaaggaaaagcaggcctgtggatccgaggtgggcagccactgggcttcttgggcacccacgctgttgatgttgatgttgctctcacagtatgactggtgctcctggatccccaacgctcctccaactatgcgggccccaccacccacggccaagggtgtggtcaccatcgagcagatcgtggatactctaccagaccgtggccgatcatgttggcatctaggtgcagtgtgggccttgagccagtttcaagaaaatgaggtgagaccaggcactgttggaaacaccgtagatcactctagttttaacccattctccagcgcacggctttggggctctgactcaagctagaaacctgcagtcagaaatcctgatttctaaggtggagctattaggagttgggggtggggttactccaccccaggtcctcagcgtgggtctgagccctgggcaggatggcagtgggaggcacaggctctgctcggagtcaccagaggggtgtgtgtgtgtgtgtgtgtgtgtgtgtgtgtgtgtgtgtgtgtgtgctatcaccctgtaccccatactcagttgataaatctattccacatggtttcctgactccccacaaggagaattagagcttcctagtttcacttaggtatccttcacagctatgctaagtgcaggtcttggtggaatcagccctttgcaatgtctcagcgctgttcctatcaaagtaccttcagcttgtcccagacctgctaatgtgctggctctatgctcagtaggaccagaggagttgttcctaggagactaatttcctggtcaaaggatgtcaagtctgacttagcttctccaaactacaccccagatctcccacccccttccagccactgaacactttgcagacattggtgtaagcacattcccctgcaggatggccacctctccatgcccaggacccctgccggtctcacgccttaaaaggaagatgagaaagacaaagggcaggccagcaggaccctgtggccacagaacttcagagctgggagcttccaggttcccttgcaccttaccttgtctaaaaaaaaacacctggcaacaaggaagtaacttcctttaagatctccagcccttagcttttcatagggcaaaggaggtagcatttgcataatctaagttttaaaaaagaaacaaacttaatttgcatatttatgagatctaagtgtgatatgtgtatgcattggtgctgctcagttatggcatctgatctatcacctcaaacaagttacttccttgtgtccttgtgtaacattcagactcttctagtttagagatatactacagcattaactatttaccctaccatgctgtagaacactgcttaggcctcctttgaatatactaatttgccatttttcctcctctgtggtaacttctttgagatgaccaggcaggactgccttactgttttagatagggtctcactatgtagccctggctggcctggaactctcagagacctgctagcgtttgcctcccaaatgctgagattaaagacatgtatgtgccactgcacctgttgagacagcagtttttagtatcctgtatatggtatttgcatctgcttctttcccagttcatctaagttccaaagacaggattttgtatggccacgtagtgttccatcgagcattctcagtgggtagcaaaggtcaaaggtgataagagtaggtcctcctccttccccccccctccccccaaaggaagcctcttagagttagacaaggcctatcagtctataaggtaccctcttaaactctttccatgtctgtcgcagctgtttctaggcatgtacccagaggagcatttcattgagaagccagtgaaggaagccatgatccgattccgcaagaacctggaggccatcgtcagcgtgatcgccgagcgcaataagaacaaaaagctcccctactactacctgtcaccagacaggattcccaacagcgtagccatctaaggccttgcctccctacccagcagctctctgggaaggccagtggctttattagccagatcccagcttgcctggcaggctctgggtcgatcttcctgcagctggtgcctcttccaagctcgaagtgctgctcttgggcctaggtggtctggttgaaactgaaggctgttgtaggatggggagacatcacagagcctcagcatgtgctacttcttcagtggacacagttgaggaacctcccaggcagggcagagatgtgcagctgtgtcccccagcccagctcagtgcctcgtcactcggtagcatcagaataagtgacaactgttctggctgggtcaggggtactttattctatttatgcttcctccaattgcttgcatagagtaggtgcttagagaaagttcttggattaagagtttgttataaaataaacttcatttaaaacaggtgtcataccacatgctgaggtccagtcacccccactcccacccccaccaaaatcactgttctctttcgatcaacaataaaaaagctggtctactacctcctccaactgacaaggtctttgccccccactccataccagtggccctttctgcttttgtagacaatacaggtattctaaattaaacacacaaatctaaagatctgaatttatgctatgattcatatatgagaacacat